(19) United States
(12) Patent Application Publication
(10) Pub. No.: US 2024/0376090 A1
(43) Pub. Date: Nov. 14, 2024

(54) ISOXAZOLE DERIVATIVES AS MODULATORS OF THE 5-HT2A SEROTONIN RECEPTOR USEFUL FOR THE TREATMENT OF DISORDERED RELATED THERETO

(71) Applicant: Arena Pharmaceuticals, Inc., New York, NY (US)

(72) Inventors: Graeme Semple, San Diego, CA (US); Thuy-Anh Tran, San Diego, CA (US)

(73) Assignee: Arena Pharmaceuticals, Inc., New York, NY (US)

(21) Appl. No.: 18/249,774

(22) PCT Filed: Oct. 26, 2021
(86) PCT No.: PCT/US2021/056694

§ 371 Date: Apr. 20, 2023

Related U.S. Application Data
(60) Provisional application No. 63/133,046, filed on Dec. 31, 2020.
(60) Provisional application No. 63/106,156, filed on Oct. 27, 2020.

(51) Int. Cl. C07D 413/14; A61K 31/422; A61K 31/4245; A61K 31/427; A61K 31/438; A61K 31/4439; A61K 31/454; A61K 31/4545; A61K 31/496; A61K 31/4985; A61K 31/5377; A61K 31/553; C07D 261/08; C07D 413/12; C07D 417/14; C07D 491/08; C07D 491/107; C07D 498/04

(52) U.S. Cl. CPC C07D 413/14; A61K 31/422; A61K 31/4245; A61K 31/427; A61K 31/438; A61K 31/4439; A61K 31/454; A61K 31/4545; A61K 31/496; A61K 31/4985; A61K 31/5377; A61K 31/553; C07D 261/08; C07D 413/12; C07D 417/14; C07D 491/08; C07D 491/107; C07D 498/04

(57) ABSTRACT

Provided is a compound of Formula (I) or a pharmaceutically acceptable salt thereof, that is a modulator of 5-HT_{2A} and can be used in treating diseases and disorders associated with 5-HT_{2A} serotonin receptor expression and/or activity. Thus, also provided are methods of treating 5HT_{2A}-related diseases and disorders.

### TECHNICAL FIELD

The present disclosure relates to compounds and pharmaceutical compositions thereof that modulate the activity of the 5-HT_{2A} serotonin receptor. The compounds and pharmaceutical compositions thereof are useful in the treatment of diseases or disorders associated with the 5-HT_{2A} serotonin receptor.

### BACKGROUND

Receptors for serotonin (5-hydroxytryptamine, 5-HT) are an important class of G protein coupled receptors. Serotonin receptors are divided into seven subfamilies, referred to as 5-HT₁ through 5-HT₇, inclusive. These subfamilies are further divided into subtypes. For example, the 5-HT₂ subfamily is divided into three receptor subtypes: 5-HT_{2A}, 5-HT_{2B}, and 5-HT_{2C}. Certain modulators of 5-HT_{2A} serotonin receptor activity are useful in the treatment of platelet aggregation, coronary artery disease, myocardial infarction, transient ischemic attack, angina, stroke, atrial fibrillation, blood clot formation, or symptoms thereof.

There is a need for compounds that can be used to treat disorders related to the 5-HT_{2A} serotonin receptor, including disorders of the cardiovascular system. In particular, there is a need for compounds that possess physical and chemical stability and favorable pharmacokinetic properties.

### SUMMARY

The present disclosure relates in some embodiments to a compound of Formula (I):

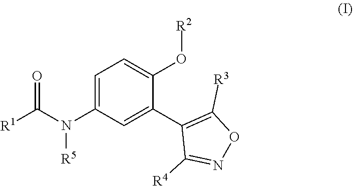

or a pharmaceutically acceptable salt thereof, wherein R¹, R², R³, R⁴, and R⁵ are as defined herein.

In some embodiments, the present disclosure relates to a pharmaceutical composition comprising a compound of Formula (I) or a pharmaceutically acceptable salt thereof, and a pharmaceutically acceptable carrier.

In some embodiments, the present disclosure relates to a method of treating a 5HT_{2A}-related disorder in an individual in need thereof, comprising prescribing and/or administering a therapeutically effective amount of a compound or pharmaceutical composition as disclosed herein.

In some embodiments, the 5HT_{2A}-related disorder is selected from the group consisting of coronary artery disease, myocardial infarction, transient ischemic attack, angina, stroke, blood clot formation, atrial fibrillation, or symptoms thereof.

In some embodiments, the 5HT_{2A}-related disorder is related to conditions associated with platelet aggregation.

In some embodiments, the 5HT_{2A}-related disorder pertains to methods for reducing the risk of blood clot formation.

In some embodiments, the 5HT_{2A}-related disorder pertains to methods for reducing the risk of blood clot formation in an angioplasty or coronary bypass surgery.

In some embodiments, the 5HT_{2A}-related disorder pertains to methods for reducing the risk of blood clot formation in an individual suffering from atrial fibrillation.

In some embodiments, the 5HT_{2A}-related disorder is related to the effects of percutaneous coronary intervention (PCI), such as microvascular obstruction (MVO), myocardial injury, reduced cardiac function (such as reduced left ventricular (LV) function), or a major adverse cardiac event (MACE) following PCI. In some embodiments, the 5HT_{2A}-related disorder is related to MVO following PCI for acute coronary syndrome (ACS). In some embodiments, the ACS is ST-elevation myocardial infarction (STEMI), non-ST-elevation myocardial infarction (NSTEMI), or unstable angina. In some embodiments, the 5HT_{2A}-related disorder is MVO or myocardial injury.

In some embodiments, the 5HT_{2A}-related disorder is Raynaud's (also referred to as Raynaud/Raynaud's syndrome, Raynaud/Raynaud's disease, and/or Raynaud/Raynaud's phenomenon). In some embodiments, the 5HT_{2A}-related disorder is secondary Raynaud's. In some embodiments, the 5HT_{2A}-related disorder is Raynaud's secondary to systemic sclerosis (SSc-RP).

Also provided in the present disclosure is a method of modulating a 5-HT_{2A} receptor in a cell, comprising contacting the receptor with a compound of Formula (I) or pharmaceutically acceptable salt thereof.

Other features and advantages of the processes, formulations, and uses provided herein will be apparent from the following detailed description and from the claims.

### DETAILED DESCRIPTION

### Definitions

For clarity and consistency, the following definitions will be used throughout this patent document.

The term “agonists” is intended to mean moieties that interact and activate the receptor, such as the 5-HT_{2A} serotonin receptor, and initiate a physiological or pharmacological response characteristic of that receptor. For example, when moieties activate the intracellular response upon binding to the receptor, or enhance GTP binding to membranes.

The term “antagonists” is intended to mean moieties that competitively bind to the receptor at the same site as agonists (for example, the endogenous ligand), but which do not activate the intracellular response initiated by the active form of the receptor, and can thereby inhibit the intracellular responses by agonists or partial agonists. Antagonists do not diminish the baseline intracellular response in the absence of an agonist or partial agonist.

The term “contact or contacting” is intended to mean bringing the indicated moieties together, whether in an in vitro system or an in vivo system. Thus, “contacting” a 5-HT_{2A} serotonin receptor with a compound of the invention includes the administration of a compound of the present invention to an individual, preferably a human, having a 5-HT_{2A} serotonin receptor, as well as, for example, introducing a compound of the invention into a sample containing a cellular or more purified preparation containing a 5-HT_{2A} serotonin receptor.

The term “inverse agonists” is intended to mean moieties that bind to the endogenous form of the receptor or to the constitutively activated form of the receptor, and which inhibit the baseline intracellular response initiated by the active form of the receptor below the normal base level of activity which is observed in the absence of agonists or partial agonists, or decrease GTP binding to membranes. Preferably, the baseline intracellular response is inhibited in the presence of the inverse agonist by at least 30%, more preferably by at least 50%, and most preferably by at least 75%, as compared with the baseline response in the absence of the inverse agonist.

The term “modulate or modulating” is intended to mean an increase or decrease in the amount, quality, response or effect of a particular activity, function, or molecule.

As used herein, “administering” means to provide a compound or other therapy, remedy, or treatment such that an individual internalizes a compound.

The term “prescribing” refers to order, authorize, or recommend the use of a drug or other therapy, remedy, or treatment. In some embodiments, a health care provider orally advises, recommends, or authorizes the use of a compound, dosage regimen, or other treatment to an individual. The health care provider may or may not provide a written prescription for the compound, dosage regimen, or treatment. Further, the health care provider may or may not provide the compound or treatment to the individual. For example, the health care provider can advise the individual where to obtain the compound without providing the compound. In some embodiments, a health care provider can provide a written prescription for the compound, dosage regimen, or treatment to the individual. A prescription can be written on paper or recorded on electronic media. In addition, a prescription can be called in (oral) or faxed in (written) to a pharmacy or a dispensary. In some embodiments, a sample of the compound or treatment is given to the individual. As used herein, giving a sample of a compound constitutes an implicit prescription for the compound. Different health care systems around the world use different methods for prescribing and administering compounds or treatments, and these methods are encompassed by the disclosure herein.

A health care provider can include, for example, a physician, nurse, nurse practitioner, or other health care professional who can prescribe or administer compounds (drugs) for the disorders disclosed herein. In addition, a health care provider can include anyone who can recommend, prescribe, administer, or prevent an individual from receiving a compound or drug, including, for example, an insurance provider.

The terms “in need of treatment” and “in need thereof” when referring to treatment, are used interchangeably to mean a judgment made by a caregiver (e.g., physician, nurse, or nurse practitioner in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by the compounds of the disclosure. Accordingly, the compounds of the disclosure can be used in a protective or preventive manner; or compounds of the disclosure can be used to alleviate, inhibit, or ameliorate the disease, condition, or disorder.

The term “individual” refers to any animal, including mammals such as mice, rats, and other rodents, rabbits, dogs, cats, swine, cattle, sheep, horses, primates, and humans. In some embodiments, “individual” refers to humans.

The term “composition” refers to a compound or crystalline form thereof, including but not limited to, salts, solvates, and hydrates of a compound of the present invention, in combination with at least one additional component, such as, a composition obtained/prepared during synthesis, pre-formulation, in-process testing (i.e., TLC, HPLC, NMR samples), and the like.

The term “hydrate” as used herein means a compound of the invention or a salt thereof that further includes a stoichiometric or non-stoichiometric amount of water bound by non-covalent intermolecular forces.

The term “pharmaceutical composition” refers to a specific composition comprising at least one active ingredient, including but not limited to, salts, solvates, and hydrates of compounds of the present disclosure, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The phrase “pharmaceutically acceptable salts, solvates, and hydrates” when referring to a compound/compounds as described herein embraces pharmaceutically acceptable solvates and/or hydrates of the compound/compounds, pharmaceutically acceptable salts of the compound/compounds, as well as pharmaceutically acceptable solvates and/or hydrates of pharmaceutically acceptable salts of the compound/compounds. It is also understood that when the phrase “pharmaceutically acceptable solvates and hydrates” or the phrase “pharmaceutically acceptable solvate or hydrate” is used when referring to a compound/compounds as described herein that are salts, it embraces pharmaceutically acceptable solvates and/or hydrates of such salts. It is also understood by a person of ordinary skill in the art that hydrates are a subgenus of solvates.

The term “compound,” as used herein, is meant to include all stereoisomers, geometric isomers, tautomers, and isotopes of the structures depicted, unless otherwise specified. All compounds, and pharmaceutically acceptable salts thereof, can be found together with other substances such as water and solvents (e.g., in the form of hydrates and solvates) or can be isolated. For example, the term “solvate,” as used herein, means a compound or a salt thereof that further includes a stoichiometric or non-stoichiometric amount of a solvent bound by non-covalent intermolecular forces. Exemplary solvents are volatile, non-toxic, and/or acceptable for administration to humans in trace amounts. The term “hydrate” as used herein means a compound or a salt thereof that further includes a stoichiometric or non-stoichiometric amount of water bound by non-covalent intermolecular forces. The term “compound” is also meant to be agnostic as to how the compound is formed, be it synthetically or biologically. For example, a compound of the present disclosure can be produced in the body through metabolism.

The terms “prevent,” “preventing,” and “prevention” refer to the elimination or reduction of the occurrence or onset of one or more symptoms associated with a particular disorder. For example, the terms “prevent,” “preventing,” and “prevention” can refer to the administration of therapy on a prophylactic or preventative basis to an individual who may ultimately manifest at least one symptom of a disorder but who has not yet done so. Such individuals can be identified on the basis of risk factors that are known to correlate with the subsequent occurrence of the disease, such as the presence of a biomarker. Alternatively, prevention therapy can be administered as a prophylactic measure without prior identification of a risk factor. Delaying the onset of the at least one episode and/or symptom of a disorder can also be considered prevention or prophylaxis.

The terms “treat,” “treating,” and “treatment” refer to the administration of therapy to an individual who already manifests, or who has previously manifested, at least one symptom of a disease, disorder, condition, dependence, or behavior. For example, “treating” can include any of the following with respect to a disease, disorder, condition, dependence, or behavior: alleviating, abating, ameliorating, improving, inhibiting (e.g., arresting the development), relieving, or causing regression. “Treating” can also include treating the symptoms, preventing additional symptoms, preventing the underlying physiological causes of the symptoms, or stopping the symptoms (either prophylactically and/or therapeutically) of a disease, disorder, condition, dependence, or behavior. For example, the term “treating” in reference to a disorder means a reduction in severity of one or more symptoms associated with a particular disorder. Therefore, treating a disorder does not necessarily mean a reduction in severity of all symptoms associated with a disorder and does not necessarily mean a complete reduction in the severity of one or more symptoms associated with a disorder.

The term “therapeutically effective amount” refers to the amount of active compound or pharmaceutical agent that elicits the biological or medicinal response in a tissue, system, animal, or human that is being sought by an individual, researcher, veterinarian, medical doctor, or other clinician or caregiver, which can include one or more of the following:
(1) preventing the disorder, for example, preventing a disease, condition, or disorder in an individual who may be predisposed to the disease, condition, or disorder but does not yet experience or display the relevant pathology or symptomatology;
(2) inhibiting the disorder, for example, inhibiting a disease, condition, or disorder in an individual who is experiencing or displaying the relevant pathology or symptomatology (i.e., arresting further development of the pathology and/or symptomatology); and
(3) ameliorating the disorder, for example, ameliorating a disease, condition, or disorder in an individual who is experiencing or displaying the relevant pathology or symptomatology (i.e., reversing the pathology and/or symptomatology).

In some embodiments, the term “therapeutically effective amount” refers to the amount of active compound or pharmaceutical agent that elicits the biological or medicinal response in a tissue, system, animal, or human that is being sought by an individual, researcher, veterinarian, medical doctor, or other clinician or caregiver, which includes preventing the disorder, for example, preventing a disease, condition, or disorder in an individual who may be predisposed to the disease, condition, or disorder but does not yet experience or display the relevant pathology or symptomatology.

In some embodiments, the term “therapeutically effective amount” refers to the amount of active compound or pharmaceutical agent that elicits the biological or medicinal response in a tissue, system, animal, or human that is being sought by an individual, researcher, veterinarian, medical doctor, or other clinician or caregiver, which includes inhibiting the disorder, for example, inhibiting a disease, condition, or disorder in an individual who is experiencing or displaying the relevant pathology or symptomatology (i.e., arresting further development of the pathology and/or symptomatology).

In some embodiments, the term “therapeutically effective amount” refers to the amount of active compound or pharmaceutical agent that elicits the biological or medicinal response in a tissue, system, animal, or human that is being sought by an individual, researcher, veterinarian, medical doctor, or other clinician or caregiver, which includes ameliorating the disorder, for example, ameliorating a disease, condition, or disorder in an individual who is experiencing or displaying the relevant pathology or symptomatology (i.e., reversing the pathology and/or symptomatology).

As used herein, “alkyl” means a branched, or straight chain chemical group containing only carbon and hydrogen, such as methyl, ethyl, n-propyl, iso-propyl, n-butyl, iso-butyl, sec-butyl, tert-butyl, n-pentyl, iso-pentyl, sec-pentyl and neo-pentyl. Alkyl groups can either be unsubstituted or substituted with one or more substituents. In some embodiments, alkyl groups include 1 to 9 carbon atoms (for example, 1 to 6 carbon atoms, 1 to 4 carbon atoms, or 1 to 2 carbon atoms).

As used herein, “alkylene” means a bivalent branched, or straight chain chemical group containing only carbon and hydrogen, such as methylene, ethylene, n-propylene, iso-propylene, n-butylene, iso-butylene, sec-butylene, tert-butylene, n-pentylene, iso-pentylene, sec-pentylene and neo-pentylene. Alkylene groups can either be unsubstituted or substituted with one or more substituents. In some embodiments, alkylene groups include 1 to 9 carbon atoms (for example, 1 to 6 carbon atoms, 1 to 4 carbon atoms, or 1 to 2 carbon atoms).

As used herein, “alkoxy” means an alkyl-O— group in which the alkyl group is as described herein. Exemplary alkoxy groups include methoxy, ethoxy, n-propoxy, i-propoxy, n-butoxy, s-butoxy, t-butoxy, pentoxy, hexoxy and heptoxy, and also the linear or branched positional isomers thereof.

As used herein, “cycloalkyl” means a non-aromatic cyclic ring system containing only carbon atoms in the ring system backbone, such as cyclopropyl, cyclobutyl, cyclopentyl, cyclohexyl, and cyclohexenyl. Cycloalkyl may include multiple fused rings. Cycloalkyl may have any degree of saturation provided that none of the rings in the ring system are aromatic. Cycloalkyl groups can either be unsubstituted or substituted with one or more substituents. In some embodiments, cycloalkyl groups include 3 to 10 carbon atoms, for example, 3 to 6 carbon atoms.

As used herein, “aryl” means a mono-, bi-, tri- or polycyclic group with only carbon atoms present in the ring backbone having 5 to 14 ring atoms, alternatively 5, 6, 9, or 10 ring atoms; and having 6, 10, or 14 pi electrons shared in a cyclic array; wherein at least one ring in the system is aromatic. Aryl groups can either be unsubstituted or substituted with one or more substituents. Examples of aryl include phenyl, naphthyl, tetrahydronaphthyl, and 2,3-dihydro-1H-indenyl. In some embodiments, the aryl is phenyl.

As used herein, “halo,” “halide,” or “halogen” refers to a chloro, bromo, fluoro, or iodo atom radical. In some embodiments, a halo is a chloro, bromo or fluoro. For example, a halide can be fluoro.

As used herein, “haloalkyl” means a hydrocarbon substituent, which is a linear or branched alkyl substituted with one or more chloro, bromo, fluoro, and/or iodo atom(s). In some embodiments, a haloalkyl is a fluoroalkyl, wherein one or more of the hydrogen atoms have been substituted by fluoro. In some embodiments, haloalkyls are of 1 to 3 carbons in length (e.g., 1 to 2 carbons in length or 1 carbon in length).

As used herein, “haloalkylene” means a bivalent branched, or straight chain alkylene substituted with one or more chloro, bromo, fluoro, and/or iodo atom(s), such as chloromethylene, dichloromethylene, 1,1-dichloroethylene, and 1,2-dichloroehtylene. Alkylene groups can either be unsubstituted or substituted with one or more substituents. In some embodiments, alkylene groups include 1 to 9 carbon atoms (for example, 1 to 6 carbon atoms, 1 to 4 carbon atoms, or 1 to 2 carbon atoms).

As used herein, “oxo” means ═O, wherein the double bond is to a carbon atom.

As used herein, the term “heteroaryl” means a mono- or bicyclic group having 5 to 10 ring atoms, such as 5, 6, 8, 9, or 10 ring atoms, such as 5, 6, 9, or 10 ring atoms; wherein at least one ring in the system is aromatic, and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrrolo[2,3-6]pyridinyl, quinazolinyl, quinolinyl, thieno[2,3-c]pyridinyl, pyrazolo[3,4-6]pyridinyl, pyrazolo[3,4-c]pyridinyl, pyrazolo[4,3-c]pyridine, pyrazolo[4,3-6]pyridinyl, tetrazolyl, chromane, 2,3-dihydrobenzofuran, tetrahydroquinoline, and isoindoline. In some embodiments, the heteroaryl is selected from thienyl, pyridinyl, furyl, pyrazolyl, imidazolyl, isoindolinyl, pyranyl, pyrazinyl, and pyrimidinyl.

As used herein, “heterocyclyl” means a 3-14 membered, such as 3-11 membered, such as 3-8 membered nonaromatic mono-, bi- or tricyclic group comprising at least one heteroatom in the ring system backbone. Bicyclic and tricyclic heterocyclyl groups may include fused ring systems, spirocyclic ring systems, and bridged ring systems and may include multiple fused rings. In some embodiments, heterocyclyls have one to four heteroatom(s) independently selected from N, O, and S. In some embodiments, heterocyclyls have one to three heteroatom(s) independently selected from N, O, and S. In some embodiments, heterocyclyls have one to two heteroatom(s) independently selected from N, O, and S. In some embodiments, monocyclic heterocyclyls are 3-membered rings. In some embodiments, monocyclic heterocyclyls are 4-membered rings. In some embodiments, monocyclic heterocyclyls are 5-membered rings. In some embodiments, monocyclic heterocyclyls are 6-membered rings. In some embodiments, monocyclic heterocyclyls are 7-membered rings. As used herein, “monocyclic heterocyclyl” means a single nonaromatic cyclic ring comprising at least one heteroatom in the ring system backbone. Examples of heterocyclyls include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, and thiomorpholinyl. In some embodiments, the heterocyclyl is selected from azetidinyl, morpholinyl, piperazinyl, pyrrolidinyl, and tetrahydropyridinyl. As used herein, “bicyclic heterocyclyl” means a nonaromatic bicyclic ring system comprising at least one heteroatom in the ring system backbone. Examples of bicyclic heterocyclyls include 2-azabicyclo[1.1.0] butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, 2-oxa-5-azabicyclo[2.2.1]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, and 2-azabicyclo[2.2.2]octane. As used herein, “spirocyclic heterocyclyl” means a nonaromatic bicyclic ring system comprising at least one heteroatom in the ring system backbone and with the rings connected through just one atom. Examples of spirocyclic heterocyclyls include 2-azaspiro[2.2]pentane, 2-oxa-6-azaspiro[3.3]heptane, 4-azaspiro[2.5]octane, 1-azaspiro[3.5]nonane, 2-azaspiro[3.5]nonane, azaspiro[4.4]nonane, 6-azaspiro[2.6]nonane, 7-azaspiro[3.5]nonane, 2-1,7-diazaspiro[3.5]nonane, 2,7-diazaspiro[3.5]nonane, 1,7-diazaspiro[4.5]decane, 2,5-diazaspiro[3.6]decane, 1-oxa-8-azaspiro[4.5]decane, 2-oxa-8-azaspiro[4.5]decane.

### Compound of Formula (I)

Provided herein is a compound of Formula (I)

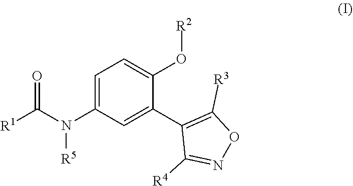

and pharmaceutically acceptable salts thereof, wherein:
R¹ is selected from C₁-C₆ alkyl, C₃-C₆ cycloalkyl, phenyl, 5-10 membered heteroaryl, 5-9 membered heterocycloalkyl, (C₁-C₃ alkylene)-(C₃-C₆ cycloalkyl), (C₁-C₃ alkylene)-phenyl, (C₁-C₃ haloalkylene)-phenyl, (C₁-C₃ alkylene)-(5-10 membered heteroaryl), (C₁-C₃ alkylene)-(5-9 membered heterocycloalkyl), (C₁-C₃ alkylene)-O—(C₃-C₆ cycloalkyl), and (C₁-C₃ alkylene)-NH—(C₃-C₆ cycloalkyl), wherein the alkyl, alkylene, cycloalkyl, phenyl, heteroaryl, and heterocycloalkyl are each optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl;
R² is selected from 4-6 membered heterocycloalkyl, (C₁-C₃ alkylene)-(4-10 membered heterocycloalkyl), and (C₁-C₃ alkylene)-NR^{2A}R^{2B}, wherein the alkylene and heterocycloalkyl are each optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl);
R^{2A} and R^{2B} are each independently selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-S(═O)—(C₁-C₃ alkyl), (C₁-C₃ alkylene)-SO₂—(C₁-C₃ alkyl), and C(═NH)(C₁-C₃ alkyl);
or R^{2A} and R^{2B}, taken together with the nitrogen to which they are attached, form a 3-10 membered heterocycloalkyl ring optionally substituted with one or more substituents independently selected from halogen, oxo, —OH, C₁-C₃ alkyl, C₁-C₃ haloalkyl, —O—(C₁-C₃ alkyl), (C₁-C₃ alkylene)-C(O)OH, —C(O)H, —C(O)(C₁-C₃ alkyl), —C(O)(C₁-C₃ alkylene)-OH, —C(O)C(O)OH, and —SO₂(C₁-C₃ alkyl), and optionally containing one additional heteroatom selected from the group of N, O, and S;

R³ and R⁴ are each independently selected from H, C₁-C₆ alkyl, and C₁-C₆ haloalkyl; and R⁵ is selected from H and C₁-C₆ alkyl.

The group R¹

In some embodiments, R¹ is selected from C₁-C₆ alkyl, C₃-C₆ cycloalkyl, phenyl, 5-10 membered heteroaryl, 5-9 membered heterocycloalkyl, (C₁-C₃ alkylene)-(C₃-C₆ cycloalkyl), (C₁-C₃ alkylene)-phenyl, (C₁-C₃ haloalkylene)-phenyl, (C₁-C₃ alkylene)-(5-10 membered heteroaryl), (C₁-C₃ alkylene)-(5-9 membered heterocycloalkyl), (C₁-C₃ alkylene)-O—(C₃-C₆ cycloalkyl), and (C₁-C₃ alkylene)-NH—(C₃-C₆ cycloalkyl), wherein the alkyl, alkylene, cycloalkyl, phenyl, heteroaryl, and heterocycloalkyl are each optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments, R¹ is C₁-C₆ alkyl optionally substituted with one or more substituents independently selected from halogen and —O—(C₁-C₃ alkyl).

In some embodiments, R¹ is C₁-C₄ alkyl optionally substituted with one or more substituents independently selected from fluorine, methoxy, and ethoxy.

In some embodiments, R¹ is methyl optionally substituted with one or more substituents independently selected from fluorine, methoxy, and ethoxy.

In some embodiments, R¹ is ethyl optionally substituted with one or more substituents independently selected from fluorine, methoxy, and ethoxy.

In some embodiments, R¹ is C₃-C₆ cycloalkyl optionally substituted with one or more substituents independently selected from halogen, —OH, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments, R¹ is C₃-C₆ cycloalkyl optionally substituted with one or more substituents independently selected from fluorine, fluoromethyl, methoxy, ethoxy, methoxymethyl, and cyclopropyl.

In some embodiments, R¹ is cyclopropyl optionally substituted with one or more substituents independently selected from fluorine, fluoromethyl, methoxy, ethoxy, methoxymethyl, and cyclopropyl.

In some embodiments, R¹ is cyclobutyl optionally substituted with one or more substituents independently selected from fluorine and methoxy.

In some embodiments, R¹ is phenyl optionally substituted with one or more substituents independently selected from halogen, —CN, —NH₂, C₁-C₃ alkyl, (C₁-C₃ haloalkyl), and —O—(C₁-C₃ alkyl).

In some embodiments, R¹ is phenyl optionally substituted with one or more substituents independently selected from fluorine, —CN, methyl, trifluoromethyl, and methoxy.

In some embodiments, R¹ is (C₁-C₃ alkylene)-(C₃-C₆ cycloalkyl), wherein the alkylene and cycloalkyl are each optionally substituted with one or more substituents independently selected from halogen, C₁-C₃ alkyl, and —O—(C₁-C₃ alkyl).

In some embodiments, R¹ is (C₁-C₃ alkylene)-(cyclopropyl), wherein the alkylene and cyclopropyl are each optionally substituted with one or more substituents independently selected from halogen, C₁-C₃ alkyl, and —O—(C₁-C₃ alkyl).

In some embodiments, R¹ is (C₁-C₃ alkylene)-(cyclopropyl), wherein the alkylene and cyclopropyl are each optionally substituted with methoxy.

In some embodiments, R¹ is (C₁-C₃ alkylene)-phenyl, wherein the alkylene and phenyl are each optionally substituted with one or more substituents independently selected from halogen, —NH₂, C₁-C₃ alkyl, C₁-C₃ haloalkyl, and —O—(C₁-C₃ alkyl).

In some embodiments, R¹ is (C₁-C₃ alkylene)-phenyl, wherein the alkylene and phenyl are each optionally substituted with one or more substituents independently selected from fluorine, methyl, and methoxy.

In some embodiments, R¹ is (C₁-C₃ haloalkylene)-phenyl, wherein the phenyl is optionally substituted with one or more substituents independently selected from halogen, —NH₂, C₁-C₃ alkyl, C₁-C₃ haloalkyl, and —O—(C₁-C₃ alkyl).

In some embodiments, R¹ is (CH₂F)-phenyl, wherein the phenyl is optionally substituted with one or more substituents independently selected from halogen, —NH₂, C₁-C₃ alkyl, C₁-C₃ haloalkyl, and —O—(C₁-C₃ alkyl).

In some embodiments, R¹ is 5-10 membered heteroaryl optionally substituted with one or more substituents independently selected from halogen, C₁-C₃ alkyl, and —O—(C₁-C₃ alkyl).

In some embodiments, R¹ is 5-6 membered heteroaryl optionally substituted with one or more substituents independently selected from chlorine, fluorine, methyl, ethyl, isopropyl, and methoxy.

In some embodiments, R¹ is 5-6 membered heteroaryl selected from 1H-pyrrolyl, 1H-pyrazolyl, furanyl, isoxazolyl, oxazolyl, 1,2,4-oxadiazolyl, 1,2,5-oxadiazolyl, isothiazolyl, thiazolyl, 1,2,3-thiadiazolyl, 1H-1,2,4-triazolyl, pyridinyl, and pyrimidinyl, each of which is optionally substituted with one or more substituents independently selected from chlorine, fluorine, methyl, ethyl, isopropyl, and methoxy.

In some embodiments R¹ is selected from 1H-pyrrolyl, 1-methyl-1H-pyrrolyl, 3-fluoro-1-methyl-1H-pyrrolyl, 1H-pyrazolyl, 1-methyl-1H-pyrazolyl, 2-methyl-1H-pyrazolyl, 3-ethyl-1H-pyrazolyl, 3-isopropyl-1H-pyrazolyl, 1,4-dimethyl-1H-pyrazolyl, 1,5-dimethyl-1H-pyrazolyl, 4-fluoro-1-methyl-1H-pyrazolyl, 4-fluoro-2-methyl-1H-pyrazolyl, 3-ethyl-1-methyl-1H-pyrazolyl, furanyl, 2-methylfuranyl, 5-methylfuranyl, isoxazolyl, 3-methylisoxazolyl, 4-methylisoxazolyl, 5-methylisoxazolyl, oxazolyl, 2-methyloxazolyl, 4-methyloxazolyl, 5-methyloxazolyl, 2,4-dimethyloxazolyl, 2,5-dimethyloxazolyl, 1,2,4-oxadiazolyl, 3-methyl-1,2,4-oxadiazolyl, 5-methyl-1,2,4-oxadiazolyl, 3-methyl-1,2,5-oxadiazolyl, isothiazolyl, 3-methylisothiazolyl, 4-methylisothiazolyl, 5-methylisothiazolyl, thiazolyl, 2-methylthiazolyl, 4-methylthiazolyl, 5-methylthiazolyl, 2,4-dimethylthiazolyl, 2,5-dimethylthiazolyl, 4-methyl-1,2,3-thiadiazolyl, 2-isopropyl-4-methylthiazolyl, 1-methyl-1H-1,2,4-triazolyl, pyridinyl, 3-methoxypyridinyl, 4-methoxypyridinyl, 5-methoxypyridinyl, 4-chloropyridinyl, 3-fluoropyridinyl, 4-fluoropyridinyl, 5-fluoropyridinyl, pyrimidinyl, and 5-fluoropyrimidinyl.

In some embodiments, R¹ is (C₁-C₃ alkylene)-(5-10 membered heteroaryl) optionally substituted with one or more substituents independently selected from halogen, C₁-C₃ alkyl, and —O—(C₁-C₃ alkyl).

In some embodiments, R¹ is (C₁-C₃ alkylene)-(5-10 membered heteroaryl) optionally substituted with one or more substituents independently selected from fluorine, methyl, ethyl, isopropyl, and methoxy.

In some embodiments, R¹ is (C₁-C₃ alkylene)-(5-10 membered heteroaryl), wherein the 5-10 membered heteroaryl is selected from 1,2,3,4-tetrahydropyrrolo[1,2-a]pyrazine, 4,5,6,7-tetrahydropyrazolo[1,5-a]pyrazine, 5,6,7,8-tetrahydroimidazo[1,2-a]pyrazine, 5,6,7,8-tetrahydro-[1,2,4]triazolo[4,3-a]pyrazine, 1H-pyrrolyl, 1H-pyrazolyl, furanyl, isoxazolyl, oxazolyl, 1,2,4-oxadiazolyl, 1,2,5-oxadiazolyl, isothiazolyl, thiazolyl, 1,2,3-thiadiazolyl, 1H-1,2,4-triazolyl, and pyridinyl, each of which is optionally substituted with one or more substituents independently selected from fluorine, methyl, ethyl, isopropyl, and methoxy.

In some embodiments, R¹ is (C₁-C₃ alkylene)-(5-10 membered heteroaryl), wherein the 5-10 membered heteroaryl is selected from 1H-pyrrolyl, 1-methyl-1H-pyrrolyl, 1H-pyrazolyl, 1-methyl-1H-pyrazolyl, 1,5-dimethyl-1H-pyrazolyl, isoxazolyl, 3-methylisoxazolyl, oxazolyl, 2-methyloxazolyl, 4-methyloxazolyl, thiazolyl, 2-methylthiazolyl, 4-methylthiazolyl, 5-methylthiazolyl, isothiazolyl, 1,2,4-oxadiazolyl, 3-methyl-1,2,4-oxadiazolyl, 5-methyl-1,2,4-oxadiazolyl, pyridinyl, 3-fluoropyridinyl, 1,2,3,4-tetrahydropyrrolo[1,2-a]pyrazinyl, 4,5,6,7-tetrahydropyrazolo[1,5-a]pyrazinyl, 5,6,7,8-tetrahydroimidazo[1,2-a]pyrazine, and 5,6,7,8-tetrahydro-[1,2,4]triazolo[4,3-a]pyrazine.

In some embodiments, R¹ is 5-9 membered heterocycloalkyl optionally substituted with one or more substituents independently selected from halogen and C₁-C₃ alkyl.

In some embodiments, R¹ is 5-9 membered heterocycloalkyl selected from tetrahydro-2H-pyranyl, tetrahydrofuranyl, 6-oxaspiro[2.5]octanyl, and 3-oxabicyclo[3.1.0]hexanyl, each of which is optionally substituted with one or more substituents independently selected from halogen and C₁-C₃ alkyl.

In some embodiments, R¹ is 6-oxaspiro[2.5]octanyl.

In some embodiments, R¹ is (C₁-C₃ alkylene)-(5-9 membered heterocycloalkyl), wherein the heterocycloalkyl is optionally substituted with one or more substituents independently selected from halogen and C₁-C₃ alkyl.

In some embodiments, R¹ is (C₁-C₃ alkylene)-(5-6 membered heterocycloalkyl), wherein the 5-6 membered heterocycloalkyl is selected from pyrrolidinyl and tetrahydrofuranyl, each of which is optionally substituted with one or more substituents independently selected from halogen and C₁-C₃ alkyl.

In some embodiments, R¹ is (C₁-C₃ alkylene)-O—(C₃-C₆ cycloalkyl), wherein the cycloalkyl is optionally substituted with one or more substituents independently selected from halogen and C₁-C₃ alkyl.

In some embodiments, R¹ is (C₁-C₃ alkylene)-NH—(C₃-C₆ cycloalkyl), wherein the cycloalkyl is optionally substituted with one or more substituents independently selected from halogen and C₁-C₃ alkyl.

The group R²

In some embodiments, R² is selected from 4-6 membered heterocycloalkyl, (C₁-C₃ alkylene)-(4-10 membered heterocycloalkyl), and (C₁-C₃ alkylene)-NR^{2A}R^{2B}, wherein the alkylene and heterocycloalkyl are each optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl).

In some embodiments, R² is 4-6 membered heterocycloalkyl optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)(C₁-C₃ alkyl), —C(O)(C₁-C₃ alkylene)-OH, —C(O)C(O)OH, and —SO₂(C₁-C₃ alkyl).

In some embodiments, R² is selected from azetidinyl, pyrrolidinyl, and piperidinyl, each of which is optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)(C₁-C₃ alkyl), —C(O)(C₁-C₃ alkylene)-OH, —C(O)C(O)OH, and —SO₂(C₁-C₃ alkyl).

In some embodiments, R² is selected from azetidinyl, pyrrolidinyl, and piperidinyl, each of which is optionally substituted with one or more C₁-C₃ alkyl.

In some embodiments, R² is azetidinyl optionally substituted with methyl or ethyl.

In some embodiments, R² is (C₁-C₃ alkylene)-(4-10 membered heterocycloalkyl), wherein the heterocycloalkyl is optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl).

In some embodiments, R² is (C₁-C₃ alkylene)-(4-10 membered heterocycloalkyl) and the 4-10 membered heterocycloalkyl is selected from azetidinyl, pyrrolidinyl, piperidinyl, piperazinyl, morpholinyl, 3-azabicyclo[3.1.0]hexanyl, 2-oxa-5-azabicyclo[2.2.1]heptanyl, 3-oxa-6-azabicyclo[3.1.1]heptanyl, octahydropyrrolo[1,2-a]pyrazinyl, hexahydro-3H-oxazolo[3,4-a]pyrazinyl, 1,4-oxazepanyl, 2-oxa-6-azaspiro[3.3]heptanyl, 6-oxa-1-azaspiro[3.3]heptanyl, 1,7-diazaspiro[3.5]nonanyl, 2,7-diazaspiro[3.5]nonanyl, 1-oxa-8-azaspiro[4.5]decanyl, and 2-oxa-8-azaspiro[4.5]decanyl, each of which is optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl).

In some embodiments, R² is an optionally substituted (C₁-C₃ alkyl)-(4-10 membered heterocycloalkyl), where the 4-10 membered heterocycloalkyl is selected from azetidinyl, azetidinyl-3-ol, 3-fluoroazetidinyl, pyrrolidinyl, pyrrolidinyl-3-ol, 3-methoxypyrrolidinyl, 2-(pyrrolidin-3-yl)acetic acid, piperidinyl, piperidinyl-4-ol, 2-(piperidin-4-yl)acetic acid, 4-methoxypiperidinyl, piperazinyl, piperazinyl-1-carbaldehyde, 1-methylpiperazinyl-2-one, 1-(piperazin-1-yl)ethan-1-one, 1-(methylsulfonyl)piperazinyl, 2-hydroxy-1-(piperazin-1-yl)ethan-1-one, 2-oxo-2-(piperazin-1-yl)acetic acid, morpholinyl, 3-azabicyclo[3.1.0]hexanyl, 2-oxa-5-azabicyclo[2.2.1]heptanyl, 3-oxa-6-azabicyclo[3.1.1]heptanyl, octahydropyrrolo[1,2-a]pyrazinyl, hexahydropyrrolo[1,2-a]pyrazinyl-6(2H)-one, hexahydro-3H-oxazolo[3,4-a]pyrazinyl, hexahydro-3H-oxazolo[3,4-a]pyrazinyl-3-one, 1,4-oxazepanyl-7-one, 2-oxa-6-azaspiro[3.3]heptanyl, 6-oxa-1-azaspiro[3.3]heptanyl, 1,7-diazaspiro[3.5]nonanyl, 1,7-diazaspiro[3.5]nonanyl-2-one, 2,7-diazaspiro[3.5]nonanyl, 2,7-diazaspiro[3.5]nonanyl-1-one, 1-oxa-8-azaspiro[4.5]decanyl, 1-oxa-8-azaspiro[4.5]decanyl-2-one, 2-oxa-8-azaspiro[4.5]decanyl, and 2-oxa-8-azaspiro[4.5]decanyl-1-one.

In some embodiments, R² is (C₁-C₃ alkyl)-(4-10 membered heterocycloalkyl), where the 4-10 membered heterocycloalkyl is azetidinyl optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl). In some embodiments, R² is (C₁-C₃ alkyl)-(4-10 membered heterocycloalkyl), where the 4-10 membered heterocycloalkyl is azetidinyl. In some embodiments, R² is (C₁-C₃ alkyl)-(4-10 membered heterocycloalkyl), where the 4-10 membered heterocycloalkyl is azetidinyl-3-ol. In some embodiments, R² is (C₁-C₃ alkyl)-(4-10 membered heterocycloalkyl), where the 4-10 membered heterocycloalkyl is 3-fluoroazetidinyl.

In some embodiments, R² is (C₁-C₃ alkyl)-(4-10 membered heterocycloalkyl), where the 4-10 membered heterocycloalkyl is pyrrolidinyl optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl). In some embodiments, R² is (C₁-C₃ alkyl)-(4-10 membered heterocycloalkyl), where the 4-10 membered heterocycloalkyl is pyrrolidinyl. In some embodiments, R² is (C₁-C₃ alkyl)-(4-10 membered heterocycloalkyl), where the 4-10 membered heterocycloalkyl is pyrrolidinyl-3-ol. In some embodiments, R² is (C₁-C₃ alkyl)-(4-10 membered heterocycloalkyl), where the 4-10 membered heterocycloalkyl is 3-methoxypyrrolidinyl. In some embodiments, R² is (C₁-C₃ alkyl)-(4-10 membered heterocycloalkyl), where the 4-10 membered heterocycloalkyl is 2-(pyrrolidin-3-yl)acetic acid.

In some embodiments, R² is (C₁-C₃ alkyl)-(4-10 membered heterocycloalkyl), where the 4-10 membered heterocycloalkyl is piperidinyl optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl). In some embodiments, R² is (C₁-C₃ alkyl)-(4-10 membered heterocycloalkyl), where the 4-10 membered heterocycloalkyl is piperidinyl. In some embodiments, R² is (C₁-C₃ alkyl)-(4-10 membered heterocycloalkyl), where the 4-10 membered heterocycloalkyl is piperidinyl-4-ol. In some embodiments, R² is (C₁-C₃ alkyl)-(4-10 membered heterocycloalkyl), where the 4-10 membered heterocycloalkyl is 2-(piperidin-4-yl)acetic acid. In some embodiments, R² is (C₁-C₃ alkyl)-(4-10 membered heterocycloalkyl), where the 4-10 membered heterocycloalkyl is 4-methoxypiperidinyl.

In some embodiments, R² is (C₁-C₃ alkyl)-(4-10 membered heterocycloalkyl), where the 4-10 membered heterocycloalkyl is piperazinyl optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl). In some embodiments, R² is (C₁-C₃ alkyl)-(4-10 membered heterocycloalkyl), where the 4-10 membered heterocycloalkyl is piperazinyl. In some embodiments, R² is (C₁-C₃ alkyl)-(4-10 membered heterocycloalkyl), where the 4-10 membered heterocycloalkyl is piperazinyl-1-carbaldehyde. In some embodiments, R² is (C₁-C₃ alkyl)-(4-10 membered heterocycloalkyl), where the 4-10 membered heterocycloalkyl is 1-methylpiperazinyl-2-one. In some embodiments, R² is (C₁-C₃ alkyl)-(4-10 membered heterocycloalkyl), where the 4-10 membered heterocycloalkyl is 1-(piperazin-1-yl)ethan-1-one. In some embodiments, R² is (C₁-C₃ alkyl)-(4-10 membered heterocycloalkyl), where the 4-10 membered heterocycloalkyl is 1-(methylsulfonyl)piperazinyl. In some embodiments, R² is (C₁-C₃ alkyl)-(4-10 membered heterocycloalkyl), where the 4-10 membered heterocycloalkyl is 2-hydroxy-1-(piperazin-1-yl)ethan-1-one. In some embodiments, R² is (C₁-C₃ alkyl)-(4-10 membered heterocycloalkyl), where the 4-10 membered heterocycloalkyl is 2-oxo-2-(piperazin-1-yl)acetic acid.

In some embodiments, R² is (C₁-C₃ alkyl)-(4-10 membered heterocycloalkyl), where the 4-10 membered heterocycloalkyl is morpholinyl optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl). In some embodiments, R² is (C₁-C₃ alkyl)-(4-10 membered heterocycloalkyl), where the 4-10 membered heterocycloalkyl is morpholinyl.

In some embodiments, R² is (C₁-C₃ alkyl)-(4-10 membered heterocycloalkyl), where the 4-10 membered heterocycloalkyl is 3-azabicyclo[3.1.0]hexanyl.

In some embodiments, R² is (C₁-C₃ alkyl)-(4-10 membered heterocycloalkyl), where the 4-10 membered heterocycloalkyl is 2-oxa-5-azabicyclo[2.2.1]heptanyl.

In some embodiments, R² is (C₁-C₃ alkyl)-(4-10 membered heterocycloalkyl), where the 4-10 membered heterocycloalkyl is 3-oxa-6-azabicyclo[3.1.1]heptanyl.

In some embodiments, R² is (C₁-C₃ alkyl)-(4-10 membered heterocycloalkyl), where the 4-10 membered heterocycloalkyl is octahydropyrrolo[1,2-a]pyrazinyl.

In some embodiments, R² is (C₁-C₃ alkyl)-(4-10 membered heterocycloalkyl), where the 4-10 membered heterocycloalkyl is hexahydropyrrolo[1,2-a]pyrazinyl-6(2H)-one.

In some embodiments, R² is (C₁-C₃ alkyl)-(4-10 membered heterocycloalkyl), where the 4-10 membered heterocycloalkyl is hexahydro-3H-oxazolo[3,4-a]pyrazinyl.

In some embodiments, R² is (C₁-C₃ alkyl)-(4-10 membered heterocycloalkyl), where the 4-10 membered heterocycloalkyl is hexahydro-3H-oxazolo[3,4-a]pyrazinyl-3-one.

In some embodiments, R² is (C₁-C₃ alkyl)-(4-10 membered heterocycloalkyl), where the 4-10 membered heterocycloalkyl is 1,4-oxazepanyl-7-one.

In some embodiments, R² is (C₁-C₃ alkyl)-(4-10 membered heterocycloalkyl), where the 4-10 membered heterocycloalkyl is 2-oxa-6-azaspiro[3.3]heptanyl.

In some embodiments, R² is (C₁-C₃ alkyl)-(4-10 membered heterocycloalkyl), where the 4-10 membered heterocycloalkyl is 6-oxa-1-azaspiro[3.3]heptanyl.

In some embodiments, R² is (C₁-C₃ alkyl)-(4-10 membered heterocycloalkyl), where the 4-10 membered heterocycloalkyl is 1,7-diazaspiro[3.5]nonanyl.

In some embodiments, R² is (C₁-C₃ alkyl)-(4-10 membered heterocycloalkyl), where the 4-10 membered heterocycloalkyl is 1,7-diazaspiro[3.5]nonanyl-2-one.

In some embodiments, R² is (C₁-C₃ alkyl)-(4-10 membered heterocycloalkyl), where the 4-10 membered heterocycloalkyl is 2,7-diazaspiro[3.5]nonanyl.

In some embodiments, R² is (C₁-C₃ alkyl)-(4-10 membered heterocycloalkyl), where the 4-10 membered heterocycloalkyl is 2,7-diazaspiro[3.5]nonanyl-1-one.

In some embodiments, R² is (C₁-C₃ alkyl)-(4-10 membered heterocycloalkyl), where the 4-10 membered heterocycloalkyl is 1-oxa-8-azaspiro[4.5]decanyl.

In some embodiments, R² is (C₁-C₃ alkyl)-(4-10 membered heterocycloalkyl), where the 4-10 membered heterocycloalkyl is 1-oxa-8-azaspiro[4.5]decanyl-2-one.

In some embodiments, R² is (C₁-C₃ alkyl)-(4-10 membered heterocycloalkyl), where the 4-10 membered heterocycloalkyl is 2-oxa-8-azaspiro[4.5]decanyl.

In some embodiments, R² is (C₁-C₃ alkyl)-(4-10 membered heterocycloalkyl), where the 4-10 membered heterocycloalkyl is 2-oxa-8-azaspiro[4.5]decanyl-1-one.

In some embodiments, R² is (C₁-C₃ alkyl)-(4-10 membered heterocycloalkyl), where the 4-10 membered heterocycloalkyl is In some embodiments, R² is (C₁-C₃ alkylene)-NR^{2A}R^{2B}.

In some embodiments, R² is (C₁-C₃ alkyl)-(4-10 membered heterocycloalkyl), where the 4-10 membered heterocycloalkyl is In some embodiments, R² is CH₂—NR^{2A}R^{2B}.

In some embodiments, R² is (C₁-C₃ alkyl)-(4-10 membered heterocycloalkyl), where the 4-10 membered heterocycloalkyl is In some embodiments, R² is (CH₂)₂—NR^{2A}R^{2B}.

In some embodiments, R² is (C₁-C₃ alkyl)-(4-10 membered heterocycloalkyl), where the 4-10 membered heterocycloalkyl is In some embodiments, R² is (CH₂)₃—NR^{2A}R^{2B}.

The groups R^{2A} and R^{2B}

In some embodiments, R^{2A} and R^{2B} are each independently selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-S(═O)—(C₁-C₃ alkyl), (C₁-C₃ alkylene)-SO₂—(C₁-C₃ alkyl), and C(═NH)(C₁-C₃ alkyl);
or R^{2A} and R^{2B}, taken together with the nitrogen to which they are attached, form a 3-10 membered heterocycloalkyl ring optionally substituted with one or more substituents independently selected from halogen, oxo, —OH, C₁-C₃ alkyl, C₁-C₃ haloalkyl, —O—(C₁-C₃ alkyl), (C₁-C₃ alkylene)-C(O)OH, —C(O)H, —C(O)(C₁-C₃ alkyl), —C(O)(C₁-C₃ alkylene)-OH, —C(O)C(O)OH, and —SO₂(C₁-C₃ alkyl), and optionally containing one additional heteroatom selected from the group of N, O, and S.

In some embodiments, R^{2A} is selected from H and C₁-C₃ alkyl.

In some embodiments, R^{2B} is selected from C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-S(═O)—(C₁-C₃ alkyl), (C₁-C₃ alkylene)-SO₂—(C₁-C₃ alkyl), and C(═NH)(C₁-C₃ alkyl).

In some embodiments, R^{2A} is H and R^{2B} is selected from (C₁-C₃ alkylene)-O—(C₁-C₃ alkyl) and C(═NH)(C₁-C₃ alkyl).

In some embodiments, R^{2A} is Hand R^{2B} is selected from 2-methoxyethyl and ethan-1-imine.

In some embodiments, R^{2A} is C₁-C₃ alkyl and R^{2B} is selected from C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-S(═O)—(C₁-C₃ alkyl), and (C₁-C₃ alkylene)-SO₂—(C₁-C₃ alkyl).

In some embodiments, R^{2A} is C₁-C₃ alkyl and R^{2B} is selected from methyl, ethyl, propyl, cyclopropylmethyl, cyclobutyl, and cyclobutylmethyl.

In some embodiments, R^{2A} and R^{2B}, taken together with the nitrogen to which they are attached, form an aziridinyl ring optionally substituted with one or more C₁-C₃ alkyl.

In some embodiments, R^{2A} and R^{2B}, taken together with the nitrogen to which they are attached, form an azetidinyl ring optionally substituted with one or more substituents independently selected from halogen, —OH, and C₁-C₃ alkyl.

In some embodiments, R^{2A} and R^{2B}, taken together with the nitrogen to which they are attached, form an azetidinyl ring optionally substituted with fluorine.

In some embodiments, R^{2A} and R^{2B}, taken together with the nitrogen to which they are attached, form an azetidinyl ring optionally substituted with —OH.

In some embodiments, R^{2A} and R^{2B}, taken together with the nitrogen to which they are attached, form a pyrrolidinyl ring optionally substituted with one or more substituents independently selected from —OH, C₁-C₃ alkyl, —O—(C₁-C₃ alkyl), and (C₁-C₃ alkylene)-C(O)OH.

In some embodiments, R^{2A} and R^{2B}, taken together with the nitrogen to which they are attached, form a pyrrolidinyl ring optionally substituted with —OH.

In some embodiments, R^{2A} and R^{2B}, taken together with the nitrogen to which they are attached, form a pyrrolidinyl ring optionally substituted with methoxy.

In some embodiments, R^{2A} and R^{2B}, taken together with the nitrogen to which they are attached, form a pyrrolidinyl ring optionally substituted with (C₂ alkylene)-C(O)OH.

In some embodiments, R^{2A} and R^{2B}, taken together with the nitrogen to which they are attached, form a piperidinyl ring optionally substituted with one or more substituents independently selected from —OH, C₁-C₃ alkyl, O—(C₁-C₃ alkyl), and (C₁-C₃ alkylene)-C(O)OH.

In some embodiments, R^{2A} and R^{2B}, taken together with the nitrogen to which they are attached, form a piperidinyl ring optionally substituted with —OH.

In some embodiments, R^{2A} and R^{2B}, taken together with the nitrogen to which they are attached, form a piperidinyl ring optionally substituted with methoxy.

In some embodiments, R^{2A} and R^{2B}, taken together with the nitrogen to which they are attached, form a piperidinyl ring optionally substituted with (C₂ alkylene)-C(O)OH.

In some embodiments, R^{2A} and R^{2B}, taken together with the nitrogen to which they are attached, form a morpholinyl ring optionally substituted with one or more C₁-C₃ alkyl.

In some embodiments, R^{2A} and R^{2B}, taken together with the nitrogen to which they are attached, form a thiomorpholinyl ring optionally substituted with one or more C₁-C₃ alkyl.

In some embodiments, R^{2A} and R^{2B}, taken together with the nitrogen to which they are attached, form a piperazinyl ring optionally substituted with one or more substituents independently selected from oxo, C₁-C₃ alkyl, —C(O)H, —C(O)(C₁-C₃ alkyl), —C(O)(C₁-C₃ alkylene)-OH, —C(O)C(O)OH, and —SO₂(C₁-C₃ alkyl).

In some embodiments, R^{2A} and R^{2B}, taken together with the nitrogen to which they are attached, form a piperazinyl ring optionally substituted with —C(O)H.

In some embodiments, R^{2A} and R^{2B}, taken together with the nitrogen to which they are attached, form a piperazinyl ring optionally substituted with —C(O)CH₃.

In some embodiments, R^{2A} and R^{2B}, taken together with the nitrogen to which they are attached, form a piperazinyl ring optionally substituted with —C(O)CH₂—OH.

In some embodiments, R^{2A} and R^{2B}, taken together with the nitrogen to which they are attached, form a piperazinyl ring optionally substituted with —C(O)C(O)OH.

In some embodiments, R^{2A} and R^{2B}, taken together with the nitrogen to which they are attached, form a piperazinyl ring optionally substituted with —SO₂CH₃.

In some embodiments, R^{2A} and R^{2B}, taken together with the nitrogen to which they are attached, form a piperazinyl ring optionally substituted with oxo and methyl.

In some embodiments, R^{2A} and R^{2B}, taken together with the nitrogen to which they are attached, form a 3-azabicyclo[3.1.0]hexanyl ring optionally substituted with one or more C₁-C₃ alkyl.

In some embodiments, R^{2A} and R^{2B}, taken together with the nitrogen to which they are attached, form a 2-oxa-5-azabicyclo[2.2.1]heptanyl ring optionally substituted with one or more C₁-C₃ alkyl.

In some embodiments, R^{2A} and R^{2B}, taken together with the nitrogen to which they are attached, form a 1,4-oxazepanyl ring optionally substituted with one or more substituents independently selected from oxo and C₁-C₃ alkyl.

In some embodiments, R^{2A} and R^{2B}, taken together with the nitrogen to which they are attached, form a 1,4-oxazepanyl ring optionally substituted with oxo.

In some embodiments, R^{2A} and R^{2B}, taken together with the nitrogen to which they are attached, form a 2-oxa-6-azaspiro[3.3]heptanyl ring optionally substituted with one or more C₁-C₃ alkyl.

In some embodiments, R^{2A} and R^{2B}, taken together with the nitrogen to which they are attached, form a 6-oxa-1-azaspiro[3.3]heptanyl ring optionally substituted with one or more C₁-C₃ alkyl.

In some embodiments, R^{2A} and R^{2B}, taken together with the nitrogen to which they are attached, form a 1,7-diazaspiro[3.5]nonanyl ring optionally substituted with one or more substituents independently selected from oxo and C₁-C₃ alkyl.

In some embodiments, R^{2A} and R^{2B}, taken together with the nitrogen to which they are attached, form a 1,7-diazaspiro[3.5]nonanyl ring optionally substituted with oxo.

In some embodiments, R^{2A} and R^{2B}, taken together with the nitrogen to which they are attached, form a 2,7-diazaspiro[3.5]nonanyl ring optionally substituted with one or more substituents independently selected from oxo and C₁-C₃ alkyl.

In some embodiments, R^{2A} and R^{2B}, taken together with the nitrogen to which they are attached, form a 2,7-diazaspiro[3.5]nonanyl ring optionally substituted with oxo.

In some embodiments, R^{2A} and R^{2B}, taken together with the nitrogen to which they are attached, form a 1-oxa-8-azaspiro[4.5]decanyl ring optionally substituted with one or more substituents independently selected from oxo and C₁-C₃ alkyl.

In some embodiments, R^{2A} and R^{2B}, taken together with the nitrogen to which they are attached, form a 1-oxa-8-azaspiro[4.5]decanyl ring optionally substituted with oxo.

In some embodiments, R^{2A} and R^{2B}, taken together with the nitrogen to which they are attached, form a 2-oxa-8-azaspiro[4.5]decanyl ring optionally substituted with one or more substituents independently selected from oxo and C₁-C₃ alkyl.

In some embodiments, R^{2A} and R^{2B}, taken together with the nitrogen to which they are attached, form a 2-oxa-8-azaspiro[4.5]decanyl ring optionally substituted with oxo.

In some embodiments, R^{2A} and R^{2B}, taken together with the nitrogen to which they are attached, form an octahydropyrrolo[1,2-a]pyrazinyl ring optionally substituted with one or more substituents independently selected from oxo and C₁-C₃ alkyl.

In some embodiments, R^{2A} and R^{2B}, taken together with the nitrogen to which they are attached, form an octahydropyrrolo[1,2-a]pyrazinyl ring optionally substituted with OXO.

In some embodiments, R^{2A} and R^{2B}, taken together with the nitrogen to which they are attached, form a hexahydro-3H-oxazolo[3,4-a]pyrazinyl ring optionally substituted with one or more substituents independently selected from oxo and C₁-C₃ alkyl.

In some embodiments, R^{2A} and R^{2B}, taken together with the nitrogen to which they are attached, form a hexahydro-3H-oxazolo[3,4-a]pyrazinyl ring optionally substituted with oxo.

The groups R³ and R⁴

In some embodiments, R³ and R⁴ are each independently selected from H, C₁-C₆ alkyl, and C₁-C₆ haloalkyl.

In some embodiments, one of R³ and R⁴ is H and the other is C₁-C₆ alkyl.

In some embodiments, one of R³ and R⁴ is H and the other is methyl.

In some embodiments, R³ and R⁴ are each C₁-C₆ alkyl.

In some embodiments, R³ and R⁴ are each C₁-C₃ alkyl.

In some embodiments, R³ and R⁴ are each methyl.

In some embodiments, R³ and R⁴ are each H.

The group R⁵

In some embodiments, R⁵ is selected from H and C₁-C₆ alkyl.

In some embodiments, R⁵ is H.

In some embodiments, R⁵ is C₁-C₆ alkyl.

In some embodiments, R⁵ is C₁-C₃ alkyl.

In some embodiments, R⁵ is methyl.

In some embodiments, the compound of Formula (I) is a compound of Formula (Ia):

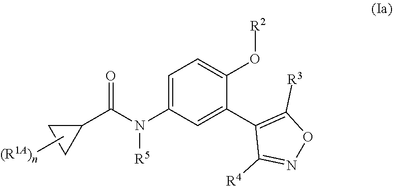

and pharmaceutically acceptable salts thereof, wherein:
each R^{1A} is independently selected from halogen, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl;
R² is selected from 4-6 membered heterocycloalkyl, (C₁-C₃ alkylene)-(4-10 membered heterocycloalkyl), and (C₁-C₃ alkylene)-NR^{2A}R^{2B}, wherein the alkylene and heterocycloalkyl are each optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl);
R^{2A} and R^{2B} are each independently selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-SO₂—(C₁-C₃ alkyl), and C(═NH)(C₁-C₃ alkyl);
or R^{2A} and R^{2B}, taken together with the nitrogen to which they are attached, form a 3-10 membered heterocycloalkyl ring optionally substituted with one or more substituents independently selected from halogen, oxo, —OH, C₁-C₃ alkyl, C₁-C₃ haloalkyl, —O—(C₁-C₃ alkyl), (C₁-C₃ alkylene)-C(O)OH, —C(O)H, —C(O)(C₁-C₃ alkyl), —C(O)(C₁-C₃ alkylene)-OH, —C(O)C(O)OH, and —SO₂(C₁-C₃ alkyl), and optionally containing one additional heteroatom selected from the group of N, O, and S;
R³ and R⁴ are each independently selected from H, C₁-C₆ alkyl, and C₁-C₆ haloalkyl;
R⁵ is selected from H and C₁-C₆ alkyl; and
n is 0, 1, 2, 3, 4, or 5.

In some embodiments of the compound of Formula (Ia):
R^{1A} is selected from H, halogen, —OH, C₁-C₃ haloalkyl, C₃-C₆ cycloalkyl, —O—(C₁-C₃ alkyl), (C₁-C₃ alkylene)-O—(C₁-C₃ alkyl), and phenyl;
R² is selected from 4-6 membered heterocycloalkyl, (C₁-C₃ alkylene)-(4-10 membered heterocycloalkyl), and (C₁-C₃ alkylene)-NR^{2A}R^{2B}, wherein the alkylene and heterocycloalkyl are each optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl);
R^{2A} and R^{2B} are each independently selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-SO₂—(C₁-C₃ alkyl), and C(═NH)(C₁-C₃ alkyl);
or R^{2A} and R^{2B}, taken together with the nitrogen to which they are attached, form a 3-10 membered heterocycloalkyl ring optionally substituted with one or more substituents independently selected from oxo, —OH, C₁-C₃ alkyl, —O—(C₁-C₃ alkyl), (C₁-C₃ alkylene)-C(O)OH, and —C(O)(C₁-C₃ alkyl), and optionally containing one additional heteroatom selected from the group of N, O, and S;
R³ and R⁴ are each independently selected from H and C₁-C₃ alkyl;
R⁵ is selected from H and C₁-C₃ alkyl; and
n is 0, 1, or 2.

In some embodiments of the compound of Formula (Ia), R^{1A} is H.

In some embodiments of the compound of Formula (Ia), R^{1A} is halogen. In some embodiments, R^{1A} is fluorine.

In some embodiments of the compound of Formula (Ia), R^{1A} is C₁-C₃ haloalkyl. In some embodiments, R^{1A} is fluoromethyl.

In some embodiments of the compound of Formula (Ia), R^{1A} is C₃-C₆ cycloalkyl. In some embodiments of the compound of Formula (Ia), R^{1A} is cyclopropyl.

In some embodiments of the compound of Formula (Ia), R^{1A} is —O—(C₁-C₃ alkyl). In some embodiments, R^{1A} is methoxy. In some embodiments, R^{1A} is ethoxy.

In some embodiments of the compound of Formula (Ia), RA is (C₁-C₃ alkylene)-O—(C₁-C₃ alkyl). In some embodiments of the compound of Formula (Ia), R^{1A} is —CH₂—O—CH₃.

In some embodiments of the compound of Formula (Ia), one of R³ and R⁴ is H and the other is C₁-C₃ alkyl.

In some embodiments of the compound of Formula (Ia), R³ and R⁴ are each C₁-C₃ alkyl.

In some embodiments of the compound of Formula (Ia), R³ and R⁴ are each methyl.

In some embodiments of the compound of Formula (Ia), R⁵ is H.

In some embodiments of the compound of Formula (Ia), R⁵ is methyl.

In some embodiments of the compound of Formula (Ia), n is 0.

In some embodiments of the compound of Formula (Ia), n is 1.

In some embodiments of the compound of Formula (Ia), n is 2.

In some embodiments, the compound of Formula (I) is a compound of Formula (Ia′):

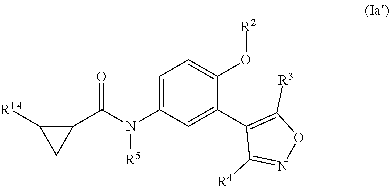

and pharmaceutically acceptable salts thereof, wherein:
R^{1A} is selected from halogen, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl;
R² is selected from 4-6 membered heterocycloalkyl, (C₁-C₃ alkylene)-(4-10 membered heterocycloalkyl), and (C₁-C₃ alkylene)-NR^{2A}R^{2B}, wherein the alkylene and heterocycloalkyl are each optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl);
R^{2A} and R^{2B} are each independently selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-SO₂—(C₁-C₃ alkyl), and C(═NH)(C₁-C₃ alkyl);
or R^{2A} and R^{2B}, taken together with the nitrogen to which they are attached, form a 3-10 membered heterocycloalkyl ring optionally substituted with one or more substituents independently selected from halogen, oxo, —OH, C₁-C₃ alkyl, C₁-C₃ haloalkyl, —O—(C₁-C₃ alkyl), (C₁-C₃ alkylene)-C(O)OH, —C(O)H, —C(O)(C₁-C₃ alkyl), —C(O)(C₁-C₃ alkylene)-OH, —C(O)C(O)OH, and —SO₂(C₁-C₃ alkyl), and optionally containing one additional heteroatom selected from the group of N, O, and S;
R³ and R⁴ are each independently selected from H, C₁-C₆ alkyl, and C₁-C₆ haloalkyl; and
R⁵ is selected from H and C₁-C₆ alkyl.

In some embodiments of the compound of Formula (Ia′):
R^{1A} is selected from H, halogen, —OH, C₁-C₃ haloalkyl, C₃-C₆ cycloalkyl, —O—(C₁-C₃ alkyl), (C₁-C₃ alkylene)-O—(C₁-C₃ alkyl), and phenyl;
R² is selected from 4-6 membered heterocycloalkyl, (C₁-C₃ alkylene)-(4-10 membered heterocycloalkyl), and (C₁-C₃ alkylene)-NR^{2A}R^{2B}, wherein the alkylene and heterocycloalkyl are each optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)(C₁-C₃ alkyl), —C(O)(C₁-C₃ alkylene)-OH, —C(O)C(O)OH, and —SO₂(C₁-C₃ alkyl);
R^{2A} and R^{2B} are each independently selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-SO₂—(C₁-C₃ alkyl), and C(═NH)(C₁-C₃ alkyl);
or R^{2A} and R^{2B}, taken together with the nitrogen to which they are attached, form a 3-10 membered heterocycloalkyl ring optionally substituted with one or more substituents independently selected from oxo, —OH, C₁-C₃ alkyl, —O—(C₁-C₃ alkyl), (C₁-C₃ alkylene)-C(O)OH, and —C(O)(C₁-C₃ alkyl), and optionally containing one additional heteroatom selected from the group of N, O, and S;
R³ and R⁴ are each independently selected from H and C₁-C₆ alkyl; and
R⁵ is selected from H and C₁-C₆ alkyl.

In some embodiments of the compound of Formula (Ia′), R^{1A} is H.

In some embodiments of the compound of Formula (Ia′), R^{1A} is halogen. In some embodiments, R^{1A} is fluorine.

In some embodiments of the compound of Formula (Ia′), R^{1A} is C₁-C₃ haloalkyl. In some embodiments, R^{1A} is fluoromethyl.

In some embodiments of the compound of Formula (Ia′), R^{1A} is C₃-C₆ cycloalkyl. In some embodiments of the compound of Formula (Ia′), R^{1A} is cyclopropyl.

In some embodiments of the compound of Formula (Ia′), R^{1A} is —O—(C₁-C₃ alkyl). In some embodiments, R^{1A} is ethoxy.

In some embodiments of the compound of Formula (Ia′), R^{1A} is (C₁-C₃ alkylene)-O—(C₁-C₃ alkyl). In some embodiments of the compound of Formula (Ia′), R^{1A} is —CH₂—O—CH₃.

In some embodiments of the compound of Formula (Ia′), one of R³ and R⁴ is H and the other is C₁-C₃ alkyl.

In some embodiments of the compound of Formula (Ia′), R³ and R⁴ are each C₁-C₃ alkyl.

In some embodiments of the compound of Formula (Ia′), R³ and R⁴ are each methyl.

In some embodiments of the compound of Formula (Ia′), R⁵ is H.

In some embodiments of the compound of Formula (Ia′), R⁵ is methyl.

In some embodiments, the compound of Formula (I) is a compound of Formula (Ia″):

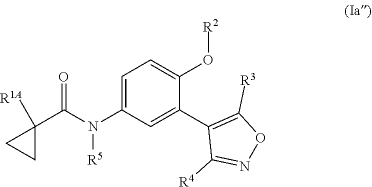

and pharmaceutically acceptable salts thereof, wherein:
R^{1A} is selected from halogen, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl;
R² is selected from 4-6 membered heterocycloalkyl, (C₁-C₃ alkylene)-(4-10 membered heterocycloalkyl), and (C₁-C₃ alkylene)-NR^{2A}R^{2B}. wherein the alkylene and heterocycloalkyl are each optionally substituted with one or more substituents independently selected from halogen, oxo, —OH, (C₁-C₃ alkyl), —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl);
R^{2A} and R^{2B} are each independently selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-SO₂—(C₁-C₃ alkyl), and C(═NH)(C₁-C₃ alkyl);
or R^{2A} and R^{2B}, taken together with the nitrogen to which they are attached, form a 3-10 membered heterocycloalkyl ring optionally substituted with one or more substituents independently selected from halogen, oxo, —OH, C₁-C₃ alkyl, C₁-C₃ haloalkyl, —O—(C₁-C₃ alkyl), (C₁-C₃ alkylene)-C(O)OH, —C(O)H, —C(O)(C₁-C₃ alkyl), —C(O)(C₁-C₃ alkylene)-OH, —C(O)C(O)OH, and —SO₂(C₁-C₃ alkyl), and optionally containing one additional heteroatom selected from the group of N, O, and S;
R³ and R⁴ are each independently selected from H, C₁-C₆ alkyl, and C₁-C₆ haloalkyl; and
R⁵ is selected from H and C₁-C₆ alkyl.

In some embodiments of the compound of Formula (Ia″):
R^{1A} is selected from H, halogen, —OH, C₁-C₃ haloalkyl, C₃-C₆ cycloalkyl, —O—(C₁-C₃ alkyl), (C₁-C₃ alkylene)-O—(C₁-C₃ alkyl), and phenyl;
R² is selected from 4-6 membered heterocycloalkyl, (C₁-C₃ alkylene)-(4-10 membered heterocycloalkyl), and (C₁-C₃ alkylene)-NR^{2A}R^{2B}, wherein the alkylene and heterocycloalkyl are each optionally substituted with one or more substituents independently selected from halogen, oxo, —OH, C₁-C₃ alkyl, —O—(C₁-C₃ alkyl), (C₁-C₃ alkyl)-C(O)OH, —C(O)H, —C(O)(C₁-C₃ alkyl), —C(O)(C₁-C₃ alkyl)-OH, —C(O)C(O)OH, and —SO₂(C₁-C₃ alkyl);
R^{2A} and R^{2B} are each independently selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-SO₂—(C₁-C₃ alkyl), and C(═NH)(C₁-C₃ alkyl);
or R^{2A} and R^{2B}, taken together with the nitrogen to which they are attached, form a 3-10 membered heterocycloalkyl ring optionally substituted with one or more substituents independently selected from oxo, —OH, C₁-C₃ alkyl, —O—(C₁-C₃ alkyl), (C₁-C₃ alkylene)-C(O)OH, and —C(O)(C₁-C₃ alkyl), and optionally containing one additional heteroatom selected from the group of N, O, and S;
R³ and R⁴ are each independently selected from H and C₁-C₆ alkyl; and
R⁵ is selected from H and C₁-C₆ alkyl.

In some embodiments of the compound of Formula (Ia″), R^{1A} is H.

In some embodiments of the compound of Formula (Ia″), R^{1A} is halogen. In some embodiments, R^{1A} is fluorine.

In some embodiments of the compound of Formula (Ia″), R^{1A} is C₁-C₃ haloalkyl. In some embodiments, R^{1A} is fluoromethyl.

In some embodiments of the compound of Formula (Ia″), R^{1A} is C₃-C₆ cycloalkyl. In some embodiments of the compound of Formula (Ia″), R^{1A} is cyclopropyl.

In some embodiments of the compound of Formula (Ia″), R^{1A} is —O—(C₁-C₃ alkyl). In some embodiments, R^{1A} is methoxy. In some embodiments, R^{1A} is ethoxy.

In some embodiments of the compound of Formula (Ia″), R^{1A} is (C₁-C₃ alkylene)-O—(C₁-C₃ alkyl). In some embodiments of the compound of Formula (Ia″), R^{1A} is —CH₂—O—CH₃.

In some embodiments of the compound of Formula (Ia″), one of R³ and R⁴ is H and the other is C₁-C₃ alkyl.

In some embodiments of the compound of Formula (Ia″), R³ and R⁴ are each C₁-C₃ alkyl.

In some embodiments of the compound of Formula (Ia″), R³ and R⁴ are each methyl.

In some embodiments of the compound of Formula (Ia″), R⁵ is H.

In some embodiments of the compound of Formula (Ia″), R⁵ is methyl.

In some embodiments of the compounds of Formulas (Ia), (Ia′), and (Ia″), R² is 4-6 membered heterocycloalkyl optionally substituted with one or more substituents independently selected from halogen and C₁-C₃ alkyl. In some embodiments, R² is selected from azetidinyl, pyrrolidinyl, and piperidinyl, each of which is optionally substituted with one or more substituents independently selected from halogen and C₁-C₃ alkyl. In some embodiments, R² is azetidinyl. In some embodiments, R² is azetidinyl substituted with methyl. In some embodiments, R² is azetidinyl substituted with ethyl. In some embodiments, R² is pyrrolidinyl. In some embodiments, R² is piperidinyl.

In some embodiments of the compounds of Formulas (Ia), (Ia′), and (Ia″), R² is (C₁-C₃ alkylene)-(4-10 membered heterocycloalkyl), wherein the alkylene and heterocycloalkyl are each optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl).

In some embodiments of the compounds of Formulas (Ia), (Ia′), and (Ia″), R² is (C₁ alkylene)-(4-6 membered heterocycloalkyl). In some embodiments, the 4-6 membered heterocycloalkyl is selected from azetidinyl, pyrrolidinyl, piperidinyl, and morpholinyl. In some embodiments, the 4-6 membered heterocycloalkyl is azetidinyl. In some embodiments, the 4-6 membered heterocycloalkyl is pyrrolidinyl. In some embodiments, the 4-6 membered heterocycloalkyl is piperidinyl. In some embodiments, the 4-6 membered heterocycloalkyl is morpholinyl.

In some embodiments of the compounds of Formulas (Ia), (la′), and (Ia″), R² is (C₂ alkylene)-(4-10 membered heterocycloalkyl), wherein the heterocycloalkyl is optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)(C₁-C₃ alkyl), —C(O)C(O)OH, and —SO₂(C₁-C₃ alkyl).

In some embodiments of the compounds of Formulas (Ia), (la′), and (Ia″), R² is (C₂ alkylene)-(4-10 membered heterocycloalkyl), wherein the heterocycloalkyl is optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)(C₁-C₃ alkyl), —C(O)C(O)OH, and —SO₂(C₁-C₃ alkyl), and wherein the heterocycloalkyl is connected to the (C₂ alkylene)- at a carbon atom of the heterocycloalkyl, wherein the carbon atom of the heterocycloalkyl has (S) stereochemistry.

In some embodiments of the compounds of Formulas (Ia), (Ia′), and (Ia″), R² is (C₂ alkylene)-(4-10 membered heterocycloalkyl), wherein the heterocycloalkyl is optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)(C₁-C₃ alkyl), —C(O)C(O)OH, and —SO₂(C₁-C₃ alkyl), and wherein the heterocycloalkyl is connected to the (C₂ alkylene)- at a carbon atom of the heterocycloalkyl, wherein the carbon atom of the heterocycloalkyl has (R) stereochemistry.

In some embodiments of the compounds of Formulas (Ia), (Ia′), and (Ia″), R² is (C₂ alkylene)-(azetidinyl), wherein the azetidinyl is optionally substituted with halogen or —OH. In some embodiments, R² is (C₂ alkylene)-(azetidinyl), wherein the azetidinyl is optionally substituted with fluorine. In some embodiments, R² is (C₂ alkylene)-(azetidinyl), wherein the azetidinyl is optionally substituted with —OH. In some embodiments, R² is (C₂ alkylene)-(azetidinyl).

In some embodiments of the compounds of Formulas (Ia), (Ia′), and (Ia″), R² is (C₂ alkylene)-(pyrrolidinyl), wherein the pyrrolidinyl is optionally substituted with —OH, —O—(C₁-C₃ alkyl) or (C₁-C₃ alkylene)-C(O)OH. In some embodiments, R² is (C₂ alkylene)-(pyrrolidinyl), wherein the pyrrolidinyl is optionally substituted with —OH. In some embodiments, R² is (C₂ alkylene)-(pyrrolidinyl), wherein the pyrrolidinyl is optionally substituted with methoxy. In some embodiments, R² is (C₂ alkylene)-(pyrrolidinyl).

In some embodiments of the compounds of Formulas (Ia), (Ia′), and (Ia″), R² is (C₂ alkylene)-(piperidinyl), wherein the piperidinyl is optionally substituted with —OH, —O—(C₁-C₃ alkyl) or (C₁-C₃ alkylene)-C(O)OH. In some embodiments, R² is (C₂ alkylene)-(piperidinyl), wherein the piperidinyl is optionally substituted with —OH. In some embodiments, R² is (C₂ alkylene)-(piperidinyl), wherein the piperidinyl is optionally substituted with methoxy. In some embodiments, R² is (C₂ alkylene)-(piperidinyl).

In some embodiments of the compounds of Formulas (Ia), (la′), and (Ia″), R² is (C₂ alkylene)-(piperazinyl), wherein the piperazinyl is optionally substituted with one or more substituents independently selected from oxo, C₁-C₃ alkyl, —C(O)H, —C(O)OH, —C(O)(C₁-C₃ alkyl), —C(O)(C₁-C₃ alkylene)-OH, —C(O)C(O)OH, and —SO₂(C₁-C₃ alkyl). In some embodiments, R² is (C₂ alkylene)-(piperazinyl), wherein the piperazinyl is optionally substituted with oxo and C₁-C₃ alkyl. In some embodiments, R² is (C₂ alkylene)-(piperazinyl), wherein the piperazinyl is optionally substituted with oxo and methyl. In some embodiments, R² is (C₂ alkylene)-(piperazinyl), wherein the piperazinyl is optionally substituted with —C(O)(C₁-C₃ alkyl). In some embodiments, R² is (C₂ alkylene)-(piperazinyl), wherein the piperazinyl is optionally substituted with —C(O)CH₃. In some embodiments, R² is (C₂ alkylene)-(piperazinyl).

In some embodiments of the compounds of Formulas (Ia), (Ia′), and (Ia″), R² is (C₂ alkylene)-(morpholinyl).

In some embodiments of the compounds of Formulas (Ia), (Ia′), and (Ia″), R² is (C₂ alkylene)-(1,4-oxazepanyl) optionally substituted with oxo. In some embodiments, R² is (C₂ alkylene)-(1,4-oxazepan-7-one). In some embodiments, R² is (C₂ alkylene)-(1,4-oxazepanyl).

In some embodiments of the compounds of Formulas (Ia), (Ia′), and (Ia″), R² is (C₂ alkylene)-(3-azabicyclo[3.1.0]hexanyl).

In some embodiments of the compounds of Formulas (Ia), (Ia′), and (Ia″), R² is (C₂ alkylene)-(2-oxa-5-azabicyclo[2.2.1]heptanyl).

In some embodiments of the compounds of Formulas (Ia), (Ia′), and (Ia″), R² is (C₂ alkylene)-(3-oxa-6-azabicyclo[3.3.1]heptanyl).

In some embodiments of the compounds of Formulas (Ia), (Ia′), and (Ia″), R² is (C₂ alkylene)-(2-oxa-6-azaspiro[3.3]heptanyl).

In some embodiments of the compounds of Formulas (Ia), (Ia′), and (Ia″), R² is (C₂ alkylene)-(6-oxa-1-azaspiro[3.3]heptane).

In some embodiments of the compounds of Formulas (Ia), (la′), and (Ia″), R² is (C₂ alkylene)-(octahydropyrrolo[1,2-a]pyrazinyl) optionally substituted with oxo. In some embodiments, R² is (C₂ alkylene)-(hexahydropyrrolo[1,2-a]pyrazinyl-6(2H)-one).

In some embodiments of the compounds of Formulas (Ia), (Ia′), and (Ia″), R² is (C₂ alkylene)-(hexahydro-3H-oxazolo[3,4-a]pyrazinyl) optionally substituted with oxo. In some embodiments, R² is (C₂ alkylene)-(hexahydro-3H-oxazolo[3,4-a]pyrazinyl-3-one).

In some embodiments of the compounds of Formulas (Ia), (Ia′), and (Ia″), R² is (C₂ alkylene)-(1,7-diazaspiro[3.5]nonanyl) optionally substituted with oxo. In some embodiments, R² is (C₂ alkylene)-(1,7-diazaspiro[3.5]nonanyl-2-one).

In some embodiments of the compounds of Formulas (Ia), (Ia′), and (Ia″), R² is (C₂ alkylene)-(2,7-diazaspiro[3.5]nonanyl) optionally substituted with oxo. In some embodiments, R² is (C₂ alkylene)-(2,7-diazaspiro[3.5]nonanyl-1-one).

In some embodiments of the compounds of Formulas (Ia), (Ia′), and (Ia″), R² is (C₂ alkylene)-(1-oxa-8-azaspiro[4.5]decanyl) optionally substituted with oxo. In some embodiments, R² is (C₂ alkylene)-(1-oxa-8-azaspiro[4.5]decanyl-2-one).

In some embodiments of the compounds of Formulas (Ia), (Ia′), and (Ia″), R² is (C₂ alkylene)-(2-oxa-8-azaspiro[4.5]decanyl) optionally substituted with oxo. In some embodiments, R² is (C₂ alkylene)-(2-oxa-8-azaspiro[4.5]decanyl-1-one).

In some embodiments of the compounds of Formulas (Ia), (Ia′), and (Ia″), R² is (C₁-C₃ alkylene)-NR^{2A}R^{2B}, wherein R^{2A} and R^{2B} are each independently selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-SO₂—(C₁-C₃ alkyl), and C(═NH)(C₁-C₃ alkyl).

In some embodiments of the compounds of Formulas (Ia), (Ia′), and (Ia″), R² is (C₁-C₃ alkylene)-NR^{2A}R^{2B}, wherein R^{2A} is H and R^{2B} is selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-SO₂—(C₁-C₃ alkyl), and C(═NH)(C₁-C₃ alkyl).

In some embodiments of the compounds of Formulas (Ia), (Ia′), and (Ia″), R² is (C₁-C₃ alkylene)-NR^{2A}R^{2B}, wherein R^{2A} is H and R^{2B} is selected from (C₁-C₃ alkylene)-O—(C₁-C₃ alkyl) and C(═NH)(C₁-C₃ alkyl).

In some embodiments of the compounds of Formulas (Ia), (Ia′), and (Ia″), R² is (C₁-C₃ alkylene)-NR^{2A}R^{2B}, wherein R^{2A} is H and R^{2B} is selected from 2-methoxyethyl and C(═NH)CH₃.

In some embodiments of the compounds of Formulas (Ia), (Ia′), and (Ia″), R² is (C₁-C₃ alkylene)-NR^{2A}R^{2B}, wherein R^{2A} is C₁-C₃ alkyl and R^{2B} is selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-SO₂—(C₁-C₃ alkyl), and C(═NH)(C₁-C₃ alkyl).

In some embodiments of the compounds of Formulas (Ia), (Ia′), and (Ia″), R² is (C₁-C₃ alkylene)-NR^{2A}R^{2B}, wherein R^{2A} is C₁-C₃ alkyl and R^{2B} is selected from C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), and (C₁-C₃ alkylene)-SO₂—(C₁-C₃ alkyl).

In some embodiments of the compounds of Formulas (Ia), (Ia′), and (Ia″), R² is (C₁-C₃ alkylene)-NR^{2A}R^{2B}, wherein R^{2A} is C₁-C₃ alkyl and R^{2B} is C₁-C₃ alkyl. In some embodiments, R^{2A} is C₁-C₃ alkyl and R^{2B} is selected from methyl, ethyl, and propyl.

In some embodiments of the compounds of Formulas (Ia), (Ia′), and (Ia″), R² is (C₁-C₃ alkylene)-NR^{2A}R^{2B}, wherein R^{2A} is C₁-C₃ alkyl and R^{2B} is C₃-C₆ cycloalkyl. In some embodiments, R^{2A} is C₁-C₃ alkyl and R^{2B} is cyclopropyl or cyclobutyl.

In some embodiments of the compounds of Formulas (Ia), (Ia′), and (Ia″), R² is (C₁-C₃ alkylene)-NR^{2A}R^{2B}, wherein R^{2A} is C₁-C₃ alkyl and R^{2B} is (C₁-C₃ alkylene)-(C₃-C₆ cycloalkyl). In some embodiments, R^{2A} is C₁-C₃ alkyl and R^{2B} is (C₂ alkylene)-cyclopropyl.

In some embodiments of the compounds of Formulas (Ia), (Ia′), and (Ia″), R² is (C₁-C₃ alkylene)-NR^{2A}R^{2B}, wherein R^{2A} and R^{2B}, taken together with the nitrogen to which they are attached, form a 3-10 membered heterocycloalkyl ring optionally substituted with one or more substituents independently selected from halogen, oxo, —OH, C₁-C₃ alkyl, C₁-C₃ haloalkyl, —O—(C₁-C₃ alkyl), (C₁-C₃ alkylene)-C(O)OH, —C(O)H, —C(O)(C₁-C₃ alkyl), —C(O)(C₁-C₃ alkylene)-OH, —C(O)C(O)OH, and —SO₂(C₁-C₃ alkyl), and optionally containing one additional heteroatom selected from the group of N, O, and S.

In some embodiments of the compounds of Formulas (Ia), (la′), and (Ia″), R² is (C₁-C₃ alkylene)-NR^{2A}R^{2B}, wherein R^{2A} and R^{2B}, taken together with the nitrogen to which they are attached, form a 3-10 membered heterocycloalkyl ring optionally substituted with one or more substituents independently selected from oxo, —OH, C₁-C₃ alkyl, —O—(C₁-C₃ alkyl), (C₁-C₃ alkylene)-C(O)OH, and —C(O)(C₁-C₃ alkyl), and optionally containing one additional heteroatom selected from the group of N, O, and S.

In some embodiments of the compounds of Formulas (Ia), (Ia′), and (Ia″), R² is (C₁-C₃ alkylene)-NR^{2A}R^{2B}, wherein R^{2A} and R^{2B}, taken together with the nitrogen to which they are attached, form an aziridinyl ring optionally substituted with one or more C₁-C₃ alkyl.

In some embodiments of the compounds of Formulas (Ia), (Ia′), and (Ia″), R² is (C₁-C₃ alkylene)-NR^{2A}R^{2B}, wherein R^{2A} and R^{2B}, taken together with the nitrogen to which they are attached, form an azetidinyl ring optionally substituted with one or more C₁-C₃ alkyl.

In some embodiments of the compounds of Formulas (Ia), (Ia′), and (Ia″), R² is (C₁-C₃ alkylene)-NR^{2A}R^{2B}, wherein R^{2A} and R^{2B}, taken together with the nitrogen to which they are attached, form a pyrrolidinyl ring optionally substituted with one or more substituents independently selected from —OH, C₁-C₃ alkyl, and —O—(C₁-C₃ alkyl).

In some embodiments of the compounds of Formulas (Ia), (Ia′), and (Ia″), R² is (C₁-C₃ alkylene)-NR^{2A}R^{2B}, wherein R^{2A} and R^{2B}, taken together with the nitrogen to which they are attached, form a piperidinyl ring optionally substituted with one or more substituents independently selected from —OH, C₁-C₃ alkyl, —O—(C₁-C₃ alkyl), and (C₁-C₃ alkylene)-C(O)OH.

In some embodiments of the compounds of Formulas (Ia), (Ia′), and (Ia″), R² is (C₁-C₃ alkylene)-NR^{2A}R^{2B}, wherein R^{2A} and R^{2B}, taken together with the nitrogen to which they are attached, form a morpholinyl ring optionally substituted with one or more C₁-C₃ alkyl.

In some embodiments of the compounds of Formulas (Ia), (Ia′), and (Ia″), R² is (C₁-C₃ alkylene)-NR^{2A}R²⁸, wherein R^{2A} and R^{2B}, taken together with the nitrogen to which they are attached, form a thiomorpholinyl ring optionally substituted with one or more C₁-C₃ alkyl.

In some embodiments of the compounds of Formulas (Ia), (Ia′), and (Ia″), R² is (C₁-C₃ alkylene)-NR^{2A}R^{2B}, wherein R^{2A} and R^{2B}, taken together with the nitrogen to which they are attached, form a piperazinyl ring optionally substituted with one or more substituents independently selected from oxo, C₁-C₃ alkyl, and —C(O)(C₁-C₃ alkyl).

In some embodiments of the compounds of Formulas (Ia), (Ia′), and (Ia″), R² is (C₁-C₃ alkylene)-NR^{2A}R^{2B}, wherein R^{2A} and R^{2B}, taken together with the nitrogen to which they are attached, form an oxazepanyl ring optionally substituted with one or more substituents independently selected from oxo and C₁-C₃ alkyl.

In some embodiments, the compound of Formula (I) is a compound of Formula (Ib):

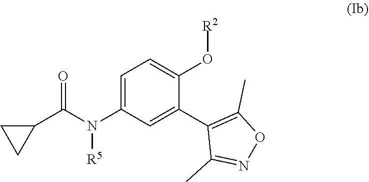

and pharmaceutically acceptable salts thereof, wherein:
R² is selected from 4-6 membered heterocycloalkyl, (C₁-C₃ alkylene)-(4-10 membered heterocycloalkyl), and (C₁-C₃ alkylene)-NR^{2A}R^{2B}, wherein the alkylene and heterocycloalkyl are each optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl);
R^{2A} and R^{2B} are each independently selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-SO₂—(C₁-C₃ alkyl), and C(═NH)(C₁-C₃ alkyl);
or R^{2A} and R^{2B}, taken together with the nitrogen to they are attached, form a 3-10 membered heterocycloalkyl ring optionally substituted with one or more C₁-C₃ alkyl, and optionally containing one additional heteroatom selected from the group of N, O, and S; and
R⁵ is selected from H and C₁-C₆ alkyl.

In some embodiments of the compound of Formula (Ib), R² is 4-6 membered heterocycloalkyl optionally substituted with one or more substituents independently selected from halogen and C₁-C₃ alkyl. In some embodiments, R² is selected from azetidinyl, pyrrolidinyl, and piperidinyl, each of which is optionally substituted with one or more substituents independently selected from halogen and (C₁-C₃ alkyl). In some embodiments, R² is azetidinyl. In some embodiments, R² is azetidinyl substituted with methyl. In some embodiments, R² is azetidinyl substituted with ethyl. In some embodiments, R² is pyrrolidinyl. In some embodiments, R² is piperidinyl.

In some embodiments of the compound of Formula (Ib), R² is (C₁-C₃ alkylene)-(4-10 membered heterocycloalkyl), wherein the alkylene and heterocycloalkyl are each optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl).

In some embodiments of the compound of Formula (Ib), R² is (C₁ alkylene)-(4-6 membered heterocycloalkyl). In some embodiments, the 4-6 membered heterocycloalkyl is selected from azetidinyl, pyrrolidinyl, piperidinyl. In some embodiments, the 4-6 membered heterocycloalkyl is azetidinyl. In some embodiments, the 4-6 membered heterocycloalkyl is pyrrolidinyl. In some embodiments, the 4-6 membered heterocycloalkyl is piperidinyl. In some embodiments, the 4-6 membered heterocycloalkyl is morpholinyl.

In some embodiments of the compound of Formula (Ib), R² is (C₂ alkylene)-(4-10 membered heterocycloalkyl), wherein the heterocycloalkyl is optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)(C₁-C₃ alkyl), —C(O)C(O)OH, and —SO₂(C₁-C₃ alkyl).

In some embodiments of the compound of Formula (Ib), R² is (C₂ alkylene)-(azetidinyl), wherein the azetidinyl is optionally substituted with halogen or —OH. In some embodiments, R² is (C₂ alkylene)-(azetidinyl), wherein the azetidinyl is optionally substituted with fluorine. In some embodiments, R² is (C₂ alkylene)-(azetidinyl), wherein the azetidinyl is optionally substituted with —OH. In some embodiments, R² is (C₂ alkylene)-(azetidinyl).

In some embodiments of the compound of Formula (Ib), R² is (C₂ alkylene)-(pyrrolidinyl), wherein the pyrrolidinyl is optionally substituted with —OH, —O—(C₁-C₃ alkyl) or (C₁-C₃ alkylene)-C(O)OH. In some embodiments, R² is (C₂ alkylene)-(pyrrolidinyl), wherein the pyrrolidinyl is optionally substituted with —OH. In some embodiments, R² is (C₂ alkylene)-(pyrrolidinyl), wherein the pyrrolidinyl is optionally substituted with methoxy. In some embodiments, R² is (C₂ alkylene)-(pyrrolidinyl).

In some embodiments of the compound of Formula (Ib), R² is (C₂ alkylene)-(piperidinyl), wherein the piperidinyl is optionally substituted with —OH, —O—(C₁-C₃ alkyl) or (C₁-C₃ alkylene)-C(O)OH. In some embodiments, R² is (C₂ alkylene)-(piperidinyl), wherein the piperidinyl is optionally substituted with —OH. In some embodiments, R² is (C₂ alkylene)-(piperidinyl), wherein the piperidinyl is optionally substituted with methoxy. In some embodiments, R² is (C₂ alkylene)-(piperidinyl).

In some embodiments of the compound of Formula (Ib), R² is (C₂ alkylene)-(piperazinyl), wherein the piperazinyl is optionally substituted with one or more substituents independently selected from oxo, C₁-C₃ alkyl, —C(O)H, —C(O)OH, —C(O)(C₁-C₃ alkyl), —C(O)(C₁-C₃ alkylene)-OH, —C(O)C(O)OH, and —SO₂(C₁-C₃ alkyl). In some embodiments, R² is (C₂ alkylene)-(piperazinyl), wherein the piperazinyl is optionally substituted with oxo and C₁-C₃ alkyl. In some embodiments, R² is (C₂ alkylene)-(piperazinyl), wherein the piperazinyl is optionally substituted with oxo and methyl. In some embodiments, R² is (C₂ alkylene)-(piperazinyl), wherein the piperazinyl is optionally substituted with —C(O)(C₁-C₃ alkyl). In some embodiments, R² is (C₂ alkylene)-(piperazinyl), wherein the piperazinyl is optionally substituted with —C(O)CH₃. In some embodiments, R² is (C₂ alkylene)-(piperazinyl).

In some embodiments of the compound of Formula (Ib), R² is (C₂ alkylene)-(morpholinyl).

In some embodiments of the compounds of Formula (Ib), R² is (C₂ alkylene)-(1,4-oxazepanyl) optionally substituted with oxo. In some embodiments, R² is (C₂ alkylene)-(1,4-oxazepan-7-one). In some embodiments, R² is (C₂ alkylene)-(1,4-oxazepanyl).

In some embodiments of the compound of Formula (Ib), R² is (C₂ alkylene)-(3-azabicyclo[3.1.0]hexanyl).

In some embodiments of the compound of Formula (Ib), R² is (C₂ alkylene)-(2-oxa-5-azabicyclo[2.2.1]heptanyl).

In some embodiments of the compounds of Formula (Ib), R² is (C₂ alkylene)-(3-oxa-6-azabicyclo[3.3.1]heptanyl).

In some embodiments of the compound of Formula (Ib), R² is (C₂ alkylene)-(2-oxa-6-azaspiro[3.3]heptanyl).

In some embodiments of the compounds of Formula (Ib), R² is (C₂ alkylene)-(6-oxa-1-azaspiro[3.3]heptane).

In some embodiments of the compound of Formula (Ib), R² is (C₂ alkylene)-(octahydropyrrolo[1,2-a]pyrazinyl) optionally substituted with oxo. In some embodiments, R² is (C₂ alkylene)-(hexahydropyrrolo[1,2-a]pyrazinyl-6(2H)-one).

In some embodiments of the compound of Formula (Ib), R² is (C₂ alkylene)-(hexahydro-3H-oxazolo[3,4-a]pyrazinyl) optionally substituted with oxo. In some embodiments, R² is (C₂ alkylene)-(hexahydro-3H-oxazolo[3,4-a]pyrazinyl-3-one).

In some embodiments of the compound of Formula (Ib), R² is (C₂ alkylene)-(1,7-diazaspiro[3.5]nonanyl) optionally substituted with oxo. In some embodiments, R² is (C₂ alkylene)-(1,7-diazaspiro[3.5]nonanyl-2-one).

In some embodiments of the compound of Formula (Ib), R² is (C₂ alkylene)-(2,7-diazaspiro[3.5]nonane) optionally substituted with oxo. In some embodiments, R² is (C₂ alkylene)-(2,7-diazaspiro[3.5]nonan-1-one).

In some embodiments of the compound of Formula (Ib), R² is (C₂ alkylene)-(1-oxa-8-azaspiro[4.5]decanyl) optionally substituted with oxo. In some embodiments, R² is (C₂ alkylene)-(1-oxa-8-azaspiro[4.5]decanyl-2-one).

In some embodiments of the compound of Formula (Ib), R² is (C₂ alkylene)-(2-oxa-8-azaspiro[4.5]decanyl) optionally substituted with oxo. In some embodiments, R² is (C₂ alkylene)-(2-oxa-8-azaspiro[4.5]decanyl-1-one).

In some embodiments of the compound of Formula (Ib), R² is (C₁-C₃ alkylene)-NR^{2A}R^{2B}, wherein R^{2A} and R^{2B} are each independently selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-SO₂—(C₁-C₃ alkyl), and C(═NH)(C₁-C₃ alkyl).

In some embodiments of the compound of Formula (Ib), R² is (C₁-C₃ alkylene)-NR^{2A}R^{2B}, wherein R^{2A} is H and R^{2B} is selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-SO₂—(C₁-C₃ alkyl), and C(═NH)(C₁-C₃ alkyl).

In some embodiments of the compound of Formula (Ib), R² is (C₁-C₃ alkylene)-NR^{2A}R^{2B}, wherein R^{2A} is Hand R^{2B} is selected from (C₁-C₃ alkylene)-O—(C₁-C₃ alkyl) and

C(═NH)(C₁-C₃ alkyl).

In some embodiments of the compound of Formula (Ib), R² is (C₁-C₃ alkylene)-NR^{2A}R^{2B}, wherein R^{2A} is H and R^{2B} is selected from 2-methoxyethyl and C(═NH)CH₃.

In some embodiments of the compound of Formula (Ib), R² is (C₁-C₃ alkyl)-NR^{2A}R^{2B}, wherein R^{2A} is C₁-C₃ alkyl and R^{2B} is selected from H, C₁-C₃ alkyl, C₁-C₃ haloalkyl, C₃-C₆ cycloalkyl, (C₁-C₃ alkyl)-O—(C₁-C₃ alkyl), (C₁-C₃ alkyl)-OH, (C₁-C₃ alkyl)-(C₃-C₆ cycloalkyl), (C₁-C₃ alkyl)-SO₂—(C₁-C₃ alkyl), and C(═NH)(C₁-C₃ alkyl).

In some embodiments of the compound of Formula (Ib), R² is (C₁-C₃ alkylene)-NR^{2A}R^{2B}, wherein R^{2A} is C₁-C₃ alkyl and R^{2B} is selected from C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), and (C₁-C₃ alkylene)-SO₂—(C₁-C₃ alkyl).

In some embodiments of the compound of Formula (Ib), R² is (C₁-C₃ alkylene)-NR^{2A}R^{2B}, wherein R^{2A} is C₁-C₃ alkyl and R^{2B} is C₁-C₃ alkyl. In some embodiments, R^{2A} is C₁-C₃ alkyl and R^{2B} is selected from methyl, ethyl, and propyl.

In some embodiments of the compound of Formula (Ib), R² is (C₁-C₃ alkylene)-NR^{2A}R^{2B}, wherein R^{2A} is C₁-C₃ alkyl and R^{2B} is C₃-C₆ cycloalkyl. In some embodiments, R^{2A} is C₁-C₃ alkyl and R^{2B} is cyclopropyl or cyclobutyl.

In some embodiments of the compounds of Formula (Ib), R² is (C₁-C₃ alkylene)-NR^{2A}R^{2B}, wherein R^{2A} is C₁-C₃ alkyl and R^{2B} is (C₁-C₃ alkylene)-(C₃-C₆ cycloalkyl). In some embodiments, R^{2A} is C₁-C₃ alkyl and R^{2B} is (C₂ alkylene)-cyclopropyl.

In some embodiments of the compounds of Formula (Ib), R² is (C₁-C₃ alkylene)-NR^{2A}R^{2B}, wherein R^{2A} and R^{2B}, taken together with the nitrogen to which they are attached, form a 3-10 membered heterocycloalkyl ring optionally substituted with one or more substituents independently selected from halogen, oxo, —OH, C₁-C₃ alkyl, C₁-C₃ haloalkyl, —O—(C₁-C₃ alkyl), (C₁-C₃ alkylene)-C(O)OH, —C(O)H, —C(O)(C₁-C₃ alkyl), —C(O)(C₁-C₃ alkylene)-OH, —C(O)C(O)OH, and —SO₂(C₁-C₃ alkyl), and optionally containing one additional heteroatom selected from the group of N, O, and S.

In some embodiments of the compounds of Formula (Ib), R² is (C₁-C₃ alkylene)-NR^{2A}R^{2B}, wherein R^{2A} and R^{2B}, taken together with the nitrogen to which they are attached, form a 3-10 membered heterocycloalkyl ring optionally substituted with one or more substituents independently selected from oxo, —OH, C₁-C₃ alkyl, —O—(C₁-C₃ alkyl), (C₁-C₃ alkylene)-C(O)OH, and —C(O)(C₁-C₃ alkyl), and optionally containing one additional heteroatom selected from the group of N, O, and S.

In some embodiments of the compound of Formula (Ib), R² is (C₁-C₃ alkylene)-NR^{2A}R^{2B}, wherein R^{2A} and R^{2B}, taken together with the nitrogen to which they are attached, form an aziridinyl ring optionally substituted with one or more C₁-C₃ alkyl.

In some embodiments of the compound of Formula (Ib), R² is (C₁-C₃ alkylene)-NR^{2A}R^{2B}, wherein R^{2A} and R^{2B}, taken together with the nitrogen to which they are attached, form an azetidinyl ring optionally substituted with one or more C₁-C₃ alkyl.

In some embodiments of the compound of Formula (Ib), R² is (C₁-C₃ alkylene)-NR^{2A}R^{2B}, wherein R^{2A} and R^{2B}, taken together with the nitrogen to which they are attached, form a pyrrolidinyl ring optionally substituted with one or more substituents independently selected from —OH, C₁-C₃ alkyl, and —O—(C₁-C₃ alkyl).

In some embodiments of the compound of Formula (Ib), R² is (C₁-C₃ alkylene)-NR^{2A}R^{2B}, wherein R^{2A} and R^{2B}, taken together with the nitrogen to which they are attached, form a piperidinyl ring optionally substituted with one or more substituents independently selected from —OH, C₁-C₃ alkyl, —O—(C₁-C₃ alkyl), and (C₁-C₃ alkylene)-C(O)OH.

In some embodiments of the compound of Formula (Ib), R² is (C₁-C₃ alkylene)-NR^{2A}R^{2B}, wherein R^{2A} and R^{2B}, taken together with the nitrogen to which they are attached, form a morpholinyl ring optionally substituted with one or more C₁-C₃ alkyl.

In some embodiments of the compound of Formula (Ib), R² is (C₁-C₃ alkylene)-NR^{2A}R^{2B}, wherein R^{2A} and R^{2B}, taken together with the nitrogen to which they are attached, form a thiomorpholinyl ring optionally substituted with one or more C₁-C₃ alkyl.

In some embodiments of the compounds of Formula (Ib), R² is (C₁-C₃ alkylene)-NR^{2A}R^{2B}, wherein R^{2A} and R^{2B}, taken together with the nitrogen to which they are attached, form a piperazinyl ring optionally substituted with one or more substituents independently selected from oxo, C₁-C₃ alkyl, and —C(O)(C₁-C₃ alkyl).

In some embodiments of the compounds of Formula (Ib), R² is (C₁-C₃ alkylene)-NR^{2A}R^{2B}, wherein R^{2A} and R^{2B}, taken together with the nitrogen to which they are attached, form an oxazepanyl ring optionally substituted with one or more substituents independently selected from oxo and C₁-C₃ alkyl.

In some embodiments of the compound of Formula (Ib), R⁵ is H.

In some embodiments of the compound of Formula (Ib), R⁵ is methyl.

In some embodiments, the compound of Formula (I) is a compound of Formula (Ic):

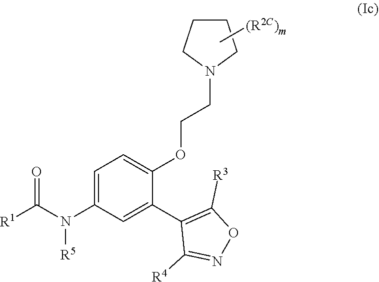

and pharmaceutically acceptable salts thereof, wherein:
R¹ is selected from C₁-C₆ alkyl, C₃-C₆ cycloalkyl, phenyl, 5-10 membered heteroaryl, 5-9 membered heterocycloalkyl, (C₁-C₃ alkylene)-(C₃-C₆ cycloalkyl), (C₁-C₃ alkylene)-phenyl, (C₁-C₃ haloalkylene)-phenyl, (C₁-C₃ alkylene)-(5-10 membered heteroaryl), (C₁-C₃ alkylene)-(5-9 membered heterocycloalkyl), (C₁-C₃ alkylene)-O—(C₃-C₆ cycloalkyl), and (C₁-C₃ alkylene)-NH—(C₃-C₆ cycloalkyl), wherein the alkyl, alkylene, cycloalkyl, phenyl, heteroaryl, and heterocycloalkyl are each optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl;
each R^{2C} is independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl);
R³ and R⁴ are each independently selected from H, C₁-C₆ alkyl, and C₁-C₆ haloalkyl;
R⁵ is selected from H and C₁-C₆ alkyl; and
m is 0, 1, 2, 3, 4, 5, or 6.

In some embodiments of the compound of Formula (Ic):
R¹ is selected from C₁-C₆ alkyl, C₃-C₆ cycloalkyl, phenyl, 5-10 membered heteroaryl, 5-9 membered heterocycloalkyl, (C₁-C₃ alkylene)-(C₃-C₆ cycloalkyl), (C₁-C₃ alkylene)-phenyl, (C₁-C₃ haloalkylene)-phenyl, (C₁-C₃ alkylene)-(5-10 membered heteroaryl), (C₁-C₃ alkylene)-(5-9 membered heterocycloalkyl), (C₁-C₃ alkylene)-O—(C₃-C₆ cycloalkyl), and (C₁-C₃ alkylene)-NH—(C₃-C₆ cycloalkyl), wherein the alkyl, alkylene, cycloalkyl, phenyl, heteroaryl, and heterocycloalkyl are each optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl;
each R^{2C} is independently selected from halogen, —OH, C₁-C₃ alkyl, and —O—(C₁-C₃ alkyl);
R³ and R⁴ are each independently selected from H and C₁-C₃ alkyl;
R⁵ is selected from H and C₁-C₃ alkyl; and
m is 0, 1, or 2.

In some embodiments of the compound of Formula (Ic), R¹ is C₁-C₆ alkyl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (Ic), R¹ is C₁-C₆ alkyl optionally substituted with —O—(C₁-C₃ alkyl).

In some embodiments of the compound of Formula (Ic), R¹ is 2-ethoxyethyl.

In some embodiments of the compound of Formula (Ic), R¹ is C₃-C₆ cycloalkyl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (Ic), R¹ is cyclopropyl optionally substituted with halogen, C₃-C₆ cycloalkyl, and —O—(C₁-C₃ alkyl).

In some embodiments of the compound of Formula (Ic), R¹ is cyclopropyl.

In some embodiments of the compound of Formula (Ic), R¹ is cyclopropyl substituted with fluorine.

In some embodiments of the compound of Formula (Ic), R¹ is cyclopropyl substituted with cyclopropyl.

In some embodiments of the compound of Formula (Ic), R¹ is cyclopropyl substituted with ethoxy.

In some embodiments of the compound of Formula (Ic), R¹ is 5-10 membered heteroaryl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (Ic), R¹ is 5-6 membered heteroaryl optionally substituted with one or more substituents independently selected from halogen and C₁-C₃ alkyl.

In some embodiments of the compound of Formula (Ic), R¹ is pyridine.

In some embodiments of the compound of Formula (Ic), R¹ is pyridine optionally substituted with halogen.

In some embodiments of the compound of Formula (Ic), R¹ is pyridine substituted with chlorine.

In some embodiments of the compound of Formula (Ic), R¹ is pyridine substituted with fluorine.

In some embodiments of the compound of Formula (Ic), R¹ is thiazolyl optionally substituted with one or more substituents independently selected from C₁-C₃ alkyl.

In some embodiments of the compound of Formula (Ic), R¹ is 4-methylthiazolyl.

In some embodiments of the compound of Formula (Ic), R¹ is 2,4-dimethylthiazolyl.

In some embodiments of the compound of Formula (Ic), R¹ is isoxazolyl optionally substituted with one or more substituents independently selected from C₁-C₃ alkyl.

In some embodiments of the compound of Formula (Ic), R¹ is 4-methylisoxazolyl.

In some embodiments of the compound of Formula (Ic), R¹ is oxazolyl optionally substituted with one or more substituents independently selected from C₁-C₃ alkyl.

In some embodiments of the compound of Formula (Ic), R¹ is 4-methyloxazolyl.

In some embodiments of the compound of Formula (Ic), R¹ is 1H-pyrazolyl optionally substituted with one or more substituents independently selected from halogen and C₁-C₃ alkyl.

In some embodiments of the compound of Formula (Ic), R¹ is 1-methyl-1H-pyrazolyl.

In some embodiments of the compound of Formula (Ic), R¹ is 4-fluoro-1-methyl-1H-pyrazolyl.

In some embodiments of the compound of Formula (Ic), R^{2C} is —OH.

In some embodiments of the compound of Formula (Ic), R^{2C} is —O—(C₁-C₃ alkyl).

In some embodiments of the compound of Formula (Ic), R^{2C} is methoxy.

In some embodiments of the compound of Formula (Ic), one of R³ and R⁴ is H and the other is C₁-C₃ alkyl.

In some embodiments of the compound of Formula (Ic), R³ and R⁴ are each C₁-C₃ alkyl.

In some embodiments of the compound of Formula (Ic), R³ and R⁴ are each methyl.

In some embodiments of the compound of Formula (Ic), R⁵ is H.

In some embodiments of the compound of Formula (Ic), R⁵ is methyl.

In some embodiments of the compound of Formula (Ic), m is 0.

In some embodiments of the compound of Formula (Ic), m is 1.

In some embodiments of the compound of Formula (Ic), m is 2.

In some embodiments, the compound of Formula (I) is a compound of Formula (Id):

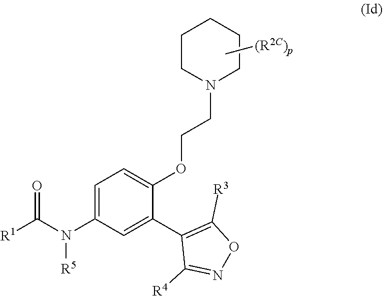

and pharmaceutically acceptable salts thereof, wherein:
R¹ is selected from C₁-C₆ alkyl, C₃-C₆ cycloalkyl, phenyl, 5-10 membered heteroaryl, 5-9 membered heterocycloalkyl, (C₁-C₃ alkylene)-(C₃-C₆ cycloalkyl), (C₁-C₃ alkylene)-phenyl, (C₁-C₃ haloalkylene)-phenyl, (C₁-C₃ alkylene)-(5-10 membered heteroaryl), (C₁-C₃ alkylene)-(5-9 membered heterocycloalkyl), (C₁-C₃ alkylene)-O—(C₃-C₆ cycloalkyl), and (C₁-C₃ alkylene)-NH—(C₃-C₆ cycloalkyl), wherein the alkyl, alkylene, cycloalkyl, phenyl, heteroaryl, and heterocycloalkyl are each optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl;
each R^{2C} is independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl);
R³ and R⁴ are each independently selected from H, C₁-C₆ alkyl, and C₁-C₆ haloalkyl;
R⁵ is selected from H and C₁-C₆ alkyl; and
m is 0, 1, 2, 3, 4, 5, or 6.

In some embodiments of the compound of Formula (Id):
R¹ is selected from C₁-C₆ alkyl, C₃-C₆ cycloalkyl, phenyl, 5-10 membered heteroaryl, 5-9 membered heterocycloalkyl, (C₁-C₃ alkylene)-(C₃-C₆ cycloalkyl), (C₁-C₃ alkylene)-phenyl, (C₁-C₃ haloalkylene)-phenyl, (C₁-C₃ alkylene)-(5-10 membered heteroaryl), (C₁-C₃ alkylene)-(5-9 membered heterocycloalkyl), (C₁-C₃ alkylene)-O—(C₃-C₆ cycloalkyl), and (C₁-C₃ alkylene)-NH—(C₃-C₆ cycloalkyl), wherein the alkyl, alkylene, cycloalkyl, phenyl, heteroaryl, and heterocycloalkyl are each optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl;
each R^{2C} is independently selected from halogen, —OH, C₁-C₃ alkyl, —O—(C₁-C₃ alkyl), and (C₁-C₃ alkylene)-C(O)OH;
R³ and R⁴ are each independently selected from H and C₁-C₃ alkyl;
R⁵ is selected from H and C₁-C₃ alkyl; and
m is 0, 1, or 2.

In some embodiments of the compound of Formula (Id), R¹ is C₁-C₆ alkyl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (Id), R¹ is C₃-C₆ cycloalkyl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (Id), R¹ is cyclopropyl optionally substituted with halogen, C₃-C₆ cycloalkyl, and —O—(C₁-C₃ alkyl).

In some embodiments of the compound of Formula (Id), R¹ is cyclopropyl.

In some embodiments of the compound of Formula (Id), R¹ is 5-10 membered heteroaryl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (Id), R¹ is 5-6 membered heteroaryl optionally substituted with one or more substituents independently selected from halogen and C₁-C₃ alkyl.

In some embodiments of the compound of Formula (Id), R^{2C} is —OH.

In some embodiments of the compound of Formula (Id), R^{2C} is —O—(C₁-C₃ alkyl).

In some embodiments of the compound of Formula (Id), R^{2C} is methoxy.

In some embodiments of the compound of Formula (Id), R^{2C} is (C₁-C₃ alkylene)-C(O)OH.

In some embodiments of the compound of Formula (Id), R^{2C} is —CH₂—C(O)OH.

In some embodiments of the compound of Formula (Id), one of R³ and R⁴ is H and the other is C₁-C₃ alkyl.

In some embodiments of the compound of Formula (Id), R³ and R⁴ are each C₁-C₃ alkyl.

In some embodiments of the compound of Formula (Id), R³ and R⁴ are each methyl.

In some embodiments of the compound of Formula (Id), R⁵ is H.

In some embodiments of the compound of Formula (Id), R⁵ is methyl.

In some embodiments of the compound of Formula (Id), p is 0.

In some embodiments of the compound of Formula (Id), p is 1.

In some embodiments of the compound of Formula (Id), p is 2.

In some embodiments, the compound of Formula (I) is a compound of Formula (Ie):

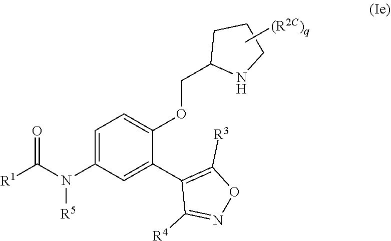

and pharmaceutically acceptable salts thereof, wherein:
R¹ is selected from C₁-C₆ alkyl, C₃-C₆ cycloalkyl, phenyl, 5-10 membered heteroaryl, 5-9 membered heterocycloalkyl, (C₁-C₃ alkylene)-(C₃-C₆ cycloalkyl), (C₁-C₃ alkylene)-phenyl, (C₁-C₃ haloalkylene)-phenyl, (C₁-C₃ alkylene)-(5-10 membered heteroaryl), (C₁-C₃ alkylene)-(5-9 membered heterocycloalkyl), (C₁-C₃ alkylene)-O—(C₃-C₆ cycloalkyl), and (C₁-C₃ alkylene)-NH—(C₃-C₆ cycloalkyl), wherein the alkyl, alkylene, cycloalkyl, phenyl, heteroaryl, and heterocycloalkyl are each optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl;
each R^{2C} is independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl);
R³ and R⁴ are each independently selected from H, C₁-C₆ alkyl, and C₁-C₆ haloalkyl;
R⁵ is selected from H and C₁-C₆ alkyl; and
q is 0, 1, 2, 3, 4, 5, or 6.

In some embodiments of the compound of Formula (Ie), R¹ is C₁-C₆ alkyl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (Ie), R¹ is C₁-C₆ alkyl optionally substituted with one or more substituents independently selected from halogen.

In some embodiments of the compound of Formula (Ie), R¹ is C₃-C₆ cycloalkyl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (Ie), R¹ is C₃-C₆ cycloalkyl optionally substituted with one or more substituents independently selected from halogen, C₁-C₃ haloalkyl, —O—(C₁-C₃ alkyl), and (C₁-C₃ alkylene)-O—(C₁-C₃ alkyl).

In some embodiments of the compound of Formula (Ie), R¹ is cyclopropyl optionally substituted with one or more substituents independently selected from halogen, C₁-C₃ haloalkyl, —O—(C₁-C₃ alkyl), and (C₁-C₃ alkylene)-O—(C₁-C₃ alkyl).

In some embodiments of the compound of Formula (Ie), R¹ is cyclopropyl.

In some embodiments of the compound of Formula (Ie), R¹ is cyclopropyl substituted with fluorine.

In some embodiments of the compound of Formula (Ie), R¹ is cyclopropyl substituted with fluoromethyl.

In some embodiments of the compound of Formula (Ie), R¹ is cyclopropyl substituted with methylmethoxy.

In some embodiments of the compound of Formula (Ie), R¹ is cyclopropyl substituted with ethoxy.

In some embodiments of the compound of Formula (Ie), R¹ is phenyl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (Ie), R¹ is 5-10 membered heteroaryl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (Ie), R¹ is 5-9 membered heterocycloalkyl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (Ie), R¹ is (C₁-C₃ alkylene)-(C₃-C₆ cycloalkyl) optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (Ie), R¹ is (C₁-C₃ alkylene)-(C₃-C₆ cycloalkyl) optionally substituted with one or more substituents independently selected from halogen and —O—(C₁-C₃ alkyl).

In some embodiments of the compound of Formula (Ie), R¹ is (C₁-C₃ alkylene)-cyclopropyl.

In some embodiments of the compound of Formula (Ie), R¹ is (C₁-C₃ alkylene)-phenyl, wherein the alkylene and phenyl are each optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (Ie), R¹ is (C₁-C₃ alkylene)-(5-10 membered heteroaryl) optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (Ie), R¹ is (C₁-C₃ alkylene)-(5-9 membered heterocycloalkyl) optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (Ie), one of R³ and R⁴ is H and the other is C₁-C₃ alkyl.

In some embodiments of the compound of Formula (Ie), R³ and R⁴ are each C₁-C₃ alkyl.

In some embodiments of the compound of Formula (Ie), R³ and R⁴ are each methyl.

In some embodiments of the compound of Formula (Ie), R⁵ is H.

In some embodiments of the compound of Formula (Ie), R⁵ is methyl.

In some embodiments of the compound of Formula (Ie), q is 0.

In some embodiments of the compound of Formula (Ie), q is 1.

In some embodiments of the compound of Formula (Ie), q is 2.

In some embodiments, the compound of Formula (I) is a compound of Formula (If):

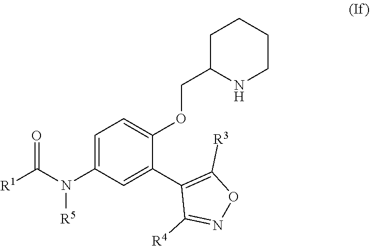

and pharmaceutically acceptable salts thereof, wherein:
R¹ is selected from C₁-C₆ alkyl, C₃-C₆ cycloalkyl, phenyl, 5-10 membered heteroaryl, 5-9 membered heterocycloalkyl, (C₁-C₃ alkylene)-(C₃-C₆ cycloalkyl), (C₁-C₃ alkylene)-phenyl, (C₁-C₃ haloalkylene)-phenyl, (C₁-C₃ alkylene)-(5-10 membered heteroaryl), (C₁-C₃ alkylene)-(5-9 membered heterocycloalkyl), (C₁-C₃ alkylene)-O—(C₃-C₆ cycloalkyl), and (C₁-C₃ alkylene)-NH—(C₃-C₆ cycloalkyl), wherein the alkyl, alkylene, cycloalkyl, phenyl, heteroaryl, and heterocycloalkyl are each optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl;
R³ and R⁴ are each independently selected from H, C₁-C₆ alkyl, and C₁-C₆ haloalkyl; and
R⁵ is selected from H and C₁-C₆ alkyl.

In some embodiments of the compound of Formula (If), R¹ is C₁-C₆ alkyl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (If), R¹ is C₁-C₆ alkyl optionally substituted with one or more substituents independently selected from halogen.

In some embodiments of the compound of Formula (If), R¹ is 2-fluoropropan-2-yl.

In some embodiments of the compound of Formula (If), R¹ is C₃-C₆ cycloalkyl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (If), R¹ is C₃-C₆ cycloalkyl optionally substituted with one or more substituents independently selected from halogen, C₁-C₃ haloalkyl, —O—(C₁-C₃ alkyl), and (C₁-C₃ alkylene)-O—(C₁-C₃ alkyl).

In some embodiments of the compound of Formula (If), R¹ is cyclopropyl optionally substituted with one or more substituents independently selected from halogen, C₁-C₃ haloalkyl, —O—(C₁-C₃ alkyl), and (C₁-C₃ alkylene)-O—(C₁-C₃ alkyl).

In some embodiments of the compound of Formula (If), R¹ is cyclopropyl.

In some embodiments of the compound of Formula (If), R¹ is cyclopropyl substituted with fluorine.

In some embodiments of the compound of Formula (If), R¹ is cyclopropyl substituted with fluoromethyl.

In some embodiments of the compound of Formula (If), R¹ is cyclopropyl substituted with methylmethoxy.

In some embodiments of the compound of Formula (If), R¹ is cyclopropyl substituted with ethoxy.

In some embodiments of the compound of Formula (If), R¹ is phenyl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (If), R¹ is phenyl optionally substituted with one or more substituents independently selected from halogen and —O—(C₁-C₃ alkyl).

In some embodiments of the compound of Formula (If), R¹ is phenyl.

In some embodiments of the compound of Formula (If), R¹ is phenyl substituted with fluorine.

In some embodiments of the compound of Formula (If), R¹ is phenyl substituted with methoxy.

In some embodiments of the compound of Formula (If), R¹ is 5-10 membered heteroaryl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (If), R¹ is 5-10 membered heteroaryl optionally substituted with one or more substituents independently selected from halogen and C₁-C₃ alkyl.

In some embodiments of the compound of Formula (If), R¹ is 1H-pyrrolyl optionally substituted with one or more substituents independently selected from halogen and C₁-C₃ alkyl.

In some embodiments of the compound of Formula (If), R¹ is 1H-pyrrolyl.

In some embodiments of the compound of Formula (If), R¹ is 1-methyl-1H-pyrrolyl.

In some embodiments of the compound of Formula (If), R¹ is 1H-pyrazolyl optionally substituted with one or more substituents independently selected from halogen in and C₁-C₃ alkyl.

In some embodiments of the compound of Formula (If), R¹ is 1H-pyrazolyl.

In some embodiments of the compound of Formula (If), R¹ is 1-methyl-1H-pyrazolyl.

In some embodiments of the compound of Formula (If), R¹ is 1,4-dimethyl-1H-pyrazolyl.

In some embodiments of the compound of Formula (If), R¹ is 1,5-dimethyl-1H-pyrazolyl.

In some embodiments of the compound of Formula (If), R¹ is 3-ethyl-1-methyl-1H-pyrazolyl.

In some embodiments of the compound of Formula (If), R¹ is 4-fluoro-1-methyl-1H-pyrazolyl.

In some embodiments of the compound of Formula (If), R¹ is isoxazolyl optionally substituted with one or more substituents independently selected from halogen and C₁-C₃ alkyl.

In some embodiments of the compound of Formula (If), R¹ is isoxazolyl.

In some embodiments of the compound of Formula (If), R¹ is 3-methylisoxazolyl.

In some embodiments of the compound of Formula (If), R¹ is 4-methylisoxazolyl.

In some embodiments of the compound of Formula (If), R¹ is 5-methylisoxazolyl.

In some embodiments of the compound of Formula (If), R¹ is oxazolyl optionally substituted with one or more substituents independently selected from halogen and C₁-C₃ alkyl.

In some embodiments of the compound of Formula (If), R¹ is oxazolyl.

In some embodiments of the compound of Formula (If), R¹ is 4-methyloxazolyl.

In some embodiments of the compound of Formula (If), R¹ is 5-methyloxazolyl.

In some embodiments of the compound of Formula (If), R¹ is 2,4-dimethyloxazolyl.

In some embodiments of the compound of Formula (If), R¹ is 2,5-dimethyloxazolyl.

In some embodiments of the compound of Formula (If), R¹ is isothiazolyl optionally substituted with one or more substituents independently selected from halogen and C₁-C₃ alkyl.

In some embodiments of the compound of Formula (If), R¹ is isothiazolyl.

In some embodiments of the compound of Formula (If), R¹ is 3-methylisothiazolyl.

In some embodiments of the compound of Formula (If), R¹ is 4-methylisothiazolyl.

In some embodiments of the compound of Formula (If), R¹ is thiazolyl optionally substituted with one or more substituents independently selected from halogen and C₁-C₃ alkyl.

In some embodiments of the compound of Formula (If), R¹ is thiazolyl.

In some embodiments of the compound of Formula (If), R¹ is 4-methylthiazolyl.

In some embodiments of the compound of Formula (If), R¹ is 5-methylthiazolyl.

In some embodiments of the compound of Formula (If), R¹ is 2,4-dimethylthiazolyl.

In some embodiments of the compound of Formula (If), R¹ is 2,5-dimethylthiazolyl.

In some embodiments of the compound of Formula (If), R¹ is 2-isopropyl-4-methylthiazolyl.

In some embodiments of the compound of Formula (If), R¹ is 1H-1,2,4-triazolyl optionally substituted with one or more substituents independently selected from halogen and C₁-C₃ alkyl.

In some embodiments of the compound of Formula (If), R¹ is 1H-1,2,4-triazolyl.

In some embodiments of the compound of Formula (If), R¹ is 1-methyl-1H-1,2,4-triazolyl.

In some embodiments of the compound of Formula (If), R¹ is 1,2,5-oxadiazolyl optionally substituted with one or more substituents independently selected from halogen and C₁-C₃ alkyl.

In some embodiments of the compound of Formula (If), R¹ is 1,2,5-oxadiazolyl.

In some embodiments of the compound of Formula (If), R¹ is 3-methyl-1,2,5-oxadiazolyl.

In some embodiments of the compound of Formula (If), R¹ is 1,2,3-thiadiazolyl optionally substituted with one or more substituents independently selected from halogen and C₁-C₃ alkyl.

In some embodiments of the compound of Formula (If), R¹ is 1,2,3-thiadiazolyl.

In some embodiments of the compound of Formula (If), R¹ is 4-methyl-1,2,3-thiadiazolyl.

In some embodiments of the compound of Formula (If), R¹ is 5-9 membered heterocycloalkyl optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (If), R¹ is 6-oxaspiro[2.5]octanyl.

In some embodiments of the compound of Formula (If), R¹ is (C₁-C₃ alkylene)-(C₃-C₆ cycloalkyl) optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (If), R¹ is (C₁-C₃ alkylene)-(C₃-C₆ cycloalkyl) optionally substituted with one or more substituents independently selected from halogen and —O—(C₁-C₃ alkyl).

In some embodiments of the compound of Formula (If), R¹ is (C₁-C₃ alkylene)-cyclopropyl.

In some embodiments of the compound of Formula (If), R¹ is (C₁-C₃ alkylene)-cyclopropyl substituted with methoxy.

In some embodiments of the compound of Formula (If), R¹ is (C₁-C₃ alkylene)-cyclobutyl.

In some embodiments of the compound of Formula (If), R¹ is (C₁-C₃ alkylene)-cyclobutyl substituted with methoxy.

In some embodiments of the compound of Formula (If), R¹ is (C₁-C₃ alkylene)-phenyl, wherein the alkylene and phenyl are each optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (If), R¹ is (C₁-C₃ alkylene)-phenyl, wherein the alkylene and phenyl are each optionally substituted with one or more substituents independently selected from halogen and —O—(C₁-C₃ alkyl).

In some embodiments of the compound of Formula (If), R¹ is (C₁-C₃ alkylene)-phenyl.

In some embodiments of the compound of Formula (If), R¹ is (CH₂F)-phenyl.

In some embodiments of the compound of Formula (If), R¹ is (C₁-C₃ alkylene)-phenyl substituted with fluorine.

In some embodiments of the compound of Formula (If), R¹ is (C₁-C₃ alkylene)-phenyl substituted with methoxy.

In some embodiments of the compound of Formula (If), R¹ is (C₁-C₃ alkylene)-(5-10 membered heteroaryl) optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (If), R¹ is (C₁-C₃ alkylene)-(5-10 membered heteroaryl) optionally substituted with one or more substituents independently selected from halogen and C₁-C₃ alkyl.

In some embodiments of the compound of Formula (If), R¹ is (C₁-C₃ alkylene)-pyridinyl optionally substituted with one or more substituents independently selected from halogen and C₁-C₃ alkyl.

In some embodiments of the compound of Formula (If), R¹ is (C₁-C₃ alkylene)-pyridinyl.

In some embodiments of the compound of Formula (If), R¹ is (C₁-C₃ alkylene)-pyridinyl substituted with fluorine.

In some embodiments of the compound of Formula (If), R¹ is (C₁-C₃ alkylene)-thiazolyl optionally substituted with one or more substituents independently selected from halogen and C₁-C₃ alkyl.

In some embodiments of the compound of Formula (If), R¹ is (C₁-C₃ alkylene)-thiazolyl.

In some embodiments of the compound of Formula (If), R¹ is (C₁-C₃ alkylene)-4-methylthizaolyl.

In some embodiments of the compound of Formula (If), R¹ is (C₁-C₃ alkylene)-isothiazolyl optionally substituted with one or more substituents independently selected from halogen and C₁-C₃ alkyl.

In some embodiments of the compound of Formula (If), R¹ is (C₁-C₃ alkylene)-isothiazolyl.

In some embodiments of the compound of Formula (If), R¹ is (C₁-C₃ alkylene)-(5-9 membered heterocycloalkyl) optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl.

In some embodiments of the compound of Formula (If), R¹ is (C₁-C₃ alkylene)-tetrahydrofuranyl.

In some embodiments of the compound of Formula (If), one of R³ and R⁴ is H and the other is C₁-C₃ alkyl.

In some embodiments of the compound of Formula (If), R³ and R⁴ are each C₁-C₃ alkyl.

In some embodiments of the compound of Formula (If), R³ and R⁴ are each methyl.

In some embodiments of the compound of Formula (If), R⁵ is H.

In some embodiments of the compound of Formula (If), R⁵ is methyl.

In some embodiments, the compound of Formula (I), or a pharmaceutically acceptable salt thereof, is selected from a compound shown in Table 1.

TABLE 1

| Cmpd. | | |
|---|---|---|
| No. | Chemical Structure | Chemical Name |
| 100 | 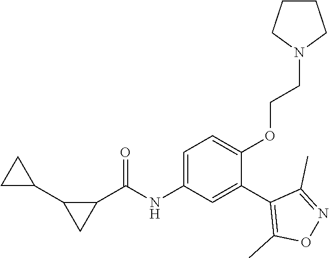 [SMILES: Cc1noc(C)c1-c1cc(NC(=O)C2CC2C2CC2)ccc1OCCN1CCCC1] | N-(3-(3,5-dimethylisoxazol-4-yl)-4-(2-(pyrrolidin-1- yl)ethoxy)phenyl)-[1,1′-bi(cyclopropane)]-2-carboxamide |
| 101 | 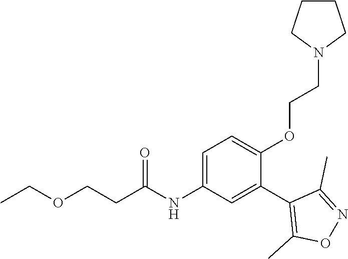 [SMILES: CCOCCC(=O)Nc1ccc(OCCN2CCCC2)c(-c2c(C)noc2C)c1] | N-(3-(3,5-dimethylisoxazol-4-yl)-4-(2-(pyrrolidin-1- yl)ethoxy)phenyl)-3-ethoxypropanamide |
| 102 | 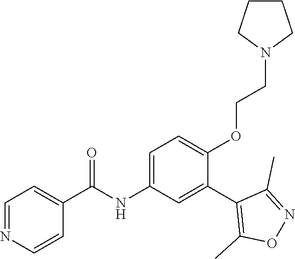 [SMILES: Cc1noc(C)c1-c1cc(NC(=O)c2ccncc2)ccc1OCCN1CCCC1] | N-(3-(3,5-dimethylisoxazol-4-yl)-4-(2-(pyrrolidin-1- yl)ethoxy)phenyl)isonicotinamide |
| 103 | 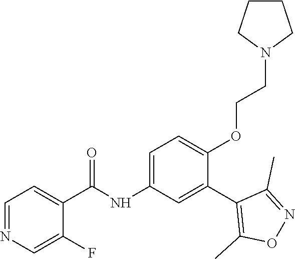 [SMILES: Cc1noc(C)c1-c1cc(NC(=O)c2ccncc2F)ccc1OCCN1CCCC1] | N-(3-(3,5-dimethylisoxaozl-4-yl)-4-(2-(pyrrolidin-1- yl)ethoxy)phenyl)-3-fluoroisonicotinamide |
| 104 | 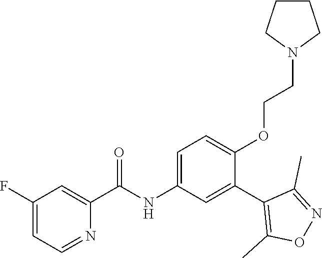 [SMILES: Cc1noc(C)c1-c1cc(NC(=O)c2cc(F)ccn2)ccc1OCCN1CCCC1] | N-(3-(3,5-dimethylisoxazol-4-yl)-4-(2-(pyrrolidin-1- yl)ethoxy)phenyl)-4-fluoropicolinamide |
| 105 | 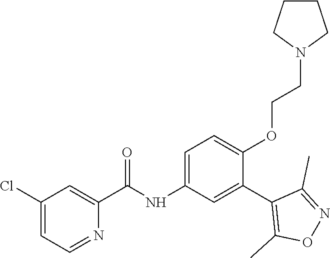 [SMILES: Cc1noc(C)c1-c1cc(NC(=O)c2cc(Cl)ccn2)ccc1OCCN1CCCC1] | 4-chloro-N-(3-(3,5-dimethylisoxazol-4-yl)-4-(2-(pyrrolidin-1- yl)ethoxy)phenyl)picolinamide |
| 106 | 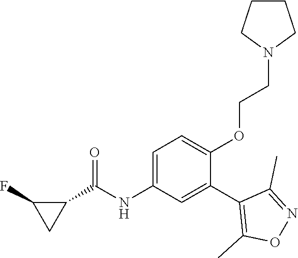 [SMILES: Cc1noc(C)c1-c1cc(NC(=O)[C@@H]2C[C@H]2F)ccc1OCCN1CCCC1] | (1S,2R)-N-[3-(3,5-dimethylisoxazol-4-yl)-4-(2-pyrrolidin-1- ylethoxy)phenyl]-2-fluoro-cyclopropanecarboxamide |
| 107 | 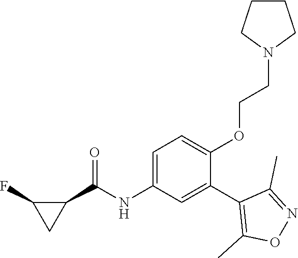 [SMILES: Cc1noc(C)c1-c1cc(NC(=O)[C@H]2C[C@H]2F)ccc1OCCN1CCCC1] | (1R,2R)-N-(3-(3,5-dimethylisoxazol-4-yl)-4-(2-(pyrrolidin-1- yl)ethoxy)phenyl)-2-fluorocyclopropane-1-carboxamide |
| 108 | 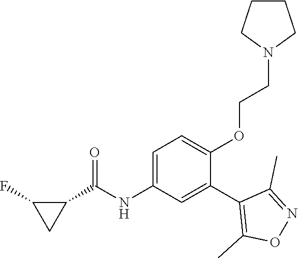 [SMILES: Cc1noc(C)c1-c1cc(NC(=O)[C@@H]2C[C@@H]2F)ccc1OCCN1CCCC1] | (1S,2S)-N-(3-(3,5-dimethylisoxazol-4-yl)-4-(2-(pyrrolidin-1- yl)ethoxy)phenyl)-2-fluorocyclopropane-1-carboxamide |
| 109 | 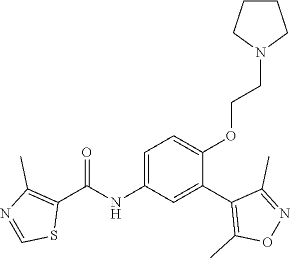 [SMILES: Cc1ncsc1C(=O)Nc1ccc(OCCN2CCCC2)c(-c2c(C)noc2C)c1] | N-(3-(3,5-dimethylisoxaozl-4-yl)-4-(2-(pyrrolidin-1- yl)ethoxy)phenyl)-4-methylthiazole-5-carboxamide |
| 110 | 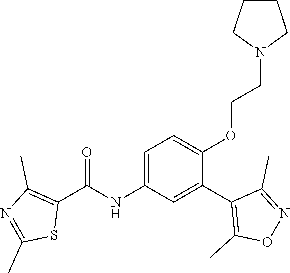 [SMILES: Cc1nc(C)c(C(=O)Nc2ccc(OCCN3CCCC3)c(-c3c(C)noc3C)c2)s1] | N-(3-(3,5-dimethylisoxazol-4-yl)-4-(2-(pyrrolidin-1- yl)ethoxy)phenyl)-2,4-dimethylthiazole-5-carboxamide |
| 111 | 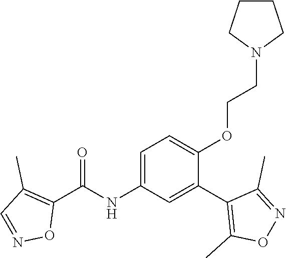 [SMILES: Cc1cnoc1C(=O)Nc1ccc(OCCN2CCCC2)c(-c2c(C)noc2C)c1] | N-(3-(3,5-dimethylisoxazol-4-yl)-4-(2-(pyrrolidin-1- yl)ethoxy)phenyl)-4-methylisoxazole-5-carboxamide |
| 112 | 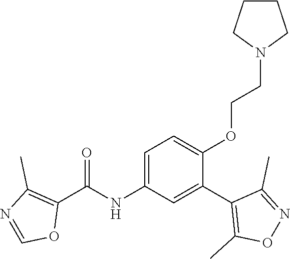 [SMILES: Cc1ncoc1C(=O)Nc1ccc(OCCN2CCCC2)c(-c2c(C)noc2C)c1] | N-(3-(3,5-dimethylisoxazol-4-yl)-4-(2-(pyrrolidin-1- yl)ethoxy)phenyl)-4-methyloxazole-5-carboxamide |
| 113 | 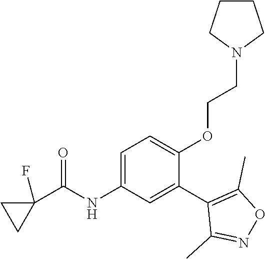 [SMILES: Cc1noc(C)c1-c1cc(NC(=O)C2(F)CC2)ccc1OCCN1CCCC1] | N-[3-(3,5-dimethylisoxazol-4-yl)-4-(2-pyrrolidin-1- ylethoxy)phenyl]-1-fluoro-cyclopropanecarboxamide |
| 114 | 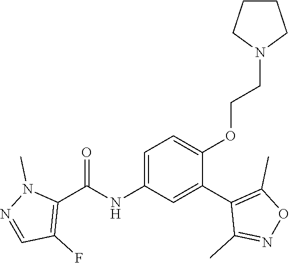 [SMILES: Cc1noc(C)c1-c1cc(NC(=O)c2c(F)cnn2C)ccc1OCCN1CCCC1] | N-(3-(3,5-dimethylisoxazol-4-yl)-4-(2-(pyrrolidin-1- yl)ethoxy)phenyl)-4-fluoro-1-methyl-1H-pyrazole-5- carboxamide |
| 115 | 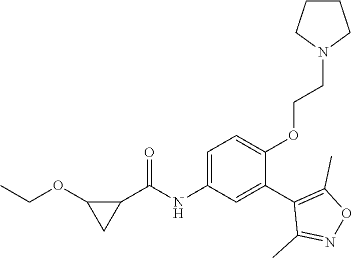 [SMILES: CCOC1CC1C(=O)Nc1ccc(OCCN2CCCC2)c(-c2c(C)noc2C)c1] | N-(3-(3,5-dimethylisoxazol-4-yl)-4-(2-(pyrrolidin-1- yl)ethoxy)phenyl)-2-ethoxycyclopropane-1-carboxamide |
| 116 | 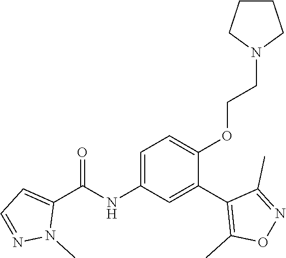 [SMILES: Cc1noc(C)c1-c1cc(NC(=O)c2ccnn2C)ccc1OCCN1CCCC1] | N-[3-(3,5-dimethylisoxazol-4-yl)-4-(2-pyrrolidin-1- ylethoxy)phenyl]-2-methyl-pyrazole-3-carboxamide |
| 117 | 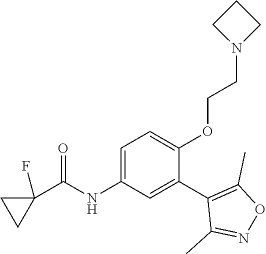 [SMILES: Cc1noc(C)c1-c1cc(NC(=O)C2(F)CC2)ccc1OCCN1CCC1] | N-[4-[2-(azetidin-1-yl)ethoxy]-3-(3,5-dimethylisoxazol-4- yl)phenyl]-1-fluoro-cyclopropanecarboxamide |
| 118 | 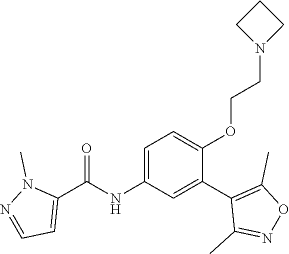 [SMILES: Cc1noc(C)c1-c1cc(NC(=O)c2ccnn2C)ccc1OCCN1CCC1] | N-(4-(2-(azetidin-1-yl)ethoxy)-3-(3,5-dimethylisoxazol-4- yl)pehnyl)-1-methyl-1H-pyrazole-5-carboxamide |
| 119 | 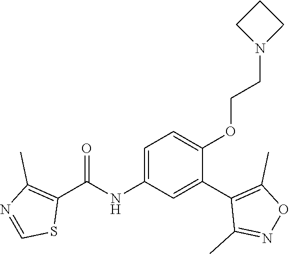 [SMILES: Cc1ncsc1C(=O)Nc1ccc(OCCN2CCC2)c(-c2c(C)noc2C)c1] | N-(4-(2-(azetidin-1-yl)ethoxy)-3-(3,5-dimethylisoxazol-4- yl)phenyl)-4-methylthiazole-5-carboxamide |
| 120 | 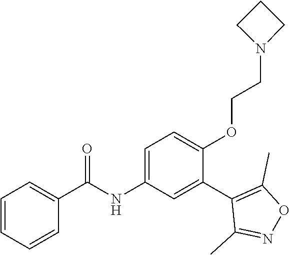 [SMILES: Cc1noc(C)c1-c1cc(NC(=O)c2ccccc2)ccc1OCCN1CCC1] | N-(4-(2-(azetidin-1-yl)ethoxy)-3-(3,5-dimethylisoxazol-4- yl)phenyl)benzamide |
| 121 | 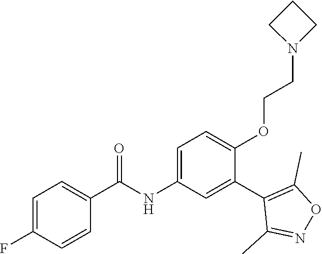 [SMILES: Cc1noc(C)c1-c1cc(NC(=O)c2ccc(F)cc2)ccc1OCCN1CCC1] | N-(4-(2-(azetidin-1-yl)ethoxy)-3-(3,5-dimethylisoxazol-4- yl)phenyl)-4-fluorobenzamide |
| 122 | 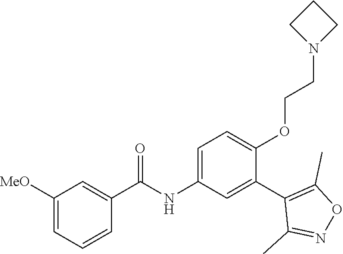 [SMILES: COc1cccc(C(=O)Nc2ccc(OCCN3CCC3)c(-c3c(C)noc3C)c2)c1] | N-(4-(2-(azetidin-1-yl)ethoxy)-3-(3,5-dimethylisoxazol-4- yl)phenyl)-3-methoxybenzamide |
| 123 | 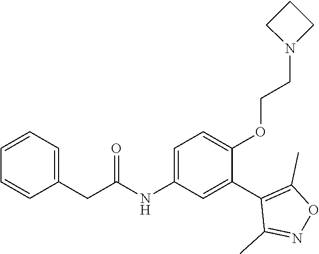 [SMILES: Cc1noc(C)c1-c1cc(NC(=O)Cc2ccccc2)ccc1OCCN1CCC1] | N-(4-(2-(azetidin-1-yl)ethoxy)-3-(3,5-dimethylisoxazol-4- yl)phenyl)-2-phenylacetamide |
| 124 | 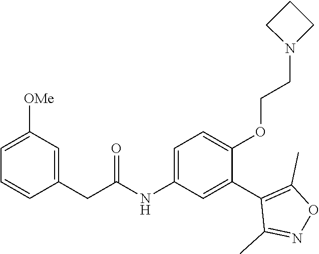 [SMILES: COc1cccc(CC(=O)Nc2ccc(OCCN3CCC3)c(-c3c(C)noc3C)c2)c1] | N-(4-(2-(azetidin-1-yl)ethoxy)-3-(3,5-dimethylisoxazol-4- yl)phenyl)-2-(3-methoxyphenyl)acetamide |
| 125 | 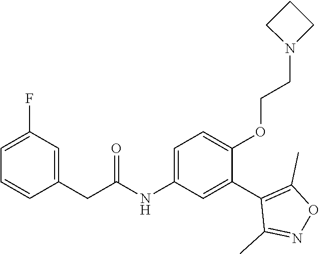 [SMILES: Cc1noc(C)c1-c1cc(NC(=O)Cc2cccc(F)c2)ccc1OCCN1CCC1] | N-(4-(2-(azetidin-1-yl)ethoxy)-3-(3,5-dimethylisoxazol-4- yl)phenyl)-2-(3-fluorophenyl)acetamide |
| 126 | 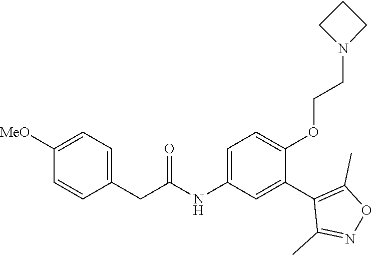 [SMILES: COc1ccc(CC(=O)Nc2ccc(OCCN3CCC3)c(-c3c(C)noc3C)c2)cc1] | N-(4-(2-(azetidin-1-yl)ethoxy)-3-(3,5-dimethylisoxazol-4- yl)phenyl)-2-(4-methoxyphenyl)acetamide |
| 127 | 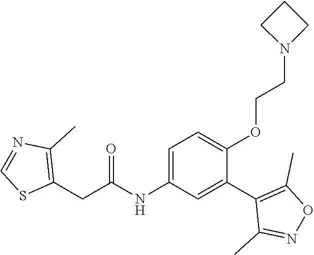 [SMILES: Cc1ncsc1CC(=O)Nc1ccc(OCCN2CCC2)c(-c2c(C)noc2C)c1] | N-(4-(2-(azetidin-1-yl)ethoxy)-3-(3,5-dimethylisoxazol-4- yl)phenyl)-2-(4-methylthiazol-5-yl)acetamide |
| 128 | 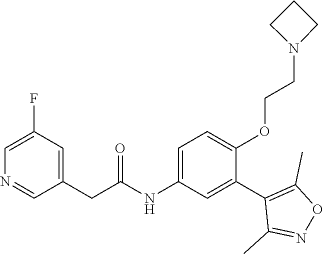 [SMILES: Cc1noc(C)c1-c1cc(NC(=O)Cc2cncc(F)c2)ccc1OCCN1CCC1] | N-(4-(2-(azetidin-1-yl)ethoxy)-3-(3,5-dimethylisoxazol-4- yl)phenyl)-2-(5-fluoropyridin-3-yl)acetamide |
| 129 | 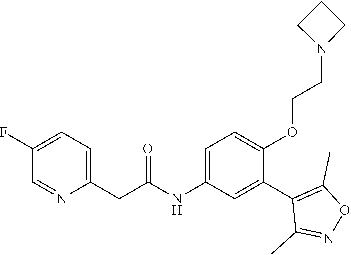 [SMILES: Cc1noc(C)c1-c1cc(NC(=O)Cc2ccc(F)cn2)ccc1OCCN1CCC1] | N-(4-(2-(azetidin-1-yl)ethoxy)-3-(3,5-dimethylisoxazol-4- yl)phenyl)-2-(5-fluoropyridin-2-yl)acetamide |
| 130 | 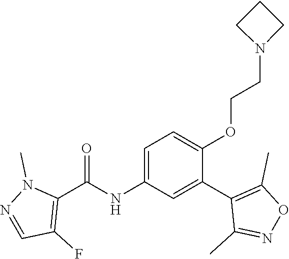 [SMILES: Cc1noc(C)c1-c1cc(NC(=O)c2c(F)cnn2C)ccc1OCCN1CCC1] | N-(4-(2-(azetidin-1-yl)ethoxy)-3-(3,5-dimethylisoxazol-4- yl)phenyl)-4-fluoro-1-methyl-1H-pyrazole-5-carboxamide |
| 131 | 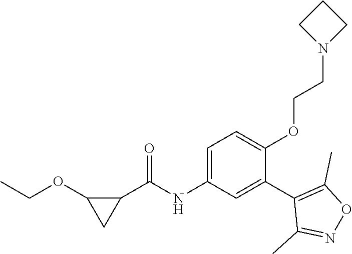 [SMILES: CCOC1CC1C(=O)Nc1ccc(OCCN2CCC2)c(-c2c(C)noc2C)c1] | N-(4-(2-(azetidin-1-yl)ethoxy)-3-(3,5-dimethylisoxazol-4- yl)phenyl)-2-ethoxycyclopropane-1-carboxamide |
| 132 | 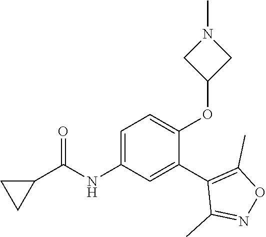 [SMILES: Cc1noc(C)c1-c1cc(NC(=O)C2CC2)ccc1OC1CN(C)C1] | N-[3-(3,5-dimethylisoxazol-4-yl)-4-(1-methylazetidin-3-yl)oxy- phenyl]cyclopropanecarboxamide |
| 133 | 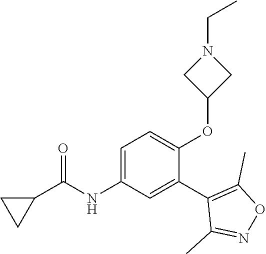 [SMILES: CCN1CC(Oc2ccc(NC(=O)C3CC3)cc2-c2c(C)noc2C)C1] | N-(3-(3,5-dimethylisoxazol-4-yl)-4-((1-ethylazetidin-3- yl)oxy)phenyl)cyclopropanecarboxamide |
| 134 | 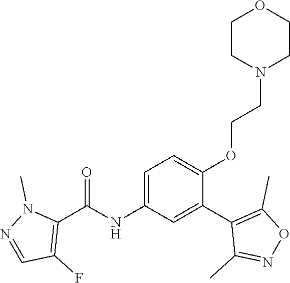 [SMILES: Cc1noc(C)c1-c1cc(NC(=O)c2c(F)cnn2C)ccc1OCCN1CCOCC1] | N-[3-(3,5-dimethylisoxazol-4-yl)-4-(2- morpholinoethoxy)phenyl]-4-fluoro-2-methyl-pyrazole-3- carboxamide |
| 135 | 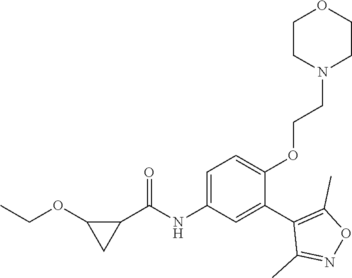 [SMILES: CCOC1CC1C(=O)Nc1ccc(OCCN2CCOCC2)c(-c2c(C)noc2C)c1] | N-[3-(3,5-dimethylisoxazol-4-yl)-4-(2- morpholinoethoxy)phenyl]-2-ethoxy-cyclopropanecarboxamide |
| 136 | 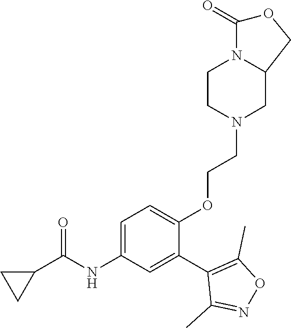 [SMILES: Cc1noc(C)c1-c1cc(NC(=O)C2CC2)ccc1OCCN1CCN2C(=O)OCC2C1] | N-[3-(3,5-dimethylisoxazol-4-yl)-4-[2-(3-oxo-5,6,8,8a- tetrahydro-1H-pyrazolo[3,4-a]pyrazin-7- yl)ethoxy]phenyl]cyclopropanecarboxamide |
| 137 | 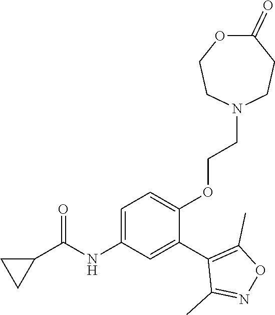 [SMILES: Cc1noc(C)c1-c1cc(NC(=O)C2CC2)ccc1OCCN1CCOC(=O)CC1] | N-[3-(3,5-dimethylisoxazol-4-yl)-4-[2-(7-oxo-1,4-oxazepan-4- yl)ethoxy]phenyl]cyclopropanecarboxamide |
| 138 | 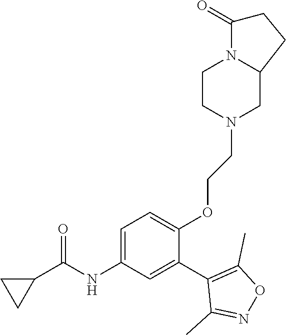 [SMILES: Cc1noc(C)c1-c1cc(NC(=O)C2CC2)ccc1OCCN1CCN2C(=O)CCC2C1] | N-(3-(3,5-dimethylisoxazol-4-yl)-4-(2-(6- oxohexahydropyrrolo[1,2-a]pyrazin-2(1H)- yl)ethoxy)phenyl)cyclopropanecarboxamide |
| 139 | 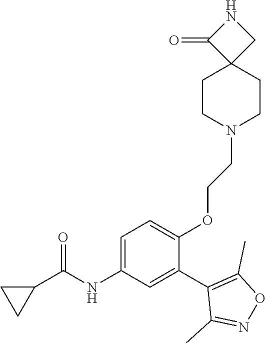 [SMILES: Cc1noc(C)c1-c1cc(NC(=O)C2CC2)ccc1OCCN1CCC2(CC1)CNC2=O] | N-(3-(3,5-dimethylisoxazol-4-yl)-4-(2-(1-oxo-2,7- diazaspiro[3.5]nonan-7- yl)ethoxy)phenyl)cyclopropanecarboxamide |
| 140 | 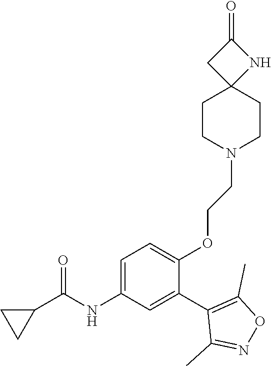 [SMILES: Cc1noc(C)c1-c1cc(NC(=O)C2CC2)ccc1OCCN1CCC2(CC1)CC(=O)N2] | N-(3-(3,5-dimethylisoxazol-4-yl)-4-(2-(2-oxo-1,7- diazaspiro[3.5]nonan-7- yl)ethoxy)phenyl)cyclopropanecarboxamide |
| 141 | 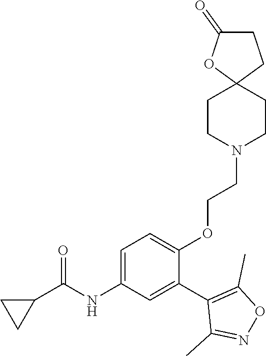 [SMILES: Cc1noc(C)c1-c1cc(NC(=O)C2CC2)ccc1OCCN1CCC2(CCC(=O)O2)CC1] | N-(3-(3,5-dimethylisoxazol-4-yl)-4-(2-(2-oxo-1-oxa-8- azaspiro[4.5]decan-8- yl)ethoxy)phenyl)cyclopropanecarboxamide |
| 142 | 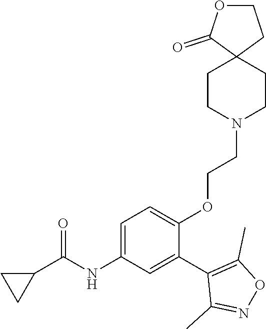 [SMILES: Cc1noc(C)c1-c1cc(NC(=O)C2CC2)ccc1OCCN1CCC2(CCOC2=O)CC1] | N-(3-(3,5-dimethylisoxazol-4-yl)-4-(2-(1-oxo-2-oxa-8- azaspiro[4.5]decan-8- yl)ethoxy)phenyl)cyclopropanecarboxamide |
| 143 | 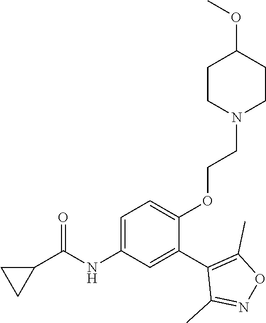 [SMILES: COC1CCN(CCOc2ccc(NC(=O)C3CC3)cc2-c2c(C)noc2C)CC1] | N-(3-(3,5-dimethylisoxazol-4-yl)-4-(2-(4-methoxypiperidin-1- yl)ethoxy)phenyl)cyclopropanecarboxamide |
| 144 | 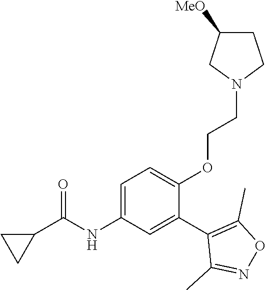 [SMILES: CO[C@H]1CCN(CCOc2ccc(NC(=O)C3CC3)cc2-c2c(C)noc2C)C1] | (S)-N-(3-(3,5-dimethylisoxazol-4-yl)-4-(2-(3- methoxypyrrolidin-1- yl)ethoxy)phenyl)cyclopropanecarboxamide |
| 145 | 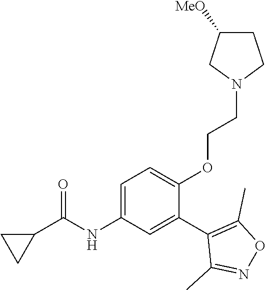 [SMILES: CO[C@@H]1CCN(CCOc2ccc(NC(=O)C3CC3)cc2-c2c(C)noc2C)C1] | (R)-N-(3-(3,5-dimethylisoxazol-4-yl)-4-(2-(3- methoxypyrrolidin-1- yl)ethoxy)phenyl)cyclopropanecarboxamide |
| 146 | 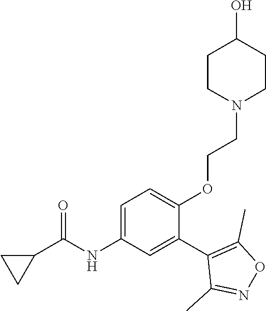 [SMILES: Cc1noc(C)c1-c1cc(NC(=O)C2CC2)ccc1OCCN1CCC(O)CC1] | N-(3-(3,5-dimethylisoxazol-4-yl)-4-(2-(4-hydroxypiperidin-1- yl)ethoxy)phenyl)cyclopropanecarobxamide |
| 147 | 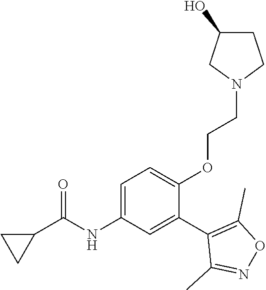 [SMILES: Cc1noc(C)c1-c1cc(NC(=O)C2CC2)ccc1OCCN1CC[C@H](O)C1] | (S)-N-(3-(3,5-dimethylisoxazol-4-yl)-4-(2-(3- hydroxypyrrolidin-1- yl)ethoxy)phenyl)cyclopropanecarboxamide |
| 148 | 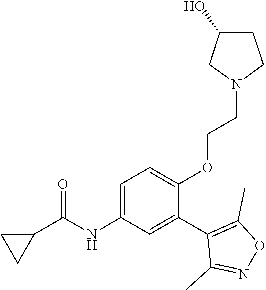 [SMILES: Cc1noc(C)c1-c1cc(NC(=O)C2CC2)ccc1OCCN1CC[C@@H](O)C1] | (R)-N-(3-(3,5-dimethylisoxazol-4-yl)-4-(2-(3- hydroxypyrrolidin-1- yl)ethoxy)phenyl)cyclopropanecarboxamide |
| 149 | 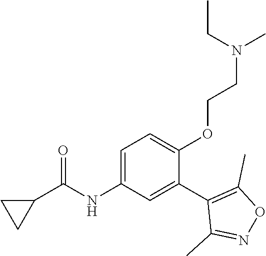 [SMILES: CCN(C)CCOc1ccc(NC(=O)C2CC2)cc1-c1c(C)noc1C] | N-(3-(3,5-dimethylisoxazol-4-yl)-4-(2- (ethyl(methyl)amino)ethoxy)phenyl)cyclopropanecarboxamide |
| 150 | 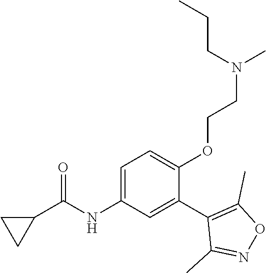 [SMILES: CCCN(C)CCOc1ccc(NC(=O)C2CC2)cc1-c1c(C)noc1C] | N-(3-(3,5-dimethylisoxazol-4-yl)-4-(2- (methyl(propyl)amino)ethoxy)phenyl)cyclopropanecarboxamide |
| 151 | 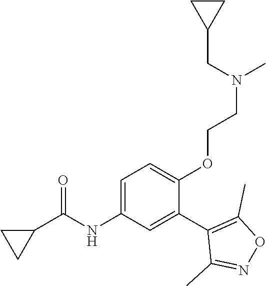 [SMILES: Cc1noc(C)c1-c1cc(NC(=O)C2CC2)ccc1OCCN(C)CC1CC1] | N-(4-(2-((cyclopropylmethyl)(methyl)amino)-ethoxy)-3-(3,5- dimethylisoxazol-4-yl)phenyl)cyclopropanecarboxamide |
| 152 | 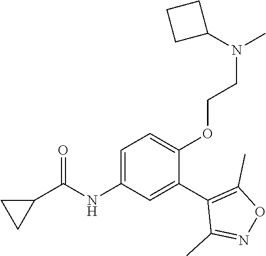 [SMILES: Cc1noc(C)c1-c1cc(NC(=O)C2CC2)ccc1OCCN(C)C1CCC1] | N-(4-(2-(cyclobutyl(methyl)amino)ethoxy)-3-(3,5- dimethylisoxazol-4-yl)phenyl)cyclopropanecarboxamide |
| 153 | 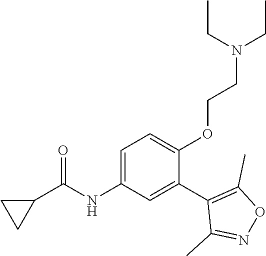 [SMILES: CCN(CC)CCOc1ccc(NC(=O)C2CC2)cc1-c1c(C)noc1C] | N-(4-(2-(diethylamino)ethoxy)-3-(3,5-dimethylisoxazol-4- yl)phenyl)cyclopropanecarboxamide |
| 154 | 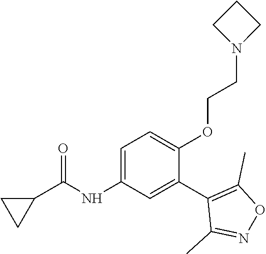 [SMILES: Cc1noc(C)c1-c1cc(NC(=O)C2CC2)ccc1OCCN1CCC1] | N-[4-[2-(azetidin-1-yl)ethoxy]-3-(3,5-dimethylisoxazol-4- yl)phenyl]cyclopropanecarboxamide |
| 155 | 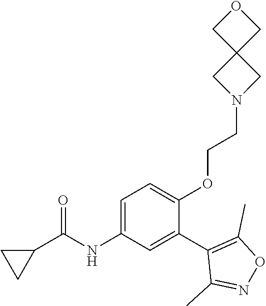 [SMILES: Cc1noc(C)c1-c1cc(NC(=O)C2CC2)ccc1OCCN1CC2(COC2)C1] | N-[3-(3,5-dimethylisoxazol-4-yl)-4-[2-(2-oxa-6- azaspiro[3.3]heptan-6- yl)ethoxy]phenyl]cyclopropanecarboxamide |
| 156 | 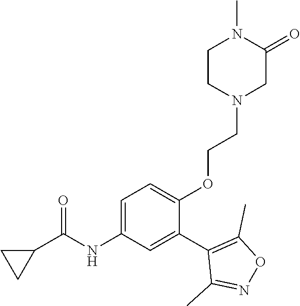 [SMILES: Cc1noc(C)c1-c1cc(NC(=O)C2CC2)ccc1OCCN1CCN(C)C(=O)C1] | N-[3-(3,5-dimethylisoxazol-4-yl)-4-[2-(4-methyl-3-oxo- piperazin-1-yl)ethoxy]phenyl]cycloproanecarboxamide |
| 157 | 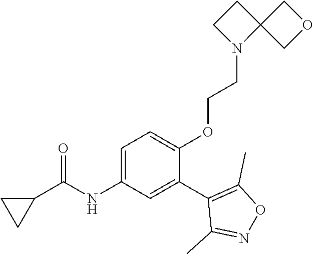 [SMILES: Cc1noc(C)c1-c1cc(NC(=O)C2CC2)ccc1OCCN1CCC12COC2] | N-[3-(3,5-dimethylisoxazol-4-yl)-4-[2-(6-oxa-1- azaspiro[3.3]heptan-1- yl)ethoxy]phenyl]cyclopropanecarboxamide |
| 158 | 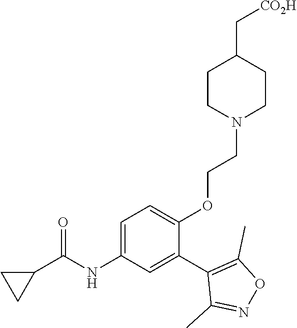 [SMILES: Cc1noc(C)c1-c1cc(NC(=O)C2CC2)ccc1OCCN1CCC(CC(=O)O)CC1] | 2-[1-[2-[4-(cyclopropanecarbonylamino)-2-(3,5- dimethylisoxazol-4-yl)phenoxy]ethyl]-4-piperidyl]acetic acid |
| 159 | 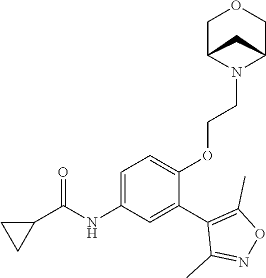 [SMILES: Cc1noc(C)c1-c1cc(NC(=O)C2CC2)ccc1OCCN1[C@@H]2COC[C@H]1C2] | N-[3-(3,5-dimethylisoxazol-4-yl)-4-[2-[(1R,5S)-3-oxa-6- azabicyclo[3.1.1]heptan-6- yl]ethoxy]phenyl]cyclopropanecarboxamide |
| 160 | 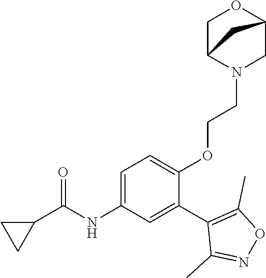 [SMILES: Cc1noc(C)c1-c1cc(NC(=O)C2CC2)ccc1OCCN1C[C@@H]2C[C@H]1CO2] | N-(4-(2-((1S,4S)-2-oxa-5-azabicyclo[2.2.1]heptan-5-yl)ethoxy)- 3-(3,5-dimethylisoxazol-4-yl)phenyl)cyclopropanecarboxamide |
| 161 | 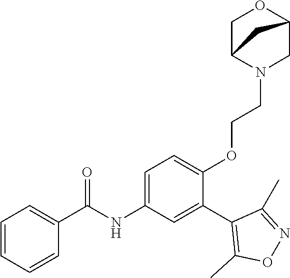 [SMILES: Cc1noc(C)c1-c1cc(NC(=O)c2ccccc2)ccc1OCCN1C[C@@H]2C[C@H]1CO2] | N-[3-(3,5-dimethylisoxazol-4-yl)-4-[2-[(1S,4S)-2-oxa-5- azabicyclo[2.2.1]heptan-5-yl]ethoxy]phenyl]benzamide |
| 162 | 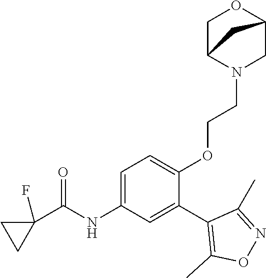 [SMILES: Cc1noc(C)c1-c1cc(NC(=O)C2(F)CC2)ccc1OCCN1C[C@@H]2C[C@H]1CO2] | N-[3-(3,5-dimethylisoxazol-4-yl)-4-[2-[(1S,4S)-2-oxa-5- azabicyclo[2.2.1]heptan-5-yl]ethoxy]phenyl]-1-fluoro- cyclopropanecarboxamide |
| 163 | 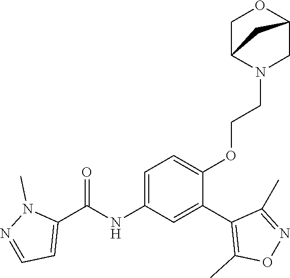 [SMILES: Cc1noc(C)c1-c1cc(NC(=O)c2ccnn2C)ccc1OCCN1C[C@@H]2C[C@H]1CO2] | N-[3-(3,5-dimethylisoxaozl-4-yl)-4-[2-[(1S,4S)-2-oxa-5- azabicyclo[2.2.1]heptan-5-yl]ethoxy]phenyl]-2-methyl- pyrazole-3-carboxamide |
| 164 | 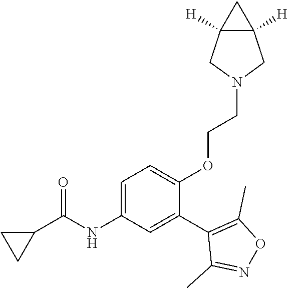 [SMILES: Cc1noc(C)c1-c1cc(NC(=O)C2CC2)ccc1OCCN1C[C@H]2C[C@H]2C1] | N-[4-[2-[(1R,5S)-3-azabicyclo[3.1.0]hexan-3-yl]ethoxy]-3-(3,5- dimethylisoxazol-4-yl)phenyl]cyclopropanecarboxamide |
| 165 | 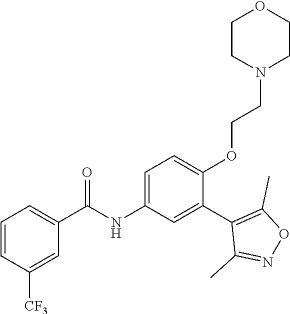 [SMILES: Cc1noc(C)c1-c1cc(NC(=O)c2cccc(C(F)(F)F)c2)ccc1OCCN1CCOCC1] | N-[3-(3,5-dimethylisoxazol-4-yl)-4-(2- morpholinoethoxy)phenyl]-3-(trifluoromethyl)benzamide |
| 166 | 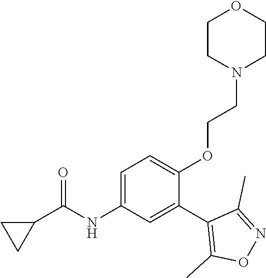 [SMILES: Cc1noc(C)c1-c1cc(NC(=O)C2CC2)ccc1OCCN1CCOCC1] | N-[3-(3,5-dimethylisoxazol-4-yl)-4-(2- morpholinoethoxy)phenyl]cyclopropanecarboxamide |
| 167 | 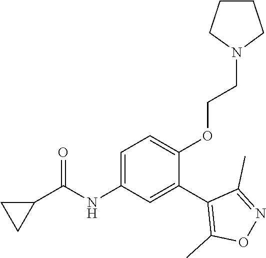 [SMILES: Cc1noc(C)c1-c1cc(NC(=O)C2CC2)ccc1OCCN1CCCC1] | N-[3-(3,5-dimethylisoxazol-4-yl)-4-(2-pyrrolidin-1- ylethoxy)phenyl]cyclopropanecarboxamide |
| 168 | 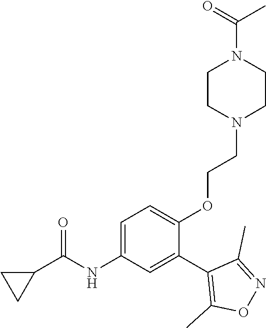 [SMILES: CC(=O)N1CCN(CCOc2ccc(NC(=O)C3CC3)cc2-c2c(C)noc2C)CC1] | N-[4-[2-(4-acetylpiperazin-1-yl)ethoxy]-3-(3,5- dimethylisoxazol-4-yl)phenyl]cyclopropanecarboxamide |
| 169 | 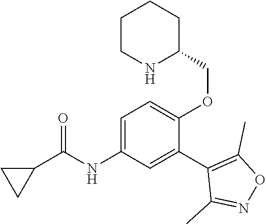 [SMILES: Cc1noc(C)c1-c1cc(NC(=O)C2CC2)ccc1OC[C@H]1CCCCN1] | N-[3-(3,5-dimethylisoxazol-4-yl)-4-[[(2R)-2- piperidyl]methoxy]phenyl]cyclopropanecarboxamide |
| 170 | 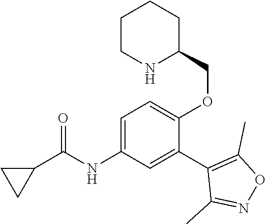 [SMILES: Cc1noc(C)c1-c1cc(NC(=O)C2CC2)ccc1OC[C@@H]1CCCCN1] | (S)-N-(3-(3,5-dimethylisoxazol-4-yl)-4-(piperidin-2- ylmethoxy)phenyl)cyclopropanecarboxamide |
| 171 | 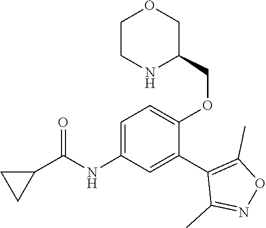 [SMILES: Cc1noc(C)c1-c1cc(NC(=O)C2CC2)ccc1OC[C@@H]1COCCN1] | (S)-N-(3-(3,5-dimethylisoxazol-4-yl)-4-(morpholin-3- ylmethoxy)phenyl)cyclopropanecarboxamide |
| 172 | 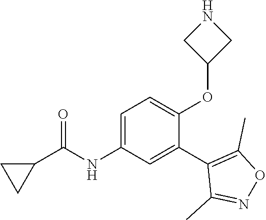 [SMILES: Cc1noc(C)c1-c1cc(NC(=O)C2CC2)ccc1OC1CNC1] | N-(4-(azetidin-3-yloxy)-3-(3,5-dimethylisoxazol-4- yl)phenyl)cyclopropanecarboxamide |
| 173 | 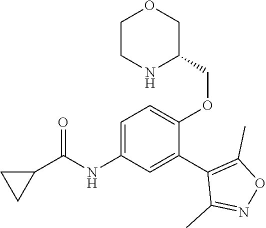 [SMILES: Cc1noc(C)c1-c1cc(NC(=O)C2CC2)ccc1OC[C@H]1COCCN1] | (R)-N-(3-(3,5-dimethylisoxazol-4-yl)-4-(morpholin-3- ylmethoxy)phenyl)cyclopropanecarboxamide |
| 174 | 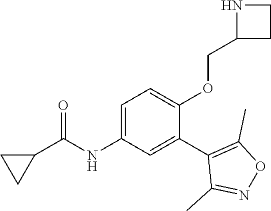 [SMILES: Cc1noc(C)c1-c1cc(NC(=O)C2CC2)ccc1OCC1CCN1] | N-[4-(azetidin-2-ylmethoxy)-3-(3,5-dimethylisoxazol-4- yl)phenyl]cyclopropanecarboxamide |
| 175 | 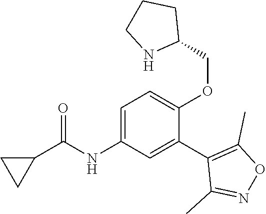 [SMILES: Cc1noc(C)c1-c1cc(NC(=O)C2CC2)ccc1OC[C@H]1CCCN1] | (R)-N-(3-(3,5-dimethylisoxazol-4-yl)-4-(pyrrolidin-2- ylmethoxy)phenyl)cyclopropanecarboxamide |
| 176 | 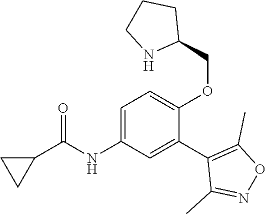 [SMILES: Cc1noc(C)c1-c1cc(NC(=O)C2CC2)ccc1OC[C@@H]1CCCN1] | (S)-N-(3-(3,5-dimethylisoxazol-4-yl)-4-(pyrrolidin-2- ylmethoxy)phenyl)cyclopropanecarboxamide |
| 177 | 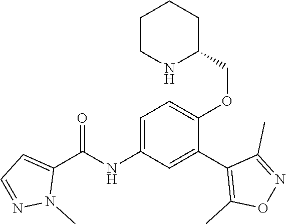 [SMILES: Cc1noc(C)c1-c1cc(NC(=O)c2ccnn2C)ccc1OC[C@H]1CCCCN1] | N-[3-(3,5-dimethylisoxazol-4-yl)-4-[[(2R)-2- piperidyl]methoxy]phenyl]-2-methyl-pyridinerazole-3- carboxamide |
| 178 | 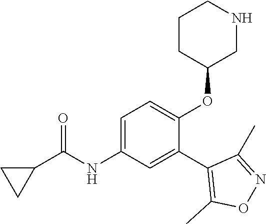 [SMILES: Cc1noc(C)c1-c1cc(NC(=O)C2CC2)ccc1O[C@H]1CCCNC1] | N-[3-(3,5-dimethylisoxazol-4-yl)-4-[[(3S)-3- piperidyl]oxy]phenyl]cyclopropanecarboxamide |
| 179 | 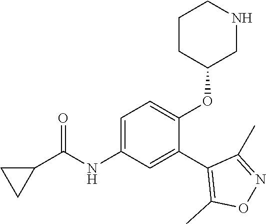 [SMILES: Cc1noc(C)c1-c1cc(NC(=O)C2CC2)ccc1O[C@@H]1CCCNC1] | (R)-N-(3-(3,5-dimethylisoxazol-4-yl)-4-(piperidin-3- yloxy)phenyl)cyclopropanecarboxamide |
| 180 | 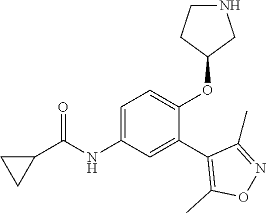 [SMILES: Cc1noc(C)c1-c1cc(NC(=O)C2CC2)ccc1O[C@H]1CCNC1] | (S)-N-(3-(3,5-dimethylisoxazol-4-yl)-4-(pyrrolidin-3- yloxy)phenyl)cyclopropanecarboxamide |
| 181 | 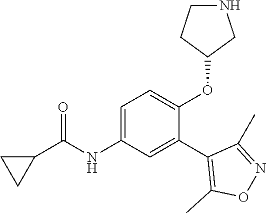 | (R)-N-(3-(3,5-dimethylisoxazol-4-yl)-4-(pyrrolidin-3- yloxy)phenyl)cyclopropanecarboxamide |
| 182 | 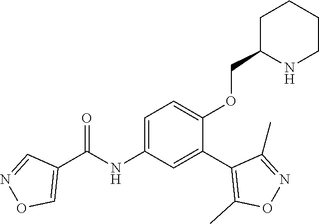 [SMILES: Cc1noc(C)c1-c1cc(NC(=O)c2cnoc2)ccc1OC[C@H]1CCCCN1] | N-[3-(3,5-dimethylisoxazol-4-yl)-4-[[(2R)-2- piperidyl]methoxy]phenyl]isoxazole-4-carboxamide |
| 183 | 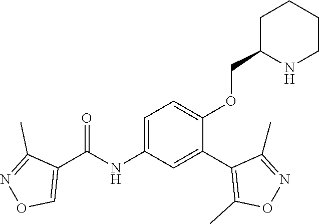 [SMILES: Cc1nocc1C(=O)Nc1ccc(OC[C@H]2CCCCN2)c(-c2c(C)noc2C)c1] | (R)-N-(3-(3,5-dimethylisoxazol-4-yl)-4-(piperidin-2- ylmethoxy)phenyl)-3-methylisoxazole-4-carboxamide |
| 184 | 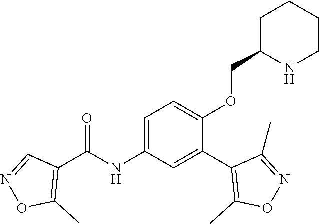 [SMILES: Cc1noc(C)c1-c1cc(NC(=O)c2cnoc2C)ccc1OC[C@H]1CCCCN1] | (R)-N-(3-(3,5-dimethylisoxazol-4-yl)-4-(piperidin-2- ylmethoxy)phenyl)-5-methylisoxazole-4-carboxamide |
| 185 | 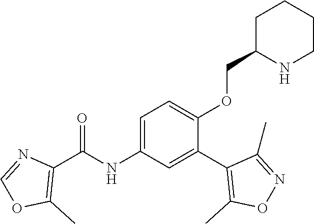 [SMILES: Cc1noc(C)c1-c1cc(NC(=O)c2ncoc2C)ccc1OC[C@H]1CCCCN1] | (R)-N-(3-(3,5-dimethylisoxazol-4-yl)-4-(piperidin-2- ylmethoxy)phenyl)-5-methyloxazole-4-carboxamide |
| 186 | 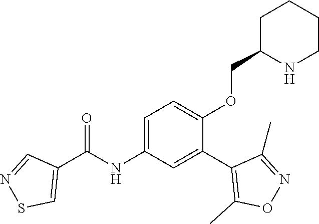 [SMILES: Cc1noc(C)c1-c1cc(NC(=O)c2cnsc2)ccc1OC[C@H]1CCCCN1] | (R)-N-(3-(3,5-dimethylisoxazol-4-yl)-4-(piperidin-2- ylmethoxy)phenyl)isothiazole-4-carboxamide |
| 187 | 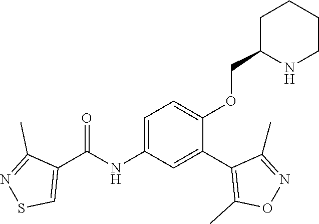 [SMILES: Cc1nscc1C(=O)Nc1ccc(OC[C@H]2CCCCN2)c(-c2c(C)noc2C)c1] | (R)-N-(3-(3,5-dimethylisoxazol-4-yl)-4-(piperidin-2- ylmethoxy)phenyl)-3-methylisothiazole-4-carboxamide |
| 188 | 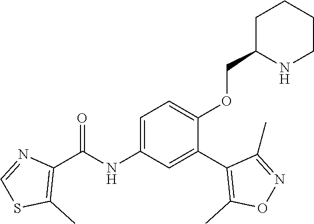 [SMILES: Cc1noc(C)c1-c1cc(NC(=O)c2ncsc2C)ccc1OC[C@H]1CCCCN1] | (R)-N-(3-(3,5-dimethylisoxazol-4-yl)-4-(piperidin-2- ylmethoxy)phenyl)-5-methylthiazole-4-carboxamide |
| 189 | 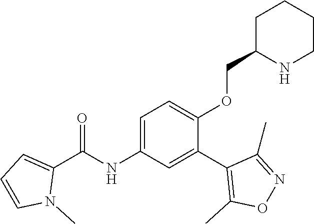 [SMILES: Cc1noc(C)c1-c1cc(NC(=O)c2cccn2C)ccc1OC[C@H]1CCCCN1] | (R)-N-(3-(3,5-dimethylisoxazol-4-yl)-4-(piperidin-2- ylmethoxy)phenyl)-1-methyl-1H-pyrrole-2-carboxamide |
| 190 | 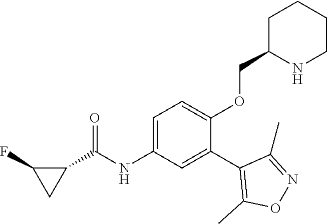 [SMILES: Cc1noc(C)c1-c1cc(NC(=O)[C@@H]2C[C@H]2F)ccc1OC[C@H]1CCCCN1] | (1S,2R)-N-(3-(3,5-dimethylisoxazol-4-yl)-4-(((R)-piperidin-2- yl)methoxy)phenyl)-2-fluorocyclopropane-1-carboxamide |
| 191 | 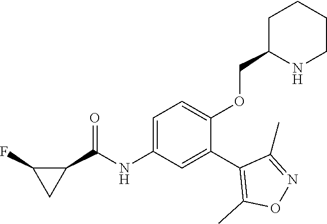 [SMILES: Cc1noc(C)c1-c1cc(NC(=O)[C@H]2C[C@H]2F)ccc1OC[C@H]1CCCCN1] | (1R,2R)-N-(3-(3,5-dimethylisoxazol-4-yl)-4-(((R)-piperidin-2- yl)methoxy)phenyl)-2-fluorocyclopropane-1-carboxamide |
| 192 | 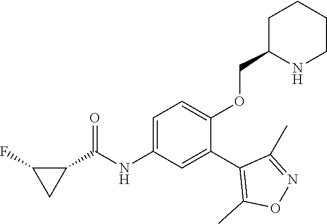 [SMILES: Cc1noc(C)c1-c1cc(NC(=O)[C@@H]2C[C@@H]2F)ccc1OC[C@H]1CCCCN1] | (1S,2S)-N-(3-(3,5-dimethylisoxazol-4-yl)-4-(((R)-piperidin-2- yl)methoxy)phenyl)-2-fluorocyclopropane-1-carboxamide |
| 193 | 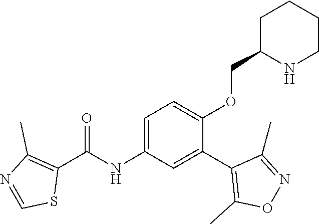 [SMILES: Cc1ncsc1C(=O)Nc1ccc(OC[C@H]2CCCCN2)c(-c2c(C)noc2C)c1] | (R)-N-(3-(3,5-dimethylisoxazol-4-yl)-4-(piperidin-2- ylmethoxy)phenyl)-4-methylthiazole-5-carboxamide |
| 194 | 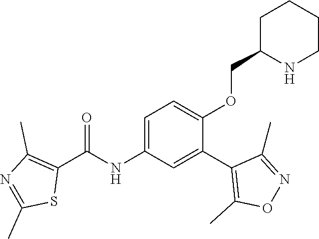 [SMILES: Cc1nc(C)c(C(=O)Nc2ccc(OC[C@H]3CCCCN3)c(-c3c(C)noc3C)c2)s1] | (R)-N-(3-(3,5-dimethylisoxazol-4-yl)-4-(piperidin-2- ylmethoxy)phenyl)-2,4-dimethylthiazole-5-carboxamide |
| 195 | 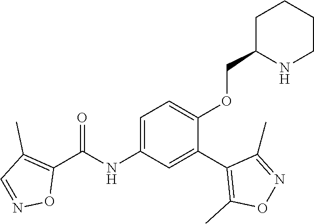 [SMILES: Cc1cnoc1C(=O)Nc1ccc(OC[C@H]2CCCCN2)c(-c2c(C)noc2C)c1] | (R)-N-(3-(3,5-dimethylisoxazol-4-yl)-4-(piperidin-2- ylmethoxy)phenyl)-4-methylisoxazole-5-carboxamide |
| 196 | 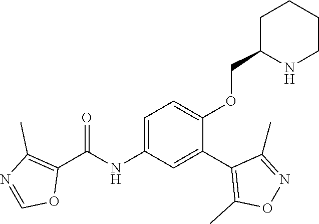 [SMILES: Cc1ncoc1C(=O)Nc1ccc(OC[C@H]2CCCCN2)c(-c2c(C)noc2C)c1] | (R)-N-(3-(3,5-dimethylisoxazol-4-yl)-4-(piperidin-2- ylmethoxy)phenyl)-4-methyloxazole-5-carboxamide |
| 197 | 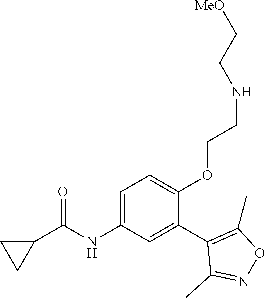 [SMILES: COCCNCCOc1ccc(NC(=O)C2CC2)cc1-c1c(C)noc1C] | N-[3-(3,5-dimethylisoxazol-4-yl)-4-[2-(2- methoxyethylamino)ethoxy]phenyl]cyclopropanecarboxamide |
| 198 | 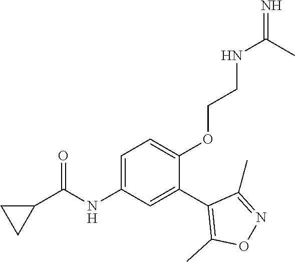 [SMILES: CC(=N)NCCOc1ccc(NC(=O)C2CC2)cc1-c1c(C)noc1C] | N-(4-(2-acetimidamido)-3-(3,5-dimethylisoxazol-4- yl)phenyl)cyclopropanecarboxamide |
| 199 | 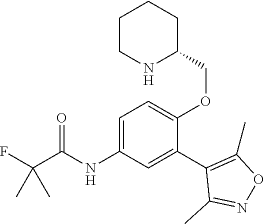 [SMILES: Cc1noc(C)c1-c1cc(NC(=O)C(C)(C)F)ccc1OC[C@H]1CCCCN1] | N-[3-(3,5-dimethylisoxazol-4-yl)-4-[[(2R)-2- piperidyl]methoxy]phenyl]-2-fluoro-2-methyl-propanamide |
| 200 | 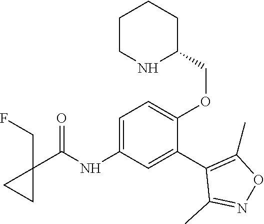 [SMILES: Cc1noc(C)c1-c1cc(NC(=O)C2(CF)CC2)ccc1OC[C@H]1CCCCN1] | (R)-N-(3-(3,5-dimethylisoxazol-4-yl)-4-(piperidin-2- ylmethoxy)phenyl)-1-(fluoromethyl)cyclopropane-1- carboxamide |
| 201 | 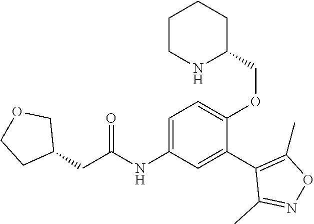 [SMILES: Cc1noc(C)c1-c1cc(NC(=O)C[C@@H]2CCOC2)ccc1OC[C@H]1CCCCN1] | N-(3-(3,5-dimethylisoxazol-4-yl)-4-(((R)-piperidin-2- yl)methoxy)phenyl)-2-((S)-tetrahydrofuran-3-yl)acetamide |
| 202 | 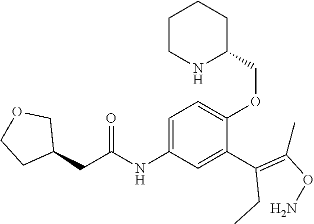 [SMILES: CC/C(=C(/C)ON)c1cc(NC(=O)C[C@H]2CCOC2)ccc1OC[C@H]1CCCCN1] | N-(3-((E)-2-(aminooxy)pent-2-en-3-yl)-4-(((R)-piperidin-2- yl)methoxy)phenyl)-2-((R)-tetrahydrofuran-3-yl)acetamide |
| 203 | 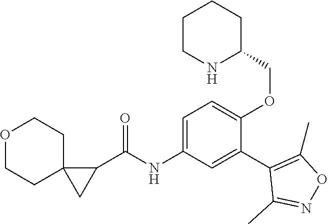 [SMILES: Cc1noc(C)c1-c1cc(NC(=O)C2CC23CCOCC3)ccc1OC[C@H]1CCCCN1] | N-(3-(3,5-dimethylisoxazol-4-yl)-4-(((R)-piperidin-2- yl)methoxy)phenyl)-6-oxaspiro[2.5]octane-1-carboxamide |
| 204 | 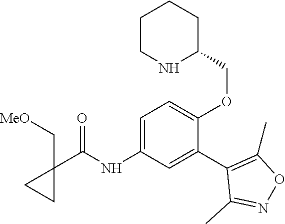 [SMILES: COCC1(C(=O)Nc2ccc(OC[C@H]3CCCCN3)c(-c3c(C)noc3C)c2)CC1] | (R)-N-(3-(3,5-dimethylisoxazol-4-yl)-4-(piperidin-2- ylmethoxy)phenyl)-1-(methoxymethyl)cyclopropane-1- carboxamide |
| 205 | 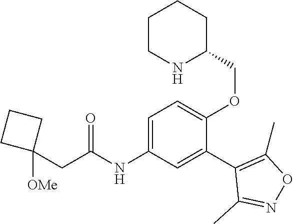 [SMILES: COC1(CC(=O)Nc2ccc(OC[C@H]3CCCCN3)c(-c3c(C)noc3C)c2)CCC1] | (R)-N-(3-(3,5-dimethylisoxazol-4-yl)-4-(piperidin-2- ylmethoxy)phenyl)-2-(1-methoxycyclobutyl)acetamide |
| 206 | 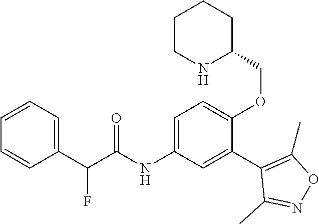 [SMILES: Cc1noc(C)c1-c1cc(NC(=O)C(F)c2ccccc2)ccc1OC[C@H]1CCCCN1] | N -(3-(3,5-dimethylisoxazol-4-yl)-4-(((R)-piperidin-2- yl)methoxy)phenyl)-2-fluoro-2-phenylacetamide |
| 207 | 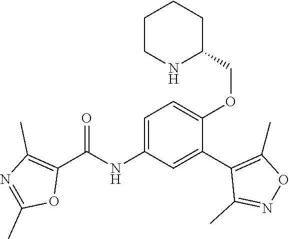 [SMILES: Cc1nc(C)c(C(=O)Nc2ccc(OC[C@H]3CCCCN3)c(-c3c(C)noc3C)c2)o1] | (R)-N-(3-(3,5-dimethylisoxazol-4-yl)-4-(piperidin-2- ylmethoxy)phenyl)-2,4-dimethyloxazole-5-carboxamide |
| 208 | 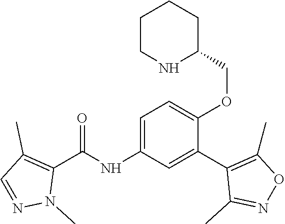 [SMILES: Cc1cnn(C)c1C(=O)Nc1ccc(OC[C@H]2CCCCN2)c(-c2c(C)noc2C)c1] | (R)-N-(3-(3,5-dimethylisoxazol-4-yl)-4-(piperidin-2- ylmethoxy)phenyl)-1,4-dimethyl-1H-pyrazole-5-carboxamide |
| 209 | 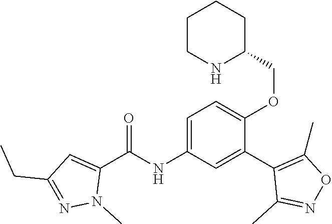 [SMILES: CCc1cc(C(=O)Nc2ccc(OC[C@H]3CCCCN3)c(-c3c(C)noc3C)c2)n(C)n1] | (R)-N-(3-(3,5-dimethylisoxazol-4-yl)-4-(piperidin-2- ylmethoxy)phenyl)-3-ethyl-1-methyl-1H-pyrazole-5- carboxamide |
| 210 | 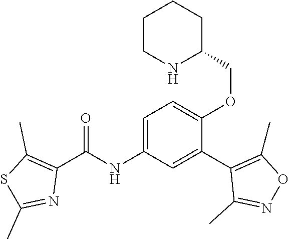 [SMILES: Cc1nc(C(=O)Nc2ccc(OC[C@H]3CCCCN3)c(-c3c(C)noc3C)c2)c(C)s1] | (R)-N-(3-(3,5-dimethylisoxazol-4-yl)-4-(piperidin-2- ylmethoxy)phenyl)-2,5-dimethylthiazole-4-carboxamide |
| 211 | 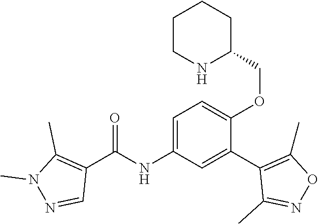 [SMILES: Cc1noc(C)c1-c1cc(NC(=O)c2cnn(C)c2C)ccc1OC[C@H]1CCCCN1] | (R)-N-(3-(3,5-dimethylisoxazol-4-yl)-4-(piperidin-2- ylmethoxy)phenyl)-1,5-dimethyl-1H-pyrazole-4-carboxamide |
| 212 | 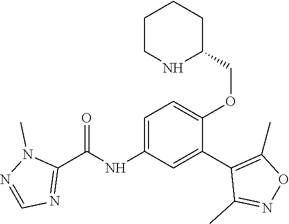 [SMILES: Cc1noc(C)c1-c1cc(NC(=O)c2ncnn2C)ccc1OC[C@H]1CCCCN1] | (R)-N-(3-(3,5-dimethylisoxazol-4-yl)-4-(piperidin-2- ylmethoxy)phenyl)-1-methyl-1H-1,2,4-triazole-5-carboxamide |
| 213 | 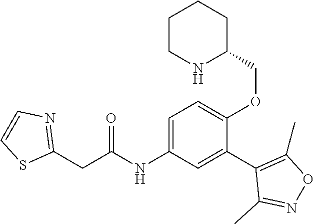 [SMILES: Cc1noc(C)c1-c1cc(NC(=O)Cc2nccs2)ccc1OC[C@H]1CCCCN1] | (R)-N-(3-(3,5-dimethylisoxazol-4-yl)-4-(piperidin-2- ylmethoxy)phenyl)-2-(thiazol-2-yl)acetamide |
| 214 | 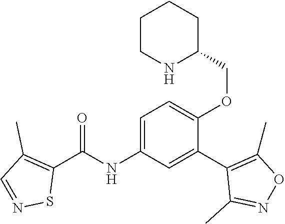 [SMILES: Cc1cnsc1C(=O)Nc1ccc(OC[C@H]2CCCCN2)c(-c2c(C)noc2C)c1] | (R)-N-(3-(3,5-dimethylisoxazol-4-yl)-4-(piperidin-2- ylmethoxy)phenyl)-4-methylisothiazole-5-carboxamide |
| 215 | 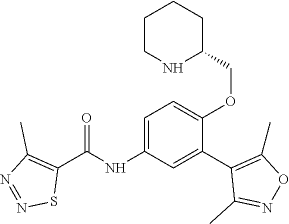 [SMILES: Cc1nnsc1C(=O)Nc1ccc(OC[C@H]2CCCCN2)c(-c2c(C)noc2C)c1] | (R)-N-(3-(3,5-dimethylisoxazol-4-yl)-4-(piperidin-2- ylmethoxy)phenyl)-4-methyl-1,2,3-thiadiazole-5-carboxamide |
| 216 | 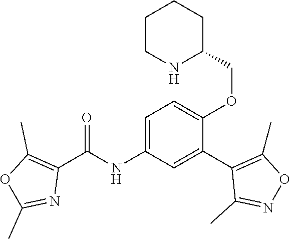 [SMILES: Cc1nc(C(=O)Nc2ccc(OC[C@H]3CCCCN3)c(-c3c(C)noc3C)c2)c(C)o1] | (R)-N-(3-(3,5-dimethylisoxazol-4-yl)-4-(piperidin-2- ylmethoxy)phenyl)-2,5-dimethyloxazole-4-carboxamide |
| 217 | 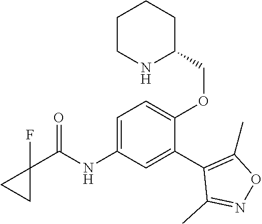 [SMILES: Cc1noc(C)c1-c1cc(NC(=O)C2(F)CC2)ccc1OC[C@H]1CCCCN1] | (R)-N-(3-(3,5-dimethylisoxazol-4-yl)-4-(piperidin-2- ylmethoxy)phenyl)-1-fluorocyclopropane-1-carboxamide |
| 218 | 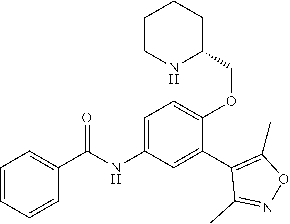 [SMILES: Cc1noc(C)c1-c1cc(NC(=O)c2ccccc2)ccc1OC[C@H]1CCCCN1] | (R)-N-(3-(3,5-dimethylisoxazol-4-yl)-4-(piperidin-2- ylmethoxy)phenyl)benzamide |
| 219 | 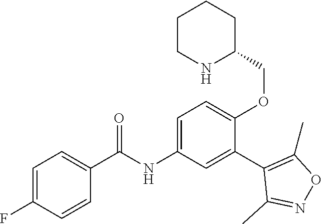 [SMILES: Cc1noc(C)c1-c1cc(NC(=O)c2ccc(F)cc2)ccc1OC[C@H]1CCCCN1] | (R)-N-(3-(3,5-dimethylisoxazol-4-yl)-4-(piperidin-2- ylmethoxy)phenyl)-4-fluorobenzamide |
| 220 | 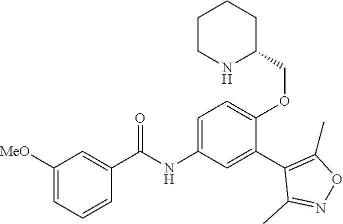 [SMILES: COc1cccc(C(=O)Nc2ccc(OC[C@H]3CCCCN3)c(-c3c(C)noc3C)c2)c1] | (R)-N-(3-(3,5-dimethylisoxazol-4-yl)-4-(piperidin-2- ylmethoxy)phenyl)-3-methoxybenzamide |
| 221 | 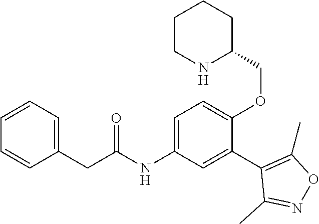 [SMILES: Cc1noc(C)c1-c1cc(NC(=O)Cc2ccccc2)ccc1OC[C@H]1CCCCN1] | (R)-N-(3-(3,5-dimethylisoxazol-4-yl)-4-(piperidin-2- ylmethoxy)phenyl)-2-phenylacetamide |
| 222 | 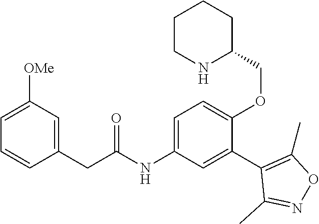 [SMILES: COc1cccc(CC(=O)Nc2ccc(OC[C@H]3CCCCN3)c(-c3c(C)noc3C)c2)c1] | (R)-N-(3-(3,5-dimethylisoxazol-4-yl)-4-(piperidin-2- ylmethoxy)phenyl)-2-(3-methoxyphenyl)acetamide |
| 223 | 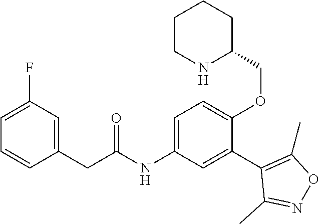 [SMILES: Cc1noc(C)c1-c1cc(NC(=O)Cc2cccc(F)c2)ccc1OC[C@H]1CCCCN1] | (R)-N-(3-(3,5-dimethylisoxazol-4-yl)-4-(piperidin-2- ylmethoxy)phenyl)-2-(3-fluorophenyl)acetamide |
| 224 | 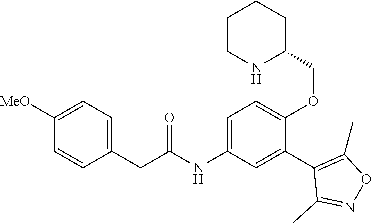 [SMILES: COc1ccc(CC(=O)Nc2ccc(OC[C@H]3CCCCN3)c(-c3c(C)noc3C)c2)cc1] | (R)-N-(3-(3,5-dimethylisoxazol-4-yl)-4-(piperidin-2- ylmethoxy)phenyl)-2-(4-methoxyphenyl)acetamide |
| 225 | 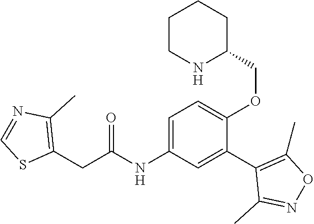 [SMILES: Cc1ncsc1CC(=O)Nc1ccc(OC[C@H]2CCCCN2)c(-c2c(C)noc2C)c1] | (R)-N-(3-(3,5-dimethylisoxazol-4-yl)-4-(piperidin-2- ylmethoxy)phenyl)-2-(4-methylthiazol-5-yl)acetamide |
| 226 | 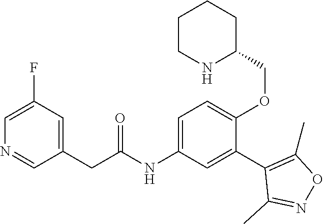 [SMILES: Cc1noc(C)c1-c1cc(NC(=O)Cc2cncc(F)c2)ccc1OC[C@H]1CCCCN1] | (R)-N-(3-(3,5-dimethylisoxazol-4-yl)-4-(piperidin-2- ylmethoxy)phenyl)-2-(5-fluoropyridin-3-yl)acetamide |
| 227 | 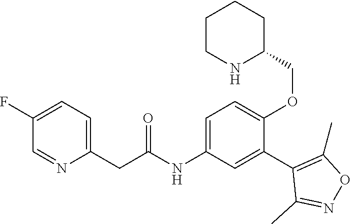 [SMILES: Cc1noc(C)c1-c1cc(NC(=O)Cc2ccc(F)cn2)ccc1OC[C@H]1CCCCN1] | (R)-N-(3-(3,5-dimethylisoxazol-4-yl)-4-(piperidin-2- ylmethoxy)phenyl)-2-(5-fluoropyridin-2-yl)acetamide |
| 228 | 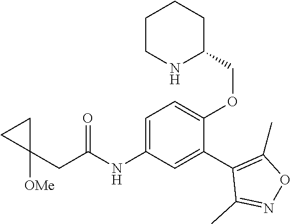 [SMILES: COC1(CC(=O)Nc2ccc(OC[C@H]3CCCCN3)c(-c3c(C)noc3C)c2)CC1] | (R)-N-(3-(3,5-dimethylisoxazol-4-yl)-4-(piperidin-2- ylmethoxy)phenyl)-2-(1-methoxycyclopropyl)acetamide |
| 229 | 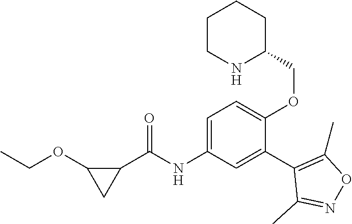 [SMILES: CCOC1CC1C(=O)Nc1ccc(OC[C@H]2CCCCN2)c(-c2c(C)noc2C)c1] | N-(3-(3,5-dimethylisoxazol-4-yl)-4-(((R)-piperidin-2- yl)methoxy)phenyl)-2-ethoxycyclopropane-1-carboxamide |
| 230 | 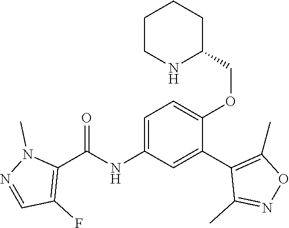 [SMILES: Cc1noc(C)c1-c1cc(NC(=O)c2c(F)cnn2C)ccc1OC[C@H]1CCCCN1] | (R)-N-(3-(3,5-dimethylisoxazol-4-yl)-4-(piperiidn-2- ylmethoxy)phenyl)-4-fluoro-1-methyl-1H-pyrazole-5- carboxamide |
| 231 | 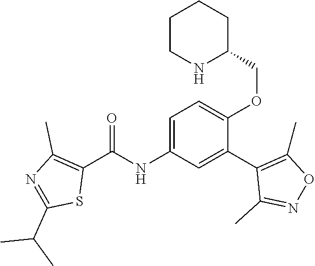 [SMILES: Cc1nc(C(C)C)sc1C(=O)Nc1ccc(OC[C@H]2CCCCN2)c(-c2c(C)noc2C)c1] | (R)-N-(3-(3,5-dimethylisoxazol-4-yl)-4-(piperidin-2- ylmethoxy)phenyl)-2-isopropyl-4-methylthiazole-5- carboxamide |
| 232 | 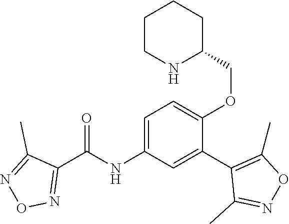 [SMILES: Cc1nonc1C(=O)Nc1ccc(OC[C@H]2CCCCN2)c(-c2c(C)noc2C)c1] | (R)-N-(3-(3,5-dimethylisoxazol-4-yl)-4-(piperidin-2- ylmethoxy)phenyl)-4-methyl-1,2,5-oxadiazole-3-carboxamide |
| 233 | 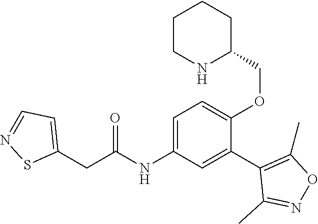 [SMILES: Cc1noc(C)c1-c1cc(NC(=O)Cc2ccns2)ccc1OC[C@H]1CCCCN1] | (R)-N-(3-(3,5-dimethylisoxazol-4-yl)-4-(piperidin-2- ylmethoxy)phenyl)-2-(isothiazol-5-yl)acetamide |
| 234 | 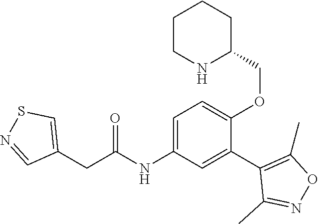 [SMILES: Cc1noc(C)c1-c1cc(NC(=O)Cc2cnsc2)ccc1OC[C@H]1CCCCN1] | (R)-N-(3-(3,5-dimethylisoxazol-4-yl)-4-(piperidin-2- ylmethoxy)phenyl)-2-(isothiazol-4-yl)acetamide |
| 235 | 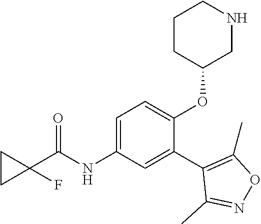 [SMILES: Cc1noc(C)c1-c1cc(NC(=O)C2(F)CC2)ccc1O[C@@H]1CCCNC1] | (R)-N-(3-(3,5-dimethylisoxazol-4-yl)-4-(piperidin-3- yloxy)phenyl)-1-fluorocyclopropane-1-carboxamide |
| 236 | 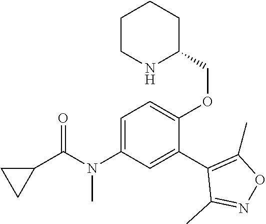 [SMILES: Cc1noc(C)c1-c1cc(N(C)C(=O)C2CC2)ccc1OC[C@H]1CCCCN1] | N-[3-(3,5-dimethylisoxazol-4-yl)-4-[[(2R)-2- piperidyl]methoxy]phenyl]-N-methyl-cyclopropanecarboxamide |
| 237 | 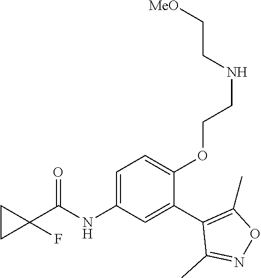 [SMILES: COCCNCCOc1ccc(NC(=O)C2(F)CC2)cc1-c1c(C)noc1C] | N-[3-(3,5-dimethylisoxazol-4-yl)-4-[2-(2- methoxyethylamino)ethoxy]phenyl]-1-fluoro- cyclopropanecarboxamide |
| 238 | 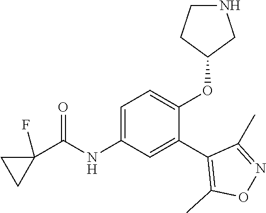 [SMILES: Cc1noc(C)c1-c1cc(NC(=O)C2(F)CC2)ccc1O[C@@H]1CCNC1] | (R)-N-(3-(3,5-dimethylisoxazol-4-yl)-4-(pyrrolidin-3- yloxy)phenyl)-1-fluorocyclopropane-1-carboxamide |
| 239 | 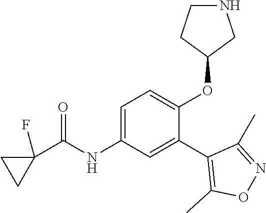 [SMILES: Cc1noc(C)c1-c1cc(NC(=O)C2(F)CC2)ccc1O[C@H]1CCNC1] | (S)-N-(3-(3,5-dimethylisoxazol-4-yl)-4-(pyrrolidin-3- yloxy)phenyl)-1-fluorocyclopropane-1-carboxamide |
| 240 | 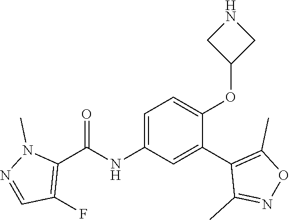 [SMILES: Cc1noc(C)c1-c1cc(NC(=O)c2c(F)cnn2C)ccc1OC1CNC1] | N-(4-(azetidin-3-yloxy)-3-(3,5-dimethylisoxazol-4-yl)phenyl)- 4-fluoro-1-methyl-1H-pyrazole-5-carboxamide |
| 241 | 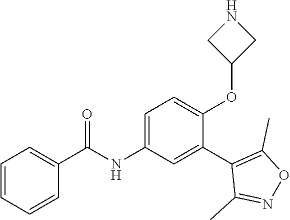 [SMILES: Cc1noc(C)c1-c1cc(NC(=O)c2ccccc2)ccc1OC1CNC1] | N-(4-(azetidin-3-yloxy)-3-(3,5-dimethylisoxazol-4- yl)phenyl)benzamide |
| 242 | 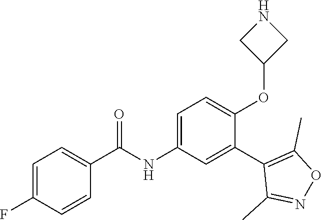 [SMILES: Cc1noc(C)c1-c1cc(NC(=O)c2ccc(F)cc2)ccc1OC1CNC1] | N-(4-(azetidin-3-yloxy)-3-(3,5-dimethylisoxazol-4-yl)phenyl)- 4-fluorobenzamide |
| 243 | 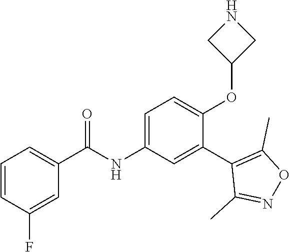 [SMILES: Cc1noc(C)c1-c1cc(NC(=O)c2cccc(F)c2)ccc1OC1CNC1] | N-(4-(azetidin-3-yloxy)-3-(3,5-dimethylisoxazol-4-yl)phenyl)- 3-fluorobenzamide |
| 244 | 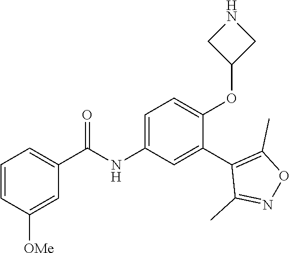 [SMILES: COc1cccc(C(=O)Nc2ccc(OC3CNC3)c(-c3c(C)noc3C)c2)c1] | N-(4-(azetidin-3-yloxy)-3-(3,5-dimethylisoxazol-4-yl)phenyl)- 3-methoxybenzamide |
| 245 | 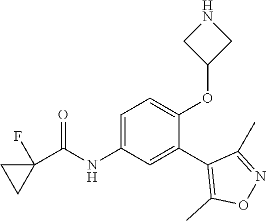 [SMILES: Cc1noc(C)c1-c1cc(NC(=O)C2(F)CC2)ccc1OC1CNC1] | N-(4-(azetidin-3-yloxy)-3-(3,5-dimethylisoxazol-4-yl)phenyl)- 1-fluorocyclopropane-1-carboxamide |

In some embodiments, the compound having the structure selected from the group consisting of a compound of Table 1 herein, or a pharmaceutically acceptable salt thereof, wherein the salt is selected from the group consisting of salts of acetic acid, benzenesulfonic acid, benzoic acid, citric acid, ethanesulfonic acid, fumaric acid, hydrochloric acid, maleic acid, malic acid, methanesulfonic acid, nitric acid, oxalic acid, phosphoric acid, succinic acid, sulfuric acid, tartaric acid, and p-toluenesulfonic acid.

In some embodiments, the compound having the structure selected from the group consisting of a compound of Table 1 herein, or a pharmaceutically acceptable salt thereof, wherein the salt is selected from the group consisting of salts of acetic acid, benzenesulfonic acid, benzoic acid, citric acid, ethanesulfonic acid, fumaric acid, hydrochloric acid, maleic acid, malic acid, methanesulfonic acid, nitric acid, oxalic acid, phosphoric acid, succinic acid, sulfuric acid, tartaric acid, and p-toluenesulfonic acid.

In some embodiments, the compound having the structure selected from the group consisting of a compound of Table 1 herein, or a pharmaceutically acceptable salt thereof, wherein the salt is selected from the group consisting of salts of benzenesulfonic acid, citric acid, fumaric acid, hydrochloric acid, methanesulfonic acid, phosphoric acid, succinic acid, sulfuric acid and p-toluenesulfonic acid.

In some embodiments, the compound having the structure selected from the group consisting of a compound of Table 1 herein, or a pharmaceutically acceptable salt thereof, wherein the salt is selected from the group consisting of salts of benzenesulfonic acid, citric acid, phosphoric acid, succinic acid, and p-toluenesulfonic acid.

In some embodiments, the compound having the structure selected from the group consisting of a compound of Table 1 herein, or a pharmaceutically acceptable salt thereof, wherein the salt is selected from the group consisting of salts of benzenesulfonic acid, citric acid, and succinic acid.

In one embodiment, the compound having the structure selected from the group consisting of a compound of Table 1 herein, or a pharmaceutically acceptable salt of acetic acid.

In one embodiment, the compound having the structure selected from the group consisting of a compound of Table 1 herein, or a pharmaceutically acceptable salt of benzenesulfonic acid.

In one embodiment, the compound having the structure selected from the group consisting of a compound of Table 1 herein, or a pharmaceutically acceptable salt of benzoic acid.

In one embodiment, the compound having the structure selected from the group consisting of a compound of Table 1 herein, or a pharmaceutically acceptable salt of citric acid.

In one embodiment, the compound having the structure selected from the group consisting of a compound of Table 1 herein, or a pharmaceutically acceptable salt of fumaric acid.

In one embodiment, the compound having the structure selected from the group consisting of a compound of Table 1 herein, or a pharmaceutically acceptable salt of hydrochloric acid.

In one embodiment, the compound having the structure selected from the group consisting of a compound of Table 1 herein, or a pharmaceutically acceptable salt of maleic acid.

In one embodiment, the compound having the structure selected from the group consisting of a compound of Table 1 herein, or a pharmaceutically acceptable salt of methanesulfonic acid.

In one embodiment, the compound having the structure selected from the group consisting of a compound of Table 1 herein, or a pharmaceutically acceptable salt of oxalic acid.

In one embodiment, the compound having the structure selected from the group consisting of a compound of Table 1 herein, or a pharmaceutically acceptable salt of phosphoric acid.

In one embodiment, the compound having the structure selected from the group consisting of a compound of Table 1 herein, or a pharmaceutically acceptable salt of succinic acid.

In one embodiment, the compound having the structure selected from the group consisting of a compound of Table 1 herein, or a pharmaceutically acceptable salt of sulfuric acid.

In one embodiment, the compound having the structure selected from the group consisting of a compound of Table 1 herein, or a pharmaceutically acceptable salt of p-toluenesulfonic acid.

Additionally, individual compounds and chemical genera of the present invention, for example those compounds found in TABLE 1 including diastereomers and enantiomers thereof, encompass all pharmaceutically acceptable salts, solvates, and particularly hydrates, thereof.

The compounds of the Formula (I) of the present invention may be prepared according to relevant published literature procedures that are used by one skilled in the art. Exemplary reagents and procedures for these reactions appear hereinafter in the working Examples. Protection and deprotection may be carried out by procedures generally known in the art (see, for example, Greene, T. W. and Wuts, P. G. M., Protecting Groups in Organic Synthesis, 3^{rd} Edition, 1999 [Wiley]; incorporated herein by reference in its entirety).

It is understood that the present invention embraces each diastereomer, each enantiomer and mixtures thereof of each compound and generic formulae disclosed herein just as if they were each individually disclosed with the specific stereochemical designation for each chiral carbon. Separation of the individual isomers (such as chiral HPLC, recrystallization of diastereomeric mixtures, and the like) or selective synthesis (such as enantiomeric selective syntheses, and the like) of the individual isomers is accomplished by application of various methods which are well known to practitioners in the art.

Indications and Methods of Prophylaxis and/or Treatment

The compounds of Formula (I), and pharmaceutically acceptable salts thereof, are useful as 5-HT_{2A} serotonin receptor modulators for the treatment of disorders associated with 5-HT_{2A} serotonin receptor expression and/or activity, such as cardiovascular disorders (for example, coronary artery disease, myocardial infarction, transient ischemic attack, angina, stroke, atrial fibrillation, platelet aggregation, and blood clot formation or symptoms thereof.

The modulators of 5-HT_{2A} receptor activity disclosed herein are believed to be useful in the treatment of several diseases and disorders, and in the amelioration of symptoms thereof. Without limitation, some of them include the following:

Antiplatelet agents (antiplatelets) are prescribed for a variety of conditions. For example, in coronary artery disease they are used to help prevent myocardial infarction or stroke in patients who are at risk of developing obstructive blood clots (e.g., coronary thrombosis).

In a myocardial infarction (heart attack), the heart muscle does not receive enough oxygen-rich blood as a result of a blockage in the coronary blood vessels. If taken while an attack is in progress or immediately afterward (preferably within 30 minutes), antiplatelets can reduce the damage to the heart.

A transient ischemic attack (“TIA” or “mini-stroke”) is a brief interruption of oxygen flow to the brain due to decreased blood flow through arteries, usually due to an obstructing blood clot. Antiplatelet drugs have been found to be effective in preventing TIAs.

Angina is a temporary and often recurring chest pain, pressure or discomfort caused by inadequate oxygen-rich blood flow (ischemia) to some parts of the heart. In patients with angina, antiplatelet therapy can reduce the effects of angina and the risk of myocardial infarction.

Stroke is an event in which the brain does not receive enough oxygen-rich blood, usually due to blockage of a cerebral blood vessel by a blood clot. In high-risk patients, taking antiplatelets regularly has been found to prevent the formation blood clots that cause first or second strokes.

Angioplasty is a catheter-based technique used to open arteries obstructed by a blood clot. Whether or not stenting is performed immediately after this procedure to keep the artery open, antiplatelets can reduce the risk of forming additional blood clots following the procedure(s).

Coronary bypass surgery is a surgical procedure in which an artery or vein is taken from elsewhere in the body and grafted to a blocked coronary artery, rerouting blood around the blockage and through the newly attached vessel. After the procedure, antiplatelets can reduce the risk of secondary blood clots.

Atrial fibrillation is the most common type of sustained irregular heart rhythm (arrythmia). Atrial fibrillation affects about two million Americans every year. In atrial fibrillation, the atria (the heart's upper chambers) rapidly fire electrical signals that cause them to quiver rather than contract normally. The result is an abnormally fast and highly irregular heartbeat. When given after an episode of atrial fibrillation, antiplatelets can reduce the risk of blood clots forming in the heart and traveling to the brain (embolism).

5-HT_{2A} receptors are expressed on smooth muscle of blood vessels and 5-HT secreted by activated platelets causes vasoconstriction as well as activation of additional platelets during clotting. There is evidence that a 5-HT_{2A} inverse agonist will inhibit platelet aggregation and thus be a potential treatment as an antiplatelet therapy (see Satimura, K, et al., Clin Cardiol 2002 Jan. 25 (1):28-32; and Wilson, H. C et al., Thromb Haemost 1991 Sep. 2; 66(3):355-60).

The 5-HT_{2A} inverse agonists disclosed herein provide beneficial improvement in microcirculation to patients in need of antiplatelet therapy by antagonizing the vasoconstrictive products of the aggregating platelets in, for example and not limitation, the indications described above. Accordingly, in some embodiments, the present invention provides methods for reducing platelet aggregation in a patient in need thereof comprising administering to said patient a composition comprising a 5-HT_{2A} inverse agonist disclosed herein. In further embodiments, the present invention provides methods for treating coronary artery disease, myocardial infarction, transient ischemic attack, angina, stroke, atrial fibrillation, or a symptom of any of the foregoing in a patient in need of said treatment, comprising administering to said patient a composition comprising a 5-HT_{2A} inverse agonist disclosed herein.

In further embodiments, the present invention provides methods for reducing risk of blood clot formation in an angioplasty or coronary bypass surgery patient, or a patient suffering from atrial fibrillation, comprising administering to a said patient a composition comprising a 5-HT_{2A} inverse agonist disclosed herein at a time where such risk exists.

In further embodiments, the present invention provides methods for reducing risk of, or treating the effects of, PCI, comprising administering to a patient a composition comprising a 5-HT_{2A} inverse agonist disclosed herein at a time where such risk exists.

In further embodiments, the present invention provides methods for the prevention or treatment of Raynaud's, comprising administering to a patient a composition comprising a 5-HT_{2A} inverse agonist disclosed herein.

### Synthetic Methods:

Example processes and intermediates of the present disclosure are provided below in Scheme 1. As will be appreciated by those skilled in the art, the compounds provided herein, including salts thereof, can be prepared using known organic synthesis techniques and can be synthesized according to any of numerous possible synthetic routes, such as that provided in Scheme 1.

The reactions for preparing compounds described herein can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

Scheme 1 provides general guidance in connection with preparing the compounds of the invention. For instance, the compound of Formula (I) can be prepared as shown in Scheme 1.

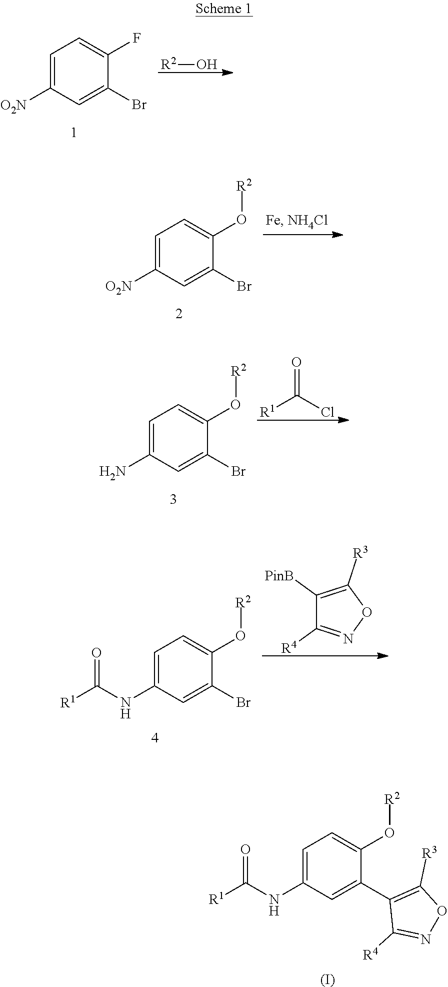

### Polymorphs and Pseudopolymorphs

The present disclosure includes polymorphs and pseudopolymorphs of the compound of Formula (I) of the present disclosure. Polymorphism is the ability of a substance to exist as two or more crystalline phases that have different arrangements and/or conformations of the molecules in the crystal lattice. Polymorphs show the same properties in the liquid or gaseous state but they behave differently in the solid state.

Besides single-component polymorphs, drugs can also exist as salts and other multicomponent crystalline phases. For example, crystalline phases can contain an API host and either solvent or water molecules, respectively, as guests. Crystalline phases that share the same API host, but differ with respect to the guests, can be referred to as pseudopolymorphs of one another.

Solvates contain molecules of the solvent of crystallization in a definite crystal lattice. Solvates, in which the solvent of crystallization is water, are termed hydrates. Because water is a constituent of the atmosphere, hydrates of drugs may be formed rather easily.

By way of example, Stahly published a polymorph screen of 245 compounds consisting of a “wide variety of structural types” that revealed about 90% of them exhibited multiple solid forms. Overall, approximately half of the compounds were polymorphic, often having one to three forms. About one-third of the compounds formed hydrates, and about one-third formed solvates. Data from cocrystal screens of 64 compounds showed that 60% formed cocrystals other than hydrates or solvates. (G. P. Stahly, Crystal Growth & Design (2007), 7(6), 1007-1026).

### Isotopes

The present disclosure includes all isotopes of atoms occurring in the compounds provided herein. Isotopes include those atoms having the same atomic number but different mass numbers. It is appreciated that certain features of the disclosure include every combination of one or more atoms in the compounds provided herein that is replaced with an atom having the same atomic number but a different mass number. One such example is the replacement of an atom that is the most naturally abundant isotope, such as ¹H or ¹²C, found in one of the compounds provided herein with a different atom that is not the most naturally abundant isotope, such as ²H or ³H (replacing ¹H), or ¹¹C, ¹³C, or ¹⁴C (replacing ¹²C). A compound where such a replacement has taken place is commonly referred to as being isotopically-labeled. Isotopic-labeling of the present compounds can be accomplished using any one of a variety of different synthetic methods know to those of ordinary skill in the art and they are readily credited with understanding the synthetic methods and available reagents needed to conduct such isotopic-labeling. By way of general example, and without limitation, isotopes of hydrogen include ²H (deuterium) and ³H (tritium). Isotopes of carbon include ¹¹C, ¹³C, and ¹⁴C. Isotopes of nitrogen include ¹³N and ¹⁵N. Isotopes of oxygen include ¹⁵O, ¹⁷O, and ¹⁸O. An isotope of fluorine includes ¹⁸F. An isotope of sulfur includes ³⁵S. An isotope of chlorine includes ³⁶Cl. Isotopes of bromine include ⁷⁵Br, ⁷⁶Br, ⁷⁷Br, and ⁸²Br. Isotopes of iodine include ¹²³I, ¹²⁴I, ¹²⁵I, and ¹³¹I. Also provided are compositions, such as those prepared during synthesis or preformulation, and pharmaceutical compositions, such as those prepared with the intent of using in a mammal for the treatment of one or more of the disorders described herein, comprising one or more of the present compounds, where the naturally occurring distribution of the isotopes in the composition is perturbed. Also provided herein are compositions and pharmaceutical compositions comprising compounds of the present disclosure where the salt is enriched at one or more positions with an isotope other than the most naturally abundant isotope. Methods are readily available to measure such isotope perturbations or enrichments, such as, mass spectrometry, and for isotopes that are radio-isotopes additional methods are available, such as radio-detectors used in connection with HPLC or GC.

One challenge in drug development is improving absorption, distribution, metabolism, excretion, and toxicity (ADMET) properties while maintaining a desired pharmacological profile. Structural changes to improve ADMET properties often alter the pharmacology of a lead compound. While the effects of deuterium substitution on ADMET properties are unpredictable, in select cases deuterium can improve a compound's ADMET properties with minimal perturbation of its pharmacology.

Another object of the present disclosure relates to radio-labeled compounds of the present disclosure that would be useful not only in radio-imaging but also in assays, both in vitro and in vivo, for localizing and quantitating 5-HT_{2A} receptors in tissue samples, including human, and for identifying 5-HT_{2A} receptor ligands by inhibition binding of a radio-labeled compound. It is a further object of this disclosure to develop novel 5-HT_{2A} receptor assays of which comprise such radio-labeled compounds.

The present invention embraces isotopically-labeled compounds of the present invention. Isotopically or radio-labeled compounds are those which are identical to compounds disclosed herein, but for the fact that one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro 5-HT_{2A} serotonin receptor labeling and competition assays, compounds that incorporate ³H, ¹⁴C, ⁸²Br, ¹²⁵I, ¹³¹I or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled compound” is a compound of Formula (I) that has incorporated at least one radionuclide; in some embodiments the radionuclide is selected from the group consisting of ³H, ¹⁴C, ¹²⁵I, ³⁵S and ⁸²Br.

Certain isotopically-labeled compounds of the present disclosure are useful in compound and/or substrate tissue distribution assays. In some embodiments, the radionuclide ³H and/or ¹⁴C isotopes are useful in these studies. Further, substitution with heavier isotopes such as deuterium (i.e., ²H) can afford certain therapeutic advantages resulting from greater metabolic stability (e.g., increased in vivo half-life or reduced dosage requirements) and hence may be preferred in some circumstances. Isotopically labeled compounds of the present disclosure can generally be prepared by following procedures analogous to those disclosed in the present disclosure, by substituting an isotopically labeled reagent for a non-isotopically labeled reagent. Moreover, it should be understood that all of the atoms represented in the compounds of the present disclosure can be either the most commonly occurring isotope of such atoms or the scarcer radio-isotope or nonradioactive isotope. Synthetic methods for incorporating radio-isotopes into organic compounds are applicable to compounds of the invention and are well known in the art.

### Methods

The compound of Formula (I) or any other Formula herein, such as Formulae (Ia), (Ia′), (Ia″), (Ib), (Ic), (Id), (Ie), or (If), and pharmaceutically acceptable salts thereof, have activity as 5-HT_{2A} receptor modulators. Accordingly, the compound of Formula (I) or any other Formula herein, such as Formulae (Ia), (Ia′), (Ia″), (Ib), (Ic), (Id), (Ie), or (If), and pharmaceutically acceptable salts thereof, can be used in methods of modulating the 5-HT_{2A} receptor by contacting the receptor with a compound of Formula (I) or any other Formula herein, such as Formulae (Ia), (Ia′), (Ia″), (Ib), (Ic), (Id), (Ie), or (If), or pharmaceutically acceptable salt thereof, or compositions thereof, as described herein. In further embodiments, a compound of Formula (I) or any other Formula herein, such as Formulae (Ia), (Ia′), (Ia″), (Ib), (Ic), (Id), (Ie), or (If), or pharmaceutically acceptable salt thereof can be used to modulate 5-HT_{2A} receptors in an individual in need of such modulation by administering a therapeutically effective amount of a compound of Formula (I) or any other Formula herein, such as Formulae (Ia), (Ia′), (Ia″), (Ib), (Ic), (Id), (Ie), or (If), or pharmaceutically acceptable salt thereof.

The present disclosure further provides methods of treating diseases associated with the 5-HT_{2A} receptor in an individual (e.g., patient) by administering to the individual in need of such treatment a therapeutically effective amount or dose of a compound of Formula (I) or any other Formula herein, such as Formulae (Ia), (Ia′), (Ia″), (Ib), (Ic), (Id), (Ie), or (If), or pharmaceutically acceptable salt thereof or a pharmaceutical composition thereof. A disease or disorder associated with or directly or indirectly linked to the expression or activity of the 5-HT_{2A} receptor is a disorder or disease in an individual which can be prevented, inhibited, ameliorated, treated or cured by modulation (e.g., agonism, antagonism, or inverse agonism) of the 5-HT_{2A} receptor, for example, by administering to the individual in need thereof a therapeutically effective amount of a compound of the present disclosure or pharmaceutically acceptable salt thereof or composition containing a compound of the present disclosure or pharmaceutically acceptable salt thereof. The disease can be any disease, disorder or condition that is directly or indirectly linked to expression or activity of the 5-HT_{2A} receptor.

Examples of diseases directly or indirectly linked to expression or activity of the 5-HT_{2A} receptor include, but are not limited to, platelet aggregation, coronary artery disease, myocardial infarction, transient ischemic attack, angina, stroke, atrial fibrillation, and blood clot formation, or symptoms thereof.

The present disclosure provides methods of treating coronary artery disease, myocardial infarction, transient ischemic attack, angina, stroke, and atrial fibrillation, comprising administering to a patient in need thereof a therapeutically effective amount of a compound of Formula (I) or any other Formula herein, such as Formulae (Ia), (Ia′), (Ia″), (Ib), (Ic), (Id), (Ie), or (If), or pharmaceutically acceptable salt thereof.

The present disclosure further provides methods for treating conditions related to platelet aggregation comprising prescribing and/or administering to a patient in need thereof a therapeutically effective amount of a compound of Formula (I) or any other Formula herein, such as Formulae (Ia), (Ia′), (Ia″), (Ib), (Ic), (Id), (Ie), or (If), or pharmaceutically acceptable salt thereof. It is understood that treatment of platelet aggregation refers to any reduction of platelet aggregation that results in an amelioration of a pathophysiological condition associated with platelet aggregation.

The present disclosure further provides methods of treating one or more conditions associated with platelet aggregation comprising prescribing and/or administering to a patient in need thereof a therapeutically effective amount of a compound of Formula (I) or any other Formula herein, such as Formulae (Ia), (Ia′), (Ia″), (Ib), (Ic), (Id), (Ie), or (If), or pharmaceutically acceptable salt thereof.

The present disclosure further provides methods of reducing the risk of blood clot formation in an angioplasty or coronary bypass surgery in an individual comprising prescribing and/or administering to a patient in need thereof a therapeutically effective amount of a compound of Formula (I) or any other Formula herein, such as Formulae (Ia), (Ia′), (Ia″), (Ib), (Ic), (Id), (Ie), or (If), or pharmaceutically acceptable salt thereof.

The present disclosure further provides methods of reducing the risk of blood clot formation in an individual suffering from atrial fibrillation comprising prescribing and/or administering to a patient a therapeutically effective amount of a compound of Formula (I) or any other Formula herein, such as Formulae (Ia), (Ia′), (Ia″), (Ib), (Ic), (Id), (Ie), or (If), or pharmaceutically acceptable salt thereof.

The present disclosure further provides methods of reducing the risk of, or treating the effects of, PCI in an individual comprising prescribing and/or administering to a patient a therapeutically effective amount of a compound of Formula (I) or any other Formula herein, such as Formulae (Ia), (Ia′), (Ia″), (Ib), (Ic), (Id), (Ie), or (If), or pharmaceutically acceptable salt thereof.

The present disclosure further provides methods of treating or preventing Raynaud's in an individual comprising prescribing and/or administering to a patient a therapeutically effective amount of a compound of Formula (I) or any other Formula herein, such as Formulae (Ia), (Ia′), (Ia″), (Ib), (Ic), (Id), (Ie), or (If), or pharmaceutically acceptable salt thereof.

In some embodiments, the method further includes the step of identifying a patient, where the patient is in need of treatment for the particular disease being treated. In some embodiments, the identifying step is performed prior to administration to the patient the therapeutically effective amount of a compound of Formula (I) or any other Formula herein, such as Formulae (Ia), (Ia′), (Ia″), (Ib), (Ic), (Id), (Ie), or (If), or pharmaceutically acceptable salt thereof.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof in a method of treatment of the human or animal body by therapy.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof in a method of treatment of a 5HT_{2A}-related disorder of the human or animal body by therapy.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof in a method of treatment of platelet aggregation, coronary artery disease, myocardial infarction, transient ischemic attack, angina, stroke, atrial fibrillation, blood clot formation, or symptoms thereof, in the human or animal body by therapy.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof in a method of treatment of coronary artery disease, myocardial infarction, transient ischemic attack, angina, stroke or atrial fibrillation in the human or animal body by therapy.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof in a method for treating platelet aggregation in the human or animal body by therapy. It is understood that treatment of platelet aggregation refers to any reduction of platelet aggregation that results in an amelioration of a pathophysiological condition associated with platelet aggregation.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof in a method for treating one or more conditions associated with platelet aggregation in the human or animal body by therapy.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof in a method for reducing the risk of blood clot formation in an angioplasty or coronary bypass surgery individual by therapy.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof in a method for reducing the risk of blood clot formation in an individual suffering from atrial fibrillation by therapy.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof in a method for reducing the risk of, or treating the effects of, PCI in an individual by therapy.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof in a method for treating or preventing Raynaud's in an individual by therapy.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof for the manufacture of a medicament for treating a 5HT_{2A}-related disorder.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof for the manufacture of a medicament for treating disease or disorder selected from platelet aggregation, coronary artery disease, myocardial infarction, transient ischemic attack, angina, stroke, atrial fibrillation, blood clot formation, or symptoms thereof.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof for the manufacture of a medicament for treating coronary artery disease, myocardial infarction, transient ischemic attack, angina, stroke, or atrial fibrillation.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof for the manufacture of a medicament for treating platelet aggregation. It is understood that treatment of platelet aggregation refers to any reduction of platelet aggregation that results in an amelioration of a pathophysiological condition associated with platelet aggregation.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof for the manufacture of a medicament for treating one or more conditions associated with platelet aggregation.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof for the manufacture of a medicament for reducing the risk of blood clot formation in an angioplasty or coronary bypass surgery individual.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof for the manufacture of a medicament for reducing the risk of blood clot formation in an individual suffering from atrial fibrillation.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof for the manufacture of a medicament for reducing the risk of, or treating the effects of, PCI in an individual.

One aspect of the present disclosure pertains to use of a compound of Formula (I) or any other Formula herein, such as Formulae (Ia), (Ia′), (Ia″), (Ib), (Ic), (Id), (Ie), or (If), or pharmaceutically acceptable salt thereof for the manufacture of a medicament for treating or preventing Raynaud's in an individual.

### Pharmaceutical Compositions

A further aspect of the present disclosure pertains to pharmaceutical compositions comprising the compound of Formula (I) or any other Formula herein, such as Formulae (Ia), (Ia′), (Ia″), (Ib), (Ic), (Id), (Ie), or (If), or pharmaceutically acceptable salt thereof as described herein and one or more pharmaceutically acceptable carriers. In some embodiments, the pharmaceutical composition comprises a compound of Formula (I) or any other Formula herein, such as Formulae (Ia), (Ia′), (Ia″), (Ib), (Ic), (Id), (Ie), or (If), and a pharmaceutically acceptable carrier. In some embodiments, the pharmaceutical composition comprises a salt of the compound of Formula (I) or any other Formula herein, such as Formulae (Ia), (Ia′), (Ia″), (Ib), (Ic), (Id), (Ie), or (If), and a pharmaceutically acceptable carrier.

Some embodiments of the present disclosure include a method of producing a pharmaceutical composition comprising admixing the compound of Formula (I) or any other Formula herein, such as Formulae (Ia), (Ia′), (Ia″), (Ib), (Ic), (Id), (Ie), or (If), or pharmaceutically acceptable salt thereof and a pharmaceutically acceptable carrier.

Some embodiments of the present disclosure include a method of producing a pharmaceutical composition comprising admixing a salt of the compound of Formula (I) or any other Formula herein, such as Formulae (Ia), (Ia′), (Ia″), (Ib), (Ic), (Id), (Ie), or (If), as disclosed herein and a pharmaceutically acceptable carrier.

Formulations can be prepared by any suitable method known to those skilled in the art. In some embodiments, the formulation is prepared by uniformly mixing the active compound(s) with liquids or finely divided solid carriers, or both, in the required proportions, and then, if necessary, forming the resulting mixture into a desired shape.

Conventional excipients, such as binding agents, fillers, acceptable wetting agents, tableting lubricants, and disintegrants can be used in tablets and capsules for oral administration. Liquid preparations for oral administration can be in the form of solutions, emulsions, aqueous or oily suspensions, and syrups. Alternatively, the oral preparations can be in the form of dry powder that can be reconstituted with water or another suitable liquid vehicle before use. Additional additives, such as suspending or emulsifying agents, non-aqueous vehicles (including edible oils), preservatives, and flavorings and colorants can be added to the liquid preparations. Parenteral dosage forms can be prepared by dissolving the compound of Formula (I) or any other Formula herein, such as Formulae (Ia), (Ia′), (Ia″), (Ib), (Ic), (Id), (Ie), or (If), or a pharmaceutically acceptable salt thereof in a suitable liquid vehicle and filter sterilizing the solution before filling and sealing an appropriate vial or ampoule. These are just a few examples of the many appropriate methods well known in the art for preparing dosage forms.

A compound of Formula (I) or any other Formula herein, such as Formulae (Ia), (Ia′), (Ia″), (Ib), (Ic), (Id), (Ie), or (If), or pharmaceutically acceptable salt thereof can be formulated into pharmaceutical compositions using techniques well known to those in the art. Suitable pharmaceutically acceptable carriers, outside those mentioned herein, are known in the art; for example, see Remington, The Science and Practice of Pharmacy, 20^{th} Ed., 2000, Lippincott Williams & Wilkins, (Editors: Gennaro, A. R., et al.).

While it is possible that, for use in treatment, the compound of Formula (I) or any other Formula herein, such as Formulae (Ia), (Ia′), (Ia″), (Ib), (Ic), (Id), (Ie), or (If), or pharmaceutically acceptable salt thereof can, in an alternative use, be administered as a raw or pure chemical, it is preferable however to present the compound or active ingredient as a pharmaceutical formulation or composition further comprising a pharmaceutically acceptable carrier.

The disclosure thus further provides pharmaceutical formulations comprising a compound of Formula (I) or any other Formula herein, such as Formulae (Ia), (Ia′), (Ia″), (Ib), (Ic), (Id), (Ie), or (If), or pharmaceutically acceptable salt thereof together with one or more pharmaceutically acceptable carriers and/or prophylactic ingredients. The carrier(s) is “acceptable” in the sense of being compatible with the other ingredients of the formulation and not overly deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with a minimum of degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compound of Formula (I) or any other Formula herein, such as Formulae (Ia), (Ia′), (Ia″), (Ib), (Ic), (Id), (Ie), or (If), or pharmaceutically acceptable salt thereof, together with a conventional adjuvant, carrier, or diluent, can thus be placed into the form of a pharmaceutical formulation and unit dosage thereof, and in such form can be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms can comprise conventional ingredients in conventional proportions, with or without additional active compounds or principles, and such unit dosage forms can contain any suitable effective amount of the active ingredient commensurate with the intended daily dosage range to be employed.

For oral administration, the pharmaceutical composition can be in the form of, for example, a tablet, capsule, suspension or liquid. In some embodiments, the pharmaceutical composition is made in the form of a dosage unit containing a particular amount of the active ingredient. Examples of such dosage units include, but are not limited to, capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethylcellulose; and with lubricants such as talc or magnesium stearate. The active ingredient can also be administered by injection as a composition where, for example, saline, dextrose or water can be used as a suitable pharmaceutically acceptable carrier.

The compound of Formula (I) or any other Formula herein, such as Formulae (Ia), (Ia′), (Ia″), (Ib), (Ic), (Id), (Ie), or (If), or pharmaceutically acceptable salt thereof can be used as an active ingredient in pharmaceutical compositions, specifically as a 5-HT_{2A} receptor modulator. The term “active ingredient” is defined in the context of a “pharmaceutical composition” and means a component of a pharmaceutical composition that provides the primary pharmacological effect, as opposed to an “inactive ingredient” which would generally be recognized as providing no pharmaceutical benefit.

The dose, when using the compound of Formula (I) or any other Formula herein, such as Formulae (Ia), (Ia′), (Ia″), (Ib), (Ic), (Id), (Ie), or (If), or pharmaceutically acceptable salt thereof, can vary within wide limits, as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compound of Formula (I) or any other Formula herein, such as Formulae (Ia), (Ia′), (Ia″), (Ib), (Ic), (Id), (Ie), or (If), or pharmaceutically acceptable salt thereof. Representative doses of the present disclosure include, but are not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg, and about 0.001 mg to about 25 mg. Multiple doses can be administered during the day, especially when relatively large amounts are deemed to be needed, for example 2, 3 or 4, doses. Depending on the individual and as deemed appropriate from the patient's physician or care-giver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations can merely be based on the weight of the animal model in comparison to another, such as a mammal, preferably a human, however, more often, these extrapolations are not simply based on weights, but rather incorporate a variety of factors. Representative factors include, but are not limited to, the type, age, weight, sex, diet, and medical condition of the patient, the severity of the disease, the route of administration, pharmacological considerations such as the activity, efficacy, pharmacokinetic and toxicology profiles of the particular compound employed, whether a drug delivery system is utilized, or whether an acute or chronic disease state is being treated or prophylaxis is conducted or on whether further active compounds are administered in addition to the compound of Formula (I) or any other Formula herein, such as Formulae (Ia), (Ia′), (Ia″), (Ib), (Ic), (Id), (Ie), or (If), or pharmaceutically acceptable salt thereof or as part of a drug combination. The dosage regimen for treating a disease or condition with the compound of Formula (I) or any other Formula herein, such as Formulae (Ia), (Ia′), (Ia″), (Ib), (Ic), (Id), (Ie), or (If), or pharmaceutically acceptable salt thereof and/or compositions of this disclosure is selected in accordance with a variety factors as described in the present disclosure. Thus, the actual dosage regimen employed can vary widely and therefore can deviate from a preferred dosage regimen. One skilled in the art will recognize that dosages and dosage regimens outside these typical ranges can be tested and, where appropriate, can be used in the methods of the present disclosure.

The desired dose can be presented in a single dose or as divided doses administered at appropriate intervals, for example, as two, three, four or more sub-doses per day. The sub-dose itself can be further divided, e.g., into a number of discrete loosely spaced administrations. The daily dose can be divided, for example, when relatively large amounts are administered as deemed appropriate, into several, for example 2, 3 or 4, partial administrations. If appropriate, depending on individual behavior, it may be necessary to deviate upward or downward from the daily dose indicated.

The compound of Formula (I) or any other Formula herein, such as Formulae (Ia), (Ia′), (Ia″), (Ib), (Ic), (Id), (Ie), or (If), or pharmaceutically acceptable salt thereof can be administrated in a wide variety of oral and parenteral dosage forms. It will be obvious to those skilled in the art that the following dosage forms can contain, as the active component, either the compound of Formula (I) or any other Formula herein, such as Formulae (Ia), (Ia′), (Ia″), (Ib), (Ic), (Id), (Ie), or (If), or a pharmaceutically acceptable salt thereof.

For preparing pharmaceutical compositions from the compound of Formula (I) or any other Formula herein, such as Formulae (Ia), (Ia′), (Ia″), (Ib), (Ic), (Id), (Ie), or (If), or pharmaceutically acceptable salt thereof, the selection of a suitable pharmaceutically acceptable carrier can be either solid, liquid or a mixture of both. Solid form preparations include powders, tablets, pills, capsules, cachets, suppositories, and dispersible granules. A solid carrier can be one or more substances which can also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size.

The powders and tablets can contain varying percentage amounts of the active compound. A representative amount in a powder or tablet can contain from about 0.5% to about 90% of the active compound. A skilled artisan would know when amounts outside of this range are necessary. Suitable carriers for powders and tablets include, but are not limited to, magnesium carbonate, magnesium stearate, talc, sugar, lactose, pectin, dextrin, starch, gelatin, tragacanth, methylcellulose, sodium carboxymethylcellulose, a low melting wax, and cocoa butter. The term “preparation” is intended to include the formulation of the active compound with an encapsulating material as carrier, providing a capsule in which the active component, with or without carriers, is surrounded by a carrier, which is thus in association with it. Similarly, cachets and lozenges are included in some embodiments. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration can be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. In addition, fatty acids, such as oleic acid, can find use in the preparation of injectables.

The compound of Formula (I) or any other Formula herein, such as Formulae (Ia), (Ia′), (Ia″), (Ib), (Ic), (Id), (Ie), or (If), or pharmaceutically acceptable salt thereof can thus be formulated for parenteral administration (e.g., by injection, for example, bolus injection or continuous infusion) and can be presented in unit dose form in ampoules, pre-filled syringes, small volume infusion or in multi-dose containers with an added preservative. The pharmaceutical compositions can take such forms as suspensions, solutions, or emulsions in oily or aqueous vehicles, and can contain formulatory agents such as suspending, stabilizing and/or dispersing agents. Alternatively, the active ingredient can be in powder form, obtained by aseptic isolation of sterile solid or by lyophilization from solution, for constitution with a suitable vehicle, e.g., sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Also included, in some embodiments, are solid form preparations which are intended to be converted, shortly before use, to liquid form preparations for oral administration. Such liquid forms can include solutions, suspensions, and emulsions. These preparations can contain, in addition to the active component, for example, colorants, flavors, stabilizers, buffers, artificial and natural sweeteners, dispersants, thickeners, and solubilizing agents.

For topical administration to the epidermis, the compound of Formula (I) or any other Formula herein, such as Formulae (Ia), (Ia′), (Ia″), (Ib), (Ic), (Id), (Ie), or (If), or pharmaceutically acceptable salt thereof can be formulated as ointments, creams or lotions, or as a transdermal patch.

Ointments and creams can, for example, be formulated with an aqueous or oily base with the addition of suitable thickening and/or gelling agents. Lotions can be formulated with an aqueous or oily base and can also contain one or more emulsifying agents, stabilizing agents, dispersing agents, suspending agents, thickening agents, or coloring agents.

Formulations suitable for topical administration in the mouth include, but are not limited to, lozenges comprising active agent in a flavored base, for example, sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations can be provided in single or multi-dose form. In the case of a dropper or pipette, administration can be achieved by the patient administering an appropriate, predetermined volume of the solution or suspension. In the case of a spray, this may be achieved, for example, by means of a metering atomizing spray pump.

Administration to the respiratory tract can also be achieved by means of an aerosol formulation in which the active ingredient is provided in a pressurized pack with a suitable propellant. If the compound of Formula (I) or any other Formula herein, such as Formulae (Ia), (Ia′), (Ia″), (Ib), (Ic), (Id), (Ie), or (If), or pharmaceutically acceptable salt thereof or pharmaceutical compositions comprising the compound of Formula (I) or any other Formula herein, such as Formulae (Ia), (Ia′), (Ia″), (Ib), (Ic), (Id), (Ie), or (If), or pharmaceutically acceptable salt thereof are administered as aerosols, for example, as nasal aerosols or by inhalation, this can be carried out, for example, using a spray, a nebulizer, a pump nebulizer, an inhalation apparatus, a metered inhaler, or a dry powder inhaler.

Pharmaceutical forms for administration of the compound of Formula (I) or any other Formula herein, such as Formulae (Ia), (Ia′), (Ia″), (Ib), (Ic), (Id), (Ie), or (If), or pharmaceutically acceptable salt thereof as an aerosol can be prepared by processes well-known to the person skilled in the art. For their preparation, for example, solutions or dispersions of the compound of Formula (I) or any other Formula herein, such as Formulae (Ia), (Ia′), (Ia″), (Ib), (Ic), (Id), (Ie), or (If), or pharmaceutically acceptable salt thereof in water, water/alcohol mixtures or suitable saline solutions can be employed using customary additives, for example benzyl alcohol or other suitable preservatives, absorption enhancers for increasing the bioavailability, solubilizers, dispersants and others, and, if appropriate, customary propellants, including, but not limited to, carbon dioxide and CFCs, such as, dichlorodifluoromethane, trichlorofluoromethane, and dichlorotetrafluoroethane. The aerosol can also contain a surfactant. In some embodiments, the surfactant is lecithin. The dose of drug can be controlled by provision of a metered valve.

In formulations intended for administration to the respiratory tract, including intranasal formulations, the compound will generally have a small particle size, for example, on the order of 10 microns or less. Such a particle size can be obtained by means known in the art, for example by micronization. When desired, formulations adapted to give sustained release of the active ingredient can be employed.

Alternatively, the active ingredients can be provided in the form of a dry powder, for example, a powder mix of the compound in a suitable powder base such as lactose, starch, or starch derivatives such as hydroxypropylmethylcellulose (HPMC) and polyvinylpyrrolidone (PVP). In some embodiments, the powder carrier will form a gel in the nasal cavity. The powder composition can be presented in unit dose form, for example in capsules or cartridges of, e.g., gelatin, or blister packs from which the powder can be administered by means of an inhaler.

In some embodiments, the pharmaceutical preparations are in unit dosage forms. In such form, the preparation is subdivided into unit doses containing appropriate quantities of the active component. The unit dosage form can be a packaged preparation, where the package contains discrete quantities of preparation, such as packeted tablets, capsules, and powders in vials or ampoules. In some embodiments, the unit dosage form is a capsule, tablet, cachet, or lozenge itself, or it is the appropriate number of any of these in packaged form.

Tablets or capsules for oral administration and liquids for intravenous administration are preferred compositions.

The compounds provided herein may optionally exist as pharmaceutically acceptable salts including pharmaceutically acceptable acid addition salts prepared from pharmaceutically acceptable non-toxic acids including inorganic and organic acids. Representative acids include, but are not limited to acetic, benzenesulfonic, benzoic, camphorsulfonic, citric, dichloroacetic, ethanesulfonic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Certain compounds provided herein which contain a carboxylic acid functional group may optionally exist as pharmaceutically acceptable salts containing non-toxic, pharmaceutically acceptable metal cations and cations derived from organic bases. Representative metals include, but are not limited to, aluminium, calcium, lithium, magnesium, potassium, sodium, zinc and the like. In some embodiments the pharmaceutically acceptable metal is sodium. Representative organic bases include, but are not limited to, benzathine (N¹,N²-dibenzylethane-1,2-diamine), chloroprocaine (2-(diethylamino)ethyl 4-(chloroamino)benzoate), choline, diethanolamine, ethylenediamine, meglumine ((2R,3R,4R,5S)-6-(methylamino)hexane-1,2,3,4,5-pentaol), procaine (2-(diethylamino)ethyl 4-aminobenzoate), and the like. Certain pharmaceutically acceptable salts are listed in Berge, et. al., Journal of Pharmaceutical Sciences, 66:1-19 (1977).

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds provided herein may form solvates with standard low molecular weight solvents using methods known to the skilled artisan.

Compounds provided herein can be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds provided herein containing one or more specialized non-toxic protective groups used in a transient manner to alter or to eliminate a property of the compound. In one general aspect, the “pro-drug” approach is utilized to facilitate oral absorption. A thorough discussion is provided in T. Higuchi and V. Stella, Pro-drugs as Novel Delivery Systems Vol. 14 of the A.C.S. Symposium Series; and in Bioreversible Carriers in Drug Design, ed. Edward B. Roche, American Pharmaceutical Association and Pergamon Press, 1987.

Some embodiments of the present disclosure include a method of producing a pharmaceutical composition for “combination therapy” comprising admixing the compound of Formula (I) or any other Formula herein, such as Formulae (Ia), (Ia′), (Ia″), (Ib), (Ic), (Id), (Ie), or (If), or pharmaceutically acceptable salt thereof together with at least one known pharmaceutical agent as described herein and a pharmaceutically acceptable carrier.

It is noted that when the 5-HT_{2A} receptor modulators are utilized as active ingredients in a pharmaceutical composition, these are not intended for use only in humans, but in other non-human mammals as well. Indeed, advances in the area of animal health-care mandate that consideration be given for the use of active agents, such as 5-HT_{2A} receptor modulators, for the treatment of a 5-HT_{2A} mediated diseases or disorders in domestic animals (e.g., cats and dogs) and in other domestic animals (e.g., such as cows, chickens, and fish). Those of ordinary skill in the art will understand the utility of such compounds in such settings.

### EXAMPLES

### Example 1—Preparation of the Compound of Formula (I)

Experimental procedures for the preparation of the compound of Formula (I) are provided below.

### N-(3-(3,5-dimethylisoxazol-4-yl)-4-(2-(pyrrolidin-1-yl)ethoxy)phenyl)-[1,1′-bi(cyclopropane)]-2-carboxamide (100)

### Preparation of 1-[2-(2-bromo-4-nitro-phenoxy)ethyl]pyrrolidine (1)

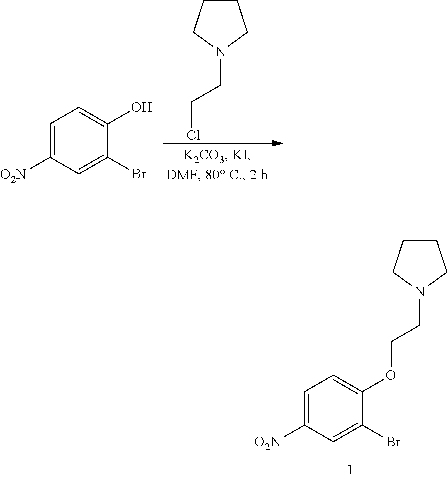 [SMILES: O=[N+]([O-])c1ccc(O)c(Br)c1 ; ClCCN1CCCC1 ; O=[N+]([O-])c1ccc(OCCN2CCCC2)c(Br)c1]

A mixture of 2-bromo-4-nitro-phenol (10 g, 45.87 mmol, 1 eq.), 1-(2-chloroethyl)pyrrolidine (15.60 g, 91.74 mmol, 2 eq., HCl), K₂CO₃ (19.02 g, 137.61 mmol, 3 eq.), and KI (761.45 mg, 4.59 mmol, 0.1 eq.) in DMF (100 mL) was degassed and purged with N₂ three times, and then the mixture was stirred at 100° C. for 16 hrs under an N₂ atmosphere (monitored by LC-MS). The reaction mixture was filtered and concentrated under reduced pressure to give a residue. The residue was dissolved in EtOAc (200 mL). To the mixture was added H₂O (100 mL). The mixture was adjusted to pH=4-5 and extracted with EtOAc (200 mL×3). The combined organic layers were dried over Na₂SO₄, filtered and concentrated under reduced pressure to give a residue. The water layer was adjusted to pH-8-9 and extracted with EtOAc (200 mL×3). The combined organic layers were dried over Na₂SO₄, filtered and concentrated under reduced pressure to give a residue. The water layer was lyophilized. The crude product was purified by reversed-phase HPLC (0.1% NH₃·H₂O). 1-[2-(2-bromo-4-nitro-phenoxy)ethyl]pyrrolidine ((1), 15 g, crude) was obtained as a white solid.

LCMS (ESI): m/z [M+H] calcd for C₁₂H₁₆Br^{79/81}N₂O₃: 315.03 and 317.02; found: 315.1 and 317.1.

### Preparation of 3-bromo-4-(2-pyrrolidin-1-ylethoxy)aniline (2)

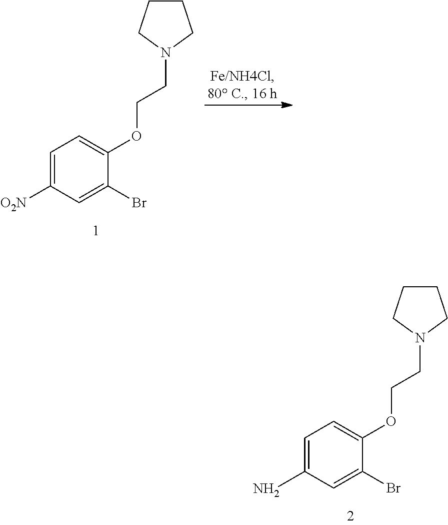 [SMILES: O=[N+]([O-])c1ccc(OCCN2CCCC2)c(Br)c1 ; Nc1ccc(OCCN2CCCC2)c(Br)c1]

A mixture of 1-[2-(2-bromo-4-nitro-phenoxy)ethyl]pyrrolidine ((1), 5 g, 15.86 mmol, 1 eq.), Fe (4.43 g, 79.32 mmol, 5 eq.), and NH₄Cl (8.49 g, 158.65 mmol, 10 eq.) in EtOH (50 mL) and H₂O (10 mL) was degassed and purged with N₂ three times, and then the mixture was stirred at 80° C. for 16 hrs under an N₂ atmosphere (monitored by LC-MS). The reaction mixture was filtered and concentrated under reduced pressure to remove EtOH (50 mL). The residue was extracted with EtOAc (200 mL×3). The combined organic layers were concentrated under reduced pressure to give a residue. 3-bromo-4-(2-pyrrolidin-1-ylethoxy)aniline ((2), 4.5 g, crude) was obtained as a black oil.

LCMS (ESI): m/z [M+H] calcd for C₁₂H₁₈Br^{79/81}N₂O: 285.05 and 287.05; found: 285.0 and 287.0.

### Preparation of 3-(3,5-dimethylisoxazol-4-yl)-4-(2-pyrrolidin-1-ylethoxy)aniline (3)

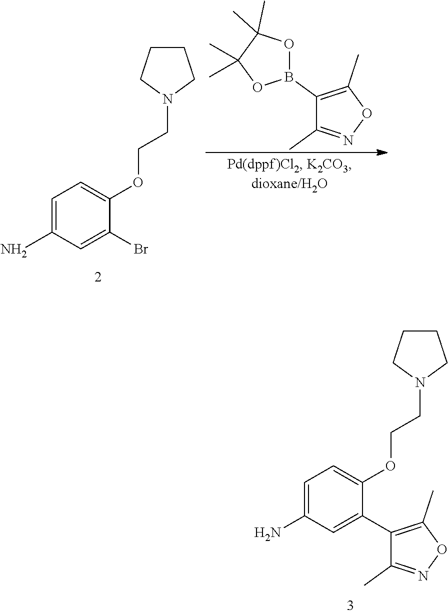

A mixture of 3-bromo-4-(2-pyrrolidin-1-ylethoxy)aniline ((2), 2 g, 7.01 mmol, 1 eq.), 3,5-dimethyl-4-(4,4,5,5-tetramethyl-1,3,2-dioxaborolan-2-yl)isoxazole (1.88 g, 8.42 mmol, 1.2 eq.), Pd(dppf)Cl₂ (256.58 mg, 350.66 μmol, 0.05 eq.), and K₂CO₃ (1.94 g, 14.03 mmol, 2 eq.) in dioxane (15 mL) and H₂O (1 mL) was degassed and purged with N₂ three times, and then the mixture was stirred at 80° C. for 16 hrs under an N₂ atmosphere (monitored by LC-MS). The reaction mixture was concentrated under reduced pressure to give a residue. The residue was purified by column chromatography (SiO₂, petroleum ether/ethyl acetate=1/1 to I/O). The residue was purified by prep-HPLC (column: Waters Xbridge C18 150*50 mm*10 um; mobile phase: [water (0.05% ammonia hydroxide v/v)-ACN]; B %: 23%-53%, 11.5 min). 3-(3,5-dimethylisoxazol-4-yl)-4-(2-pyrrolidin-1-ylethoxy)aniline ((3), 320 mg, 1.06 mmol, 15.14% yield) was obtained as a white solid.

LCMS (ESI): m/z [M+H] calcd for C₁₇H₂₄N₃O₂: 302.18; found: 302.1.

¹H NMR (400 MHZ, methanol-d₄) δ=6.90 (d, J=8.7 Hz, 1H), 6.78 (dd, J=2.8, 8.7 Hz, 1H), 6.60 (d, J=2.8 Hz, 1H), 3.99 (t, J=5.7 Hz, 2H), 2.77 (t, J=5.7 Hz, 2H), 2.54-2.45 (m, 4H), 2.28 (s, 3H), 2.14 (s, 3H), 1.74 (td, J=3.3, 6.8 Hz, 4H).

### Preparation of N-(3-(3,5-dimethylisoxazol-4-yl)-4-(2-(pyrrolidin-1-yl)ethoxy)phenyl)-[1,1′-bi(cyclopropane)]-2-carboxamide (100)

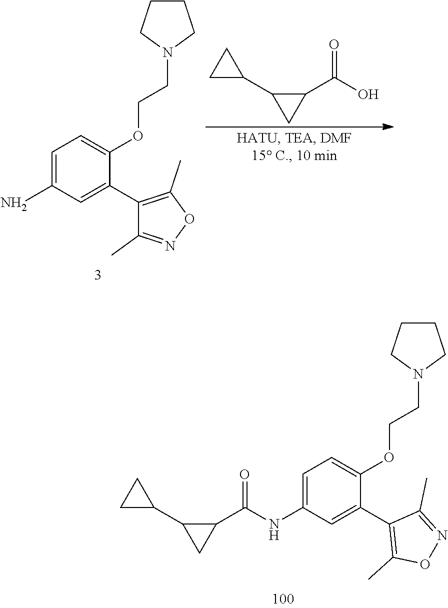

A mixture of 2-cyclopropylcyclopropanecarboxylic acid (36.83 mg, 291.99 μmol, 1.1 eq.), HATU (151.39 mg, 398.16 μmol, 1.5 eq.) and TEA (53.72 mg, 530.89 μmol, 73.89 μL, 2 eq.) in DMF (1 mL) was stirred at 25° C. for 10 min, then to the mixture was added 3-(3,5-dimethylisoxazol-4-yl)-4-(2-pyrrolidin-1-ylethoxy)aniline ((3), 80 mg, 265.44 μmol, 1 eq.) and the mixture was stirred at 25° C. for 10 min (monitored by LC-MS). The reaction mixture was concentrated under reduced pressure to give a residue. The residue was purified by prep-HPLC (column: Phenomenex Luna C18 150*25 mm*10 um; mobile phase: [water (0.1% TFA)-ACN]; B %: 25%-45%, 10 min). 2-cyclopropyl-N-[3-(3,5-dimethylisoxazol-4-yl)-4-(2-pyrrolidin-1-ylethoxy)phenyl]cyclopropanecarboxamide (84.99 mg, 193.01 μmol, 72.71% yield, 93% purity) was obtained as a yellow solid.

LCMS (ESI): m/z [M+H] calcd for C₂₄H₃₂N₃O₃: 410.24; found: 410.3.

¹H NMR (400 MHZ, methanol-d₄) δ=7.66-7.53 (m, 1H), 7.42 (d, J=2.0 Hz, 1H), 7.13 (d, J=8.9 Hz, 1H), 4.38-4.23 (m, 2H), 3.61-3.55 (m, 2H), 3.55-3.47 (m, 2H), 3.10-2.95 (m, 2H), 2.32 (s, 3H), 2.16 (s, 3H), 2.13-2.02 (m, 2H), 1.93 (br dd, J=5.0, 7.3 Hz, 2H), 1.56 (td, J=4.3, 8.3 Hz, 1H), 1.46-1.34 (m, 1H), 1.06 (td, J=4.5, 9.0 Hz, 1H), 0.90 (dt, J=4.9, 8.1 Hz, 1H), 0.73 (ddd, J=4.2, 6.4, 8.1 Hz, 1H), 0.51-0.36 (m, 2H), 0.21-0.11 (m, 2H).

### N-(3-(3,5-dimethylisoxazol-4-yl)-4-(2-(pyrrolidin-1-yl)ethoxy)phenyl)-3-ethoxypropanamide (101)

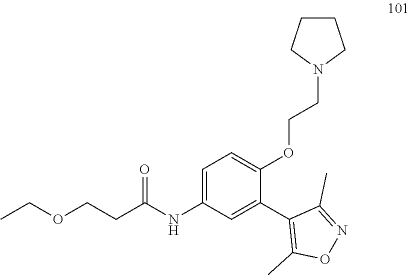 [SMILES: CCOCCC(=O)Nc1ccc(OCCN2CCCC2)c(-c2c(C)noc2C)c1]

Compound 101 was prepared according to the synthesis described for compound 100, substituting 3-ethoxypropanoic acid for 2-cyclopropylcyclopropanecarboxylic acid.

LCMS (ESI): m/z [M+H] calcd for C₂₂H₃₂N₃O₄: 402.23; found: 402.2.

¹H NMR (400 MHZ, methanol-d₄) δ=7.54 (dd, J=2.4, 8.8 Hz, 1H), 7.42 (d, J=2.4 Hz, 1H), 7.07 (d, J=9.0 Hz, 1H), 4.11 (t, J=5.6 Hz, 2H), 3.76 (t, J=6.1 Hz, 2H), 3.53 (q, J=6.9 Hz, 2H), 2.83 (t, J=5.6 Hz, 2H), 2.60 (t, J=6.1 Hz, 2H), 2.51 (br s, 4H), 2.29 (s, 3H), 2.15 (s, 3H), 1.75 (br s, 4H), 1.18 (t, J=7.0 Hz, 3H).

### N-(3-(3,5-dimethylisoxazol-4-yl)-4-(2-(pyrrolidin-1-yl)ethoxy)phenyl)isonicotinamide (102)

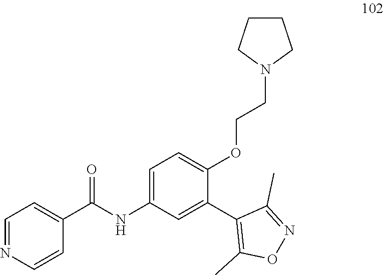 [SMILES: Cc1noc(C)c1-c1cc(NC(=O)c2ccncc2)ccc1OCCN1CCCC1]

Compound 102 was prepared according to the synthesis described for compound 100, substituting isonicotinic acid for 2-cyclopropylcyclopropanecarboxylic acid.

LCMS (ESI): m/z [M+H] calcd for C₂₃H₂₇N₄O₃: 407.20; found: 407.1.

¹H NMR (400 MHZ, methanol-d₄) δ=8.79-8.70 (m, 2H), 7.93-7.86 (m, 2H), 7.73 (dd, J=2.7, 8.9 Hz, 1H), 7.58 (d, J=2.7 Hz, 1H), 7.15 (d, J=8.9 Hz, 1H), 4.17 (t, J=5.6 Hz, 2H), 2.89 (t, J=5.6 Hz, 2H), 2.56 (br s, 4H), 2.33 (s, 3H), 2.18 (s, 3H), 1.77 (td, J=3.3, 6.8 Hz, 4H).

### N-(3-(3,5-dimethylisoxazol-4-yl)-4-(2-(pyrrolidin-1-yl)ethoxy)phenyl)-3-fluoroisonicotinamide (103)

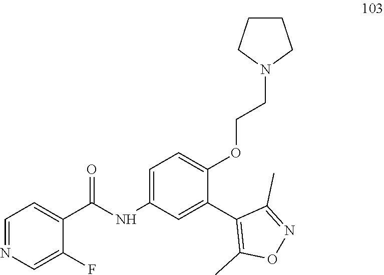 [SMILES: Cc1noc(C)c1-c1cc(NC(=O)c2ccncc2F)ccc1OCCN1CCCC1]

Compound 103 was prepared according to the synthesis described for compound 100, substituting 3-fluoroisonicotinic acid for 2-cyclopropylcyclopropanecarboxylic acid.

LCMS (ESI): m/z [M+H] calcd for C₂₃H₂₆FN₄O₃: 425.19; found: 425.2.

¹H NMR (400 MHZ, methanol-d₄) δ=8.65 (d, J=1.8 Hz, 1H), 8.56 (d, J=4.9 Hz, 1H), 7.77 (dd, J=1.8, 8.8 Hz, 1H), 7.72 (t, J=5.4 Hz, 1H), 7.59 (d, J=2.7 Hz, 1H), 7.22 (d, J=9.0 Hz, 1H), 4.41-4.31 (m, 2H), 3.66-3.58 (m, 2H), 3.52 (br d, J=5.0 Hz, 2H), 3.12-2.96 (m, 2H), 2.35 (s, 3H), 2.19 (s, 3H), 2.10 (br s, 2H), 2.02-1.87 (m, 2H).

### N-(3-(3,5-dimethylisoxazol-4-yl)-4-(2-(pyrrolidin-1-yl)ethoxy)phenyl)-4-fluoropicolinamide (104)

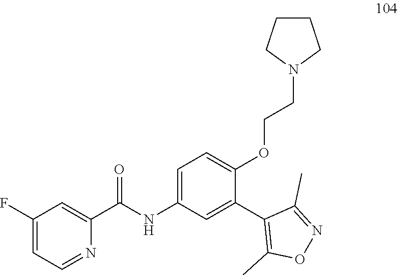 [SMILES: Cc1noc(C)c1-c1cc(NC(=O)c2cc(F)ccn2)ccc1OCCN1CCCC1]

Compound 104 was prepared according to the synthesis described for compound 100, substituting 4-fluoropicolinic acid for 2-cyclopropylcyclopropanecarboxylic acid.

LCMS (ESI): m/z [M+H] calcd for C₂₃H₂₆FN₄O₃: 425.19; found: 425.3.

¹H NMR (400 MHZ, methanol-d₄) δ=8.70 (dd, J=5.6, 8.0 Hz, 1H), 7.92 (dd, J=2.6, 9.4 Hz, 1H), 7.79 (dd, J=2.7, 8.9 Hz, 1H), 7.67 (d, J=2.7 Hz, 1H), 7.40 (ddd, J=2.6, 5.6, 8.3 Hz, 1H), 7.13 (d, J=8.9 Hz, 1H), 4.15 (t, J=5.7 Hz, 2H), 2.85 (t, J=5.7 Hz, 2H), 2.57-2.46 (m, 4H), 2.32 (s, 3H), 2.17 (s, 3H), 1.75 (td, J=3.3, 6.8 Hz, 4H).

### 4-chloro-N-(3-(3,5-dimethylisoxazol-4-yl)-4-(2-(pyrrolidin-1-yl)ethoxy)phenyl)picolinamide (105)

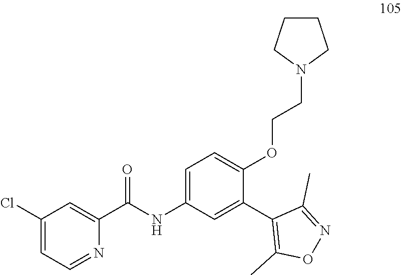 [SMILES: Cc1noc(C)c1-c1cc(NC(=O)c2cc(Cl)ccn2)ccc1OCCN1CCCC1]

Compound 105 was prepared according to the synthesis described for compound 100, substituting 4-chloropicolinic acid for 2-cyclopropylcyclopropanecarboxylic acid.

LCMS (ESI): m/z [M+H] calcd for C₂₃H₂₆Cl^{35/37}N₄O₃: 441.16 and 443.16; found: 441.1 and 443.1.

¹H NMR (400 MHZ, methanol-d₄) δ=8.67-8.61 (m, 1H), 8.19 (d, J=2.1 Hz, 1H), 7.86 (dd, J=2.7, 8.9 Hz, 1H), 7.72 (d, J=2.7 Hz, 1H), 7.68-7.65 (m, 1H), 7.21 (d, J=9.0 Hz, 1H), 4.39-4.34 (m, 2H), 3.65-3.59 (m, 2H), 3.58-3.46 (m, 2H), 3.03 (br s, 2H), 2.35 (s, 3H), 2.19 (s, 3H), 2.14-2.02 (m, 2H), 1.95 (br s, 2H).

### (1S,2R)—N-[3-(3,5-dimethylisoxazol-4-yl)-4-(2-pyrrolidin-1-ylethoxy)phenyl]-2-fluoro-cyclopropanecarboxamide (106)

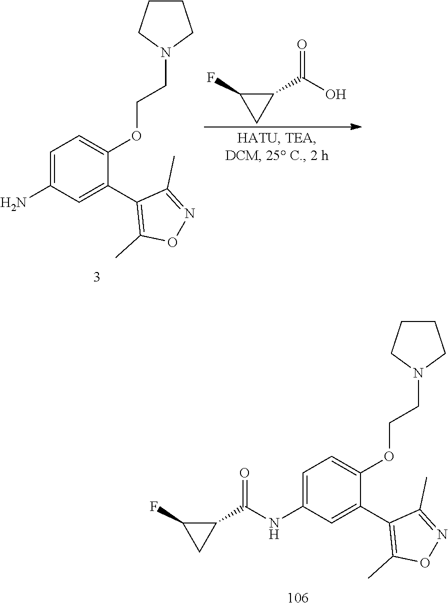

To a solution of (1S,2R)-2-fluorocyclopropanecarboxylic acid (41.44 mg, 398.17 μmol, 1.2 eq.) in DCM (1 mL) was added HATU (151.39 mg, 398.17 μmol, 1.2 eq.) and TEA (100.72 mg, 995.41 μmol, 138.55 μL, 3 eq.), then 3-(3,5-dimethylisoxazol-4-yl)-4-(2-pyrrolidin-1-ylethoxy)aniline ((3), 100 mg, 331.80 μmol, 1 eq.). The mixture was stirred at 25° C. for 2 hrs. The reaction mixture was filtered and concentrated under reduced pressure to give a residue. The residue was purified by prep-HPLC (column: Phenomenex Luna C18 150*25 mm*10 um; mobile phase: [water (0.1% TFA)-ACN]; B %: 9%-39%, 10 min). (1S,2R)—N-[3-(3,5-dimethylisoxazol-4-yl)-4-(2-pyrrolidin-1-ylethoxy)phenyl]-2-fluoro-cyclopropanecarboxamide (57.89 mg, 114.53 μmol, 34.52% yield, 99.215% purity, TFA) was obtained as a brown gum.

LCMS (ESI): m/z [M+H] calcd for C₂₁H₂₉FN₃O₃: 388.20; found: 388.1.

¹H NMR (400 MHZ, methanol-d₄) δ=7.59 (dd, J=2.6, 8.9 Hz, 1H), 7.43 (d, J=2.7 Hz, 1H), 7.14 (d, J=8.9 Hz, 1H), 4.90-4.73 (m, 1H), 4.31 (s, 2H), 3.61-3.56 (m, 2H), 3.55-3.46 (m, 2H), 3.03 (br d, J=7.0 Hz, 2H), 2.32 (s, 3H), 2.27-2.17 (m, 1H), 2.16 (s, 3H), 2.09 (br s, 2H), 1.98-1.89 (m, 2H), 1.53-1.41 (m, 1H), 1.32 (qd, J=6.5, 12.9 Hz, 1H).

### (1R,2R)—N-(3-(3,5-dimethylisoxazol-4-yl)-4-(2-(pyrrolidin-1-yl)ethoxy)phenyl)-2-fluorocyclopropane-1-carboxamide (107)

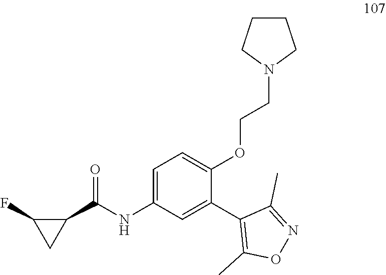 [SMILES: Cc1noc(C)c1-c1cc(NC(=O)[C@H]2C[C@H]2F)ccc1OCCN1CCCC1]

Compound 107 was prepared according to the synthesis described for compound 106, substituting (1R,2R)-2-fluorocyclopropanecarboxylic acid for (1S,2R)-2-fluorocyclopropanecarboxylic acid.

LCMS (ESI): m/z [M+H] calcd for C₂₁H₂₇FN₃O₃: 388.20; found: 388.3.

¹H NMR (400 MHZ, methanol-d₄) δ=7.62 (dd, J=2.7, 8.9 Hz, 1H), 7.46 (d, J=2.6 Hz, 1H), 7.14 (d, J=9.0 Hz, 1H), 4.94-4.75 (m, 1H), 4.36-4.27 (m, 2H), 3.62-3.56 (m, 2H), 3.51 (br s, 2H), 3.03 (br d, J=5.8 Hz, 2H), 2.33 (s, 3H), 2.17 (s, 3H), 2.09 (br s, 2H), 2.01-1.87 (m, 3H), 1.82-1.66 (m, 1H), 1.17 (tdd, J=6.4, 9.2, 12.4 Hz, 1H).

### (1S,2S)—N-(3-(3,5-dimethylisoxazol-4-yl)-4-(2-(pyrrolidin-1-yl)ethoxy)phenyl)-2-fluorocyclopropane-1-carboxamide (108)

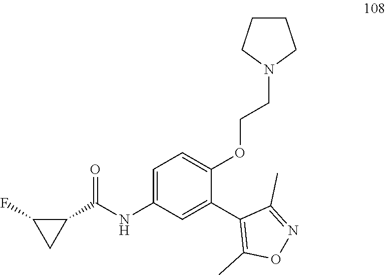 [SMILES: Cc1noc(C)c1-c1cc(NC(=O)[C@@H]2C[C@@H]2F)ccc1OCCN1CCCC1]

Compound 108 was prepared according to the synthesis described for compound 106, substituting (1S,2S)-2-fluorocyclopropanecarboxylic acid for (1S,2R)-2-fluorocyclopropanecarboxylic acid.

LCMS (ESI): m/z [M+H] calcd for C₂₁H₂₇FN₃O₃: 388.20; found: 388.1.

¹H NMR (400 MHZ, methanol-d₄) δ=7.63 (dd, J=2.7, 8.9 Hz, 1H), 7.46 (d, J=2.6 Hz, 1H), 7.14 (d, J=8.9 Hz, 1H), 4.92-4.75 (m, 1H), 4.34-4.29 (m, 2H), 3.61-3.56 (m, 2H), 3.51 (br s, 2H), 3.09-2.94 (m, 2H), 2.32 (s, 3H), 2.17 (s, 3H), 2.09 (br s, 2H), 2.01-1.89 (m, 3H), 1.80-1.68 (m, 1H), 1.22-1.10 (m, 1H).

### N-(3-(3,5-dimethylisoxazol-4-yl)-4-(2-(pyrrolidin-1-yl)ethoxy)phenyl)-4-methylthiazole-5-carboxamide (109)

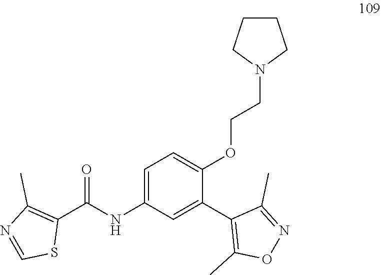 [SMILES: Cc1ncsc1C(=O)Nc1ccc(OCCN2CCCC2)c(-c2c(C)noc2C)c1]

Compound 109 was prepared according to the synthesis described for compound 106, substituting 4-methylthiazole-5-carboxylic acid for (1S,2R)-2-fluorocyclopropanecarboxylic acid.

LCMS (ESI): m/z [M+H] calcd for C₂₂H₂₇N₄O₃S: 427.17; found: 427.2.

¹H NMR (400 MHz, methanol-d₄) δ=9.01 (s, 1H), 7.63 (dd, J=2.4, 8.9 Hz, 1H), 7.48 (d, J=2.3 Hz, 1H), 7.13 (d, J=8.9 Hz, 1H), 4.16 (t, J=5.6 Hz, 2H), 2.90 (t, J=5.6 Hz, 2H), 2.69 (s, 3H), 2.57 (br s, 4H), 2.32 (s, 3H), 2.17 (s, 3H), 1.77 (td, J=3.2, 6.5 Hz, 4H).

### N-(3-(3,5-dimethylisoxazol-4-yl)-4-(2-(pyrrolidin-1-yl)ethoxy)phenyl)-2,4-dimethylthiazole-5-carboxamide (110)

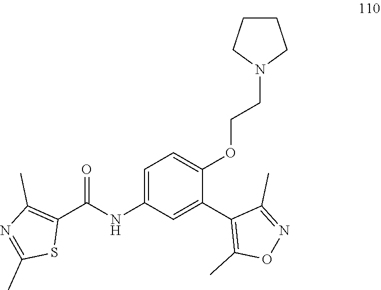 [SMILES: Cc1nc(C)c(C(=O)Nc2ccc(OCCN3CCCC3)c(-c3c(C)noc3C)c2)s1]

Compound 110 was prepared according to the synthesis described for compound 106, substituting 2,4-dimethylthiazole-5-carboxylic acid for (1S,2R)-2-fluorocyclopropanecarboxylic acid.

LCMS (ESI): m/z [M+H] calcd for C₂₃H₂₉N₄O₃S: 441.19; found: 441.3.

¹H NMR (400 MHZ, methanol-d₄) δ=7.60 (dd, J=2.6, 8.9 Hz, 1H), 7.45 (d, J=2.6 Hz, 1H), 7.11 (d, J=8.9 Hz, 1H), 4.14 (t, J=5.6 Hz, 2H), 2.85 (t, J=5.7 Hz, 2H), 2.70 (s, 3H), 2.61 (s, 3H), 2.55-2.49 (m, 4H), 2.31 (s, 3H), 2.17 (s, 3H), 1.75 (td, J=3.3, 6.9 Hz, 4H).

### N-(3-(3,5-dimethylisoxazol-4-yl)-4-(2-(pyrrolidin-1-yl)ethoxy)phenyl)-4-methylisoxazole-5-carboxamide (111)

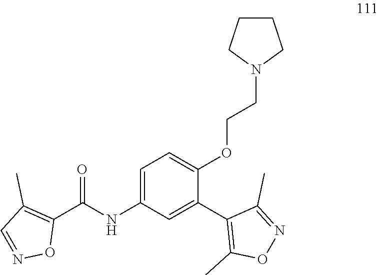 [SMILES: Cc1cnoc1C(=O)Nc1ccc(OCCN2CCCC2)c(-c2c(C)noc2C)c1]

Compound 111 was prepared according to the synthesis described for compound 106, substituting 4-methylisoxazole-5-carboxylic acid for (1S,2R)-2-fluorocyclopropanecarboxylic acid.

LCMS (ESI): m/z [M+H] calcd for C₂₂H₂₇N₄O₄: 411.20; found: 441.3.

¹H NMR (400 MHZ, methanol-d₄) δ=8.43 (s, 1H), 7.79 (dd, J=2.7, 8.9 Hz, 1H), 7.60 (d, J=2.6 Hz, 1H), 7.20 (d, J=8.9 Hz, 1H), 4.38-4.33 (m, 2H), 3.64-3.58 (m, 2H), 3.52 (br d, J=2.7 Hz, 2H), 3.04 (br d, J=4.8 Hz, 2H), 2.35 (d, J=2.4 Hz, 6H), 2.19 (s, 3H), 2.13-1.90 (m, 4H).

### N-(3-(3,5-dimethylisoxazol-4-yl)-4-(2-(pyrrolidin-1-yl)ethoxy)phenyl)-4-methyloxazole-5-carboxamide (112)

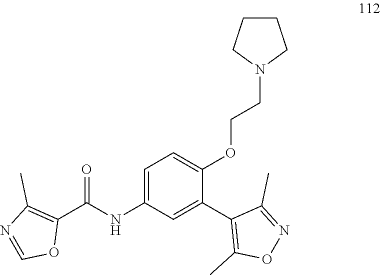 [SMILES: Cc1ncoc1C(=O)Nc1ccc(OCCN2CCCC2)c(-c2c(C)noc2C)c1]

Compound 112 was prepared according to the synthesis described for compound 106, substituting 4-methyloxazole-5-carboxylic acid for (1S,2R)-2-fluorocyclopropanecarboxylic acid.

LCMS (ESI): m/z [M+H] calcd for C₂₂H₂₇N₄O₄: 411.20; found: 411.2.

¹H NMR (400 MHZ, methanol-d₄) δ=8.24 (s, 1H), 7.67 (dd, J=2.7, 8.9 Hz, 1H), 7.52 (d, J=2.6 Hz, 1H), 7.12 (d, J=9.0 Hz, 1H), 4.15 (t, J=5.6 Hz, 2H), 2.86 (t, J=5.6 Hz, 2H), 2.55-2.51 (m, 4H), 2.50 (s, 3H), 2.32 (s, 3H), 2.17 (s, 3H), 1.75 (td, J=3.3, 6.7 Hz, 4H).

### N-[3-(3,5-dimethylisoxazol-4-yl)-4-(2-pyrrolidin-1-ylethoxy)phenyl]-1-fluoro-cyclopropanecarboxamide (113)

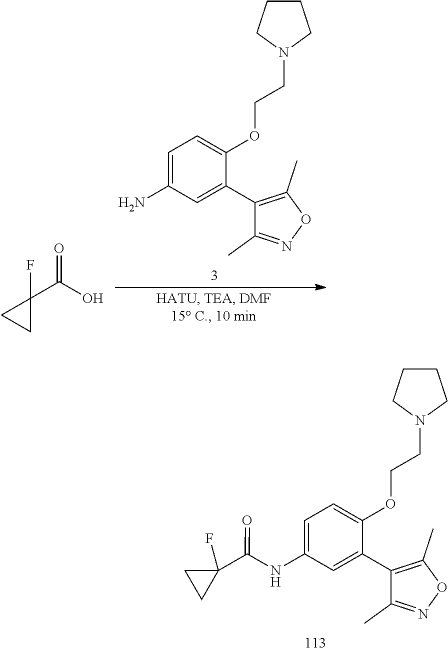 [SMILES: O=C(O)C1(F)CC1 ; Cc1noc(C)c1-c1cc(N)ccc1OCCN1CCCC1 ; Cc1noc(C)c1-c1cc(NC(=O)C2(F)CC2)ccc1OCCN1CCCC1]

To a mixture of 1-fluorocyclopropanecarboxylic acid (33.15 mg, 318.53 μmol, 1.2 eq.), HATU (121.12 mg, 318.53 μmol, 1.2 eq.) and TEA (53.72 mg, 530.89 μmol, 73.89 μL, 2 eq.) in DMF (1 mL) was added 3-(3,5-dimethylisoxazol-4-yl)-4-(2-pyrrolidin-1-ylethoxy)aniline ((3), 80 mg, 265.44 μmol, 1 eq.). The mixture was stirred at 15° C. for 10 min (monitored by LC-MS). The reaction mixture was concentrated under reduced pressure to give a residue. The residue was purified by prep-HPLC (column: Waters Xbridge 150*25 mm*5 um; mobile phase: [water (0.05% ammonia hydroxide v/v)-ACN]; B %: 33%-63%, 10 min). N-[3-(3,5-dimethylisoxazol-4-yl)-4-(2-pyrrolidin-1-ylethoxy)phenyl]-1-fluoro-cyclopropanecarboxamide (61.41 mg, 158.50 μmol, 59.71% yield, 100% purity) was obtained as a white solid.

LCMS (ESI): m/z [M+H] calcd for C₂₁H₂₇FN₃O₃: 388.20; found: 388.3.

¹H NMR (400 MHZ, methanol-d₄) δ=7.60 (dd, J=2.7, 8.9 Hz, 1H), 7.46 (d, J=2.7 Hz, 1H), 7.09 (d, J=8.9 Hz, 1H), 4.13 (t, J=5.7 Hz, 2H), 2.83 (t, J=5.7 Hz, 2H), 2.56-2.43 (m, 4H), 2.30 (s, 3H), 2.16 (s, 3H), 1.75 (td, J=3.3, 6.9 Hz, 4H), 1.42-1.38 (m, 2H), 1.36 (s, 2H).

### N-(3-(3,5-dimethylisoxazol-4-yl)-4-(2-(pyrrolidin-1-yl)ethoxy)phenyl)-4-fluoro-1-methyl-1H-pyrazole-5-carboxamide (114)

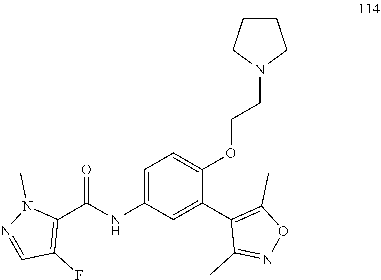 [SMILES: Cc1noc(C)c1-c1cc(NC(=O)c2c(F)cnn2C)ccc1OCCN1CCCC1]

Compound 114 was prepared according to the synthesis described for compound substituting 113, 4-fluoro-1-methyl-1H-pyrazole-5-carboxylic acid for 1-fluorocyclopropanecarboxylic acid.

LCMS (ESI): m/z [M+H] calcd for C₂₂H₂₇FN₅O₃: 428.0, found: 428.3.

¹H NMR (400 MHZ, methanol-d₄) δ=7.72 (br d, J=9.0 Hz, 1H), 7.57 (d, J=2.0 Hz, 1H), 7.47 (d, J=4.3 Hz, 1H), 7.20 (d, J=9.0 Hz, 1H), 4.35 (t, J=4.6 Hz, 2H), 4.05 (s, 3H), 3.64-3.58 (m, 2H), 3.52 (br s, 2H), 3.03 (br d, J=2.1 Hz, 2H), 2.34 (s, 3H), 2.19 (s, 3H), 2.09 (br s, 2H), 1.94 (br d, J=5.8 Hz, 2H).

### N-(3-(3,5-dimethylisoxazol-4-yl)-4-(2-(pyrrolidin-1-yl)ethoxy)phenyl)-2-ethoxycyclopropane-1-carboxamide (115)

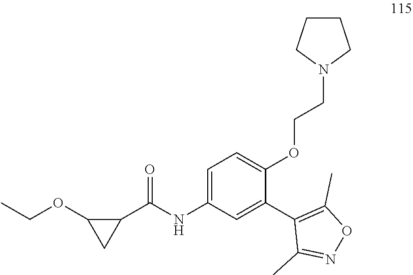 [SMILES: CCOC1CC1C(=O)Nc1ccc(OCCN2CCCC2)c(-c2c(C)noc2C)c1]

Compound 115 was prepared according to the synthesis described for compound 113, substituting 2-ethoxycyclopropane-1-carboxylic acid for 1-fluorocyclopropanecarboxylic acid.

LCMS (ESI): m/z [M+H] calcd for C₂₃H₃₂N₃O₄: 414.0, found: 414.3.

¹H NMR (400 MHZ, methanol-d₄) δ=7.51 (dd, J=2.6, 8.9 Hz, 1H), 7.40 (d, J=2.6 Hz, 1H), 7.06 (d, J=9.0 Hz, 1H), 4.11 (t, J=5.6 Hz, 2H), 3.68-3.57 (m, 3H), 2.83 (t, J=5.7 Hz, 2H), 2.52 (br t, J=5.4 Hz, 4H), 2.29 (s, 3H), 2.15 (s, 3H), 1.88 (ddd, J=1.9, 5.9, 9.4 Hz, 1H), 1.79-1.72 (m, 4H), 1.28-1.15 (m, 5H).

### N-[3-(3,5-dimethylisoxazol-4-yl)-4-(2-pyrrolidin-1-ylethoxy)phenyl]-2-methyl-pyrazole-3-carboxamide (116)

### Preparation of N-[3-bromo-4-(2-pyrrolidin-1-ylethoxy)phenyl]-2-methyl-pyrazole-3-carboxamide (4)

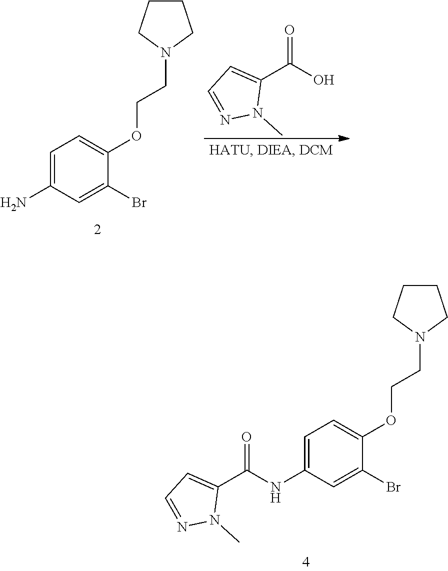

To a solution of 2-methylpyrazole-3-carboxylic acid (2.23 g, 17.71 mmol, 1.01 eq.) in DCM (50 mL) was added HATU (8.00 g, 21.04 mmol, 1.2 eq.) and DIEA (6.80 g, 52.60 mmol, 9.16 mL, 3 eq.), then 3-bromo-4-(2-pyrrolidin-1-ylethoxy)aniline ((2), 5 g, 17.53 mmol, 1 eq.) after 30 min. The mixture was stirred at 25° C. for 2 hrs. The reaction mixture was concentrated under reduced pressure to give a residue. The crude product was purified by reversed-phase HPLC (0.1% NH₃·H₂O conditions). N-[3-bromo-4-(2-pyrrolidin-1-ylethoxy)phenyl]-2-methyl-pyrazole-3-carboxamide ((4), 2.4 g, 6.10 mmol, 34.81% yield) was obtained as a black oil.

### N-[3-(3,5-dimethylisoxazol-4-yl)-4-(2-pyrrolidin-1-ylethoxy)phenyl]-2-methyl-pyrazole-3-carboxamide (116)

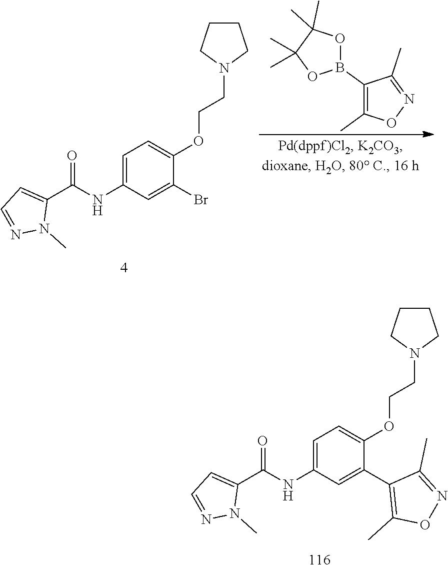

A mixture of N-[3-bromo-4-(2-pyrrolidin-1-ylethoxy)phenyl]-2-methyl-pyrazole-3-carboxamide ((4), 200 mg, 508.55 μmol, 1 eq.), 3,5-dimethyl-4-(4,4,5,5-tetramethyl-1,3,2-dioxaborolan-2-yl)isoxazole (113.45 mg, 508.55 μmol, 1 eq.), Pd(dppf)Cl₂ (37.21 mg, 50.85 μmol, 0.1 eq.) and K₂CO₃ (140.57 mg, 1.02 mmol, 2 eq.) in dioxane (3 mL) and H₂O (0.8 mL) was degassed and purged with N₂ three times, and then the mixture was stirred at 80° C. for 2 hrs under an N₂ atmosphere. To the reaction mixture was added thiourea resin and stirred at 25° C. for 16 hrs, then filtered and concentrated under reduced pressure to give a residue. The residue was purified by prep-HPLC (column: Phenomenex Synergi Max-RP 250*50 mm*10 um; mobile phase: [water (0.1% TFA)-ACN]; B %: 10%-40%, 10 min). N-[3-(3,5-dimethylisoxazol-4-yl)-4-(2-pyrrolidin-1-ylethoxy)phenyl]-2-methyl-pyrazole-3-carboxamide (77.96 mg, 147.55 μmol, 29.01% yield, 99.077% purity, TFA) was obtained as a yellow solid.

LCMS (ESI): m/z [M+H] calcd for C₂₂H₂₈N₅O₃: 410.21; found: 410.3.

¹H NMR (400 MHz, methanol-d₄) δ=7.74 (dd, J=2.6, 8.9 Hz, 1H), 7.57 (d, J=2.6 Hz, 1H), 7.51 (d, J=2.1 Hz, 1H), 7.19 (d, J=8.9 Hz, 1H), 6.96 (d, J=2.1 Hz, 1H), 4.40-4.32 (m, 2H), 3.63-3.58 (m, 2H), 3.58-3.43 (m, 2H), 3.02 (br s, 2H), 2.34 (s, 3H), 2.18 (s, 3H), 2.08 (br s, 2H), 1.94 (br d, J=3.7 Hz, 2H).

### N-[4-[2-(azetidin-1-yl)ethoxy]-3-(3,5-dimethylisoxazol-4-yl)phenyl]-1-fluoro-cyclopropanecarboxamide (117)

### Preparation of 4-(2-fluoro-5-nitro-phenyl)-3,5-dimethyl-isoxazole (5)

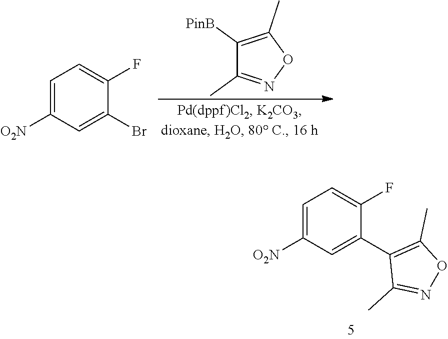

To a solution of 2-bromo-1-fluoro-4-nitro-benzene (5 g, 22.73 mmol, 1 eq.), K₂CO₃ (6.28 g, 45.46 mmol, 2 eq.) and 3,5-dimethyl-4-(4,4,5,5-tetramethyl-1,3,2-dioxaborolan-2-yl)isoxazole (5.58 g, 25.00 mmol, 1.1 eq.) in dioxane (50 mL) and H₂O (10 mL) was added Pd(dppf)Cl₂ (831.58 mg, 1.14 mmol, 0.05 eq.). The reaction mixture was stirred at 80° C. for 3 hrs. The reaction mixture was concentrated under vacuum. The residue was poured into water (100 mL). The aqueous phase was extracted with ethyl acetate (80 mL×3). The combined organic phase was washed with brine (60 mL), dried with anhydrous Na₂SO₄, filtered and concentrated under vacuum. The residue was purified by flash silica gel chromatography (ISCO®; 40 g SepaFlash® Silica Flash Column, eluent of 0˜20% ethyl acetate/petroleum ether gradient). 4-(2-fluoro-5-nitro-phenyl)-3,5-dimethyl-isoxazole ((5), 2.6 g, 11.01 mmol, 48.43% yield) was obtained as a white solid.

### Preparation of [2-(azetidin-1-yl)-2-oxo-ethyl] acetate (6)

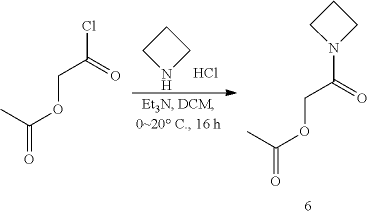

To a solution of azetidine (20 g, 213.78 mmol, 23.64 mL, 1 eq., HCl) and Et₃N (43.26 g, 427.56 mmol, 59.51 mL, 2 eq.) in DCM (200 mL) was added (2-chloro-2-oxo-ethyl) acetate (29.77 g, 218.05 mmol, 23.44 mL, 1.02 eq.) at 0° C. The reaction mixture was stirred at 20° C. for 4 hrs. Et₃N (14.54 g, 143.69 mmol, 20 mL, 6.72e-1 eq.) was added to the reaction mixture. The reaction mixture was stirred at 20° C. for 16 hrs. The reaction mixture was filtered and the filtrate was concentrated under vacuum. The residue was poured into water (100 mL). The pH of the aqueous phase was adjusted to 8. The aqueous phase was extracted with ethyl acetate (200 mL×3). The combined organic phase was washed with brine (100 mL), dried with anhydrous Na₂SO₄, filtered and concentrated under vacuum. [2-(azetidin-1-yl)-2-oxo-ethyl] acetate ((6), 13.8 g, 87.80 mmol, 41.07% yield) was obtained as a yellow oil. ¹H NMR (400 MHZ, chloroform-d) 8=4.49 (s, 2H), 4.23 (t, J=7.7 Hz, 2H), 4.12-4.04 (m, 2H), 2.41-2.28 (m, 2H), 2.15 (s, 3H).

### Preparation of 2-(azetidin-1-yl)ethanol (7)

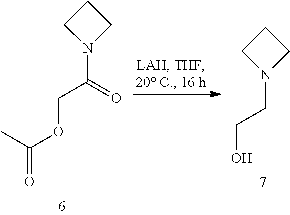

To a solution of LiAlH₄ (6.67 g, 175.61 mmol, 2 eq.) in THF (100 mL) was added [2-(azetidin-1-yl)-2-oxo-ethyl] acetate ((6), 13.8 g, 87.80 mmol, 1 eq.) in THF (20 mL) at 20° C. The reaction mixture was stirred at 20° C. for 16 hrs. The reaction mixture was quenched with Na₂SO₄·10 H₂O (50 g). The reaction mixture was diluted with EtOAc (200 mL) and stirred at 20° C. for 20 min. The mixture was filtered and the filtrate was concentrated under vacuum. 2-(azetidin-1-yl)ethanol ((7), 3.2 g, 31.64 mmol, 36.03% yield) was obtained as a yellow oil.

¹H NMR (400 MHZ, DMSO-d₆) δ=4.37-4.26 (m, 1H), 3.32-3.25 (m, 2H), 3.12-3.04 (m, 4H), 2.37 (t, J=6.3 Hz, 2H), 1.97-1.88 (m, 2H).

### Preparation of 4-[2-[2-(azetidin-1-yl)ethoxy]-5-nitro-phenyl]-3,5-dimethyl-isoxazole (8)

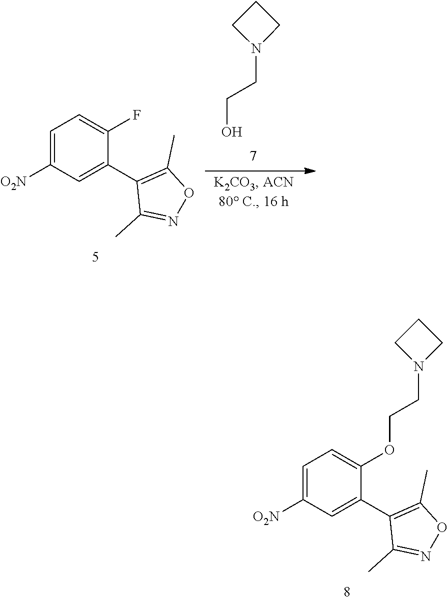 [SMILES: Cc1noc(C)c1-c1cc([N+](=O)[O-])ccc1F ; OCCN1CCC1 ; Cc1noc(C)c1-c1cc([N+](=O)[O-])ccc1OCCN1CCC1]

To a solution of 4-(2-fluoro-5-nitro-phenyl)-3,5-dimethyl-isoxazole ((5), 1.5 g, 6.35 mmol, 1 eq.) in CH₃CN (25 mL) was added Cs₂CO₃ (4.14 g, 12.70 mmol, 2 eq.) and 2-(azetidin-1-yl)ethanol ((7), 706.58 mg, 6.99 mmol, 1.1 eq.). The mixture was stirred at 80° C. for 18 hrs. The mixture was concentrated under vacuum. The crude product was purified by reversed-phase HPLC (0.1% TFA condition). 4-[2-[2-(azetidin-1-yl)ethoxy]-5-nitro-phenyl]-3,5-dimethyl-isoxazole ((8), 1.7 g, 5.36 mmol, 84.36% yield) was obtained as a white solid.

### Preparation of 4-[2-(azetidin-1-yl)ethoxy]-3-(3,5-dimethylisoxazol-4-yl)aniline (9)

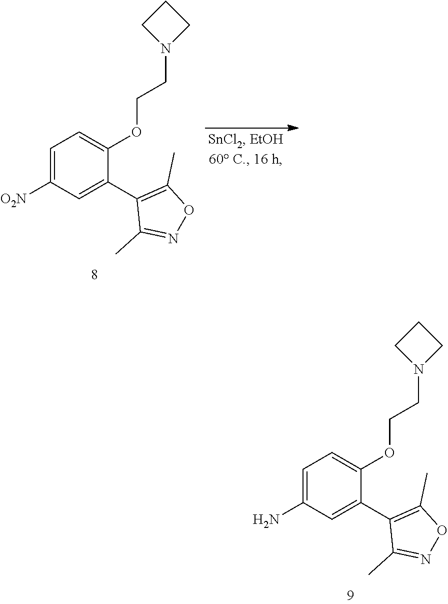 [SMILES: Cc1noc(C)c1-c1cc([N+](=O)[O-])ccc1OCCN1CCC1 ; Cc1noc(C)c1-c1cc(N)ccc1OCCN1CCC1]

To a solution of 4-[2-[2-(azetidin-1-yl)ethoxy]-5-nitro-phenyl]-3,5-dimethyl-isoxazole ((8), 1.7 g, 5.36 mmol, 1 eq.) in EtOH (20 mL) was added dichlorotin (3.05 g, 16.07 mmol, 416.87 μL, 3 eq.). The reaction mixture was stirred at 60° C. for 16 hrs. The reaction mixture was concentrated under vacuum. The residue was poured into sat. NaHCO₃ aq. (100 mL) and EtOAc (200 mL). The mixture was filtered. The aqueous phase was extracted with ethyl acetate (100 mL×2). The combined organic phase was washed with brine (50 mL), dried with anhydrous Na₂SO₄, filtered and concentrated under vacuum. 4-[2-(azetidin-1-yl)ethoxy]-3-(3,5-dimethylisoxazol-4-yl)aniline ((9), 1 g, 3.48 mmol, 64.96% yield) was obtained as a yellow solid.

¹H NMR (400 MHZ, methanol-d₄) δ=6.89-6.83 (m, 1H), 6.80-6.73 (m, 1H), 6.59 (d, J=2.7 Hz, 1H), 3.86 (t, J=5.4 Hz, 2H), 3.17 (t, J=7.2 Hz, 4H), 2.72 (t, J=5.4 Hz, 2H), 2.28 (s, 3H), 2.14 (s, 3H), 2.08-1.98 (m, 2H).

### Preparation of N-[4-[2-(azetidin-1-yl)ethoxy]-3-(3,5-dimethylisoxazol-4-yl)phenyl]-1-fluoro-cyclopropanecarboxamide (117)

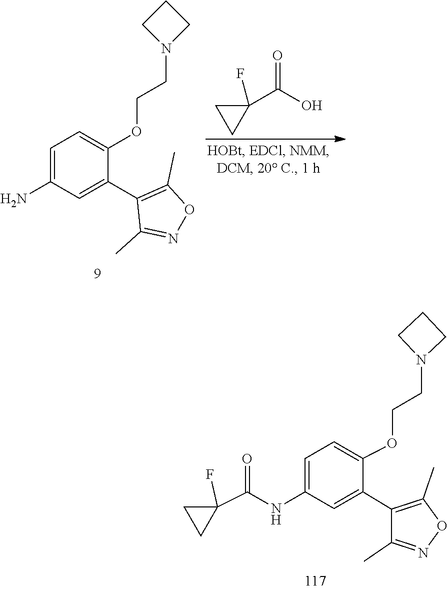 [SMILES: Cc1noc(C)c1-c1cc(N)ccc1OCCN1CCC1 ; O=C(O)C1(F)CC1 ; Cc1noc(C)c1-c1cc(NC(=O)C2(F)CC2)ccc1OCCN1CCC1]

To a solution of 4-[2-(azetidin-1-yl)ethoxy]-3-(3,5-dimethylisoxazol-4-yl)aniline ((9), 60 mg, 208.80 μmol, 1 eq.), 1-fluorocyclopropanecarboxylic acid (23.91 mg, 229.68 μmol, 1.1 eq.), EDCl (48.03 mg, 250.56 μmol, 1.2 eq.) and HOBt (33.86 mg, 250.56 μmol, 1.2 eq.) in DCM (1 mL) was added NMM (4.22 mg, 41.76 μmol, 4.59 μL, 0.2 eq.). The reaction mixture was stirred at 20° C. for 1 hr. The reaction mixture was concentrated under vacuum. The residue was purified by prep-HPLC (column: Waters Xbridge 150*25 mm*5 um; mobile phase: [water (10 mM NH₄HCO₃)-ACN]; B %: 18%-48%, 9 min.) followed by lyophilization. N-[4-[2-(azetidin-1-yl)ethoxy]-3-(3,5-dimethylisoxazol-4-yl)phenyl]-1-fluoro-cyclopropanecarboxamide (31.33 mg, 83.90 μmol, 40.18% yield, 100% purity) was obtained as an off-white solid.

LCMS (ESI): m/z [M+H] calcd for C₂₀H₂₅FN₃O₃: 374.18; found: 374.2.

¹H NMR (400 MHZ, methanol-d₄) δ ppm 7.60 (dd, J=8.86, 2.75 Hz, 1H) 7.45 (d, J=2.69 Hz, 1H) 7.05 (d, J=8.93 Hz, 1H) 4.00 (t, J=5.32 Hz, 2H) 3.18 (t, J=7.21 Hz, 4H) 2.79 (t, J=5.32 Hz, 2H) 2.30 (s, 3H) 2.16 (s, 3H) 1.99-2.09 (m, 2H) 1.38-1.43 (m, 2H) 1.36 (s, 2H).

### N-(4-(2-(azetidin-1-yl)ethoxy)-3-(3,5-dimethylisoxazol-4-yl)phenyl)-1-methyl-1H-pyrazole-5-carboxamide (118)

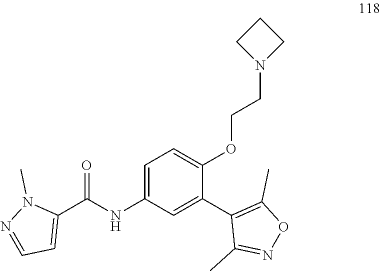 [SMILES: Cc1noc(C)c1-c1cc(NC(=O)c2ccnn2C)ccc1OCCN1CCC1]

Compound 118 was prepared according to the synthesis described for compound 117, substituting 1-methyl-1H-pyrazole-5-carboxylic acid for 1-fluorocyclopropanecarboxylic acid.

LCMS (ESI): m/z [M+H] calcd for C₂₁H₂₆N₅O₃: 396.0; found: 396.0.

¹H NMR (400 MHZ, methanol-d₄) δ=7.66 (dd, J=2.6, 8.9 Hz, 1H), 7.51 (d, J=3.2 Hz, 2H), 7.08 (d, J=8.9 Hz, 1H), 6.95 (d, J=2.1 Hz, 1H), 4.15 (s, 3H), 4.01 (t, J=5.3 Hz, 2H), 3.21-3.14 (m, 4H), 2.79 (t, J=5.3 Hz, 2H), 2.32 (s, 3H), 2.17 (s, 3H), 2.04 (quin, J=7.2 Hz, 2H).

### N-(4-(2-(azetidin-1-yl)ethoxy)-3-(3,5-dimethylisoxazol-4-yl)phenyl)-4-methylthiazole-5-carboxamide (119)

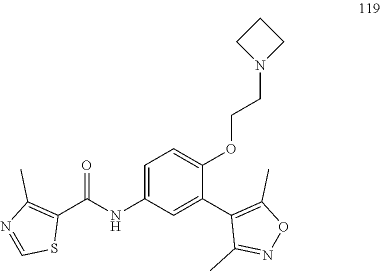 [SMILES: Cc1ncsc1C(=O)Nc1ccc(OCCN2CCC2)c(-c2c(C)noc2C)c1]

Compound 119 was prepared according to the synthesis described for compound 117, substituting 4-methylthiazole-5-carboxylic acid for 1-fluorocyclopropanecarboxylic acid.

LCMS (ESI): m/z [M+H] calcd for C₂₁H₂₅N₄O₃S: 413.0; found: 412.9.

¹H NMR (400 MHZ, methanol-d₄) δ=9.03 (s, 1H), 7.65 (dd, J=2.6, 8.9 Hz, 1H), 7.49 (d, J=2.5 Hz, 1H), 7.12 (d, J=8.9 Hz, 1H), 4.07 (t, J=5.3 Hz, 2H), 3.38-3.34 (m, 4H), 2.95 (t, J=5.2 Hz, 2H), 2.70 (s, 3H), 2.34 (s, 3H), 2.19 (s, 3H), 2.15-2.08 (m, 2H).

### N-(4-(2-(azetidin-1-yl)ethoxy)-3-(3,5-dimethylisoxazol-4-yl)phenyl)benzamide (120)

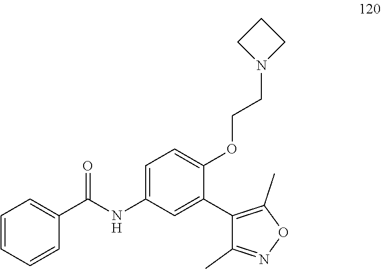 [SMILES: Cc1noc(C)c1-c1cc(NC(=O)c2ccccc2)ccc1OCCN1CCC1]

Compound 120 was prepared according to the synthesis described for compound 117, substituting benzoic acid for 1-fluorocyclopropanecarboxylic acid.

LCMS (ESI): m/z [M+H] calcd for C₂₃H₂₆N₃O₃: 392.0; found: 392.0.

¹H NMR (400 MHZ, methanol-d₄) δ=7.97-7.90 (m, 2H), 7.69 (dd, J=2.6, 8.9 Hz, 1H), 7.61-7.55 (m, 1H), 7.55-7.47 (m, 3H), 7.09 (d, J=8.9 Hz, 1H), 4.02 (t, J=5.4 Hz, 2H), 3.18 (t, J=7.2 Hz, 4H), 2.79 (t, J=5.3 Hz, 2H), 2.33 (s, 3H), 2.18 (s, 3H), 2.04 (quin, J=7.2 Hz, 2H).

### N-(4-(2-(azetidin-1-yl)ethoxy)-3-(3,5-dimethylisoxazol-4-yl)phenyl)-4-fluorobenzamide (121)

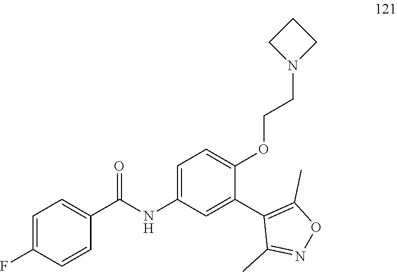 [SMILES: Cc1noc(C)c1-c1cc(NC(=O)c2ccc(F)cc2)ccc1OCCN1CCC1]

Compound 121 was prepared according to the synthesis described for compound 117, substituting 4-methylthiazole-5-carboxylic acid for 1-fluorocyclopropanecarboxylic acid.

LCMS (ESI): m/z [M+H] calcd for C₂₃H₂₅N₃O₃: 410.0; found: 410.0.

¹H NMR (400 MHZ, methanol-d₄) δ=8.05-7.95 (m, 2H), 7.67 (dd, J=2.7, 8.9 Hz, 1H), 7.52 (d, J=2.7 Hz, 1H), 7.29-7.20 (m, 2H), 7.08 (d, J=8.9 Hz, 1H), 4.02 (t, J=5.3 Hz, 2H), 3.18 (t, J=7.2 Hz, 4H), 2.79 (t, J=5.3 Hz, 2H), 2.32 (s, 3H), 2.18 (s, 3H), 2.04 (quin, J=7.2 Hz, 2H).

### N-(4-(2-(azetidin-1-yl)ethoxy)-3-(3,5-dimethylisoxazol-4-yl)phenyl)-3-methoxybenzamide (122)

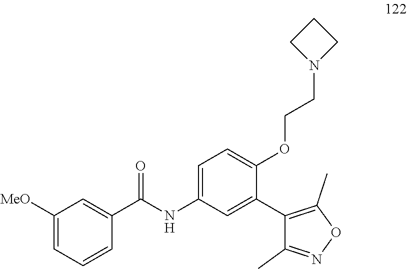 [SMILES: COc1cccc(C(=O)Nc2ccc(OCCN3CCC3)c(-c3c(C)noc3C)c2)c1]

Compound 122 was prepared according to the synthesis described for compound 117, substituting 3-methoxybenzoic acid for 1-fluorocyclopropanecarboxylic acid.

LCMS (ESI): m/z [M+H] calcd for C₂₄H₂₈N₃O₄: 422.0; found: 422.0.

¹H NMR (400 MHZ, methanol-d₄) δ=7.68 (dd, J=2.7, 8.9 Hz, 1H), 7.56-7.47 (m, 3H), 7.45-7.36 (m, 1H), 7.14 (ddd, J=1.0, 2.6, 8.2 Hz, 1H), 7.08 (d, J=9.0 Hz, 1H), 4.02 (t, J=5.4 Hz, 2H), 3.87 (s, 3H), 3.18 (t, J=7.2 Hz, 4H), 2.79 (t, J=5.3 Hz, 2H), 2.33 (s, 3H), 2.18 (s, 3H), 2.04 (quin, J=7.2 Hz, 2H).

### N-(4-(2-(azetidin-1-yl)ethoxy)-3-(3,5-dimethylisoxazol-4-yl)phenyl)-2-phenylacetamide (123)

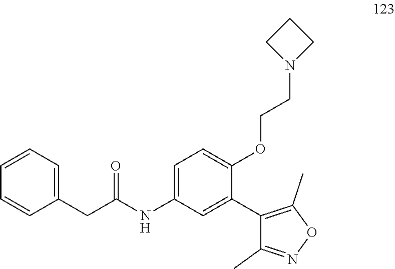 [SMILES: Cc1noc(C)c1-c1cc(NC(=O)Cc2ccccc2)ccc1OCCN1CCC1]

Compound 123 was prepared according to the synthesis described for compound 117, substituting 2-phenylacetic acid for 1-fluorocyclopropanecarboxylic acid.

LCMS (ESI): m/z [M+H] calcd for C₂₄H₂₈N₃O₃: 406.0; found: 406.0.

¹H NMR (400 MHZ, methanol-d₄) δ=7.52 (dd, J=2.6, 8.9 Hz, 1H), 7.40 (d, J=2.7 Hz, 1H), 7.39-7.20 (m, 5H), 7.02 (d, J=8.9 Hz, 1H), 3.98 (t, J=5.3 Hz, 2H), 3.66 (s, 2H), 3.17 (t, J=7.2 Hz, 4H), 2.77 (t, J=5.3 Hz, 2H), 2.28 (s, 3H), 2.14 (s, 3H), 2.03 (quin, J=7.2 Hz, 2H).

### N-(4-(2-(azetidin-1-yl)ethoxy)-3-(3,5-dimethylisoxazol-4-yl)phenyl)-2-(3-methoxyphenyl)acetamide (124)

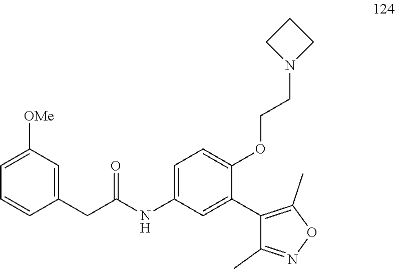 [SMILES: COc1cccc(CC(=O)Nc2ccc(OCCN3CCC3)c(-c3c(C)noc3C)c2)c1]

Compound 124 was prepared according to the synthesis described for compound 117, substituting 2-(3-methoxyphenyl)acetic acid for 1-fluorocyclopropanecarboxylic acid.

LCMS (ESI): m/z [M+H] calcd for C₃₅H₃₀N₃O₄: 436.0; found: 436.0.

¹H NMR (400 MHZ, methanol-d₄) δ=7.52 (dd, J=2.7, 8.9 Hz, 1H), 7.40 (d, J=2.6 Hz, 1H), 7.28-7.19 (m, 1H), 7.02 (d, J=8.9 Hz, 1H), 6.95-6.90 (m, 2H), 6.85-6.79 (m, 1H), 3.98 (t, J=5.3 Hz, 2H), 3.79 (s, 3H), 3.63 (s, 2H), 3.17 (t, J=7.2 Hz, 4H), 2.77 (t, J=5.4 Hz, 2H), 2.28 (s, 3H), 2.14 (s, 3H), 2.03 (quin, J=7.2 Hz, 2H).

### N-(4-(2-(azetidin-1-yl)ethoxy)-3-(3,5-dimethylisoxazol-4-yl)phenyl)-2-(3-fluorophenyl)acetamide (125)

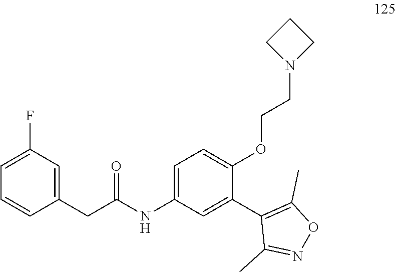 [SMILES: Cc1noc(C)c1-c1cc(NC(=O)Cc2cccc(F)c2)ccc1OCCN1CCC1]

Compound 125 was prepared according to the synthesis described for compound 117, substituting 2-(3-fluorophenyl)acetic acid for 1-fluorocyclopropanecarboxylic acid.

LCMS (ESI): m/z [M+H] calcd for C₂₄H₂₇FN₃O₃: 424.0; found: 424.0.

¹H NMR (400 MHZ, methanol-d₄) δ=7.52 (dd, J=2.7, 8.9 Hz, 1H), 7.40 (d, J=2.6 Hz, 1H), 7.28-7.19 (m, 1H), 7.02 (d, J=8.9 Hz, 1H), 6.95-6.90 (m, 2H), 6.85-6.79 (m, 1H), 3.98 (t, J=5.3 Hz, 2H), 3.68 (s, 2H), 3.17 (t, J=7.2 Hz, 4H), 2.77 (t, J=5.4 Hz, 2H), 2.28 (s, 3H), 2.14 (s, 3H), 2.03 (quin, J=7.2 Hz, 2H).

### N-(4-(2-(azetidin-1-yl)ethoxy)-3-(3,5-dimethylisoxazol-4-yl)phenyl)-2-(4-methoxyphenyl) acetamide (126)

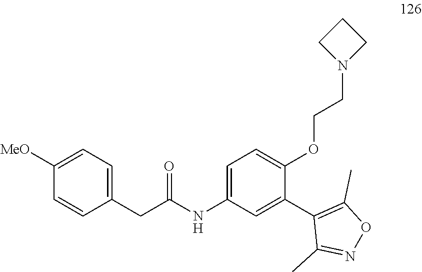 [SMILES: COc1ccc(CC(=O)Nc2ccc(OCCN3CCC3)c(-c3c(C)noc3C)c2)cc1]

Compound 126 was prepared according to the synthesis described for compound 117, substituting 2-(4-methoxyphenyl)acetic acid for 1-fluorocyclopropanecarboxylic acid.

LCMS (ESI): m/z [M+H] calcd for C₂₅H₃₀N₃O₄: 436.0; found: 436.0.

¹H NMR (400 MHZ, methanol-d₄) δ=7.55 (dd, J=2.7, 8.9 Hz, 1H), 7.42 (d, J=2.6 Hz, 1H), 7.28 (d, J=8.8 Hz, 2H), 7.05 (d, J=8.9 Hz, 1H), 6.94-6.87 (m, 2H), 4.03 (t, J=5.3 Hz, 2H), 3.79 (s, 3H), 3.61 (s, 2H), 3.37-3.34 (m, 2H), 3.31 (br s, 2H), 2.93 (br t, J=5.1 Hz, 2H), 2.31 (s, 3H), 2.16 (s, 3H), 2.14-2.07 (m, 2H).

### N-(4-(2-(azetidin-1-yl)ethoxy)-3-(3,5-dimethylisoxazol-4-yl)phenyl)-2-(4-methylthiazol-5-yl)acetamide (127)

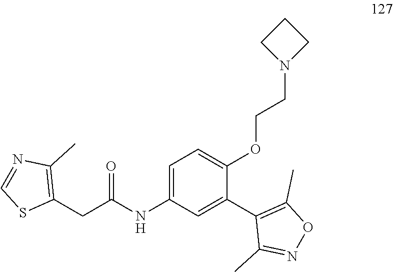 [SMILES: Cc1ncsc1CC(=O)Nc1ccc(OCCN2CCC2)c(-c2c(C)noc2C)c1]

Compound 127 was prepared according to the synthesis described for compound 117, substituting 2-(4-methylthiazol-5-yl)acetic acid for 1-fluorocyclopropanecarboxylic acid.

LCMS (ESI): m/z [M+H] calcd for C₂₂H₂₇N₄O₃S: 427.0; found: 427.0.

¹H NMR (400 MHZ, methanol-d₄) δ=8.81 (s, 1H), 7.55 (dd, J=2.6, 8.9 Hz, 1H), 7.42 (d, J=2.7 Hz, 1H), 7.06 (d, J=8.9 Hz, 1H), 4.05 (t, J=5.3 Hz, 2H), 3.90 (s, 2H), 3.39 (br t, J=7.3 Hz, 4H), 2.99 (br t, J=5.0 Hz, 2H), 2.44 (s, 3H), 2.30 (s, 3H), 2.15-2.10 (m, 5H).

### N-(4-(2-(azetidin-1-yl)ethoxy)-3-(3,5-dimethylisoxazol-4-yl)phenyl)-2-(5-fluoropyridin-3-yl)acetamide (128)

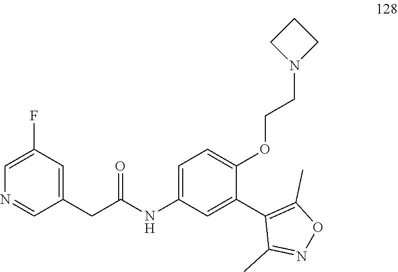 [SMILES: Cc1noc(C)c1-c1cc(NC(=O)Cc2cncc(F)c2)ccc1OCCN1CCC1]

Compound 128 was prepared according to the synthesis described for compound 117, substituting 2-(5-fluoropyridin-3-yl)acetic acid for 1-fluorocyclopropanecarboxylic acid.

LCMS (ESI): m/z [M+H] calcd for C₂₃H₂₆FN₄O₃: 425.0; found: 425.0.

¹H NMR (400 MHZ, methanol-d₄) δ=8.45-8.32 (m, 2H), 7.67 (td, J=2.2, 9.5 Hz, 1H), 7.53 (dd, J=2.7, 8.9 Hz, 1H), 7.41 (d, J=2.6 Hz, 1H), 7.03 (d, J=8.9 Hz, 1H), 3.98 (t, J=5.3 Hz, 2H), 3.79 (s, 2H), 3.17 (t, J=7.2 Hz, 4H), 2.78 (t, J=5.3 Hz, 2H), 2.29 (s, 3H), 2.14 (s, 3H), 2.03 (quin, J=7.2 Hz, 2H).

### N-(4-(2-(azetidin-1-yl)ethoxy)-3-(3,5-dimethylisoxazol-4-yl)phenyl)-2-(5-fluoropyridin-2-yl)acetamide (129)

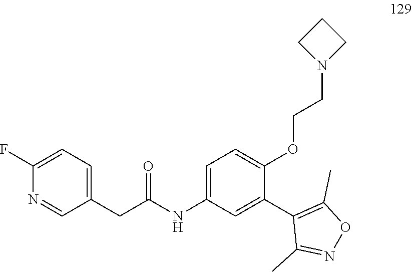 [SMILES: Cc1noc(C)c1-c1cc(NC(=O)Cc2ccc(F)nc2)ccc1OCCN1CCC1]

Compound 129 was prepared according to the synthesis described for compound 117, substituting 2-(5-fluoropyridin-2-yl)acetic acid for 1-fluorocyclopropanecarboxylic acid.

LCMS (ESI): m/z [M+H] calcd for C₂₃H₂₆FN₄O₃: 425.0; found: 425.0.

¹H NMR (400 MHZ, methanol-d₄) δ=8.41 (d, J=2.8 Hz, 1H), 7.64-7.56 (m, 1H), 7.54 (dd, J=2.8, 8.9 Hz, 1H), 7.49 (dd, J=4.5, 8.8 Hz, 1H), 7.43 (d, J=2.6 Hz, 1H), 7.03 (d, J=8.9 Hz, 1H), 3.98 (t, J=5.3 Hz, 2H), 3.89 (s, 2H), 3.22-3.10 (m, 4H), 2.78 (t, J=5.3 Hz, 2H), 2.29 (s, 3H), 2.14 (s, 3H), 2.03 (quin, J=7.2 Hz, 2H).

### N-(4-(2-(azetidin-1-yl)ethoxy)-3-(3,5-dimethylisoxazol-4-yl)phenyl)-4-fluoro-1-methyl-1H-pyrazole-5-carboxamide (130)

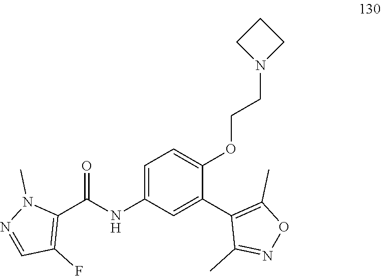 [SMILES: Cc1noc(C)c1-c1cc(NC(=O)c2c(F)cnn2C)ccc1OCCN1CCC1]

Compound 130 was prepared according to the synthesis described for compound substituting for 1-117, 4-fluoro-1-methyl-1H-pyrazole-5-carboxylic acid fluorocyclopropanecarboxylic acid.

LCMS (ESI): m/z [M+H] calcd for C₂₁H₂₅FN₅O₃: 414.0, found: 414.3.

¹H NMR (400 MHZ, methanol-d₄) δ=7.64 (dd, J=2.6, 8.9 Hz, 1H), 7.51 (d, J=2.6 Hz, 1H), 7.46 (d, J=4.4 Hz, 1H), 7.09 (d, J=9.0 Hz, 1H), 4.05 (s, 3H), 4.02 (t, J=5.3 Hz, 2H), 3.18 (t, J=7.2 Hz, 4H), 2.80 (t, J=5.3 Hz, 2H), 2.32 (s, 3H), 2.17 (s, 3H), 2.04 (quin, J=7.2 Hz, 2H).

### N-(4-(2-(azetidin-1-yl)ethoxy)-3-(3,5-dimethylisoxazol-4-yl)phenyl)-2-ethoxycyclopropane-1-carboxamide (131)

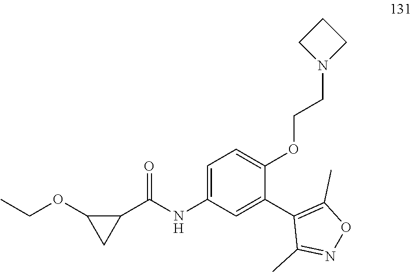 [SMILES: CCOC1CC1C(=O)Nc1ccc(OCCN2CCC2)c(-c2c(C)noc2C)c1]

Compound 131 was prepared according to the synthesis described for compound substituting acid for 1-117, 2-ethoxycyclopropane-1-carboxylic fluorocyclopropanecarboxylic acid.

LCMS (ESI): m/z [M+H] calcd for C₂₂H₃₀N₃O₄: 400.0, found: 400.3.

¹H NMR (400 MHZ, methanol-d₄) δ=7.50 (dd, J=2.8, 8.9 Hz, 1H), 7.39 (d, J=2.6 Hz, 1H), 7.02 (d, J=8.9 Hz, 1H), 3.98 (t, J=5.4 Hz, 2H), 3.63 (q, J=7.1 Hz, 2H), 3.58 (td, J=2.1, 4.3 Hz, 1H), 3.17 (t, J=7.2 Hz, 4H), 2.77 (t, J=5.4 Hz, 2H), 2.29 (s, 3H), 2.15 (s, 3H), 2.03 (quin, J=7.2 Hz, 2H), 1.88 (ddd, J=1.9, 5.9, 9.4 Hz, 1H), 1.25-1.18 (m, 5H).

### N-[3-(3,5-dimethylisoxazol-4-yl)-4-(1-methylazetidin-3-yl)oxy-phenyl]cyclopropanecarboxamide (132)

### Preparation of tert-butyl 3-[2-(3,5-dimethylisoxazol-4-yl)-4-nitro-phenoxy]azetidine-1-carboxylate (10)

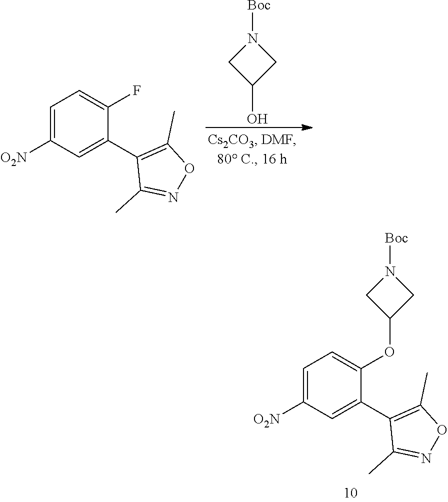 [SMILES: Cc1noc(C)c1-c1cc([N+](=O)[O-])ccc1F ; CC(C)(C)OC(=O)N1CC(O)C1 ; Cc1noc(C)c1-c1cc([N+](=O)[O-])ccc1OC1CN(C(=O)OC(C)(C)C)C1]

A mixture of 4-(2-fluoro-5-nitro-phenyl)-3,5-dimethyl-isoxazole (500 mg, 2.12 mmol, 1 eq.), tert-butyl 3-hydroxyazetidine-1-carboxylate (403.33 mg, 2.33 mmol, 1.1 eq.) and Cs₂CO₃ (1.38 g, 4.23 mmol, 2 eq.) in DMF (5 mL) was stirred at 80° C. for 16 hrs (monitored by LC-MS). The reaction mixture was filtered and concentrated under reduced pressure to give a residue. Tert-butyl 3-[2-(3,5-dimethylisoxazol-4-yl)-4-nitro-phenoxy]azetidine-1-carboxylate ((10), 800 mg, crude) was obtained as a white solid.

LCMS (ESI): m/z [M+H] calcd for C₁₉H₂₄N₃O₆: 390.16; found: 390.2.

### Preparation of tert-butyl 3-[4-amino-2-(3,5-dimethylisoxazol-4-yl)phenoxy]azetidine-1-carboxylate (11)

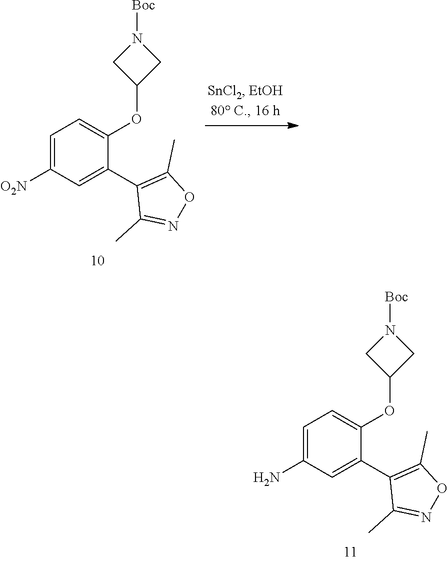 [SMILES: Cc1noc(C)c1-c1cc([N+](=O)[O-])ccc1OC1CN(C(=O)OC(C)(C)C)C1 ; Cc1noc(C)c1-c1cc(N)ccc1OC1CN(C(=O)OC(C)(C)C)C1]

A mixture of tert-butyl 3-[2-(3,5-dimethylisoxazol-4-yl)-4-nitro-phenoxy]azetidine-1-carboxylate ((10), 800 mg, 2.05 mmol, 1 eq.) and SnCl₂·2 H₂O (927.15 mg, 4.11 mmol, 2 eq.) in EtOH (10 mL) and H₂O (2 mL) was stirred at 80° C. for 16 hrs (monitored by LC-MS). The reaction mixture was filtered and concentrated under reduced pressure to give a residue. The residue was purified by column chromatography. TLC (SiO₂, DCM: MeOH=10:1). The product was adjusted to pH=7-8 with saturated NaHCO₃ aqueous and extracted with EtOAc (20 mL×3). The combined organic layers were concentrated under reduced pressure to give a residue. The residue was purified by reversed-phase HPLC (0.1% NH₃·H₂O). Tert-butyl 3-[4-amino-2-(3,5-dimethylisoxazol-4-yl)phenoxy]azetidine-1-carboxylate ((11), 380 mg, 1.06 mmol, 51.46% yield) was obtained as a black oil.

LCMS (ESI): m/z [M+H] calcd for C₁₉H₂₆N₃O₄: 360.18; found: 360.1.

### Preparation of tert-butyl 3-[4-(cyclopropanecarbonylamino)-2-(3,5-dimethylisoxazol-4-yl)phenoxy]azetidine-1-carboxylate (12)

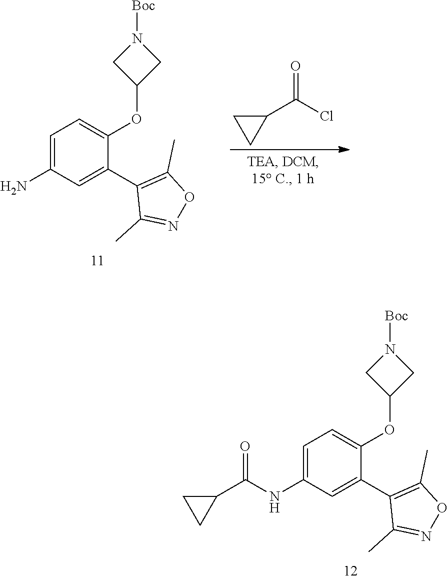 [SMILES: Cc1noc(C)c1-c1cc(N)ccc1OC1CN(C(=O)OC(C)(C)C)C1 ; O=C(Cl)C1CC1 ; Cc1noc(C)c1-c1cc(NC(=O)C2CC2)ccc1OC1CN(C(=O)OC(C)(C)C)C1]

To a mixture of tert-butyl 3-[4-amino-2-(3,5-dimethylisoxazol-4-yl)phenoxy]azetidine-1-carboxylate ((11), 380 mg, 1.06 mmol, 1 eq.) and TEA (321.78 mg, 3.18 mmol, 442.62 μL, 3 eq.) in DCM (10 mL) was added cyclopropanecarbonyl chloride (121.89 mg, 1.17 mmol, 105.99 μL, 1.1 eq.) at 0° C. The mixture was stirred at 15° C. for 10 min (monitored by LC-MS). The reaction mixture was quenched by addition of H₂O (5 mL), and then extracted with EtOAc (10 mL×3). The combined organic layers were concentrated under reduced pressure to give a residue. Tert-butyl 3-[4-(cyclopropanecarbonylamino)-2-(3,5-dimethylisoxazol-4-yl)phenoxy]azetidine-1-carboxylate ((12), 410 mg, crude) was obtained as a yellow oil.

LCMS (ESI): m/z [M+H] calcd for C₂₃H₃₀N₃O₅: 428.21; found: 428.2.

### Preparation of N-[4-(azetidin-3-yloxy)-3-(3,5-dimethylisoxazol-4-yl)phenyl]cyclopropanecarboxamide (13)

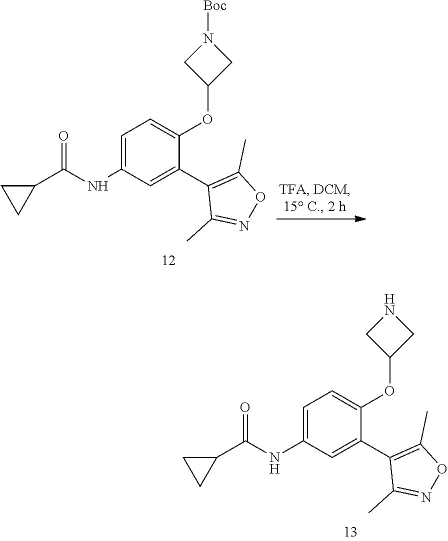 [SMILES: Cc1noc(C)c1-c1cc(NC(=O)C2CC2)ccc1OC1CN(C(=O)OC(C)(C)C)C1 ; Cc1noc(C)c1-c1cc(NC(=O)C2CC2)ccc1OC1CNC1]

A mixture of tert-butyl 3-[4-(cyclopropanecarbonylamino)-2-(3,5-dimethylisoxazol-4-yl)phenoxy]azetidine-1-carboxylate ((12), 410 mg, 959.08 μmol, 1 eq.) in TFA (1 mL) and DCM (5 mL) was stirred at 15° C. for 2 hrs (monitored by LCMS). The reaction mixture was concentrated under reduced pressure to give a residue. N-[4-(azetidin-3-yloxy)-3-(3,5-dimethylisoxazol-4-yl)phenyl]cyclopropanecarboxamide ((13), 300 mg, crude) was obtained as a black oil.

LCMS (ESI): m/z [M+H] calcd for C₁₈H₂₂N₃O₃: 328.16; found: 328.2.

### Preparation of N-[3-(3,5-dimethylisoxazol-4-yl)-4-(1-methylazetidin-3-yl)oxy-phenyl]cyclopropanecarboxamide (132)

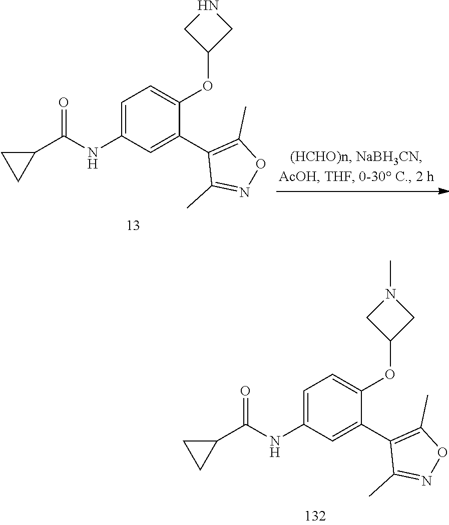 [SMILES: Cc1noc(C)c1-c1cc(NC(=O)C2CC2)ccc1OC1CNC1 ; Cc1noc(C)c1-c1cc(NC(=O)C2CC2)ccc1OC1CN(C)C1]

A mixture of N-[4-(azetidin-3-yloxy)-3-(3,5-dimethylisoxazol-4-yl)phenyl]cyclopropanecarboxamide ((13), 60 mg, 183.28 μmol, 1 eq.), HCHO (6.60 mg, 219.93 μmol, 6.06 μL, 1.2 eq.), and CH₃COOH (11.01 mg, 183.28 μmol, 10.48 μL, 1 eq.) in MeOH (1 mL) was stirred at 30° C. for 1 hr. To the mixture was added NaBH₃CN (17.28 mg, 274.91 μmol, 1.5 eq.). The mixture was stirred at 30° C. for 1 hr (monitored by LCMS). The reaction mixture was concentrated under reduced pressure to give a residue. The residue was purified by prep-HPLC (column: Waters Xbridge 150*25 mm*5 um; mobile phase: [water (0.05% ammonia hydroxide v/v)-ACN]; B %: 20%-50%, 10 min). N-[3-(3,5-dimethylisoxazol-4-yl)-4-(1-methylazetidin-3-yl)oxy-phenyl]cyclopropanecarboxamide (17.79 mg, 51.07 μmol, 27.86% yield, 98% purity) was obtained as a white solid.

LCMS (ESI): m/z [M+H] calcd for C₁₉H₂₄N₃O₃: 342.17; found: 342.2.

¹H NMR (400 MHZ, methanol-d₄) δ=7.52 (dd, J=2.6, 8.8 Hz, 1H), 7.43 (d, J=2.6 Hz, 1H), 6.79 (d, J=8.8 Hz, 1H), 4.90-4.86 (m, 1H), 4.09-3.96 (m, 2H), 3.42 (dd, J=5.1, 10.1 Hz, 2H), 2.54 (s, 3H), 2.32 (s, 3H), 2.17 (s, 3H), 1.79-1.68 (m, 1H), 0.97-0.90 (m, 2H), 0.88-0.82 (m, 2H).

### N-(3-(3,5-dimethylisoxazol-4-yl)-4-((1-ethylazetidin-3-yl)oxy)phenyl)cyclopropanecarboxamide (133)

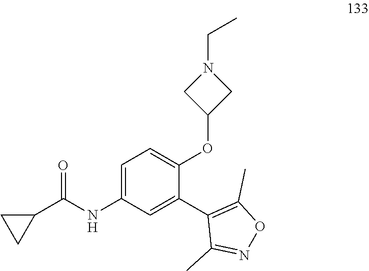 [SMILES: CCN1CC(Oc2ccc(NC(=O)C3CC3)cc2-c2c(C)noc2C)C1]

Compound 133 was prepared according to the synthesis described for compound 132.

LCMS (ESI): m/z [M+H] calcd for C₂₀H₂₆N₃O₃: 356.19; found: 356.2.

¹H NMR (400 MHZ, methanol-d₄) δ=7.59 (dd, J=2.6, 8.9 Hz, 1H), 7.46 (d, J=2.4 Hz, 1H), 6.97-6.67 (m, 1H), 5.18-5.04 (m, 1H), 4.72 (br d, J=2.4 Hz, 1H), 4.47 (br d, J=10.4 Hz, 1H), 4.33-4.18 (m, 1H), 4.13-3.96 (m, 1H), 3.28 (br d, J=5.0 Hz, 2H), 2.33 (s, 3H), 2.18 (s, 3H), 1.80-1.66 (m, 1H), 1.21 (t, J=7.2 Hz, 3H), 0.97-0.91 (m, 2H), 0.89-0.82 (m, 2H).

### N-[3-(3,5-dimethylisoxazol-4-yl)-4-(2-morpholinoethoxy)phenyl]-4-fluoro-2-methyl-pyrazole-3-carboxamide (134)

### Preparation of 4-[2-[2-(3,5-dimethylisoxazol-4-yl)-4-nitro-phenoxy]ethyl]morpholine (14)

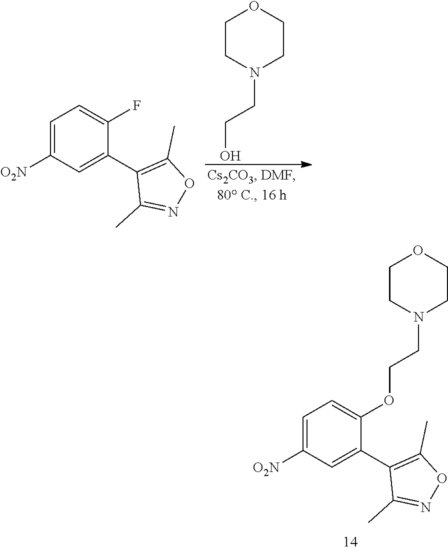

The mixture of 4-(2-fluoro-5-nitro-phenyl)-3,5-dimethyl-isoxazole (200 mg, 846.74 μmol, 1 eq.), 2-morpholinoethanol (133.28 mg, 1.02 mmol, 124.56 μL, 1.2 eq.) and Cs₂CO₃ (551.77 mg, 1.69 mmol, 2 eq.) in DMF (2 mL) was stirred at 80° C. for 16 hrs. The reaction mixture was poured into water (20 mL). The aqueous phase was extracted with EtOAc (10 mL×3). The combined organic phase was washed with brine, dried with anhydrous Na₂SO₄, filtered and the filtrate was concentrated to give a residue. The residue was purified by flash silica gel chromatography (ISCO®; 25 g SepaFlash® Silica Flash Column, eluent of 100˜20% ethyl acetate/methanol). 4-[2-[2-(3,5-dimethylisoxazol-4-yl)-4-nitro-phenoxy]ethyl]morpholine ((14), 151 mg, 434.70 μmol, 51.34% yield) was obtained as a yellow gum.

LCMS (ESI): m/z [M+H] calcd for C₁₇H₂₂N³O₅: 348.15; found: 348.2.

### Preparation of 3-(3,5-dimethylisoxazol-4-yl)-4-(2-morpholinoethoxy)aniline (15)

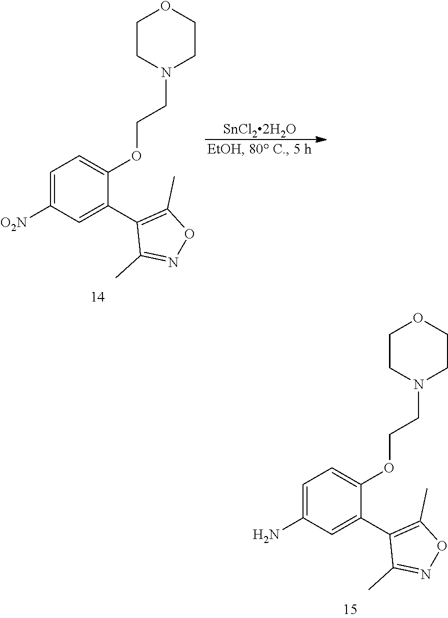 [SMILES: Cc1noc(C)c1-c1cc([N+](=O)[O-])ccc1OCCN1CCOCC1 ; Cc1noc(C)c1-c1cc(N)ccc1OCCN1CCOCC1]

To a solution of 4-[2-[2-(3,5-dimethylisoxazol-4-yl)-4-nitro-phenoxy]ethyl]morpholine ((14), 150 mg, 431.82 μmol, 1 eq.) in EtOH (2 mL) was added SnCl₂·2 H₂O (243.60 mg, 1.08 mmol, 2.5 eq.). The mixture was stirred at 80° C. for 5 hrs. LCMS desired mass was detected. The mixture was concentrated in vacuo. The crude product was purified by reversed-phase HPLC (0.1% NH₃·H₂O). 3-(3,5-dimethylisoxazol-4-yl)-4-(2-morpholinoethoxy)aniline ((15), 100 mg, crude) was obtained as a yellow solid.

### Preparation of N-[3-(3,5-dimethylisoxazol-4-yl)-4-(2-morpholinoethoxy)phenyl]-4-fluoro-2-methyl-pyrazole-3-carboxamide (134)

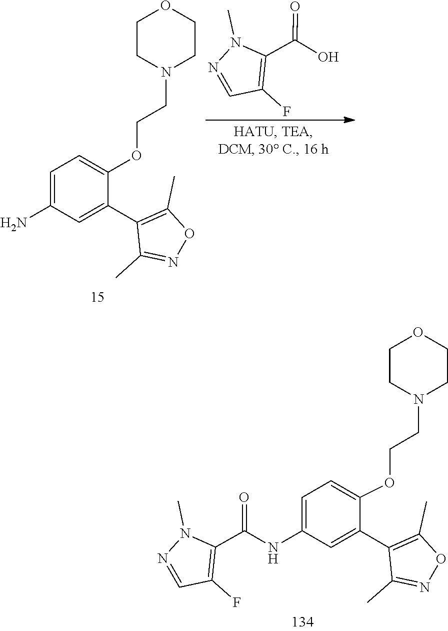

To a solution of 3-(3,5-dimethylisoxazol-4-yl)-4-(2-morpholinoethoxy)aniline ((15), 50 mg, 157.54 μmol, 1 eq.) in DCM (1 mL) was added HATU (89.85 mg, 236.31 μmol, 1.5 eq.) and TEA (47.82 mg, 472.62 μmol, 65.78 μL, 3 eq.) and 4-fluoro-2-methyl-pyrazole-3-carboxylic acid (27.24 mg, 189.05 μmol, 1.2 eq.). The mixture was stirred at 30° C. for 16 hrs. LC-MS showed the desired mass was detected. The mixture was concentrated in vacuo. The residue was purified by prep-HPLC (TFA conditions, column: Phenomenex Gemini-NX C18 75*30 mm*3 μm; mobile phase: [water (0.1% TFA)-ACN]; B %: 22%-32%, 5 min). N-[3-(3,5-dimethylisoxazol-4-yl)-4-(2-morpholinoethoxy)phenyl]-4-fluoro-2-methyl-pyrazole-3-carboxamide (23.8 mg, 41.41 μmol, 26.29% yield, 97% purity, TFA) was obtained as a white solid.

LCMS (ESI): m/z [M+H] calcd for C₂₂H₂₇FN₅O₄: 444.0, found: 444.3.

¹H NMR (400 MHZ, methanol-d₄) δ=7.74 (br d, J=8.9 Hz, 1H), 7.56 (d, J=2.1 Hz, 1H), 7.47 (d, J=4.4 Hz, 1H), 7.20 (d, J=8.9 Hz, 1H), 4.42 (br t, J=4.6 Hz, 2H), 4.05 (s, 3H), 4.02-3.62 (m, 4H), 3.60-3.53 (m, 2H), 3.30-3.15 (m, 4H), 2.34 (s, 3H), 2.18 (s, 3H).

### N-[3-(3,5-dimethylisoxazol-4-yl)-4-(2-morpholinoethoxy)phenyl]-2-ethoxy-cyclopropanecarboxamide (135)

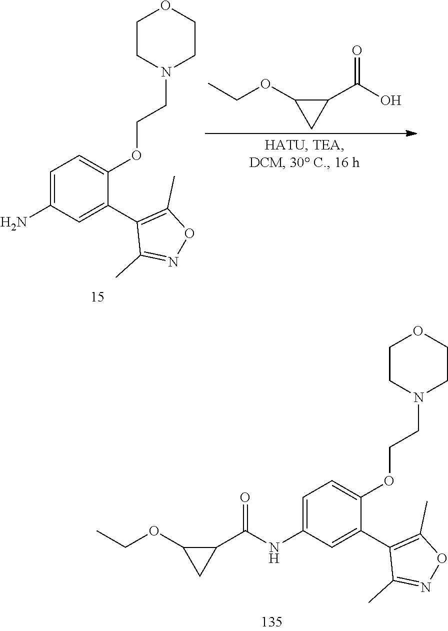

To a solution of 3-(3,5-dimethylisoxazol-4-yl)-4-(2-morpholinoethoxy)aniline ((15), 60 mg, 189.05 μmol, 1 eq.) in DCM (1 mL) was added HATU (107.82 mg, 283.57 μmol, 1.5 eq.) and TEA (38.26 mg, 378.09 μmol, 52.63 μL, 2 eq.) and 2-ethoxycyclopropanecarboxylic acid (29.52 mg, 226.86 μmol, 1.2 eq.). The mixture was stirred at 30° C. for 2 hrs. The mixture was concentrated in vacuo. The residue was purified by prep-HPLC (neutral condition, column: Phenomenex Gemini NX-C18 (75*30 mm*3 μm); mobile phase: [water (10 mM NH₄CO₃)-ACN]; B %: 15%-45%, 10 min). N-[3-(3,5-dimethylisoxazol-4-yl)-4-(2-morpholinoethoxy)phenyl]-2-ethoxy-cyclopropanecarboxamide (8.49 mg, 19.77 μmol, 10.46% yield, 100% purity) was obtained as a brown gum.

LCMS (ESI): m/z [M+H] calcd for C₂₃H₃₂N³O₅: 430.0, found: 430.3.

¹H NMR (400 MHZ, methanol-d₄) δ=7.51 (dd, J=2.8, 8.9 Hz, 1H), 7.39 (d, J=2.6 Hz, 1H), 7.06 (d, J=8.9 Hz, 1H), 4.11 (t, J=5.4 Hz, 2H), 3.65-3.57 (m, 7H), 2.70 (t, J=5.4 Hz, 2H), 2.48-2.41 (m, 4H), 2.30 (s, 3H), 2.16 (s, 3H), 1.92-1.85 (m, 1H), 1.27-1.17 (m, 5H).

### N-[3-(3,5-dimethylisoxazol-4-yl)-4-[2-(3-oxo-5,6,8,8a-tetrahydro-1H-oxazolo[3,4-a]pyrazin-7-yl)ethoxy]phenyl]cyclopropanecarboxamide (136)

### Preparation of N-(3-bromo-4-methoxy-phenyl)cyclopropanecarboxamide (16)

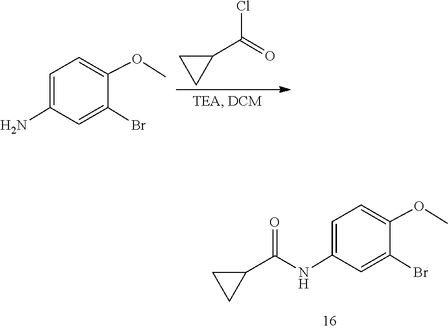

To a solution of cyclopropanecarbonyl chloride (4.14 g, 39.59 mmol, 3.60 mL, 1 eq.) in DCM (100 mL) was added TEA (8.01 g, 79.19 mmol, 11.02 mL, 2 eq.) and 3-bromo-4-methoxy-aniline (8 g, 39.59 mmol, 1 eq.) at 0° C. The mixture was stirred at 25° C. for 16 hrs. To the mixture was added HCl (1 mol, 30 mL) and was stirred at 25° C. for a few minutes. The mixture was filtered and concentrated in vacuo. N-(3-bromo-4-methoxy-phenyl)cyclopropanecarboxamide ((16), 8.5 g, 31.47 mmol, 79.47% yield) was obtained as a purple solid.

LCMS (ESI): m/z [M+H] calcd for C₁₁H₁₃Br^{79/81}NO₂: 270.0/272.0, found: 269.8/271.8.

### Preparation of N-[3-(3,5-dimethylisoxazol-4-yl)-4-methoxy-phenyl]cyclopropanecarboxamide (17)

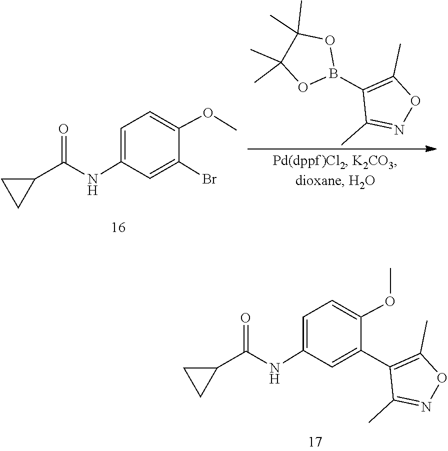 [SMILES: COc1ccc(NC(=O)C2CC2)cc1Br ; Cc1noc(C)c1B1OC(C)(C)C(C)(C)O1 ; COc1ccc(NC(=O)C2CC2)cc1-c1c(C)noc1C]

A mixture of N-(3-bromo-4-methoxy-phenyl)cyclopropanecarboxamide ((16), 4 g, 14.81 mmol, 1 eq.), 3,5-dimethyl-4-(4,4,5,5-tetramethyl-1,3,2-dioxaborolan-2-yl)isoxazole (3.96 g, 17.77 mmol, 1.2 eq.), Pd(dppf)Cl₂ (1.08 g, 1.48 mmol, 0.1 eq.), and K₂CO₃ (4.09 g, 29.62 mmol, 2 eq.) in dioxane (40 mL) and H₂O (10 mL) was degassed and purged with N₂ three times, and then the mixture was stirred at 80° C. for 5 hrs under an N² atmosphere. The mixture was concentrated in vacuo. The residue was purified by flash silica gel chromatography (ISCO®; 80 g SepaFlash® Silica Flash Column, eluent of 0˜40% ethyl acetate/petroleum ether gradient at 60 mL/min). N-[3-(3,5-dimethylisoxazol-4-yl)-4-methoxy-phenyl]cyclopropanecarboxamide ((17), 2.9 g, 10.13 mmol, 68.40% yield) was obtained as a yellow solid.

LCMS (ESI): m/z [M+H] calcd for C₁₆H₁₉N₂O₃: 287.0, found: 286.9.

### Preparation of N-[3-(3,5-dimethylisoxazol-4-yl)-4-hydroxy-phenyl]cyclopropanecarboxamide (18)

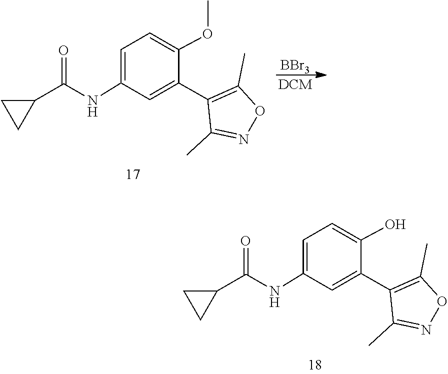 [SMILES: COc1ccc(NC(=O)C2CC2)cc1-c1c(C)noc1C ; Cc1noc(C)c1-c1cc(NC(=O)C2CC2)ccc1O]

A mixture of N-[3-(3,5-dimethylisoxazol-4-yl)-4-methoxy-phenyl]cyclopropanecarboxamide ((17), 2.9 g, 10.13 mmol, 1 eq.), BBr3 (5.07 g, 20.26 mmol, 1.95 mL, 2 eq.) in DCM (35 mL) at 0° C. was degassed and purged with N₂ three times, and then the mixture was stirred at 0-25° C. for 16 hrs under an N₂ atmosphere. The mixture was diluted with water (50 mL) and then extracted with ethyl acetate (150 mL×2). The combined organic phase was washed with brine (100 mL), dried over anhydrous Na₂SO₄, filtered and concentrated in vacuo. The crude product was purified by reversed-phase HPLC (0.1% TFA conditions). N-[3-(3,5-dimethylisoxazol-4-yl)-4-hydroxy-phenyl]cyclopropanecarboxamide (1 g, 3.67 mmol, 36.26% yield) was obtained as a white solid.

LCMS (ESI): m/z [M+H] calcd for C₁₅H₁₇N₂O₃: 273.12, found: 273.2.

### Preparation of N-[4-(2-bromoethoxy)-3-(3,5-dimethylisoxazol-4-yl)phenyl]cyclopropanecarboxamide (19)

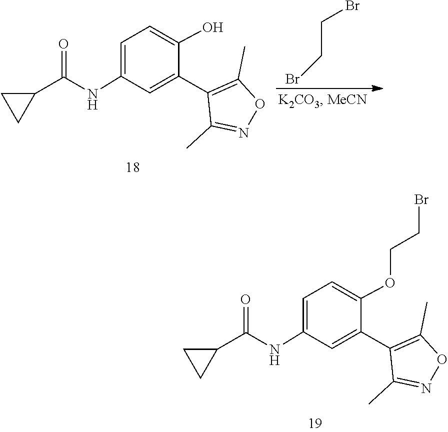

To a solution of N-[3-(3,5-dimethylisoxazol-4-yl)-4-hydroxy-phenyl]cyclopropanecarboxamide ((18), 1 g, 3.67 mmol, 1 eq.) in MeCN (10 mL) was added K₂CO₃ (4.06 g, 29.38 mmol, 8 eq.) and 1,2-dibromoethane (6.90 g, 36.72 mmol, 2.77 mL, 10 eq.). The mixture was stirred at 80° C. for 16 hrs. The mixture was diluted with water (30 mL) and then extracted with ethyl acetate (100 mL×2). The combined organic phase was washed with brine (50 mL), dried over anhydrous Na₂SO₄, filtered and concentrated in vacuo. The residue was purified by flash silica gel chromatography (ISCO®; 12 g SepaFlash® Silica Flash Column, eluent of 0˜80% ethyl acetate/petroleum ether gradient at 30 mL/min). N-[4-(2-bromoethoxy)-3-(3,5-dimethylisoxazol-4-yl)phenyl]cyclopropanecarboxamide ((19), 950 mg, 2.50 mmol, 68.21% yield) was obtained as a white solid.

LCMS (ESI): m/z [M+H] calcd for C₁₇H₂₀Br^{79/81}N²O₃: 379.06/381.06, found: 379.3/381.3.

### Preparation of N-[3-(3,5-dimethylisoxazol-4-yl)-4-[2-(3-oxo-5,6,8,8a-tetrahydro-1H-oxazolo[3,4-a]pyrazin-7-yl)ethoxy]phenyl]cyclopropanecarboxamide (136)

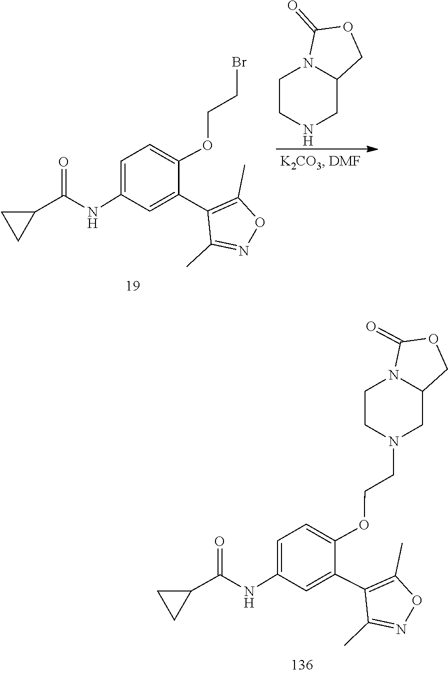

To a solution of N-[4-(2-bromoethoxy)-3-(3,5-dimethylisoxazol-4-yl)phenyl]cyclopropanecarboxamide ((19), 150 mg, 395.52 μmol, 1 eq.) in DMF (1.5 mL) was added K₂CO₃ (163.99 mg, 1.19 mmol, 3 eq.) and 1,5,6,7,8,8a-hexahydrooxazolo[3,4-a]pyrazin-3-one hydrochloride (84.78 mg, 474.62 μmol, 1.2 eq.). The mixture was stirred at 80° C. for 5 hrs. The mixture was filtered and concentrated in vacuo. The residue was purified by prep-HPLC (TFA conditions; column: Phenomenex Gemini-NX C18 75*30 mm*3 μm; mobile phase: [water (0.1% TFA)-ACN]; B %: 18%-28%, 7 min). N-[3-(3,5-dimethylisoxazol-4-yl)-4-[2-(3-oxo-5,6,8,8a-tetrahydro-1H-oxazolo[3,4-a]pyrazin-7-yl)ethoxy]phenyl]cyclopropanecarboxamide (59 mg, 106.40 μmol, 26.90% yield, 100% purity, TFA) was obtained as a yellow solid.

LCMS (ESI): m/z [M+H] calcd for C₂₂H₂₈N₂O₅: 441.0, found: 441.2.

¹H NMR (400 MHZ, methanol-d₄) δ=7.58 (dd, J=2.6, 8.9 Hz, 1H), 7.45 (d, J=2.5 Hz, 1H), 7.12 (d, J=9.0 Hz, 1H), 4.54-4.27 (m, 3H), 4.17-4.05 (m, 1H), 4.01-3.80 (m, 2H), 3.62-3.32 (m, 5H), 3.07-2.77 (m, 2H), 2.32 (s, 3H), 2.17 (s, 3H), 1.81-1.65 (m, 1H), 1.04-0.90 (m, 2H), 0.86 (td, J=3.1, 7.8 Hz, 2H).

### N-[3-(3,5-dimethylisoxazol-4-yl)-4-[2-(7-oxo-1,4-oxazepan-4-yl)ethoxy]phenyl]cyclopropanecarboxamide (137)

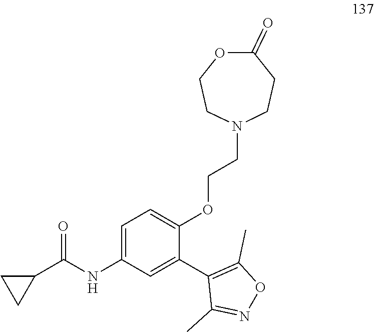 [SMILES: Cc1noc(C)c1-c1cc(NC(=O)C2CC2)ccc1OCCN1CCOC(=O)CC1]

To a solution of N-[4-(2-bromoethoxy)-3-(3,5-dimethylisoxazol-4-yl)phenyl]cyclopropanecarboxamide ((19), 80 mg, 210.94 μmol, 1 eq.) and 1,4-oxazepan-7-one (53.17 mg, 232.04 μmol, 1.1 eq., TFA) in CH₃CN (2 mL) was added K₂CO₃ (43.73 mg, 316.42 μmol, 1.5 eq.). The mixture was stirred at 80° C. for 5 hrs. The mixture was concentrated in vacuo. The residue was purified by prep-HPLC (column: Phenomenex Gemini-NX C18 75*30 mm*3 μm; mobile phase: [water (0.1% TFA)-ACN]; B %: 20%-30%, 7 min). 3-[2-[4-(cyclopropanecarbonylamino)-2-(3,5-dimethylisoxazol-4-yl)phenoxy]ethyl-(2-hydroxyethyl)amino]propanoic acid (25 mg, 45.83 μmol, 21.73% yield, TFA) was obtained as a white solid.

To a solution of 3-[2-[4-(cyclopropanecarbonylamino)-2-(3,5-dimethylisoxazol-4-yl)phenoxy]ethyl-(2-hydroxyethyl)amino]propanoic acid (25 mg, 57.94 μmol, 1 eq.) in DCM (4 mL) was added T₃P (55.31 mg, 86.91 μmol, 51.69 μL, 50% purity, 1.5 eq.) and TEA (8.79 mg, 86.91 μmol, 12.10 μL, 1.5 eq.). The mixture was stirred at 25° C. for 2 hrs. The mixture was concentrated in vacuo. The residue was purified by prep-HPLC (neutral conditions column: Phenomenex Gemini NX-C18 (75*30 mm*3 μm); mobile phase: [water (10 mM NH₄HCO₃)-ACN]; B %: 15%-45%, 8 min). N-[3-(3,5-dimethylisoxazol-4-yl)-4-[2-(7-oxo-1,4-oxazepan-4-yl)ethoxy]phenyl]cyclopropanecarboxamide (2.5 mg, 6.05 μmol, 10.44% yield, 100% purity) was obtained as a white solid.

LCMS (ESI): m/z [M+H] calcd for C₂₂H₂₈N₃O₅: 414.0, found: 414.2.

¹H NMR (400 MHZ, CD3CN) δ=8.45 (br s, 1H), 7.54 (dd, J=2.4, 9.2 Hz, 1H), 7.34 (d, J=2.8 Hz, 1H), 7.01 (d, J=8.8 Hz, 1H), 4.16 (t, J=8.4 Hz, 2H), 4.03 (t, J=5.6 Hz, 2H), 2.77-2.75 (m, 4H), 2.67-2.63 (m, 4H), 2.27 (s, 3H), 2.09 (s, 3H), 1.71-1.65 (m, 1H), 0.86-0.77 (m, 4H)

### N-(3-(3,5-dimethylisoxazol-4-yl)-4-(2-(6-oxohexahydropyrrolo[1,2-a]pyrazin-2(1H)-yl)ethoxy)phenyl)cyclopropanecarboxamide (138)

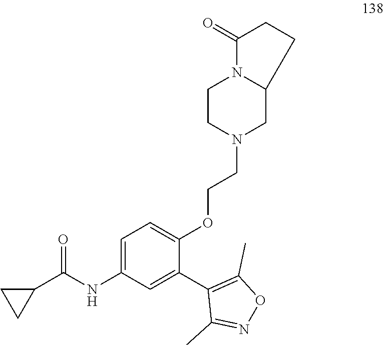 [SMILES: Cc1noc(C)c1-c1cc(NC(=O)C2CC2)ccc1OCCN1CCN2C(=O)CCC2C1]

Compound 138 was prepared according to the synthesis described for compound 136, substituting hexahydropyrrolo[1,2-a]pyrazin-6(2H)-one for 1,5,6,7,8,8a-hexahydrooxazolo[3,4-a]pyrazin-3-one hydrochloride.

LCMS (ESI): m/z [M+H] calcd for C₂₄H₃₁N₄O₄: 439.0, found: 439.2.

¹H NMR (400 MHZ, methanol-d₄) δ=7.71-7.54 (m, 1H), 7.44 (t, J=2.2 Hz, 1H), 7.13 (d, J=8.9 Hz, 1H), 4.51-4.29 (m, 2H), 4.11 (br d, J=13.6 Hz, 1H), 3.83 (br d, J=6.6 Hz, 1H), 3.62-3.44 (m, 4H), 3.15-2.92 (m, 2H), 2.85-2.72 (m, 1H), 2.53-2.37 (m, 2H), 2.35-2.23 (m, 4H), 2.17 (s, 3H), 1.74 (tt, J=4.6, 7.8 Hz, 1H), 1.68-1.53 (m, 1H), 0.99-0.91 (m, 2H), 0.86 (td, J=3.0, 7.8 Hz, 2H).

### N-(3-(3,5-dimethylisoxazol-4-yl)-4-(2-(1-oxo-2,7-diazaspiro[3.5]nonan-7-yl)ethoxy)phenyl)cyclopropanecarboxamide (139)

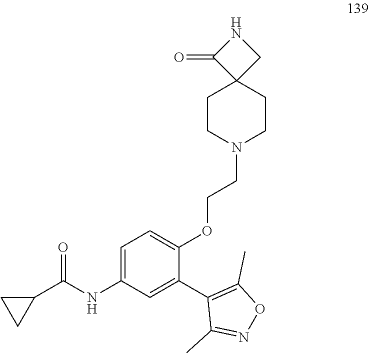 [SMILES: Cc1noc(C)c1-c1cc(NC(=O)C2CC2)ccc1OCCN1CCC2(CC1)CNC2=O]

Compound 139 was prepared according to the synthesis described for compound 136, substituting 2,7-diazaspiro[3.5]nonan-1-one for 1,5,6,7,8,8a-hexahydrooxazolo[3,4-a]pyrazin-3-one hydrochloride.

LCMS (ESI): m/z [M+H] calcd for C₂₄H₃₁N₄O₄: 439.0, found: 439.2.

¹H NMR (400 MHZ, methanol-d₄) δ=7.70-7.55 (m, 1H), 7.51-7.33 (m, 1H), 7.14 (d, J=9.0 Hz, 1H), 4.43-4.28 (m, 2H), 3.68-3.37 (m, 5H), 3.29-3.00 (m, 3H), 2.32 (s, 3H), 2.17 (s, 7H), 1.81-1.65 (m, 1H), 1.04-0.91 (m, 2H), 0.86 (td, J=3.0, 7.8 Hz, 2H).

### N-(3-(3,5-dimethylisoxazol-4-yl)-4-(2-(2-oxo-1,7-diazaspiro[3.5]nonan-7-yl)ethoxy)phenyl)cyclopropanecarboxamide (140)

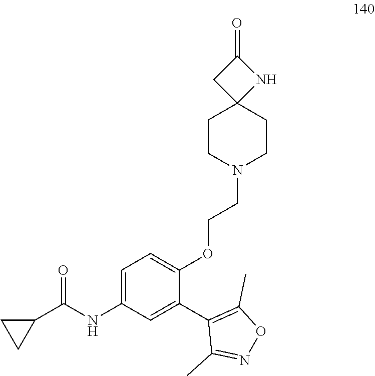 [SMILES: Cc1noc(C)c1-c1cc(NC(=O)C2CC2)ccc1OCCN1CCC2(CC1)CC(=O)N2]

Compound 140 was prepared according to the synthesis described for compound 136, substituting 1,7-diazaspiro[3.5]nonan-2-one for 1,5,6,7,8,8a-hexahydrooxazolo[3,4-a]pyrazin-3-one hydrochloride.

LCMS (ESI): m/z [M+H] calcd for C₂₅H₃₁N₄O₄: 439.0, found: 439.2.

¹H NMR (400 MHZ, methanol-d₄) δ=7.66-7.59 (m, 1H), 7.46 (d, J=2.5 Hz, 1H), 7.16 (d, J=9.0 Hz, 1H), 4.39 (br t, J=4.4 Hz, 2H), 3.64-3.45 (m, 4H), 3.20-2.96 (m, 2H), 2.83 (s, 2H), 2.34 (s, 3H), 2.18 (s, 3H), 2.16-1.90 (m, 4H), 1.82-1.72 (m, 1H), 0.99-0.85 (m, 4H).

### N-(3-(3,5-dimethylisoxazol-4-yl)-4-(2-(2-oxo-1-oxa-8-azaspiro[4.5]decan-8-yl)ethoxy)phenyl)cyclopropanecarboxamide (141)

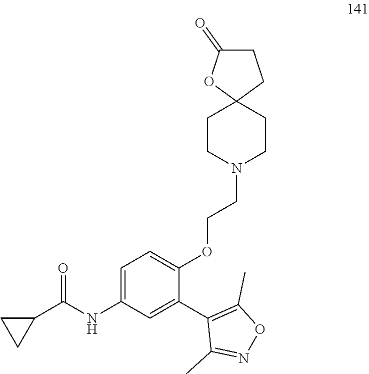 [SMILES: Cc1noc(C)c1-c1cc(NC(=O)C2CC2)ccc1OCCN1CCC2(CCC(=O)O2)CC1]

Compound 141 was prepared according to the synthesis described for compound 136, substituting 1-oxa-8-azaspiro[4.5]decan-2-one for 1,5,6,7,8,8a-hexahydrooxazolo[3,4-a]pyrazin-3-one hydrochloride.

LCMS (ESI): m/z [M+H] calcd for C₂₅H₃₅N₃O₅: 454.0, found: 454.3.

¹H NMR (400 MHZ, methanol-d₄) δ=7.53 (dd, J=2.6, 8.9 Hz, 1H), 7.40 (d, J=2.6 Hz, 1H), 7.07 (d, J=9.0 Hz, 1H), 4.12 (t, J=5.4 Hz, 2H), 2.77 (t, J=5.3 Hz, 2H), 2.66-2.57 (m, 4H), 2.54-2.45 (m, 2H), 2.31 (s, 3H), 2.17 (s, 3H), 2.06 (t, J=8.3 Hz, 2H), 1.86-1.71 (m, 5H), 0.97-0.90 (m, 2H), 0.90-0.81 (m, 2H).

### N-(3-(3,5-dimethylisoxazol-4-yl)-4-(2-(1-oxo-2-oxa-8-azaspiro[4.5]decan-8-yl)ethoxy)phenyl)cyclopropanecarboxamide (142)

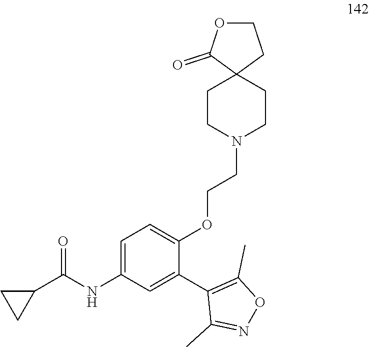 [SMILES: Cc1noc(C)c1-c1cc(NC(=O)C2CC2)ccc1OCCN1CCC2(CCOC2=O)CC1]

Compound 142 was prepared according to the synthesis described for compound 136, substituting 2-oxa-8-azaspiro[4.5]decan-1-one for 1,5,6,7,8,8a-hexahydrooxazolo[3,4-a]pyrazin-3-one hydrochloride.

LCMS (ESI): m/z [M+H] calcd for C₂₅H₃₂N₃O₅: 454.0, found: 454.3.

¹H NMR (400 MHZ, methanol-d₄) δ=7.61 (dd, J=1.8, 8.9 Hz, 1H), 7.44 (d, J=2.6 Hz, 1H), 7.14 (d, J=9.0 Hz, 1H), 4.46-4.23 (m, 4H), 3.66-3.33 (m, 5H), 3.17-2.97 (m, 1H), 2.36-1.97 (m, 10H), 1.94-1.82 (m, 2H), 1.80-1.64 (m, 1H), 1.02-0.91 (m, 2H), 0.90-0.82 (m, 2H).

### N-(3-(3,5-dimethylisoxazol-4-yl)-4-(2-(4-methoxypiperidin-1-yl)ethoxy)phenyl)cyclopropanecarboxamide (143)

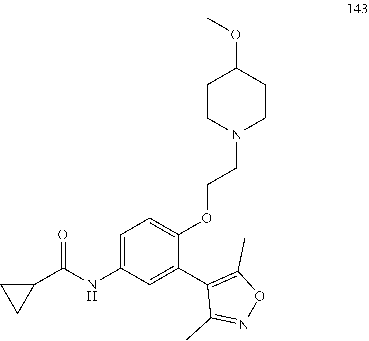 [SMILES: COC1CCN(CCOc2ccc(NC(=O)C3CC3)cc2-c2c(C)noc2C)CC1]

Compound 143 was prepared according to the synthesis described for compound 136, substituting 4-methoxypiperidine for 1,5,6,7,8,8a-hexahydrooxazolo[3,4-a]pyrazin-3-one hydrochloride.

LCMS (ESI): m/z [M+H] calcd for C₂₅H₃₂N₃O₄: 414.0, found: 414.2.

¹H NMR (400 MHZ, methanol-d₄) δ=7.60 (dd, J=2.7, 8.9 Hz, 1H), 7.44 (d, J=2.6 Hz, 1H), 7.13 (d, J=8.9 Hz, 1H), 4.54-4.22 (m, 2H), 3.59-3.32 (m, 7H), 3.2-3.0 (m, 3H), 2.32 (s, 3H), 2.22-1.95 (m, 5H), 1.8-1.45 (m, 3H), 1.00-0.91 (m, 2H), 0.90-0.78 (m, 2H).

### (S)—N-(3-(3,5-dimethylisoxazol-4-yl)-4-(2-(3-methoxypyrrolidin-1-yl)ethoxy)phenyl)cyclopropanecarboxamide (144)

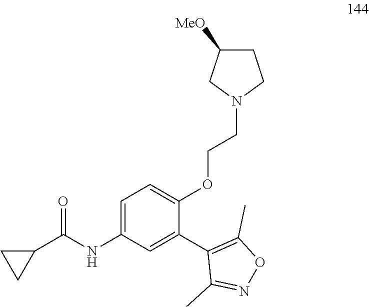 [SMILES: CO[C@H]1CCN(CCOc2ccc(NC(=O)C3CC3)cc2-c2c(C)noc2C)C1]

Compound 144 was prepared according to the synthesis described for compound 136, substituting (S)-3-methoxypyrrolidine for 1,5,6,7,8,8a-hexahydrooxazolo[3,4-a]pyrazin-3-one hydrochloride.

LCMS (ESI): m/z [M+H] calcd for C₂₂H₃₀N₃O₄: 400.0, found: 400.2.

¹H NMR (400 MHZ, methanol-d₄) δ=7.66-7.51 (m, 1H), 7.44 (t, J=2.3 Hz, 1H), 7.12 (d, J=8.9 Hz, 1H), 4.45-4.23 (m, 2H), 4.08 (br s, 1H), 3.72-3.42 (m, 4H), 3.30 (br d, J=3.4 Hz, 5H), 2.20-2.19 (m, 1H), 2.37-1.90 (m, 7H), 1.83-1.66 (m, 1H), 1.02-0.90 (m, 2H), 0.90-0.78 (m, 2H).

### (R)—N-(3-(3,5-dimethylisoxazol-4-yl)-4-(2-(3-methoxypyrrolidin-1-yl)ethoxy)phenyl)cyclopropanecarboxamide (145)

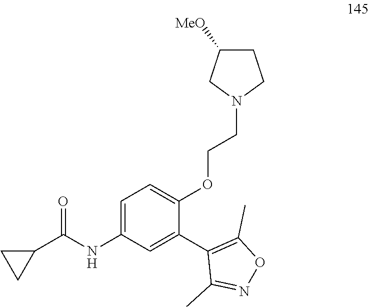 [SMILES: CO[C@@H]1CCN(CCOc2ccc(NC(=O)C3CC3)cc2-c2c(C)noc2C)C1]

Compound 145 was prepared according to the synthesis described for compound 136, substituting (R)-3-methoxypyrrolidine for 1,5,6,7,8,8a-hexahydrooxazolo[3,4-a]pyrazin-3-one hydrochloride.

LCMS (ESI): m/z [M+H] calcd for C₂₂H₃₀N₃O₄: 400.0, found: 400.2.

¹H NMR (400 MHZ, methanol-d₄) δ=7.59 (dd, J=2.6, 8.9 Hz, 1H), 7.45 (d, J=2.6 Hz, 1H), 7.12 (d, J=9.0 Hz, 1H), 4.42-4.26 (m, 2H), 4.14-3.99 (m, 1H), 3.61 (br s, 4H), 3.30-3.28 (m, 5H), 2.32-1.90 (m 8H), 1.75 (tt, J=4.6, 7.9 Hz, 1H), 0.99-0.91 (m, 2H), 0.90-0.81 (m, 2H).

### N-(3-(3,5-dimethylisoxazol-4-yl)-4-(2-(4-hydroxypiperidin-1-yl)ethoxy)phenyl)cyclopropanecarboxamide (146)

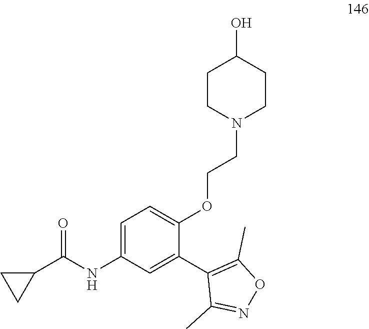 [SMILES: Cc1noc(C)c1-c1cc(NC(=O)C2CC2)ccc1OCCN1CCC(O)CC1]

Compound 146 was prepared according to the synthesis described for compound 136, substituting piperidin-4-ol for 1,5,6,7,8,8a-hexahydrooxazolo[3,4-a]pyrazin-3-one hydrochloride.

LCMS (ESI): m/z [M+H] calcd for C₂₂H₃₀N₃O₄: 400.0, found: 400.2.

¹H NMR (400 MHZ, methanol-d₄) δ=7.59 (dd, J=2.7, 8.9 Hz, 1H), 7.44 (d, J=2.6 Hz, 1H), 7.12 (d, J=8.9 Hz, 1H), 4.36 (br s, 2H), 4.02-3.66 (m, 1H), 3.56-3.49 (m, 2H), 3.44 (br d, J=1.8 Hz, 1H), 3.30-3.16 (m, 2H), 3.08-2.91 (m, 1H), 2.31 (s, 3H), 2.16 (s, 3H), 2.09-1.98 (m, 1H), 1.95-1.78 (m, 2H), 1.74 (tt, J=4.6, 7.9 Hz, 1H), 1.69-1.54 (m, 1H), 0.99-0.90 (m, 2H), 0.90-0.80 (m, 2H).

### (S)—N-(3-(3,5-dimethylisoxazol-4-yl)-4-(2-(3-hydroxypyrrolidin-1-yl)ethoxy)phenyl)cyclopropanecarboxamide (147)

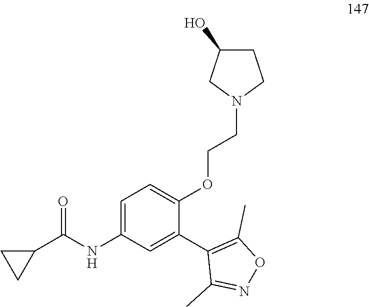 [SMILES: Cc1noc(C)c1-c1cc(NC(=O)C2CC2)ccc1OCCN1CC[C@H](O)C1]

Compound 147 was prepared according to the synthesis described for compound 136, substituting (S)-pyrrolidin-3-ol for 1,5,6,7,8,8a-hexahydrooxazolo[3,4-a]pyrazin-3-one hydrochloride.

LCMS (ESI): m/z [M+H] calcd for C₂₁H₂₈N₃O₄: 386.0, found: 386.2.

¹H NMR (400 MHZ, methanol-d₄) δ=7.60 (dd, J=2.7, 8.9 Hz, 1H), 7.44 (d, J=2.6 Hz, 1H), 7.13 (d, J=8.9 Hz, 1H), 4.47 (br d, J=1.7 Hz, 1H), 4.37-4.25 (m, 2H), 3.61 (br s, 4H), 2.92 (s, 2H), 2.37-1.83 (m, 8H), 1.74 (tt, J=4.6, 7.9 Hz, 1H), 1.00-0.90 (m, 2H), 0.90-0.79 (m, 2H).

### (R)—N-(3-(3,5-dimethylisoxazol-4-yl)-4-(2-(3-hydroxypyrrolidin-1-yl)ethoxy)phenyl)cyclopropanecarboxamide (148)

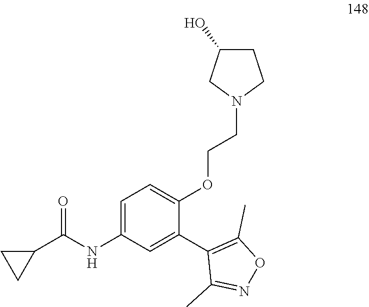 [SMILES: Cc1noc(C)c1-c1cc(NC(=O)C2CC2)ccc1OCCN1CC[C@@H](O)C1]

Compound 148 was prepared according to the synthesis described for compound 136, substituting (R)-pyrrolidin-3-ol for 1,5,6,7,8,8a-hexahydrooxazolo[3,4-a]pyrazin-3-one hydrochloride.

LCMS (ESI): m/z [M+H] calcd for C₂₁H₂₈N₃O₄: 386.0, found: 386.2.

¹H NMR (400 MHZ, methanol-d₄) δ=7.59 (dd, J=2.6, 8.9 Hz, 1H), 7.44 (d, J=2.6 Hz, 1H), 7.13 (d, J=8.9 Hz, 1H), 4.50-4.45 (m, 1H), 4.37-4.28 (m, 2H), 3.61 (br s, 4H), 3.29-2.91 (m, 2H), 2.44-1.81 (m, 8H), 1.78-1.71 (m, 1H), 0.97-0.83 (m, 4H).

### N-(3-(3,5-dimethylisoxazol-4-yl)-4-(2-(ethyl(methyl)amino)ethoxy)phenyl)cyclopropanecarboxamide (149)

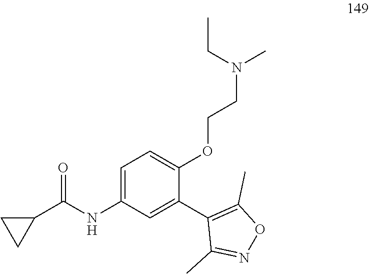 [SMILES: CCN(C)CCOc1ccc(NC(=O)C2CC2)cc1-c1c(C)noc1C]

Compound 149 was prepared according to the synthesis described for compound 136, substituting N-methylethanamine for 1,5,6,7,8,8a-hexahydrooxazolo[3,4-a]pyrazin-3-one hydrochloride.

LCMS (ESI): m/z [M+H] calcd for C₂₀H₂₈N³⁰³S: 358.0, found: 358.2.

¹H NMR (400 MHZ, methanol-d₄) δ=7.60 (dd, J=2.4, 8.9 Hz, 1H), 7.44 (d, J=2.4 Hz, 1H), 7.13 (d, J=9.0 Hz, 1H), 4.35 (q, J=4.5 Hz, 2H), 3.66-3.43 (m, 2H), 3.26-3.04 (m, 2H), 2.80 (s, 3H), 2.32 (s, 3H), 2.16 (s, 3H), 1.82-1.68 (m, 1H), 1.22 (t, J=7.3 Hz, 3H), 1.01-0.90 (m, 2H), 0.90-0.80 (m, 2H).

### N-(3-(3,5-dimethylisoxazol-4-yl)-4-(2-(methyl(propyl)amino)ethoxy)phenyl)cyclopropanecarboxamide (150)

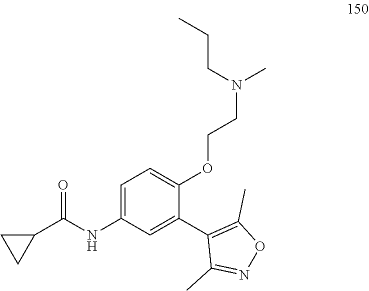 [SMILES: CCCN(C)CCOc1ccc(NC(=O)C2CC2)cc1-c1c(C)noc1C]

Compound 150 was prepared according to the synthesis described for compound 136, substituting N-methylpropan-1-amine for 1,5,6,7,8,8a-hexahydrooxazolo[3,4-a]pyrazin-3-one hydrochloride.

LCMS (ESI): m/z [M+H] calcd for C₂₁H₃₀N₃O₃: 372.0, found: 372.3.

¹H NMR (400 MHZ, methanol-d₄) δ=7.64-7.55 (m, 1H), 7.47-7.41 (m, 1H), 7.13 (d, J=8.9 Hz, 1H), 4.35 (br s, 2H), 3.65-3.42 (m, 2H), 3.17-2.92 (m, 2H), 2.82 (s, 3H), 2.32 (s, 3H), 2.17 (s, 3H), 1.81-1.56 (m, 3H), 0.98-0.89 (m, 5H), 0.86 (td, J=3.1, 7.8 Hz, 2H).

### N-(4-(2-((cyclopropylmethyl)(methyl)amino)ethoxy)-3-(3,5-dimethylisoxazol-4-yl)phenyl)cyclopropanecarboxamide (151)

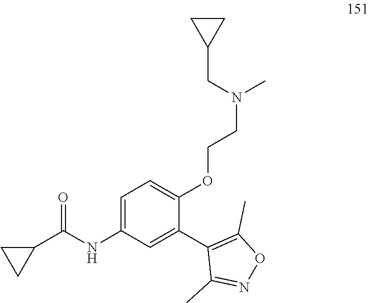 [SMILES: Cc1noc(C)c1-c1cc(NC(=O)C2CC2)ccc1OCCN(C)CC1CC1]

Compound 151 was prepared according to the synthesis described for compound 136, substituting 1-cyclopropyl-N-methylmethanamine for 1,5,6,7,8,8a-hexahydrooxazolo[3,4-a]pyrazin-3-one hydrochloride.

LCMS (ESI): m/z [M+H] calcd for C₂₃H₃₀N₃O₃: 384.0, found: 384.3.

¹H NMR (400 MHZ, methanol-d₄) δ=7.64-7.56 (m, 1H), 7.44 (d, J=1.6 Hz, 1H), 7.13 (d, J=8.9 Hz, 1H), 4.45-4.30 (m, 2H), 3.73-3.46 (m, 2H), 3.11-2.89 (m, 2H), 2.87 (s, 3H), 2.32 (s, 3H), 2.16 (s, 3H), 1.79-1.70 (m, 1H), 1.06-0.97 (m, 1H), 0.97-0.91 (m, 2H), 0.91-0.82 (m, 2H), 0.75-0.68 (m, 2H), 0.37-0.29 (m, 2H).

### N-(4-(2-(cyclobutyl(methyl)amino)ethoxy)-3-(3,5-dimethylisoxazol-4-yl)phenyl)cyclopropanecarboxamide (152)

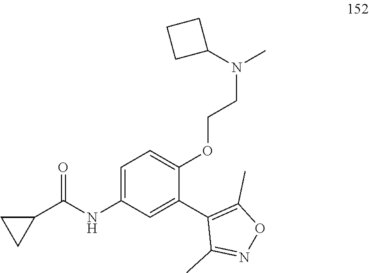 [SMILES: Cc1noc(C)c1-c1cc(NC(=O)C2CC2)ccc1OCCN(C)C1CCC1]

Compound 152 was prepared according to the synthesis described for compound 136, substituting N-methylcyclobutanamine for 1,5,6,7,8,8a-hexahydrooxazolo[3,4-a]pyrazin-3-one hydrochloride.

LCMS (ESI): m/z [M+H] calcd for C₂₃H₃₀N₃O₃: 384.0, found: 384.3.

¹H NMR (400 MHZ, methanol-d₄) δ=7.60 (dd, J=2.5, 8.9 Hz, 1H), 7.44 (d, J=1.8 Hz, 1H), 7.12 (d, J=8.9 Hz, 1H), 4.43-4.24 (m, 2H), 3.69 (quin, J=8.3 Hz, 1H), 3.52-3.34 (m, 2H), 2.68 (s, 3H), 2.32 (s, 3H), 2.24 (br s, 2H), 2.16 (s, 3H), 2.15-1.99 (m, 2H), 1.88-1.69 (m, 3H), 0.99-0.90 (m, 2H), 0.90-0.81 (m, 2H).

### N-(4-(2-(diethylamino)ethoxy)-3-(3,5-dimethylisoxazol-4-yl)phenyl)cyclopropanecarboxamide (153)

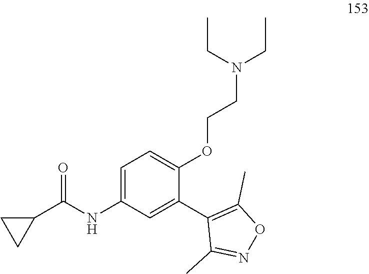 [SMILES: CCN(CC)CCOc1ccc(NC(=O)C2CC2)cc1-c1c(C)noc1C]

Compound 153 was prepared according to the synthesis described for compound 136, substituting diethylamine for 1,5,6,7,8,8a-hexahydrooxazolo[3,4-a]pyrazin-3-one hydrochloride.

LCMS (ESI): m/z [M+H] calcd for C₂₁H₃₀N₃O₃: 372.0, found: 372.2.

¹H NMR (400 MHZ, methanol-d₄) δ=7.64-7.56 (m, 1H), 7.47-7.39 (m, 1H), 7.12 (d, J=8.9 Hz, 1H), 4.40-4.28 (m, 2H), 3.61-3.49 (m, 2H), 3.16 (q, J=7.3 Hz, 4H), 2.31 (s, 3H), 2.16 (s, 3H), 1.80-1.70 (m, 1H), 1.20 (t, J=7.3 Hz, 6H), 1.00-0.89 (m, 2H), 0.89-0.80 (m, 2H).

### N-[4-[2-(azetidin-1-yl)ethoxy]-3-(3,5-dimethylisoxazol-4-yl)phenyl]cyclopropanecarboxamide (154)

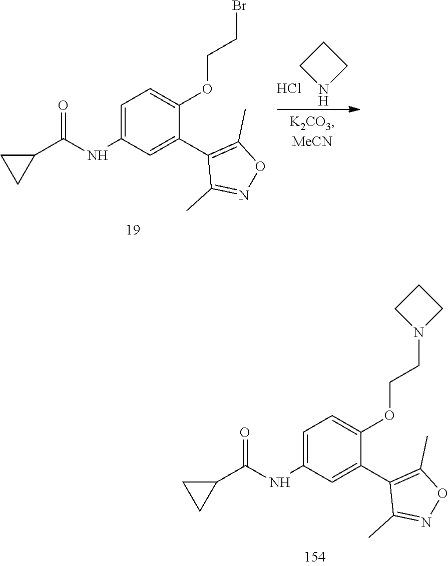

To a solution of N-[4-(2-bromoethoxy)-3-(3,5-dimethylisoxazol-4-yl)phenyl]cyclopropanecarboxamide ((19), 100 mg, 263.68 μmol, 1 eq.) and azetidine (37.00 mg, 395.52 μmol, 43.74 μL, 1.5 eq., HCl) in MeCN (2 mL) was added K₂CO₃ (109.33 mg, 791.04 μmol, 3 eq.). The reaction mixture was stirred at 80° C. for 16 hrs (monitored by LC-MS). The reaction mixture was concentrated under vacuum. The residue was purified by prep-HPLC (column: UniSil 3-100 C18 UItra (150*25 mm*3 μm); mobile phase: [water (0.225% FA)-ACN]; B %: 3%-33%, 10 min) followed by lyophilization. The residue was purified by prep-HPLC (column: Waters Xbridge 150*25 mm*5 um; mobile phase: [water (0.05% ammonia hydroxide v/v)-ACN]; B %: 35%-60%, 10 min) followed by lyophilization. N-[4-[2-(azetidin-1-yl)ethoxy]-3-(3,5-dimethylisoxazol-4-yl)phenyl]cyclopropanecarboxamide (12.87 mg, 36.21 μmol, 13.73% yield, 100% purity) was obtained as an off-white solid.

LCMS (ESI): m/z [M+H] calcd for C₂₀H₂₆N₃O₃: 356.19; found: 356.3.

¹H NMR (400 MHZ, methanol-d₄) δ ppm 0.80-0.88 (m, 2H), 0.90-0.97 (m, 2H) 1.73 (tt, J=7.90, 4.61 Hz, 1H) 2.03 (quin, J=7.19 Hz, 2H) 2.15 (s, 3H) 2.29 (s, 3H) 2.77 (t, J=5.32 Hz, 2H) 3.16 (t, J=7.19 Hz, 4H) 3.98 (t, J=5.38 Hz, 2H) 7.02 (d, J=8.88 Hz, 1H) 7.40 (d, J=2.63 Hz, 1H) 7.51 (dd, J=8.88, 2.75 Hz, 1H).

### N-[3-(3,5-dimethylisoxazol-4-yl)-4-[2-(2-oxa-6-azaspiro[3.3]heptan-6-yl)ethoxy]phenyl]cyclopropanecarboxamide (155)

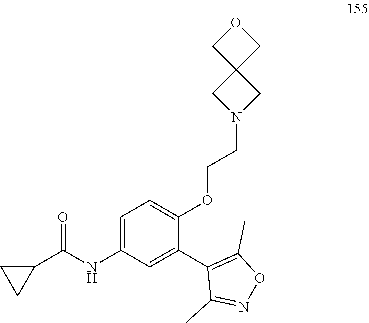 [SMILES: Cc1noc(C)c1-c1cc(NC(=O)C2CC2)ccc1OCCN1CC2(COC2)C1]

To a solution of N-[4-(2-bromoethoxy)-3-(3,5-dimethylisoxazol-4-yl)phenyl]cyclopropanecarboxamide ((19), 100 mg, 263.68 μmol, 1 eq.) and 2-oxa-6-azaspiro[3.3]heptane (39.21 mg, 395.52 μmol, 21.87 μL, 1.5 eq.) in MeCN (2 mL) was added K₂CO₃ (109.33 mg, 791.04 μmol, 3 eq.). The reaction mixture was stirred at 80° C. for 16 hrs (monitored by LC-MS). The reaction mixture was concentrated under vacuum. The residue was purified by prep-HPLC (column: UniSil 3-100 C18 UItra (150*25 mm*3 μm); mobile phase: [water (0.225% FA)-ACN]; B %: 3%-33%, 10 min) followed by lyophilization. N-[3-(3,5-dimethylisoxazol-4-yl)-4-[2-(2-oxa-6-azaspiro[3.3]heptan-6-yl)ethoxy]phenyl]cyclopropanecarboxamide (63.29 mg, 148.09 μmol, 56.16% yield, 93% purity) was obtained as a white solid.

LCMS (ESI): m/z [M+H] calcd for C₂₂H₂₈N₃O₄: 398.20; found: 398.1.

¹H NMR (400 MHZ, methanol-d₄) δ ppm 0.82-0.96 (m, 4H) 1.70-1.77 (m, 1H) 2.12-2.19 (m, 3H) 2.28-2.36 (m, 3H) 3.14-3.29 (m, 2H) 3.79 (s, 4H) 4.09 (t, J=4.88 Hz, 2H) 4.69 (s, 4H) 7.05 (d, J=8.88 Hz, 1H) 7.42 (d, J=2.50 Hz, 1H) 7.55 (dd, J=8.88, 2.63 Hz, 1H) 8.34 (br s, 1H).

### N-[3-(3,5-dimethylisoxazol-4-yl)-4-[2-(4-methyl-3-oxo-piperazin-1-yl)ethoxy]phenyl]cyclopropanecarboxamide (156)

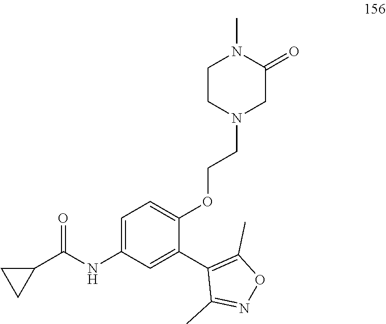 [SMILES: Cc1noc(C)c1-c1cc(NC(=O)C2CC2)ccc1OCCN1CCN(C)C(=O)C1]

To a solution of N-[4-(2-bromoethoxy)-3-(3,5-dimethylisoxazol-4-yl)phenyl]cyclopropanecarboxamide ((19), 100 mg, 263.68 μmol, 1 eq.) and 1-methylpiperazin-2-one (45.15 mg, 395.52 μmol, 21.87 μL, 1.5 eq.) in MeCN (2 mL) was added K₂CO₃ (109.33 mg, 791.04 μmol, 3 eq.). The reaction mixture was stirred at 80° C. for 16 hrs (monitored by LC-MS). The residue was concentrated under vacuum. The residue was purified by prep-HPLC (column: UniSil 3-100 C18 UItra (150*25 mm*3 μm); mobile phase: [water (0.225% FA)-ACN]; B %: 8%-38%, 10 min) followed by lyophilization. N-[3-(3,5-dimethylisoxazol-4-yl)-4-[2-(4-methyl-3-oxo-piperazin-1-yl)ethoxy]phenyl]cyclopropanecarboxamide (65 mg, 149.70 μmol, 56.77% yield, 95% purity) was obtained as a yellow solid.

LCMS (ESI): m/z [M+H] calcd for C₂₂H₂₉N₄O₄: 413.21; found: 413.1.

¹H NMR (400 MHZ, methanol-d₄) δ ppm 0.81-0.88 (m, 2H) 0.91-1.00 (m, 2H) 1.66-1.81 (m, 1H) 2.16 (s, 3H) 2.31 (s, 3H) 2.71 (t, J=5.50 Hz, 2H) 2.77 (t, J=5.25 Hz, 2H) 2.92 (s, 3H) 3.11 (s, 2H) 3.28 (t, J=5.50 Hz, 2H) 4.11 (t, J=5.25 Hz, 2H) 7.07 (d, J=8.88 Hz, 1H) 7.40 (d, J=2.50 Hz, 1H) 7.52 (dd, J=8.88, 2.50 Hz, 1H).

### N-[3-(3,5-dimethylisoxazol-4-yl)-4-[2-(6-oxa-1-azaspiro[3.3]heptan-1-yl)ethoxy]phenyl]cyclopropanecarboxamide (157)

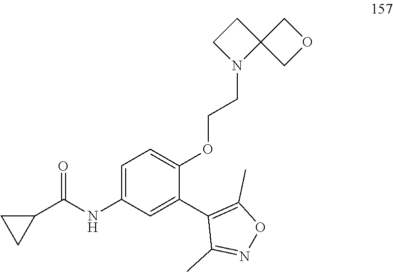 [SMILES: Cc1noc(C)c1-c1cc(NC(=O)C2CC2)ccc1OCCN1CCC12COC2]

To a solution of N-[4-(2-bromoethoxy)-3-(3,5-dimethylisoxazol-4-yl)phenyl]cyclopropanecarboxamide ((19), 100 mg, 263.68 μmol, 1 eq.) and 6-oxa-1-azaspiro[3.3]heptane oxalic acid (83.62 mg, 290.05 μmol, 1.1 eq.) in MeCN (2 mL) was added K₂CO₃ (109.33 mg, 791.04 μmol, 3 eq.). The reaction mixture was stirred at 80° C. for 16 hrs (monitored by LC-MS). The reaction mixture was concentrated under vacuum. The residue was purified by prep-HPLC (column: Phenomenex Synergi C18 150*25*10 μm; mobile phase: [water (0.225% FA)-ACN]; B %: 4%-34%, 10 min) followed by lyophilization. N-[3-(3,5-dimethylisoxazol-4-yl)-4-[2-(6-oxa-1-azaspiro[3.3]heptan-1-yl)ethoxy]phenyl]cyclopropanecarboxamide (47.77 mg, 120.19 μmol, 45.58% yield, 100% purity) was obtained as a yellow solid.

LCMS (ESI): m/z [M+H] calcd for C₂₂H₂₈N₃O₄: 398.20; found: 398.1.

¹H NMR (400 MHZ, methanol-d₄) δ ppm 0.79-0.88 (m, 2H) 0.90-0.99 (m, 2H) 1.66-1.81 (m, 1H) 2.16 (s, 3H) 2.30 (s, 3H) 2.38 (t, J=7.21 Hz, 2H) 3.08 (t, J=7.15 Hz, 2H) 3.14 (t, J=5.26 Hz, 2H) 4.12 (t, J=5.26 Hz, 2H) 4.60 (d, J=7.95 Hz, 2H) 4.85 (s, 2H) 7.07 (d, J=8.93 Hz, 1H) 7.41 (d, J=2.69 Hz, 1H) 7.53 (dd, J=8.93, 2.69 Hz, 1H) 8.19 (br s, 1H).

### 2-[1-[2-[4-(cyclopropanecarbonylamino)-2-(3,5-dimethylisoxazol-4-yl)phenoxy]ethyl]-4-piperidyl]acetic acid (158)

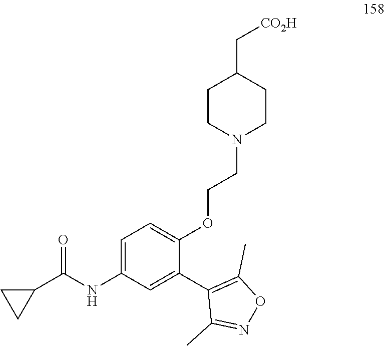 [SMILES: Cc1noc(C)c1-c1cc(NC(=O)C2CC2)ccc1OCCN1CCC(CC(=O)O)CC1]

To a solution of N-[4-(2-bromoethoxy)-3-(3,5-dimethylisoxazol-4-yl)phenyl]cyclopropanecarboxamide ((19), 100 mg, 263.68 μmol, 1 eq.) and methyl 2-(4-piperidyl)acetate hydrochloride (61.28 mg, 316.42 μmol, 1.2 eq.) in MeCN (2 mL) was added K₂CO₃ (109.33 mg, 791.04 μmol, 3 eq.). The reaction mixture was stirred at 80° C. for 16 hrs (monitored by LC-MS). The reaction mixture was concentrated under vacuum. Methyl 2-[1-[2-[4-(cyclopropanecarbonylamino)-2-(3,5-dimethylisoxazol-4-yl)phenoxy]ethyl]-4-piperidyl]acetate (120 mg, 263.42 μmol, 99.90% yield) was obtained as a white solid.

To a solution of methyl 2-[1-[2-[4-(cyclopropanecarbonylamino)-2-(3,5-dimethylisoxazol-4-yl)phenoxy]ethyl]-4-piperidyl]acetate (120 mg, 263.42 μmol, 1 eq.) in THF (1 mL) was added a solution of LiOH·H₂O (33.16 mg, 790.26 μmol, 3 eq.) in H₂O (1 mL). The reaction mixture was stirred at 25° C. for 1 hr. The reaction mixture was concentrated under vacuum. The reaction mixture was purified by prep-HPLC (column: Phenomenex Synergi C18 150*25*10 μm; mobile phase: [water (0.225% FA)-ACN]; B %: 15%-45%, 9 min) followed by lyophilization. 2-[1-[2-[4-(cyclopropanecarbonylamino)-2-(3,5-dimethylisoxazol-4-yl)phenoxy]ethyl]-4-piperidyl]acetic acid (14.38 mg, 32.57 μmol, 12.36% yield, 100% purity) was obtained as a yellow gum.

LCMS (ESI): m/z [M+H] calcd for C₂₄H₃₂N₃O₅: 442.23; found: 442.2.

¹H NMR (400 MHZ, DMSO-d₆) δ ppm 0.69-0.83 (m, 4H) 1.05-1.23 (m, 2H) 1.58 (br d, J=10.27 Hz, 3H) 1.67-1.79 (m, 1H) 1.99 (br t, J=11.19 Hz, 2H) 2.08-2.15 (m, 5H) 2.27 (s, 3H) 2.62 (br t, J=5.44 Hz, 2H) 2.82 (br d, J=11.37 Hz, 2H) 4.02 (br t, J=5.56 Hz, 2H) 7.06 (d, J=8.93 Hz, 1H) 7.40 (d, J=2.57 Hz, 1H) 7.54 (dd, J=8.93, 2.57 Hz, 1H) 10.10 (s, 1H).

### N-[3-(3,5-dimethylisoxazol-4-yl)-4-[2-[(1R,5S)-3-oxa-6-azabicyclo[3.1.1]heptan-6-yl]ethoxy]phenyl]cyclopropanecarboxamide (159)

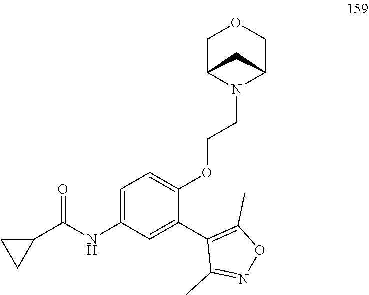 [SMILES: Cc1noc(C)c1-c1cc(NC(=O)C2CC2)ccc1OCCN1[C@@H]2COC[C@H]1C2]

To a mixture of N-[4-(2-bromoethoxy)-3-(3,5-dimethylisoxazol-4-yl)phenyl]cyclopropanecarboxamide ((19), 100 mg, 263.68 μmol, 1 eq.) and (1R,5S)-3-oxa-6-azabicyclo[3.1.1]heptane (42.90 mg, 316.42 μmol, 1.2 eq., HCl) in DMF (1 mL) was added KI (4.38 mg, 26.37 μmol, 0.1 eq.) and K₂CO₃ (145.77 mg, 1.05 mmol, 4 eq.). The mixture was stirred at 80° C. for 16 hrs (monitored by LC-MS). The mixture was filtered and the filtrate was concentrated under vacuum. The residue was purified by prep-HPLC (column: Xtimate C18 150*40 mm*10 μm; mobile phase: [water (0.05% ammonia hydroxide v/v)-ACN]; B %: 17%-47%, 10 min) followed by lyophilization. N-[3-(3,5-dimethylisoxazol-4-yl)-4-[2-[(1R,5S)-3-oxa-6-azabicyclo[3.1.1]heptan-6-yl]ethoxy]phenyl]cyclopropanecarboxamide (32.48 mg, 76.00 μmol, 28.82% yield, 93% purity) as a yellow solid was obtained.

LCMS (ESI): m/z [M+H] calcd for C₂₂H₂₈N₃O₄: 398.20; found: 398.2.

¹H NMR (400 MHZ, methanol-d₄) δ=7.53 (dd, J=2.7, 8.9 Hz, 1H), 7.37 (d, J=2.6 Hz, 1H), 7.05 (d, J=9.0 Hz, 1H), 4.16-4.04 (m, 4H), 3.57 (d, J=11.0 Hz, 2H), 3.28 (d, J=6.1 Hz, 2H), 3.02-2.97 (m, 2H), 2.61-2.54 (m, 1H), 2.26 (s, 3H), 2.12 (s, 3H), 1.79-1.70 (m, 2H), 0.96-0.91 (m, 2H), 0.87-0.81 (m, 2H).

### N-(4-(2-((1S,4S)-2-oxa-5-azabicyclo[2.2.1]heptan-5-yl)ethoxy)-3-(3,5-dimethylisoxazol-4-yl)phenyl)cyclopropanecarboxamide (160)

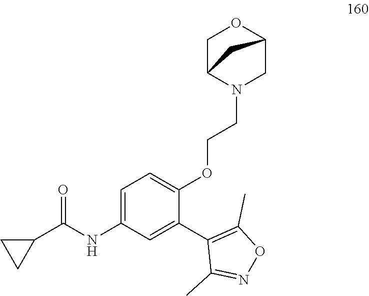 [SMILES: Cc1noc(C)c1-c1cc(NC(=O)C2CC2)ccc1OCCN1C[C@@H]2C[C@H]1CO2]

Compound 160 was prepared according to the synthesis described for compound 159, substituting (1S,4S)-2-oxa-5-azabicyclo[2.2.1]heptane for (1R,5S)-3-oxa-6-azabicyclo[3.1.1]heptane.

LCMS (ESI): m/z [M+H] calcd for C₂₂H₂₈N₃O₄: 398.20; found: 398.2.

¹H NMR (400 MHZ, methanol-d₄) δ=7.59 (dd, J=2.6, 8.9 Hz, 1H), 7.45 (d, J=2.6 Hz, 1H), 7.12 (d, J=8.9 Hz, 1H), 4.70-4.54 (m, 1H), 4.50-4.2 (m, 3H), 4.19-3.40 (m, 5H), 3.28-2.90 (m, 1H), 2.32 (s, 3H), 2.24-2.01 (m, 5H), 1.79-1.71 (m, 1H), 0.97-0.91 (m, 2H), 0.88-0.83 (m, 2H).

### N-[3-(3,5-dimethylisoxazol-4-yl)-4-[2-[(1S,4S)-2-oxa-5-azabicyclo[2.2.1]heptan-5-yl]ethoxy]phenyl]benzamide (161)

### Preparation of 2-[2-(3,5-dimethylisoxazol-4-yl)-4-nitro-phenoxy]ethanol (20)

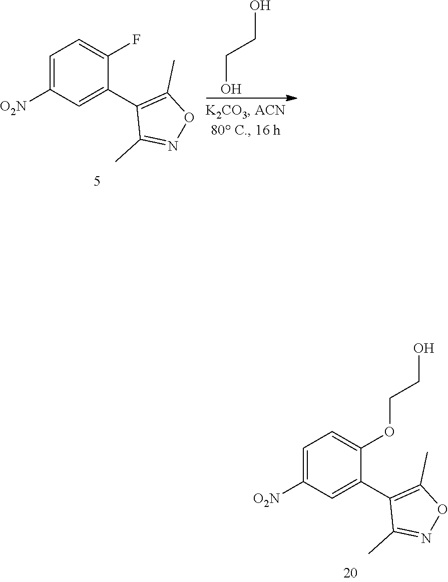

To a solution of 4-(2-fluoro-5-nitro-phenyl)-3,5-dimethyl-isoxazole ((5), 2 g, 8.47 mmol, 1 eq.) and ethylene glycol (5.26 g, 84.67 mmol, 4.73 mL, 10 eq.) in MeCN (15 mL) was added K₂CO₃ (1.76 g, 12.70 mmol, 1.5 eq.). The reaction mixture was stirred at 80° C. for 2 hrs (monitored by LC-MS). The residue was poured into water (20 mL). The aqueous phase was extracted with ethyl acetate (30 mL×3). The combined organic phase was washed with brine (30 mL), dried with anhydrous Na₂SO₄, filtered and concentrated under vacuum. The residue was purified by flash silica gel chromatography (40 g SepaFlash® Silica Flash Column, eluent of 0˜70% ethyl acetate/petroleum ether gradient). 2-[2-(3,5-dimethylisoxazol-4-yl)-4-nitro-phenoxy]ethanol ((20), 1.8 g, 6.47 mmol, 76.40% yield) was obtained as a white solid.

### Preparation of 2-[2-(3,5-dimethylisoxazol-4-yl)-4-nitro-phenoxy]ethyl 4-methylbenzenesulfonate (21)

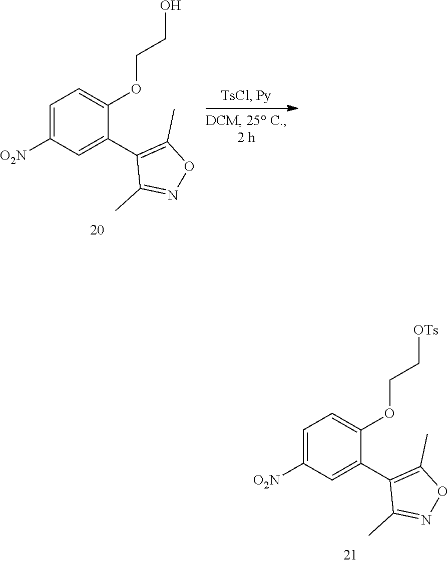 [SMILES: Cc1noc(C)c1-c1cc([N+](=O)[O-])ccc1OCCO ; Cc1ccc(S(=O)(=O)OCCOc2ccc([N+](=O)[O-])cc2-c2c(C)noc2C)cc1]

To a solution of 2-[2-(3,5-dimethylisoxazol-4-yl)-4-nitro-phenoxy]ethanol ((20), 600 mg, 2.16 mmol, 1 eq.) and TosCl (2.06 g, 10.78 mmol, 5 eq.) in DCM (5 mL) was added Py (4.90 g, 61.95 mmol, 5 mL, 28.73 eq.). The reaction mixture was stirred at 25° C. for 16 hrs (monitored by LC-MS). The residue was poured into water (20 mL). The aqueous phase was extracted with ethyl acetate (30 mL×3). The combined organic phase was washed with brine (20 mL), dried with anhydrous Na₂SO₄, filtered and concentrated under vacuum. The residue was purified by flash silica gel chromatography (25 g SepaFlash® Silica Flash Column, eluent of 0˜60% ethyl acetate/petroleum ether gradient). 2-[2-(3,5-dimethylisoxazol-4-yl)-4-nitro-phenoxy]ethyl 4-methylbenzenesulfonate ((21), 750 mg, 1.73 mmol, 80.43% yield) was obtained as a colorless oil.

### Preparation of (1S,4S)-5-[2-[2-(3,5-dimethylisoxazol-4-yl)-4-nitro-phenoxy]ethyl]-2-oxa-5-azabicyclo[2.2.1]heptane (22)

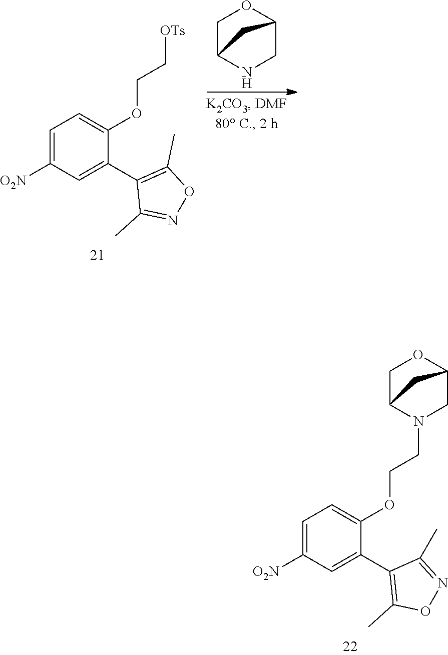 [SMILES: Cc1ccc(S(=O)(=O)OCCOc2ccc([N+](=O)[O-])cc2-c2c(C)noc2C)cc1 ; C1O[C@@H]2CN[C@H]1C2 ; Cc1noc(C)c1-c1cc([N+](=O)[O-])ccc1OCCN1C[C@@H]2C[C@H]1CO2]

To a solution of 2-[2-(3,5-dimethylisoxazol-4-yl)-4-nitro-phenoxy]ethyl 4-methylbenzenesulfonate ((21), 750 mg, 1.73 mmol, 1 eq.) and (1S,4S)-2-oxa-5-azabicyclo[2.2.1]heptane (282.19 mg, 2.08 mmol, 1.2 eq., HCl) in DMF (10 mL) was added K₂CO₃ (479.39 mg, 3.47 mmol, 2 eq.). The reaction mixture was stirred at 80° C. for 2 hrs (monitored by LC-MS). The residue was poured into water (50 mL). The aqueous phase was extracted with ethyl acetate (40 mL×3). The combined organic phase was washed with brine (30 mL), dried with anhydrous Na₂SO₄, filtered and concentrated under vacuum. The residue was purified by flash silica gel chromatography (25 g SepaFlash® Silica Flash Column, eluent of 0˜100% ethyl acetate/petroleum ether gradient). (1S,4S)-5-[2-[2-(3,5-dimethylisoxazol-4-yl)-4-nitro-phenoxy]ethyl]-2-oxa-5-azabicyclo[2.2.1]heptane ((22), 230 mg, 640.00 μmol, 36.90% yield) was obtained as a yellow oil.

¹H NMR (400 MHZ, methanol-d₄) δ=8.36 (dd, J=2.9, 9.1 Hz, 1H), 8.14 (d, J=2.9 Hz, 1H), 7.32 (d, J=9.3 Hz, 1H), 4.36 (s, 1H), 4.32-4.23 (m, 2H), 3.94 (d, J=7.6 Hz, 1H), 3.54-3.47 (m, 2H), 3.11-3.02 (m, 2H), 2.82 (dd, J=1.6, 10.4 Hz, 1H), 2.52 (d, J=10.5 Hz, 1H), 2.34 (s, 3H), 2.19 (s, 3H), 1.87-1.78 (m, 1H), 1.71 (br d, J=10.0 Hz, 1H).

### Preparation of 3-(3,5-dimethylisoxazol-4-yl)-4-[2-[(1S,4S)-2-oxa-5-azabicyclo[2.2.1]heptan-5-yl]ethoxy]aniline (23)

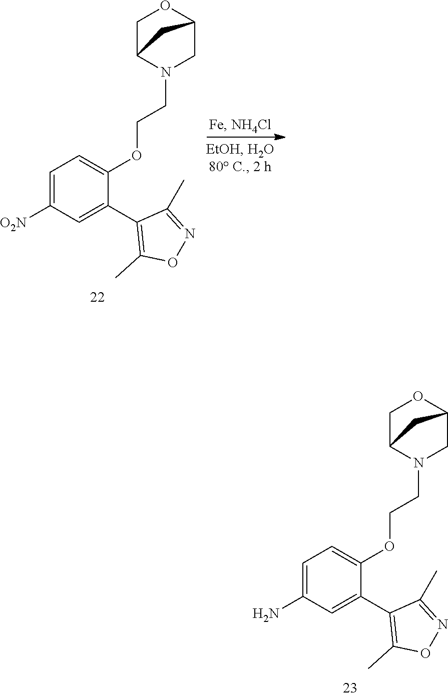 [SMILES: Cc1noc(C)c1-c1cc([N+](=O)[O-])ccc1OCCN1C[C@@H]2C[C@H]1CO2 ; Cc1noc(C)c1-c1cc(N)ccc1OCCN1C[C@@H]2C[C@H]1CO2]

To a solution of (1S,4S)-5-[2-[2-(3,5-dimethylisoxazol-4-yl)-4-nitro-phenoxy]ethyl]-2-oxa-5-azabicyclo[2.2.1]heptane ((22), 200 mg, 556.52 μmol, 1 eq.) and NH₄Cl (89.31 mg, 1.67 mmol, 3 eq.) in EtOH (2 mL) and H₂O (0.5 mL) was added Fe (46.62 mg, 834.78 μmol, 1.5 eq.) at 80° C. The reaction mixture was stirred at 80° C. for 2 hrs (monitored by LC-MS). The reaction mixture was filtered and the filtrate was concentrated under vacuum. The residue was purified by prep-HPLC (column: Welch Xtimate C18 150*25 mm*5 μm; mobile phase: [water (0.05% ammonia hydroxide v/v)-ACN]; B %: 8%-38%, 7 min) followed by concentration under vacuum. 3-(3,5-dimethylisoxazol-4-yl)-4-[2-[(1S,4S)-2-oxa-5-azabicyclo[2.2.1]heptan-5-yl]ethoxy]aniline ((23), 65 mg, 197.33 μmol, 35.46% yield) was obtained as a white solid.

### Preparation of compound N-[3-(3,5-dimethylisoxazol-4-yl)-4-[2-[(1S,4S)-2-oxa-5-azabicyclo[2.2.1]heptan-5-yl]ethoxy]phenyl]benzamide (161)

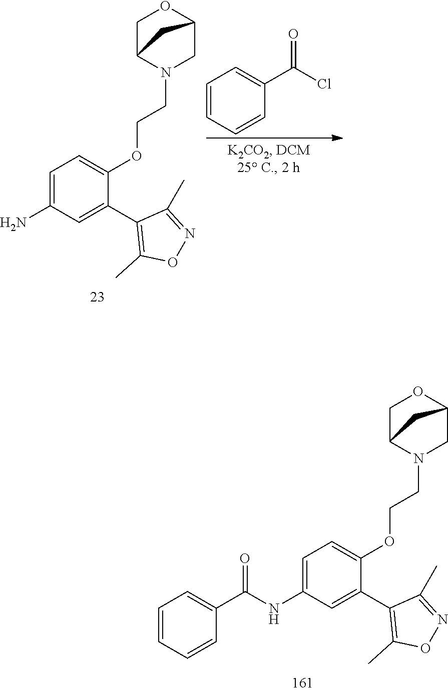

To a solution of 3-(3,5-dimethylisoxazol-4-yl)-4-[2-[(1S,4S)-2-oxa-5-azabicyclo[2.2.1]heptan-5-yl]ethoxy]aniline ((23), 55 mg, 166.97 μmol, 1 eq.) and K₂CO₃ (46.15 mg, 333.94 μmol, 2 eq.) in DCM (1 mL) was added benzoyl chloride (28.16 mg, 200.36 μmol, 23.28 μL, 1.2 eq.). The reaction mixture was stirred at 25° C. for 2 hrs (monitored by LC-MS). The reaction mixture was concentrated under vacuum. The residue was purified by prep-HPLC (column: Waters Xbridge 150*25 mm*5 μm; mobile phase: [water (10 mM NH₄HCO₃)-ACN]; B %: 24%-54%, 10 min) followed by lyophilization. N-[3-(3,5-dimethylisoxazol-4-yl)-4-[2-[(1S,4S)-2-oxa-5-azabicyclo[2.2.1]heptan-5-yl]ethoxy]phenyl]benzamide (28.3 mg, 63.32 μmol, 37.93% yield, 97% purity) was obtained as a yellow solid

LCMS (ESI): m/z [M+H] calcd for C₂₅H₂₈N₃O₄: 434.20; found: 434.2.

¹H NMR (400 MHZ, methanol-d₄) δ=7.98-7.89 (m, 2H), 7.70 (dd, J=2.6, 8.9 Hz, 1H), 7.62-7.47 (m, 4H), 7.12 (d, J=9.0 Hz, 1H), 4.34 (s, 1H), 4.12 (t, J=5.3 Hz, 2H), 3.93 (d, J=8.3 Hz, 1H), 3.54-3.44 (m, 2H), 3.06-2.89 (m, 2H), 2.81 (dd, J=1.6, 10.5 Hz, 1H), 2.51 (d, J=11.4 Hz, 1H), 2.33 (s, 3H), 2.19 (s, 3H), 1.86-1.77 (m, 1H), 1.68 (br d, J=10.0 Hz, 1H).

### N-[3-(3,5-dimethylisoxazol-4-yl)-4-[2-[(1S,4S)-2-oxa-5-azabicyclo[2.2.1]heptan-5-yl]ethoxy]phenyl]-1-fluoro-cyclopropanecarboxamide (162)

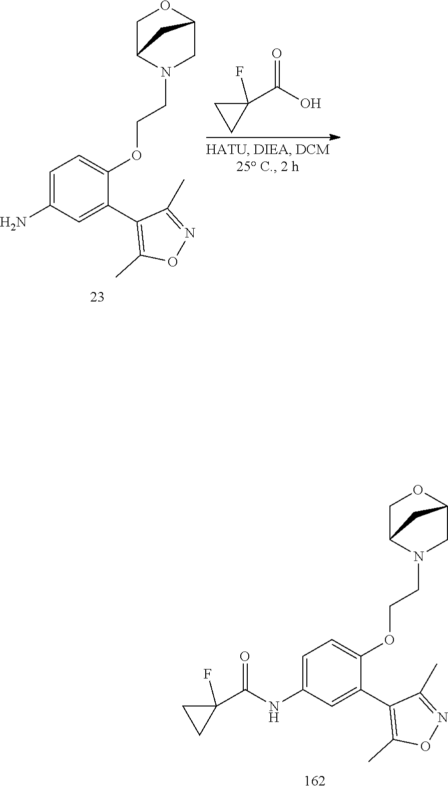 [SMILES: Cc1noc(C)c1-c1cc(N)ccc1OCCN1C[C@@H]2C[C@H]1CO2 ; O=C(O)C1(F)CC1 ; Cc1noc(C)c1-c1cc(NC(=O)C2(F)CC2)ccc1OCCN1C[C@@H]2C[C@H]1CO2]

To a solution of 1-fluorocyclopropanecarboxylic acid (20.85 mg, 200.36 μmol, 1.2 eq.) and HATU (95.23 mg, 250.46 μmol, 1.5 eq.) in DCM (1 mL) was added DIEA (43.16 mg, 333.94 μmol, 58.17 μL, 2 eq.). The reaction mixture was stirred at 25° C. for 30 min. Then 3-(3,5-dimethylisoxazol-4-yl)-4-[2-[(1S,4S)-2-oxa-5-azabicyclo[2.2.1]heptan-5-yl]ethoxy]aniline ((23), 55 mg, 166.97 μmol, 1 eq.) was added to the reaction mixture. The reaction mixture was stirred at 25° C. for 2 hrs.

LCMS showed one main peak with desired mass. The reaction mixture was concentrated under vacuum. The residue was purified by prep-HPLC (column: Phenomenex Synergi C18 150*25 mm*10 μm; mobile phase: [water (0.1% TFA)-ACN]; B %: 14%-44%, 10 min) followed by lyophilization. N-[3-(3,5-dimethylisoxazol-4-yl)-4-[2-[(1S,4S)-2-oxa-5-azabicyclo[2.2.1]heptan-5-yl]ethoxy]phenyl]-1-fluoro-cyclopropanecarboxamide (47.27 mg, 89.28 μmol, 53.47% yield, 100% purity, TFA) was obtained as a yellow solid.

LCMS (ESI): m/z [M+H] calcd for C₂₂H₂₇FN₃O₄: 416.19; found: 416.2.

¹H NMR (400 MHZ, methanol-d₄) δ=7.69 (dd, J=2.7, 8.9 Hz, 1H), 7.50 (d, J=2.8 Hz, 1H), 7.16 (d, J=9.0 Hz, 1H), 4.61 (br s, 1H), 4.45-4.33 (m, 2H), 4.30 (s, 1H), 3.92 (br s, 1H), 3.80-3.37 (m, 4H), 3.14 (br d, J=1.6 Hz, 1H), 2.32 (s, 3H), 2.26-1.99 (m, 5H), 1.44-1.34 (m, 4H).

### N-[3-(3,5-dimethylisoxazol-4-yl)-4-[2-[(1S,4S)-2-oxa-5-azabicyclo[2.2.1]heptan-5-yl]ethoxy]phenyl]-2-methyl-pyrazole-3-carboxamide (163)

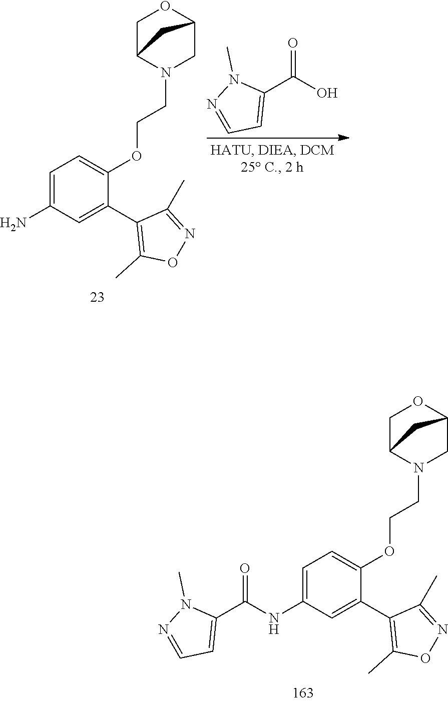

To a solution of 2-methylpyrazole-3-carboxylic acid (25.27 mg, 200.36 μmol, 1.2 eq.) and HATU (95.23 mg, 250.46 μmol, 1.5 eq.) in DCM (1 mL) was added DIEA (43.16 mg, 333.94 μmol, 58.17 μL, 2 eq.). The reaction mixture was stirred at 25° C. for 30 min. Then 3-(3,5-dimethylisoxazol-4-yl)-4-[2-[(1S,4S)-2-oxa-5-azabicyclo[2.2.1]heptan-5-yl]ethoxy]aniline ((23), 55 mg, 166.97 μmol, 1 eq.) was added to the reaction mixture. The reaction mixture was stirred at 25° C. for 2 hrs. LCMS showed one main peak with desired mass. The reaction mixture was concentrated under vacuum. The residue was purified by prep-HPLC (column: Phenomenex Synergi C18 150*25 mm*10 μm; mobile phase: [water (0.1% TFA)-ACN]; B %: 10%-40%, 10 min) followed by lyophilization. N-[3-(3,5-dimethylisoxazol-4-yl)-4-[2-[(1S,4S)-2-oxa-5-azabicyclo[2.2.1]heptan-5-yl]ethoxy]phenyl]-2-methyl-pyrazole-3-carboxamide (44.48 mg, 101.67 μmol, 60.89% yield, 100% purity) was obtained as a brown solid.

LCMS (ESI): m/z [M+H] calcd for C₂₃H₂₈NO₄: 438.21; found: 438.2.

¹H NMR (400 MHZ, methanol-d₄) δ=7.76 (dd, J=2.7, 8.9 Hz, 1H), 7.54 (dd, J=2.4, 16.9 Hz, 2H), 7.19 (d, J=9.0 Hz, 1H), 6.96 (d, J=2.1 Hz, 1H), 4.61 (br s, 1H), 4.45-4.34 (m, 2H), 4.31 (s, 1H), 4.15 (s, 3H), 3.98 (br d, J=7.9 Hz, 1H), 3.84-3.37 (m, 4H), 3.27-2.85 (m, 1H), 2.34 (s, 3H), 2.18 (s, 5H).

### N-[4-[2-[(1R,5S)-3-azabicyclo[3.1.0]hexan-3-yl]ethoxy]-3-(3,5-dimethylisoxazol-4-yl)phenyl]cyclopropanecarboxamide (164)

### Preparation of 2-[4-amino-2-(3,5-dimethylisoxazol-4-yl)phenoxy]ethanol (24)

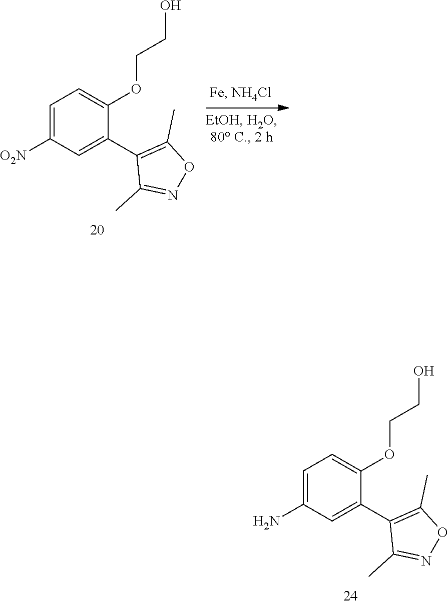 [SMILES: Cc1noc(C)c1-c1cc([N+](=O)[O-])ccc1OCCO ; Cc1noc(C)c1-c1cc(N)ccc1OCCO]

To a solution of 2-[2-(3,5-dimethylisoxazol-4-yl)-4-nitro-phenoxy]ethanol ((20), 300 mg, 1.08 mmol, 1 eq.) and NH₄Cl (115.34 mg, 2.16 mmol, 2 eq.) in EtOH (3 mL) and H₂O (1 mL) was added Fe (90.31 mg, 1.62 mmol, 1.5 eq.) at 80° C. The reaction mixture was stirred at 80° C. for 2 hrs. LCMS showed one main peak with desired mass. The reaction mixture was filtered and the filtrate was concentrated under vacuum. The residue was purified by flash silica gel chromatography (24 g SepaFlash® Silica Flash Column, eluent of 0˜100% ethyl acetate/petroleum ether gradient). 2-[4-amino-2-(3,5-dimethylisoxazol-4-yl)phenoxy]ethanol ((24), 200 mg, 805.55 μmol, 74.72% yield) was obtained as a yellow solid.

### Preparation of N-[3-(3,5-dimethylisoxazol-4-yl)-4-(2-hydroxyethoxy)phenyl]cyclopropanecarboxamide (25)

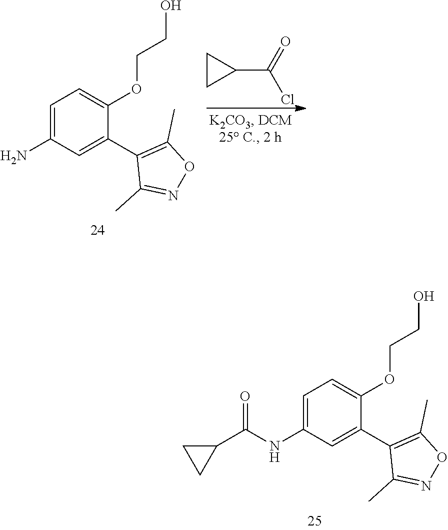

To a solution of 2-[4-amino-2-(3,5-dimethylisoxazol-4-yl)phenoxy]ethanol ((24), 200 mg, 805.55 μmol, 1 eq.) and K₂CO₃ (222.66 mg, 1.61 mmol, 2 eq.) in DCM (10 mL) was added cyclopropanecarbonyl chloride (126.31 mg, 1.21 mmol, 109.84 μL, 1.5 eq.) at 0° C. The reaction mixture was stirred at 25° C. for 1 hr. LCMS showed one main peak with desired mass. The residue was poured into water (20 mL). The aqueous phase was extracted with ethyl acetate (30 mL×3). The combined organic phase was washed with brine (20 mL), dried with anhydrous Na₂SO₄, filtered and concentrated under vacuum. The residue was purified by flash silica gel chromatography (12 g SepaFlash® Silica Flash Column, eluent of 0˜100% ethyl acetate/petroleum ether gradient). N-[3-(3,5-dimethylisoxazol-4-yl)-4-(2-hydroxyethoxy)phenyl]cyclopropanecarboxamide ((25), 210 mg, 663.82 μmol, 82.41% yield) was obtained as a white solid.

### Preparation of 2-[4-(cyclopropanecarbonylamino)-2-(3,5-dimethylisoxazol-4-yl)phenoxy]ethyl 4-methylbenzenesulfonate (26)

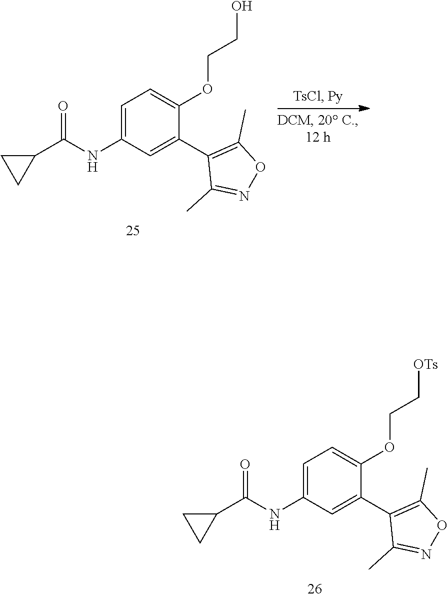 [SMILES: Cc1noc(C)c1-c1cc(NC(=O)C2CC2)ccc1OCCO ; Cc1ccc(S(=O)(=O)OCCOc2ccc(NC(=O)C3CC3)cc2-c2c(C)noc2C)cc1]

To a solution of N-[3-(3,5-dimethylisoxazol-4-yl)-4-(2-hydroxyethoxy)phenyl]cyclopropanecarboxamide ((25), 190 mg, 600.60 μmol, 1 eq.) and TosCl (572.51 mg, 3.00 mmol, 5 eq.) in DCM (3 mL) was added Py (2.94 g, 37.17 mmol, 3 mL, 61.89 eq.). The reaction mixture was stirred at 25° C. for 2 hrs. LCMS showed one main peak with desired mass. The residue was poured into water (20 mL). The aqueous phase was extracted with ethyl acetate (30 mL×3). The combined organic phase was washed with brine (30 mL), dried with anhydrous Na₂SO₄, filtered and concentrated under vacuum. The residue was purified by flash silica gel chromatography (12 g SepaFlash® Silica Flash Column, eluent of 0˜70% ethyl acetate/petroleum ether gradient). 2-[4-(cyclopropanecarbonylamino)-2-(3,5-dimethylisoxazol-4-yl)phenoxy]ethyl 4-methylbenzenesulfonate ((26), 200 mg, 425.05 μmol, 70.77% yield) was obtained as a colorless oil.

### Preparation of N-[4-[2-[(1R,5S)-3-azabicyclo[3.1.0]hexan-3-yl]ethoxy]-3-(3,5-dimethylisoxazol-4-yl)phenyl]cyclopropanecarboxamide (164)

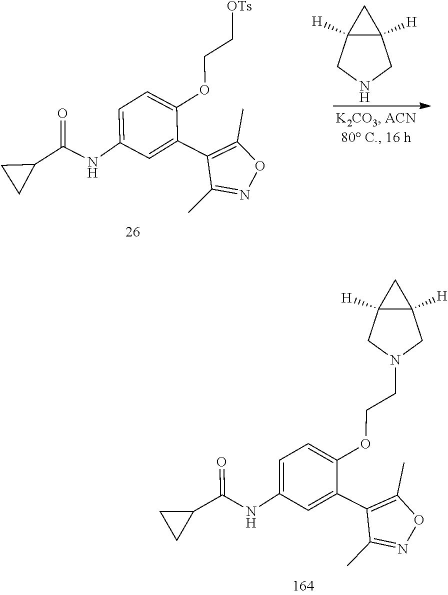 [SMILES: Cc1ccc(S(=O)(=O)OCCOc2ccc(NC(=O)C3CC3)cc2-c2c(C)noc2C)cc1 ; C1NC[C@H]2C[C@@H]12 ; Cc1noc(C)c1-c1cc(NC(=O)C2CC2)ccc1OCCN1C[C@H]2C[C@H]2C1]

To a solution of 2-[4-(cyclopropanecarbonylamino)-2-(3,5-dimethylisoxazol-4-yl)phenoxy]ethyl 4-methylbenzenesulfonate ((26), 200 mg, 425.05 μmol, 1 eq.) and 3-azabicyclo[3.1.0]hexane hydrochloride (61.00 mg, 510.06 μmol, 1.2 eq.) in ACN (3 mL) was added K₂CO₃ (117.49 mg, 850.09 μmol, 2 eq.). The reaction mixture was stirred at 80° C. for 16 hrs. LCMS showed one main peak with desired mass. The reaction mixture was filtered and the filtrate was concentrated under vacuum. The residue was purified by prep-HPLC (column: Phenomenex Gemini-NX 150*30 mm*5 μm; mobile phase: [water (0.1% TFA)-ACN]; B %: 0%-38%, 9 min) followed by lyophilization. N-[4-[2-[(1R,5S)-3-azabicyclo[3.1.0]hexan-3-yl]ethoxy]-3-(3,5-dimethylisoxazol-4-yl)phenyl]cyclopropanecarboxamide (125.3 mg, 252.88 μmol, 59.49% yield, 100% purity, TFA) was obtained as a yellow gum.

LCMS (ESI): m/z [M+H] calcd for C₂₂H₂₈N₃O₃: 382.21; found: 382.2.

¹H NMR (400 MHZ, methanol-d₄) δ=7.59 (dd, J=2.6, 8.9 Hz, 1H), 7.44 (d, J=2.8 Hz, 1H), 7.10 (d, J=9.0 Hz, 1H), 4.29 (br t, J=4.8 Hz, 2H), 3.57 (br t, J=4.8 Hz, 2H), 3.51 (d, J=11.5 Hz, 2H), 3.26 (br d, J=11.5 Hz, 2H), 2.33 (s, 3H), 2.17 (s, 3H), 1.81-1.69 (m, 3H), 1.02-0.72 (m, 5H), 0.61-0.44 (m, 1H).

### N-[3-(3,5-dimethylisoxazol-4-yl)-4-(2-morpholinoethoxy)phenyl]-3-(trifluoromethyl)benzamide (165)

### Preparation of N-(3-bromo-4-hydroxy-phenyl)-3-(trifluoromethyl)benzamide (27)

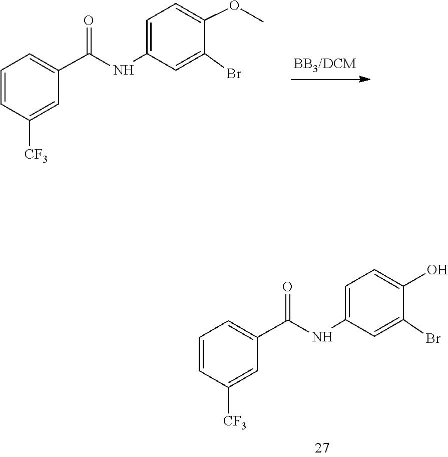 [SMILES: COc1ccc(NC(=O)c2cccc(C(F)(F)F)c2)cc1Br ; O=C(Nc1ccc(O)c(Br)c1)c1cccc(C(F)(F)F)c1]

To a mixture of N-(3-bromo-4-methoxy-phenyl)-3-(trifluoromethyl)benzamide (2.0 g, 5.35 mmol, 1 eq.) in DCM (20 mL) was added BBr3 (2.01 g, 8.02 mmol, 772.59 μL, 1.5 eq.). The mixture was stirred at 40° C. for 1 hr. The mixture was poured into saturated NaHCO₃ (100 mL) and stirred for 1 min. The aqueous phase was extracted with ethyl acetate (100 mL). The organic phase was separated and concentrated under vacuum. N-(3-bromo-4-hydroxy-phenyl)-3-(trifluoromethyl)benzamide ((27), 1.75 g, crude) as a yellow solid was used in the next step without further purification.

### Preparation of N-[3-bromo-4-(2-morpholinoethoxy)phenyl]-3-(trifluoromethyl)benzamide (28)

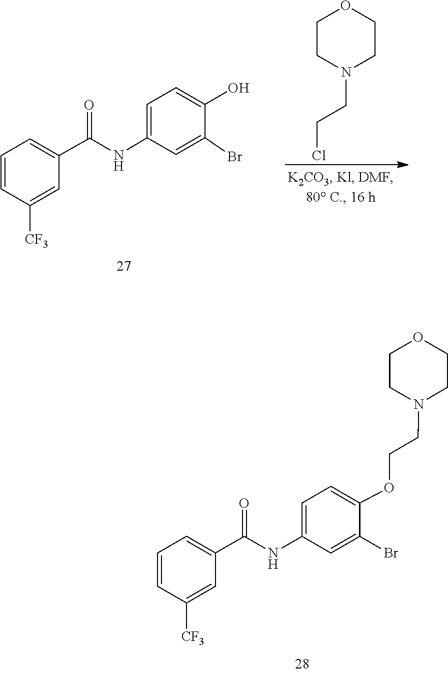 [SMILES: O=C(Nc1ccc(O)c(Br)c1)c1cccc(C(F)(F)F)c1 ; ClCCN1CCOCC1 ; O=C(Nc1ccc(OCCN2CCOCC2)c(Br)c1)c1cccc(C(F)(F)F)c1]

To a mixture of N-(3-bromo-4-hydroxy-phenyl)-3-(trifluoromethyl)benzamide ((27), 1.5 g, 4.17 mmol, 1 eq.) and K₂CO₃ (863.49 mg, 6.25 mmol, 1.5 eq.) in DMF (10 mL) was added KI (69.14 mg, 416.52 μmol, 0.1 eq.) and 4-(2-chloroethyl)morpholine (930.07 mg, 5.00 mmol, 1.2 eq., HCl). The mixture was stirred at 80° C. for 16 hrs. The residue was filtered. The crude product was purified by reversed-phase HPLC (0.1% NH₃·H₂O). N-[3-bromo-4-(2-morpholinoethoxy)phenyl]-3-(trifluoromethyl)benzamide ((28), 1.43 g, 3.01 mmol, 72.26% yield, 99.4% purity) as a yellow oil was used in the next step.

LCMS (ESI): m/z [M+H] calcd for C₂₀H₂₁Br^{79/81}N₂O₃: 471.06/473.06; found: 471.1/473.1.

### Preparation of N-[3-(3,5-dimethylisoxazol-4-yl)-4-(2-morpholinoethoxy)phenyl]-3-(trifluoromethyl)benzamide (165)

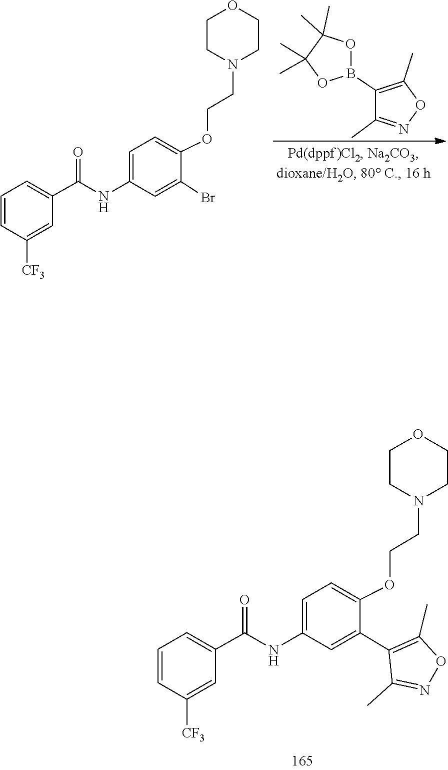

A mixture of N-[3-bromo-4-(2-morpholinoethoxy)phenyl]-3-(trifluoromethyl)benzamide ((28), 100 mg, 211.29 μmol, 1 eq.), (3,5-dimethylisoxazol-4-yl)boronic acid (35.73 mg, 253.55 μmol, 1.2 eq.), Na₂CO₃ (44.79 mg, 422.58 μmol, 2 eq.) and Pd(dppf)Cl₂ (15.46 mg, 21.13 μmol, 0.1 eq.) in dioxane (1 mL) and H₂O (0.1 mL) was degassed and purged with N₂ three times, and then the mixture was stirred at 80° C. for 16 hrs under an N₂ atmosphere. The residue was filtered. The residue was purified by prep-HPLC (column: Waters Xbridge 150*25 mm*5 μm; mobile phase: [water (0.05% ammonia hydroxide v/v)-ACN]; B %: 40%-70%, 11 min). N-[3-(3,5-dimethylisoxazol-4-yl)-4-(2-morpholinoethoxy)phenyl]-3-(trifluoromethyl)benzamide (17.35 mg, 35.09 μmol, 16.61% yield, 99% purity) was obtained as a white solid.

LCMS (ESI): m/z [M+H] calcd for C₂₅H₂₆F₃N₃O₄: 490.19; found: 490.1.

¹H NMR (400 MHZ, DMSO+D₂O) δ=10.14 (br s, 1H), 8.32-8.24 (m, 2H), 7.97 (br d, J=7.6 Hz, 1H), 7.83-7.73 (m, 2H), 7.59 (d, J=2.3 Hz, 1H), 7.18 (d, J=8.9 Hz, 1H), 4.09 (br t, J=5.6 Hz, 2H), 3.60-3.48 (m, 4H), 3.33 (br s, 2H), 2.62 (br t, J=5.6 Hz, 2H), 2.38 (br s, 4H), 2.33 (s, 3H), 2.17 (s, 3H).

### N-[3-(3,5-dimethylisoxazol-4-yl)-4-(2-morpholinoethoxy)phenyl]cyclopropanecarboxamide (166)

### Preparation of N-(3-bromo-4-hydroxy-phenyl)cyclopropanecarboxamide (29)

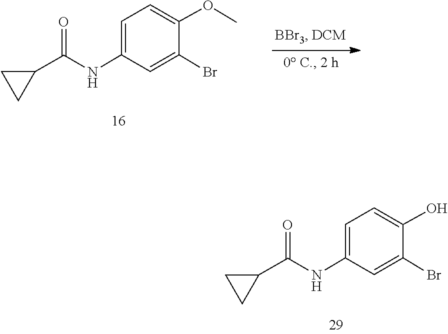

To a solution of N-(3-bromo-4-methoxy-phenyl)cyclopropanecarboxamide ((16), 5 g, 18.51 mmol, 1 eq.) in DCM (100 mL) was added BBr3 (6.96 g, 27.77 mmol, 2.68 mL, 1.5 eq.) at 0° C. under N₂. The reaction mixture was stirred at 0° C. for 2 hrs (monitored by TLC). The reaction mixture was quenched with sat. NaHCO₃ aq. (300 mL) and stirred at 15° C. for 10 min. The mixture was filtered and the filter cake was concentrated under vacuum. N-(3-bromo-4-hydroxy-phenyl)cyclopropanecarboxamide ((29), 4 g, 15.62 mmol, 84.38% yield) was obtained as a purple solid.

LCMS (ESI): m/z [M+H] calcd for C₁₀H₁₁Br^{79/81}N₃O₄: 256.1/258.1; found: 256.3/258.3.

### Preparation of N-[3-bromo-4-(2-morpholinoethoxy)phenyl]cyclopropanecarboxamide (30)

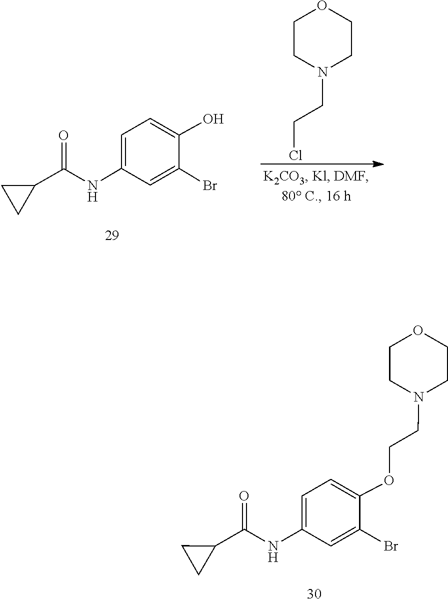 [SMILES: O=C(Nc1ccc(O)c(Br)c1)C1CC1 ; ClCCN1CCOCC1 ; O=C(Nc1ccc(OCCN2CCOCC2)c(Br)c1)C1CC1]

To a mixture of N-(3-bromo-4-hydroxy-phenyl)cyclopropanecarboxamide ((29), 3 g, 11.71 mmol, 1 eq.) and 4-(2-chloroethyl)morpholine hydrochloride (2.61 g, 14.05 mmol, 1.2 eq.) in DMF (20 mL) was added KI (194.46 mg, 1.17 mmol, 0.1 eq.) and K₂CO₃ (3.24 g, 23.42 mmol, 2 eq.). The reaction mixture was stirred at 80° C. for 16 hrs (monitored by LCMS). The residue was poured into water (50 mL). The aqueous phase was extracted with ethyl acetate (50 mL×3). The combined organic phase was washed with brine (100 mL), dried with anhydrous Na₂SO₄, filtered and concentrated under vacuum. The residue was purified by reversed-phase HPLC (0.1% NH₃·H₂O). N-[3-bromo-4-(2-morpholinoethoxy)phenyl]cyclopropanecarboxamide ((30), 2.3 g, 6.23 mmol, 53.19% yield, 100% purity) was obtained as a white solid.

¹H NMR (400 MHZ, methanol-d₄) δ=7.84 (d, J=2.6 Hz, 1H), 7.43 (dd, J=2.5, 8.9 Hz, 1H), 7.00 (d, J=8.9 Hz, 1H), 4.18 (t, J=5.4 Hz, 2H), 3.82-3.62 (m, 4H), 2.85 (t, J=5.4 Hz, 2H), 2.72-2.62 (m, 4H), 1.80-1.63 (m, 1H), 0.99-0.78 (m, 4H).

### Preparation of N-[3-(3,5-dimethylisoxazol-4-yl)-4-(2-morpholinoethoxy)phenyl]cyclopropanecarboxamide (166)

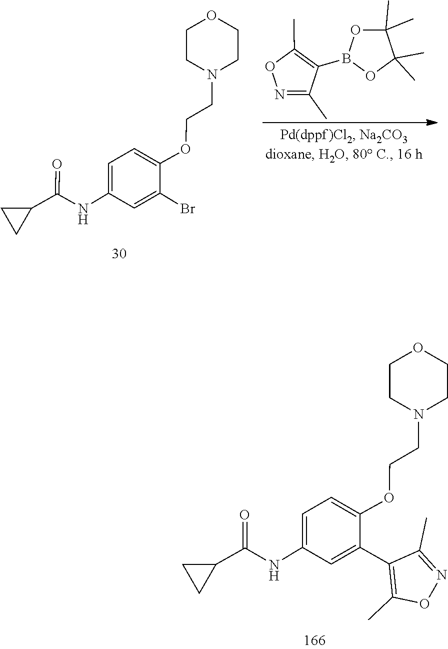

To a solution of N-[3-bromo-4-(2-morpholinoethoxy)phenyl]cyclopropanecarboxamide ((30), 50 mg, 135.41 μmol, 1 eq.), Na₂CO₃ (28.70 mg, 270.82 μmol, 2 eq.) and 3,5-dimethyl-4-(4,4,5,5-tetramethyl-1,3,2-dioxaborolan-2-yl)isoxazole (33.23 mg, 148.95 μmol, 1.1 eq.) in dioxane (2 mL) and H₂O (0.5 mL) was added Pd(dppf)Cl₂ (9.91 mg, 13.54 μmol, 0.1 eq.) under N₂. The reaction mixture was stirred at 80° C. for 16 hrs (monitored by LCMS). The residue was diluted with MeOH (2 mL), then thiourea resin was added to the mixture. The mixture was stirred at 15° C. for 16 hrs. The mixture was filtered and the filtrate was concentrated under vacuum. The residue was purified by prep-HPLC (column: Boston Green ODS 150*30 mm*5 μm; mobile phase: [water (0.225% FA)-ACN]; B %: 10%-40%, 10 min) followed by lyophilization. N-[3-(3,5-dimethylisoxazol-4-yl)-4-(2-morpholinoethoxy)phenyl]cyclopropanecarboxamide (27.41 mg, 67.56 μmol, 49.89% yield, 95% purity) was obtained as an off-white solid.

LCMS (ESI): m/z [M+H] calcd for C₂₁H₂₈N₃O₄: 386.20; found: 386.1.

¹H NMR (400 MHZ, methanol-d₄) δ ppm 0.81-0.88 (m, 2H) 0.91-0.98 (m, 2H) 1.67-1.80 (m, 1H) 2.16 (s, 3H) 2.31 (s, 3H) 2.47-2.57 (m, 4H) 2.78 (t, J=5.38 Hz, 2H) 3.60-3.69 (m, 4H) 4.13 (t, J=5.44 Hz, 2H) 7.07 (d, J=9.01 Hz, 1H) 7.40 (d, J=2.63 Hz, 1H) 7.53 (dd, J=8.88, 2.63 Hz, 1H).

### N-[3-(3,5-dimethylisoxazol-4-yl)-4-(2-pyrrolidin-1-ylethoxy)phenyl]cyclopropanecarboxamide (167)

### Preparation of N-[3-bromo-4-(2-pyrrolidin-1-ylethoxy)phenyl]cyclopropanecarboxamide (31)

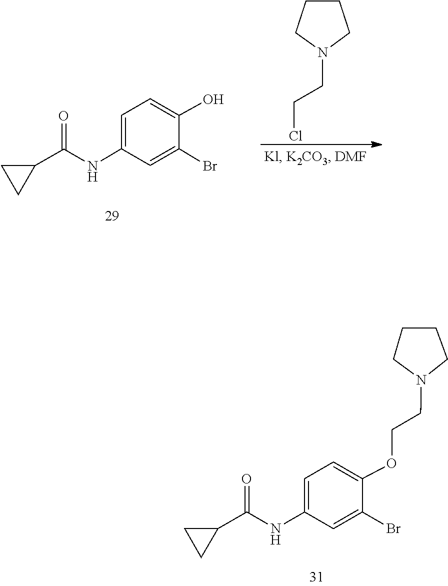 [SMILES: O=C(Nc1ccc(O)c(Br)c1)C1CC1 ; ClCCN1CCCC1 ; O=C(Nc1ccc(OCCN2CCCC2)c(Br)c1)C1CC1]

To a mixture of N-(3-bromo-4-hydroxy-phenyl)cyclopropanecarboxamide ((29), 1 g, 3.90 mmol, 1 eq.), K₂CO₃ (809.50 mg, 5.86 mmol, 1.5 eq.) and KI (324.10 mg, 1.95 mmol, 0.5 eq.) in DMF (10 mL) was added 1-(2-chloroethyl)pyrrolidine hydrochloride (967.80 mg, 4.69 mmol, 1.2 eq., HCl). The mixture was stirred at 80° C. and stirred for 16 hrs. The residue was filtered. The crude product was purified by reversed-phase HPLC (0.1% NH₃·H₂O). N-[3-bromo-4-(2-pyrrolidin-1-ylethoxy)phenyl]cyclopropanecarboxamide ((31), 300 mg, 840.76 μmol, 21.53% yield, 99% purity) as a yellow oil was used in the next step.

LCMS (ESI): m/z [M+H] calcd for C₁₆H₂₂Br^{79/81}N₂O₂: 351.08/353.08; found: 351.2/353.2.

### Preparation of N-[3-(3,5-dimethylisoxazol-4-yl)-4-(2-pyrrolidin-1-ylethoxy)phenyl]cyclopropanecarboxamide (167)

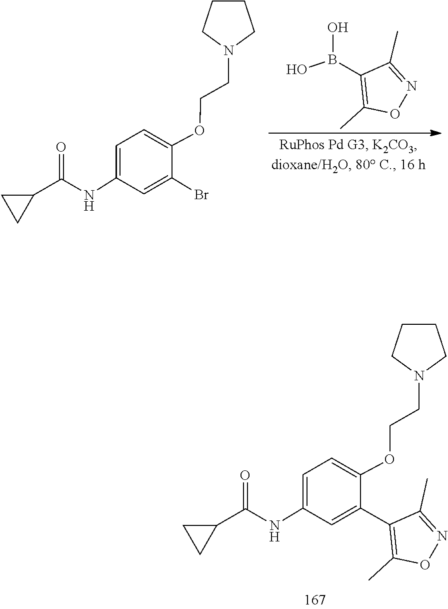

To a mixture of N-[3-bromo-4-(2-pyrrolidin-1-ylethoxy)phenyl]cyclopropanecarboxamide ((31), 100 mg, 283.08 μmol, 1 eq.), K₂CO₃ (78.25 mg, 566.17 μmol, 2 eq.) and RuPhos Pd G3 (23.68 mg, 28.31 μmol, 0.1 eq.) in dioxane (1 mL) and H₂O (0.1 mL) was added (3,5-dimethylisoxazol-4-yl)boronic acid (47.87 mg, 339.70 μmol, 1.2 eq.). The mixture was stirred at 80° C. for 16 hrs. The reaction mixture was filtered and concentrated under reduced pressure to give a residue. The residue was purified by prep-HPLC (column: Waters Xbridge 150*25 mm*5 μm; mobile phase: [water (10 mM NH₄HCO₃)-ACN]; B %: 20%-40%, 10 min). N-[3-(3,5-dimethylisoxazol-4-yl)-4-(2-pyrrolidin-1-ylethoxy)phenyl]cyclopropanecarboxamide (31.1 mg, 81.65 μmol, 28.84% yield, 97% purity) was obtained as a brown solid.

LCMS (ESI): m/z [M+H] calcd for C₂₁H₂₈N₃O₃: 370.21; found: 370.2.

¹H NMR (400 MHZ, DMSO-d₆) δ=10.12 (s, 1H), 7.54 (dd, J=2.6, 8.9 Hz, 1H), 7.41 (d, J=2.6 Hz, 1H), 7.06 (d, J=9.0 Hz, 1H), 4.01 (t, J=5.9 Hz, 2H), 2.69 (t, J=5.9 Hz, 2H), 2.45-2.36 (m, 4H), 2.27 (s, 3H), 2.10 (s, 3H), 1.78-1.69 (m, 1H), 1.66-1.57 (m, 4H), 0.80-0.73 (m, 4H).

### N-[4-[2-(4-acetylpiperazin-1-yl)ethoxy]-3-(3,5-dimethylisoxazol-4-yl)phenyl]cyclopropanecarboxamide (168)

### Preparation of N-[4-[2-(4-acetylpiperazin-1-yl)ethoxy]-3-bromo-phenyl]cyclopropanecarboxamide (32)

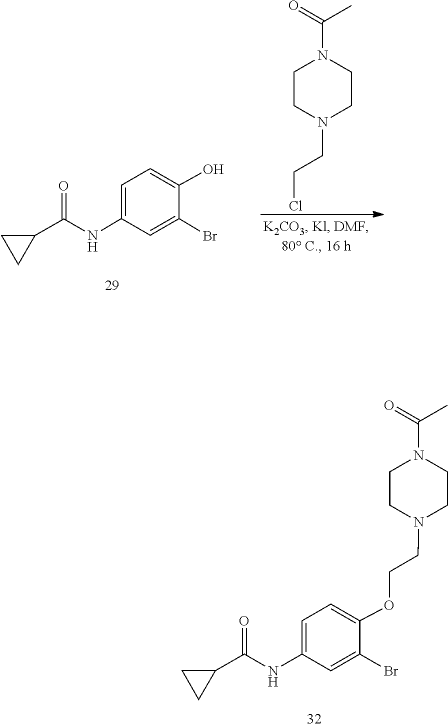 [SMILES: O=C(Nc1ccc(O)c(Br)c1)C1CC1 ; CC(=O)N1CCN(CCCl)CC1 ; CC(=O)N1CCN(CCOc2ccc(NC(=O)C3CC3)cc2Br)CC1]

To a mixture of N-(3-bromo-4-hydroxy-phenyl)cyclopropanecarboxamide ((29), 1 g, 3.90 mmol, 1 eq.), KI (324.10 mg, 1.95 mmol, 0.5 eq.) and K₂CO₃ (809.50 mg, 5.86 mmol, 1.5 eq.) in DMF (10 mL) was added 1-[4-(2-chloroethyl)piperazin-1-yl]ethanone hydrochloride (1.24 g, 4.69 mmol, 1.2 eq., HCl). The mixture was stirred at 80° C. for 16 hrs. The residue was filtered. The crude product was purified by reversed-phase HPLC (0.1% NH₃·H₂O). N-[4-[2-(4-acetylpiperazin-1-yl)ethoxy]-3-bromo-phenyl]cyclopropanecarboxamide ((32), 200 mg, 477.69 μmol, 12.23% yield, 98% purity) as a yellow oil was used in the next step.

LCMS (ESI): m/z [M+H] calcd for C₁₈H₂₅Br^{79/81}N₃O₃: 410.10/412.10; found: 410.2/412.2.

### Preparation of N-[4-[2-(4-acetylpiperazin-1-yl)ethoxy]-3-(3,5-dimethylisoxazol-4-yl)phenyl]cyclopropanecarboxamide (168)

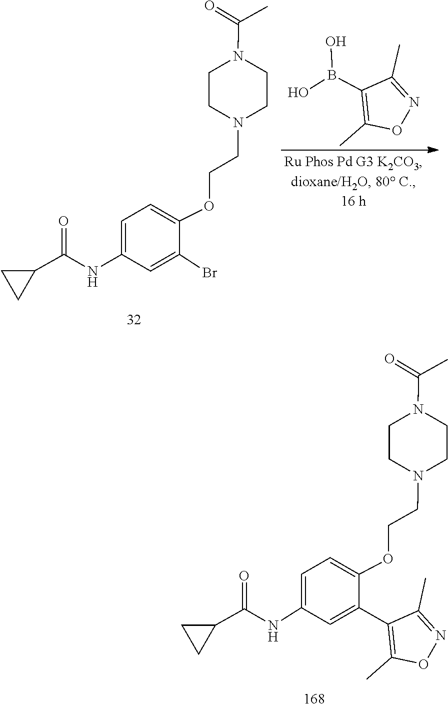

A mixture of N-[4-[2-(4-acetylpiperazin-1-yl)ethoxy]-3-bromo-phenyl]cyclopropanecarboxamide ((32), 30 mg, 73.12 μmol, 1 eq.), (3,5-dimethylisoxazol-4-yl)boronic acid (12.37 mg, 87.74 μmol, 1.2 eq.), K₂CO₃ (20.21 mg, 146.23 μmol, 2 eq.) and RuPhos Pd G3 (6.12 mg, 7.31 μmol, 0.1 eq.) in dioxane (1 mL) and H₂O (0.1 mL) was degassed and purged with N₂ three times, and then the mixture was stirred at 80° C. for 16 hrs under an N₂ atmosphere. The residue was filtered, and the organic mixture was concentrated under reduced pressure to give a residue. The residue was purified by prep-HPLC (column: Phenomenex Synergi C18 150*25*10 μm; mobile phase: [water (0.225% FA)-ACN]; B %: 5%-35%, 10 min). N-[4-[2-(4-acetylpiperazin-1-yl)ethoxy]-3-(3,5-dimethylisoxazol-4-yl)phenyl]cyclopropanecarboxamide (8.26 mg, 19.17 μmol, 26.22% yield, 99% purity) was obtained as a white solid.

LCMS (ESI): m/z [M+H] calcd for C₂₃H₃₁N₄O₂: 427.23; found: 427.2.

¹H NMR (400 MHZ, DMSO-d₆) δ=10.12 (s, 1H), 8.18 (s, 1H), 7.55 (dd, J=2.6, 8.9 Hz, 1H), 7.41 (d, J=2.6 Hz, 1H), 7.08 (d, J=9.0 Hz, 1H), 4.03 (t, J=5.6 Hz, 2H), 3.37-3.33 (m, 4H), 2.61 (t, J=5.6 Hz, 2H), 2.40-2.34 (m, 2H), 2.33-2.28 (m, 2H), 2.28 (s, 3H), 2.11 (s, 3H), 1.96 (s, 3H), 1.77-1.66 (m, 1H), 0.81-0.73 (m, 4H).

### N-[3-(3,5-dimethylisoxazol-4-yl)-4-[[(2R)-2-piperidyl]methoxy]phenyl]cyclopropanecarboxamide (169)

### Preparation of tert-butyl (2R)-2-[(2-bromo-4-nitro-phenoxy)methyl]piperidine-1-carboxylate (33)

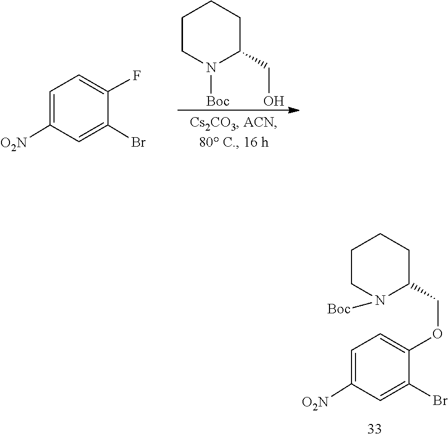

To the mixture of 2-bromo-1-fluoro-4-nitro-benzene (10 g, 45.46 mmol, 1 eq.) and tert-butyl (2R)-2-(hydroxymethyl)piperidine-1-carboxylate (9.79 g, 45.46 mmol, 1 eq.) in MeCN (100 mL) was added Cs₂CO₃ (29.62 g, 90.91 mmol, 2 eq.). The mixture was stirred at 80° C. for 16 hrs. The mixture was filtered, and the filtrate was concentrated under vacuum. The residue was purified by flash silica gel chromatography (ISCO®; 80 g SepaFlash® Silica Flash Column, eluent of 0˜30% ethyl acetate/petroleum ether gradient at 60 mL/min). Tert-butyl (2R)-2-[(2-bromo-4-nitro-phenoxy)methyl]piperidine-1-carboxylate ((33), 18 g, 43.34 mmol, 95.36% yield) was obtained as a yellow solid.

LCMS (ESI): m/z [M+H] calcd for C₁₇H₂₄Br^{79/81}N₂O₅: 415.08/417.08; found: 316.9/360.9.

¹H NMR (400 MHZ, methanol-d₄) δ=8.41 (d, J=2.7 Hz, 1H), 8.22 (dd, J=2.8, 9.2 Hz, 1H), 7.27 (d, J=9.0 Hz, 1H), 4.67-4.60 (m, 1H), 4.43-4.27 (m, 2H), 4.08-3.99 (m, 1H), 3.01 (br s, 1H), 1.95 (br d, J=6.1 Hz, 1H), 1.76-1.63 (m, 4H), 1.50-1.35 (m, 10H).

### Preparation of tert-butyl 2-[(4-amino-2-bromo-phenoxy)methyl]piperidine-1-carboxylate (34)

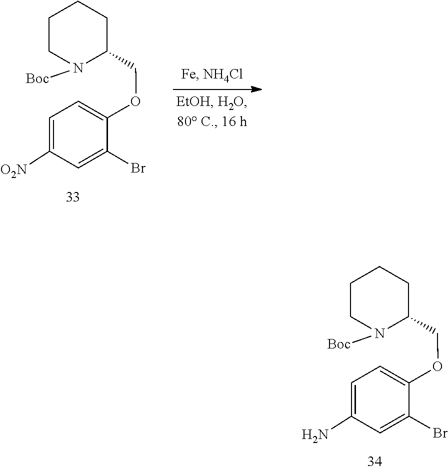 [SMILES: CC(C)(C)OC(=O)N1CCCC[C@@H]1COc1ccc([N+](=O)[O-])cc1Br ; CC(C)(C)OC(=O)N1CCCC[C@@H]1COc1ccc(N)cc1Br]

To the mixture of tert-butyl 2-[(2-bromo-4-nitro-phenoxy)methyl]piperidine-1-carboxylate ((33), 9 g, 21.67 mmol, 1 eq.) in EtOH (60 mL) and H₂O (60 mL) was added NH₄Cl (9.27 g, 173.38 mmol, 8 eq.). The mixture was stirred at 80° C. for 10 min. Fe (6.05 g, 108.36 mmol, 5 eq.) was added to the reaction mixture. The mixture was stirred at 80° C. for 1 hr. The reaction mixture was concentrated under vacuum and filtered. The filtrate was concentrated under vacuum. The residue was purified by flash silica gel chromatography (ISCO®; 80 g SepaFlash® Silica Flash Column, eluent of 0˜30% ethyl acetate/petroleum ether gradient at 60 mL/min). Tert-butyl 2-[(4-amino-2-bromo-phenoxy)methyl]piperidine-1-carboxylate ((34), 7.3 g, 18.95 mmol, 87.42% yield) was obtained as a colorless gum.

LCMS (ESI): m/z [M+H] calcd for C₁₇H₂₆Br^{79/81}N₂O₃: 385.1, 387.1, found: 385.0, 387.0.

¹H NMR (400 MHZ, methanol-d₄) δ=6.95 (d, J=2.7 Hz, 1H), 6.85 (d, J=8.7 Hz, 1H), 6.66 (dd, J=8.7, 2.7 Hz, 1H), 4.59-4.45 (m, 1H), 4.06-3.95 (m, 3H), 2.96 (br t, J=12.9 Hz, 1H), 1.97 (br d, J=10.5 Hz, 1H), 1.73-1.58 (m, 4H), 1.44 (br s, 10H).

### Preparation of tert-butyl (2R)-2-[[4-amino-2-(3,5-dimethylisoxazol-4-yl)phenoxy]methyl]piperidine-1-carboxylate (35)

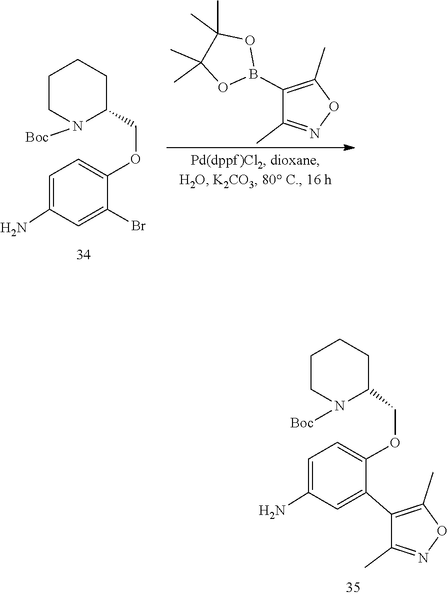

A mixture of (R)-tert-butyl 2-((4-amino-2-bromophenoxy)methyl)piperidine-1-carboxylate ((34), 9.50 g, 24.66 mmol, 1 eq.), 3,5-dimethyl-4-(4,4,5,5-tetramethyl-1,3,2-dioxaborolan-2-yl)isoxazole (6.60 g, 29.59 mmol, 1.2 eq.), Pd(dppf)Cl₂ (1.80 g, 2.47 mmol, 0.1 eq.), K₂CO₃ (3.41 g, 24.66 mmol, 1 eq.) in dioxane (100 mL) and H₂O (15 mL) was degassed and purged with N₂ three times, and then the mixture was stirred at 80° C. for 16 hrs under an N₂ atmosphere. The mixture was diluted with water (300 mL) and then extracted with ethyl acetate (500 mL×2). The combined organic phase was washed with brine (300 mL), dried over anhydrous Na₂SO₄, filtered and concentrated under vacuum. The residue was purified by flash silica gel chromatography (ISCO®; 120 g SepaFlash® Silica Flash Column, eluent of 0˜90% ethyl acetate/petroleum ether gradient at 85 mL/min). Tert-butyl (2R)-2-[[4-amino-2-(3,5-dimethylisoxazol-4-yl)phenoxy]methyl]piperidine-1-carboxylate ((35), 8.9 g, 22.17 mmol, 89.90% yield) was obtained as a yellow oil, and then became brown solid after 1 day.

LCMS (ESI): m/z [M+H] calcd for C₂₂H₃₂N₃O₄: 402.0, found: 402.3.

¹H NMR (400 MHZ, chloroform-d) δ 6.86 (d, J=8.6 Hz, 1H), 6.69 (dd, J=8.6, 2.9 Hz, 1H), 6.48 (d, J=2.9 Hz, 1H), 4.39 (br d, J=1.6 Hz, 1H), 4.04-3.93 (m, 1H), 3.92-3.78 (m, 2H), 3.51 (br s, 2H), 2.68 (td, J=12.8, 3.0 Hz, 1H), 2.29 (s, 3H), 2.16 (s, 3H), 1.70-1.33 (m, 15H).

### Preparation of tert-butyl (2R)-2-[[4-(cyclopropanecarbonylamino)-2-(3,5-dimethylisoxazol-4-yl)phenoxy]methyl]piperidine-1-carboxylate (36)

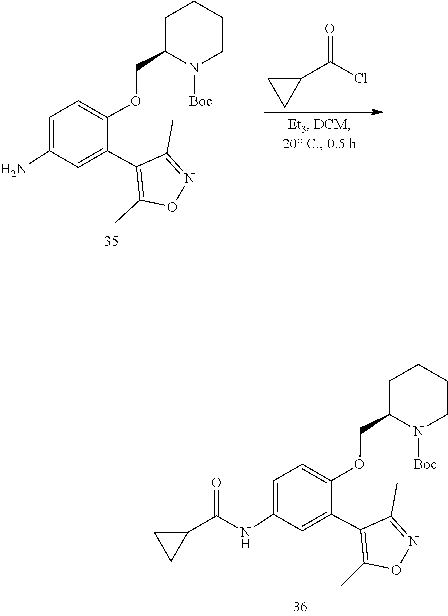 [SMILES: Cc1noc(C)c1-c1cc(N)ccc1OC[C@H]1CCCCN1C(=O)OC(C)(C)C ; O=C(Cl)C1CC1 ; Cc1noc(C)c1-c1cc(NC(=O)C2CC2)ccc1OC[C@H]1CCCCN1C(=O)OC(C)(C)C]

To a solution of tert-butyl (2R)-2-[[4-amino-2-(3,5-dimethylisoxazol-4-yl)phenoxy]methyl]piperidine-1-carboxylate ((35), 400 mg, 996.27 μmol, 1 eq.) and Et₃N (151.22 mg, 1.49 mmol, 208.00 μL, 1.5 eq.) in DCM (10 mL) was added cyclopropanecarbonyl chloride (124.97 mg, 1.20 mmol, 108.67 μL, 1.2 eq.) at 0° C. The mixture was stirred at 25° C. for 0.5 hrs. The reaction mixture was extracted with DCM (10 mL×2) and H₂O (10 mL). The combined organic layers were washed with sat. NaCl (10 mL), dried over anhydrous Na₂SO₄, filtered and concentrated under reduced pressure to give a residue. The residue was purified by column chromatography (SiO₂, petroleum ether/ethyl acetate=1:0 to 1:1). Tert-butyl (2R)-2-[[4-(cyclopropanecarbonylamino)-2-(3,5-dimethylisoxazol-4-yl)phenoxy]methyl]piperidine-1-carboxylate ((36), 430 mg, 915.73 μmol, 91.92% yield) was obtained as a yellow oil.

¹H NMR (400 MHZ, chloroform-d) δ=7.60 (br s, 1H), 7.46 (br d, J=8.4 Hz, 1H), 7.32-7.28 (m, 1H), 6.96 (d, J=8.9 Hz, 1H), 4.44 (br s, 1H), 4.04-3.90 (m, 3H), 2.76-2.65 (m, 1H), 2.28 (s, 3H), 2.15 (s, 3H), 1.67 (br d, J=13.3 Hz, 1H), 1.61-1.48 (m, 4H), 1.43 (s, 11H), 1.11-1.05 (m, 2H), 0.87-0.78 (m, 2H).

### Preparation of N-[3-(3,5-dimethylisoxazol-4-yl)-4-[[(2R)-2-piperidyl]methoxy]phenyl]cyclopropanecarboxamide (169)

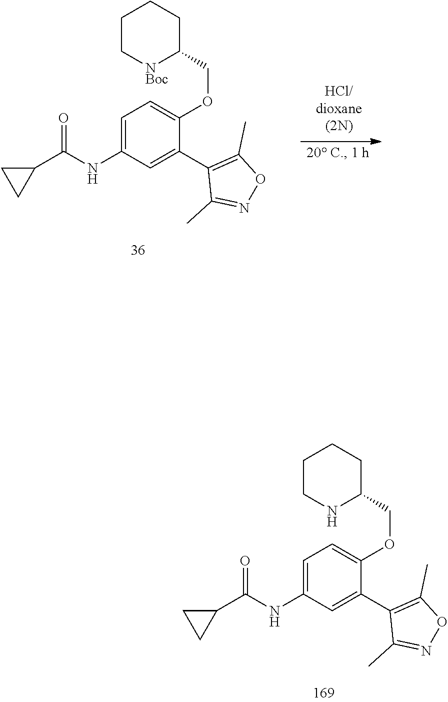 [SMILES: Cc1noc(C)c1-c1cc(NC(=O)C2CC2)ccc1OC[C@H]1CCCCN1C(=O)OC(C)(C)C ; Cc1noc(C)c1-c1cc(NC(=O)C2CC2)ccc1OC[C@H]1CCCCN1]

To a solution of tert-butyl (2R)-2-[[4-(cyclopropanecarbonylamino)-2-(3,5-dimethylisoxazol-4-yl)phenoxy]methyl]piperidine-1-carboxylate ((36), 150 mg, 319.44 μmol, 1 eq.) in dioxane (2 mL) was added HCl/dioxane (4 M, 2 mL). The mixture was stirred at 20° C. for 1 hr (monitored by LC-MS). To the reaction mixture was added sat. NaHCO₃ (2 mL) to adjust the pH to about 7, and then was concentrated under vacuum to give a residue. The residue was purified by prep-HPLC (column: Waters Xbridge 150*25 mm*5 um; mobile phase: [water (10 mM NH₄HCO₃)-ACN]; B %: 20%-50%, 10 min). N-[3-(3,5-dimethylisoxazol-4-yl)-4-[[(2R)-2-piperidyl]methoxy]phenyl]cyclopropanecarboxamide (80.87 mg, 218.89 μmol, 68.52% yield, 100% purity) was obtained as an off-white solid.

LCMS (ESI): m/z [M+H] calcd for C₂₁H₂₈N₃O₃: 370.2; found: 370.3.

¹H NMR (400 MHZ, methanol-d₄) δ=7.51 (dd, J=2.6, 8.9 Hz, 1H), 7.41 (d, J=2.7 Hz, 1H), 7.08 (d, J=8.9 Hz, 1H), 3.93-3.87 (m, 1H), 3.85-3.78 (m, 1H), 2.99 (br d, J=10.9 Hz, 1H), 2.88-2.79 (m, 1H), 2.59 (dt, J=2.7, 11.9 Hz, 1H), 2.31 (s, 3H), 2.15 (s, 3H), 1.86-1.69 (m, 2H), 1.69-1.57 (m, 2H), 1.48-1.32 (m, 2H), 1.23-1.09 (m, 1H), 1.00-0.77 (m, 4H).

### (S)—N-(3-(3,5-dimethylisoxazol-4-yl)-4-(piperidin-2-ylmethoxy)phenyl)cyclopropanecarboxamide (170)

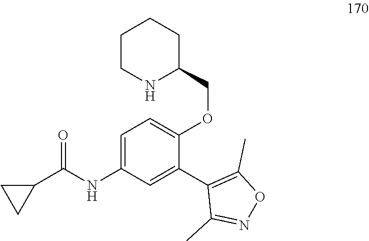 [SMILES: Cc1noc(C)c1-c1cc(NC(=O)C2CC2)ccc1OC[C@@H]1CCCCN1]

Compound 170 was prepared according to a synthesis similar to that described for compound 169, substituting tert-butyl (2S)-2-(hydroxymethyl)piperidine-1-carboxylate for tert-butyl (2R)-2-(hydroxymethyl)piperidine-1-carboxylate.

LCMS (ESI): m/z [M+H] calcd for C₂₁H₂₈N₃O₃: 370.2; found: 370.4.

¹H NMR (400 MHz, methanol-d₄. TFA salt) δ=7.59 (dd, J=2.4, 8.9 Hz, 1H), 7.42 (d, J=2.6 Hz, 1H), 7.16 (d, J=8.9 Hz, 1H), 4.09-3.99 (m, 2H), 3.50-3.34 (m, 2H), 3.09-2.97 (m, 1H), 2.33 (s, 3H), 2.17 (s, 3H), 1.91 (br d, J=10.4 Hz, 3H), 1.74 (tt, J=4.6, 7.8 Hz, 1H), 1.69-1.44 (m, 3H), 0.99-0.81 (m, 4H).

¹H NMR (400 MHZ, methanol-d₄, free base) δ=7.55 (dd, J=2.5, 8.6 Hz, 1H), 7.41 (d, J=2.3 Hz, 1H), 7.11 (d, J=8.9 Hz, 1H), 4.02-3.82 (m, 2H), 3.20-3.03 (m, 2H), 2.77 (br t, J=12.1 Hz, 1H), 2.32 (s, 3H), 2.16 (s, 3H), 1.92-1.64 (m, 4H), 1.58-1.40 (m, 2H), 1.31 (br d, J=11.1 Hz, 1H), 1.00-0.73 (m, 4H).

### (S)—N-(3-(3,5-dimethylisoxazol-4-yl)-4-(morpholin-3-ylmethoxy)phenyl)cyclopropanecarboxamide (171)

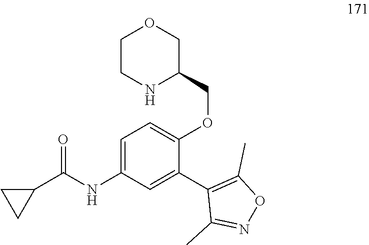 [SMILES: Cc1noc(C)c1-c1cc(NC(=O)C2CC2)ccc1OC[C@@H]1COCCN1]

Compound 171 was prepared according to a synthesis similar to that described for compound 169, substituting tert-butyl (S)-3-(hydroxymethyl)morpholine-4-carboxylate for tert-butyl (2R)-2-(hydroxymethyl)piperidine-1-carboxylate.

LCMS (ESI): m/z [M+H] calcd for C₂₀H₂₆N₃O₄: 372.18; found: 372.1.

¹H NMR (400 MHz, methanol-d₄) δ=7.53 (dd, J=2.6, 8.9 Hz, 1H), 7.41 (d, J=2.6 Hz, 1H), 7.08 (d, J=8.9 Hz, 1H), 3.93-3.84 (m, 2H), 3.81-3.71 (m, 2H), 3.51-3.40 (m, 1H), 3.25 (dd, J=9.5, 11.1 Hz, 1H), 3.06 (dtd, J=3.2, 6.1, 9.3 Hz, 1H), 2.85 (dd, J=2.8, 6.5 Hz, 2H), 2.31 (s, 3H), 2.15 (s, 3H), 1.79-1.68 (m, 1H), 0.97-0.90 (m, 2H), 0.89-0.80 (m, 2H).

### N-(4-(azetidin-3-yloxy)-3-(3,5-dimethylisoxazol-4-yl)phenyl)cyclopropanecarboxamide (172)

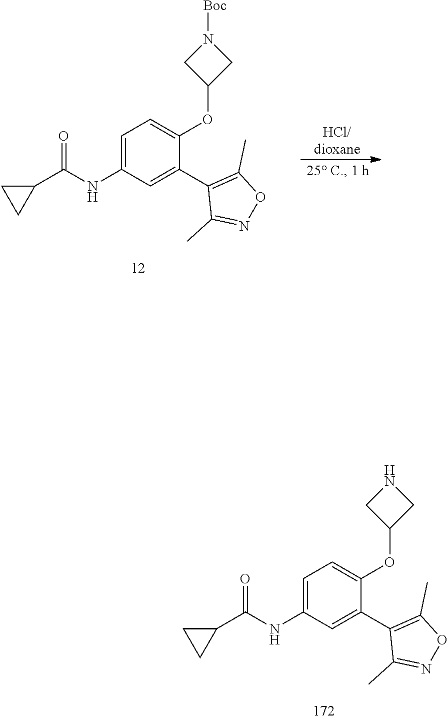 [SMILES: Cc1noc(C)c1-c1cc(NC(=O)C2CC2)ccc1OC1CN(C(=O)OC(C)(C)C)C1 ; Cc1noc(C)c1-c1cc(NC(=O)C2CC2)ccc1OC1CNC1]

To a solution of tert-butyl 3-[4-(cyclopropanecarbonylamino)-2-(3,5-dimethylisoxazol-4-yl)phenoxy]azetidine-1-carboxylate ((12), 100 mg, 233.92 μmol, 1 eq.) in DCM (2 mL) was added HCl/dioxane (4 M, 2 mL, 34.20 eq.). The reaction mixture was stirred at 25° C. for 1 hr (monitored by LCMS). The reaction mixture was concentrated under vacuum. The residue was purified by prep-HPLC (column: UniSil 3-100 C18 UItra (150*25 mm*3 μm); mobile phase: [water (0.225% FA)-ACN]; B %: 1%-30%, 10 min) followed by lyophilization. N-[4-(azetidin-3-yloxy)-3-(3,5-dimethylisoxazol-4-yl)phenyl]cyclopropanecarboxamide (49.35 mg, 143.21 μmol, 61.22% yield, 95% purity) was obtained as a white solid.

LCMS (ESI): m/z [M+H] calcd for C₁₈H₂₂N₃O₃: 328.16; found: 328.1.

¹H NMR (400 MHZ, methanol-d₄) δ ppm 0.80-0.89 (m, 2H), 0.94 (br d, J=3.50 Hz, 2H) 1.67-1.81 (m, 1H) 2.15-2.22 (m, 3H) 2.33 (s, 3H) 4.04 (br dd, J=11.88, 4.75 Hz, 2H) 4.48 (br dd, J=11.69, 6.57 Hz, 2H) 5.06-5.22 (m, 1H) 6.80 (br d, J=8.88 Hz, 1 H) 7.42-7.51 (m, 1H) 7.57 (br d, J=8.63 Hz, 1H) 8.50 (br s, 1H).

### (R)—N-(3-(3,5-dimethylisoxazol-4-yl)-4-(morpholin-3-ylmethoxy)phenyl)cyclopropanecarboxamide (173)

### Preparation of (R)—N-(4-((4-benzylmorpholin-3-yl)methoxy)-3-(3,5-dimethylisoxazol-4-yl)phenyl)cyclopropanecarboxamide (37)

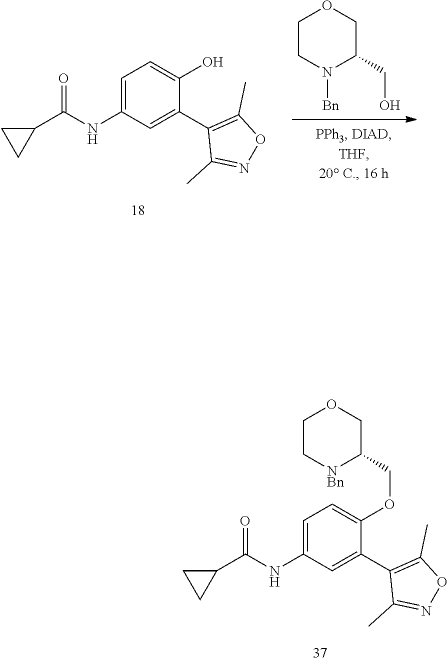 [SMILES: Cc1noc(C)c1-c1cc(NC(=O)C2CC2)ccc1O ; OC[C@H]1COCCN1Cc1ccccc1 ; Cc1noc(C)c1-c1cc(NC(=O)C2CC2)ccc1OC[C@H]1COCCN1Cc1ccccc1]

### Preparation of (R)—N-(3-(3,5-dimethylisoxazol-4-yl)-4-(morpholin-3-ylmethoxy)phenyl)cyclopropanecarboxamide (173)

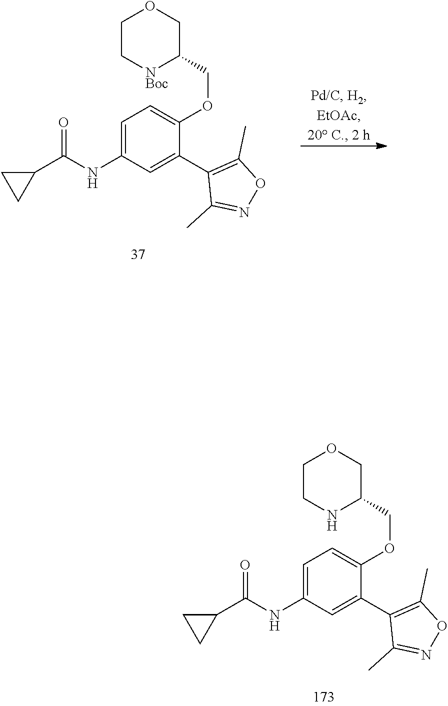 [SMILES: Cc1noc(C)c1-c1cc(NC(=O)C2CC2)ccc1OC[C@H]1COCCN1C(=O)OC(C)(C)C ; Cc1noc(C)c1-c1cc(NC(=O)C2CC2)ccc1OC[C@H]1COCCN1]

LCMS (ESI): m/z [M+H] calcd for C₂₀H₂₆N₃O₄: 372.18; found: 372.1.

¹H NMR (400 MHZ, methanol-d₄) δ=7.53 (dd, J=2.7, 8.9 Hz, 1H), 7.41 (d, J=2.7 Hz, 1H), 7.08 (d, J=9.0 Hz, 1H), 3.93-3.85 (m, 2H), 3.76 (ddd, J=3.0, 8.0, 11.2 Hz, 2H), 3.47 (td, J=6.7, 11.4 Hz, 1H), 3.26 (dd, J=9.4, 11.2 Hz, 1H), 3.16-3.03 (m, 1H), 2.89-2.84 (m, 2H), 2.31 (s, 3H), 2.15 (s, 3H), 1.78-1.69 (m, 1H), 0.96-0.91 (m, 2H), 0.87-0.82 (m, 2H).

### N-[4-(azetidin-2-ylmethoxy)-3-(3,5-dimethylisoxazol-4-yl)phenyl]cyclopropanecarboxamide (174)

### Preparation of tert-butyl 2-[[4-(cyclopropanecarbonylamino)-2-(3,5-dimethylisoxazol-4-yl)phenoxy]methyl]azetidine-1-carboxylate (38)

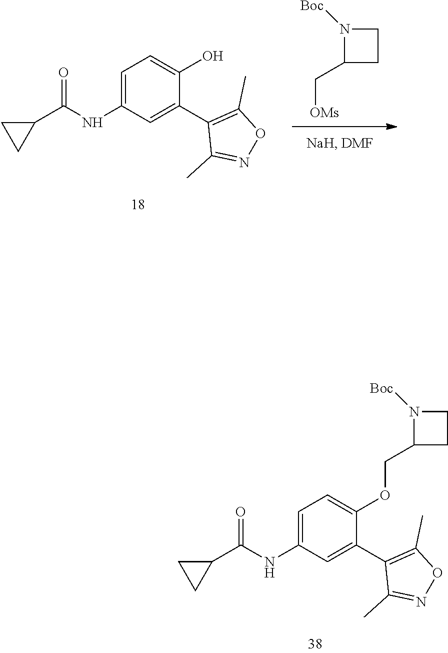 [SMILES: Cc1noc(C)c1-c1cc(NC(=O)C2CC2)ccc1O ; CC(C)(C)OC(=O)N1CCC1COS(C)(=O)=O ; Cc1noc(C)c1-c1cc(NC(=O)C2CC2)ccc1OCC1CCN1C(=O)OC(C)(C)C]

To a solution of N-[3-(3,5-dimethylisoxazol-4-yl)-4-hydroxy-phenyl]cyclopropanecarboxamide ((18), 100 mg, 367.24 μmol, 1 eq.) in DMF (2 mL) was added NaH (22.03 mg, 550.87 μmol, 60% purity, 1.5 eq.), and the reaction mixture was stirred at 25° C. for 10 mins, then tert-butyl 2-(methylsulfonyloxymethyl)azetidine-1-carboxylate (116.93 mg, 440.69 μmol, 1.2 eq.) was added to the reaction mixture. The reaction mixture was stirred at 25° C. for 2 hrs (monitored by LCMS) then was concentrated under vacuum. The residue was purified by prep-TLC (petroleum ether:ethyl acetate=1:1) to give a residue. Tert-butyl 2-[[4-(cyclopropanecarbonylamino)-2-(3,5-dimethylisoxazol-4-yl)phenoxy]methyl]azetidine-1-carboxylate ((38), 70 mg, 158.54 μmol, 43.17% yield) was obtained as a white solid.

### Preparation of N-[4-(azetidin-2-ylmethoxy)-3-(3,5-dimethylisoxazol-4-yl)phenyl]cyclopropanecarboxamide (174)

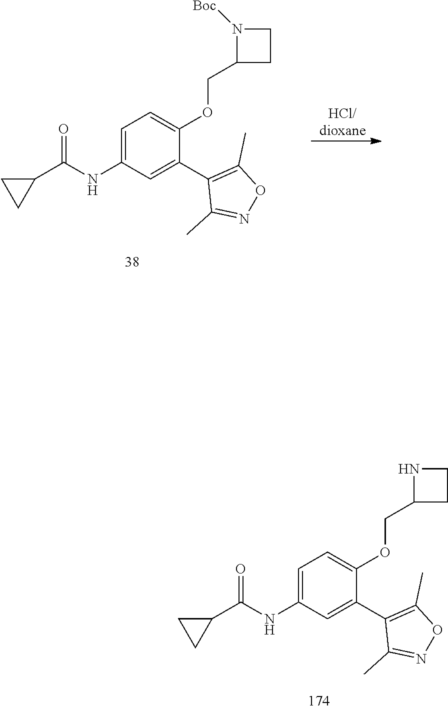 [SMILES: Cc1noc(C)c1-c1cc(NC(=O)C2CC2)ccc1OCC1CCN1C(=O)OC(C)(C)C ; Cc1noc(C)c1-c1cc(NC(=O)C2CC2)ccc1OCC1CCN1]

To a solution of tert-butyl 2-[[4-(cyclopropanecarbonylamino)-2-(3,5-dimethylisoxazol-4-yl)phenoxy]methyl]azetidine-1-carboxylate ((38), 70 mg, 158.54 μmol, 1 eq.) in DCM (2 mL) was added HCl/dioxane (4 M, 2 mL, 50.46 eq.). The reaction mixture was stirred at 25° C. for 2 hrs (monitored by LCMS). The reaction mixture was concentrated under vacuum. The residue was purified by prep-HPLC (column: Phenomenex Synergi C18 150*25*10 μm; mobile phase: [water (0.225% FA)-ACN]; B %: 4%-34%, 10 min), followed by lyophilization. N-[4-(azetidin-2-ylmethoxy)-3-(3,5-dimethylisoxazol-4-yl)phenyl]cyclopropanecarboxamide (24.4 mg, 71.47 μmol, 45.08% yield, 100% purity) was obtained as a yellow solid.

LCMS (ESI): m/z [M+H] calcd for C₁₉H₂₄N₃O₃: 342.17; found: 342.2.

¹H NMR (400 MHZ, methanol-d₄) δ=8.51 (br s, 1H), 7.59 (dd, J=2.6, 8.9 Hz, 1H), 7.44 (d, J=2.6 Hz, 1H), 7.14 (d, J=8.9 Hz, 1H), 4.78-4.68 (m, 1H), 4.38-4.26 (m, 2H), 4.06-3.96 (m, 1H), 3.78 (dt, J=6.5, 10.2 Hz, 1H), 2.62 (dtd, J=6.5, 9.3, 12.2 Hz, 1H), 2.48-2.35 (m, 1H), 2.31 (s, 3H), 2.16 (s, 3H), 1.79-1.71 (m, 1H), 0.97-0.82 (m, 4H).

### (R)—N-(3-(3,5-dimethylisoxazol-4-yl)-4-(pyrrolidin-2-ylmethoxy)phenyl)cyclopropanecarboxamide (175)

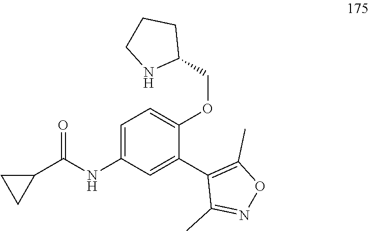 [SMILES: Cc1noc(C)c1-c1cc(NC(=O)C2CC2)ccc1OC[C@H]1CCCN1]

Compound 175 was prepared according to the synthesis described for compound 174, substituting tert-butyl (R)-2-(((methylsulfonyl)oxy)methyl)pyrrolidine-1-carboxylate for tert-butyl 2-(methylsulfonyloxymethyl)azetidine-1-carboxylate.

LCMS (ESI): m/z [M+H] calcd for C₂₀H₂₅N₃O₃: 356.19; found: 356.2.

¹H NMR (400 MHZ, methanol-d₄) δ=7.59 (dd, J=2.6, 8.9 Hz, 1H), 7.43 (d, J=2.6 Hz, 1H), 7.13 (d, J=8.9 Hz, 1H), 4.27 (dd, J=3.6, 10.7 Hz, 1H), 4.14 (dd, J=8.1, 10.6 Hz, 1H), 3.96 (dq, J=3.5, 7.8 Hz, 1H), 3.29-3.24 (m, 1H), 3.15 (td, J=7.5, 11.6 Hz, 1H), 2.32 (s, 3H), 2.24 (br d, J=7.7 Hz, 1H), 2.16 (s, 3H), 2.08-1.94 (m, 2H), 1.87-1.71 (m, 2H), 0.97-0.90 (m, 2H), 0.86 (td, J=3.1, 7.9 Hz, 2H).

### (S)—N-(3-(3,5-dimethylisoxazol-4-yl)-4-(pyrrolidin-2-ylmethoxy)phenyl)cyclopropanecarboxamide (176)

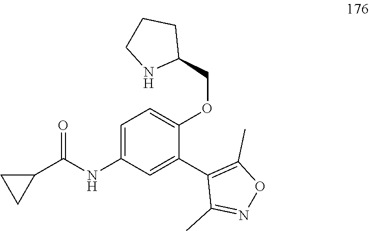 [SMILES: Cc1noc(C)c1-c1cc(NC(=O)C2CC2)ccc1OC[C@@H]1CCCN1]

Compound 176 was prepared according to the synthesis described for compound 174, substituting tert-butyl (S)-2-(((methylsulfonyl)oxy)methyl)pyrrolidine-1-carboxylate for tert-butyl 2-(methylsulfonyloxymethyl)azetidine-1-carboxylate.

LCMS (ESI): m/z [M+H] calcd for C₂₀H₂₆N₃O₃: 356.19; found: 356.2.

¹H NMR (400 MHZ, methanol-d₄) δ ppm 0.80-0.99 (m, 4H) 1.42-1.62 (m, 1H) 1.68-1.82 (m, 3H) 1.85-2.01 (m, 1H) 2.10-2.18 (m, 3H) 2.30 (s, 3H) 2.86 (t, J=6.88 Hz, 2H) 3.33-3.52 (m, 1H) 3.93 (d, J=5.75 Hz, 2H) 7.07 (d, J=9.01 Hz, 1H) 7.40 (d, J=2.63 Hz, 1H) 7.52 (dd, J=8.88, 2.63 Hz, 1H).

### N-[3-(3,5-dimethylisoxazol-4-yl)-4-[[(2R)-2-piperidyl]methoxy]phenyl]-2-methyl-pyridinerazole-3-carboxamide (177)

### Preparation of tert-butyl (2R)-2-[[2-bromo-4-[(2-methylpyridinerazole-3-carbonyl)amino]phenoxy]methyl]piperidine-1-carboxylate (39)

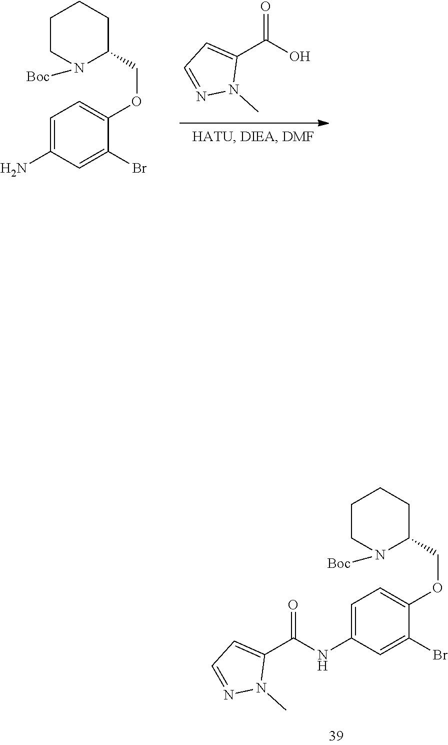

To the mixture of 2-methylpyridinerazole-3-carboxylic acid (981.95 mg, 7.79 mmol, 1.5 eq.) in DMF (40 mL) was added HATU (1.97 g, 5.19 mmol, 1 eq.) and DIEA (670.88 mg, 5.19 mmol, 904.15 μL, 1 eq.). The mixture was stirred at 25° C. for 0.5 hrs. Then tert-butyl (2R)-2-[(4-amino-2-bromo-phenoxy)methyl]piperidine-1-carboxylate (2 g, 5.19 mmol, 1 eq.) was added to the mixture. The mixture was stirred at 25° C. for 16 hrs.

The residue was poured into water (100 mL). The aqueous phase was extracted with ethyl acetate (80 mL×3). The combined organic phase was washed with brine (80 mL), dried with anhydrous Na₂SO₄, filtered and concentrated under vacuum. The residue was purified by flash silica gel chromatography (petroleum ether:ethyl acetate=0˜60%). Tert-butyl (2R)-2-[[2-bromo-4-[(2-methylpyridinerazole-3-carbonyl)amino]phenoxy]methyl]piperidine-1-carboxylate ((39), 2.5 g, 5.07 mmol, 97.61% yield) was obtained as an orange gum.

¹H NMR (400 MHZ, methanol-d₄) δ ppm 1.44-1-48 (m, 10H), 1.62-1.74 (m, 4H) 1.98 (br d, J=9.54 Hz, 1H) 2.80 (br s, 3H) 2.86 (s, 1H) 3.35 (s, 1H) 3.98-4.07 (m, 1H) 4.13-4.15 (m, 3H) 4.18 (br d, J=6.97 Hz, 1H) 4.55-4.63 (m, 1H) 6.94 (d, J=2.08 Hz, 1H) 7.08 (d, J=8.93 Hz, 1H) 7.50 (d, J=2.20 Hz, 1H) 7.56-7.62 (m, 1H) 7.95 (d, J=2.57 Hz, 1H).

### Preparation of tert-butyl (2R)-2-[[2-(3,5-dimethylisoxazol-4-yl)-4-[(2-methylpyridinerazole-3-carbonyl)amino]phenoxy]methyl]piperidine-1-carboxylate (40)

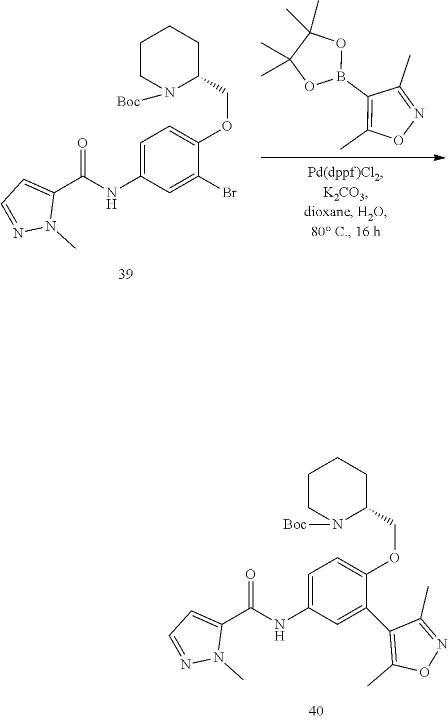

To a mixture of tert-butyl (2R)-2-[[2-bromo-4-[(2-methylpyridinerazole-3-carbonyl)amino]phenoxy]methyl]piperidine-1-carboxylate ((39), 200 mg, 405.36 μmol, 1 eq.), 3,5-dimethyl-4-(4,4,5,5-tetramethyl-1,3,2-dioxaborolan-2-yl)isoxazole (108.51 mg, 486.43 μmol, 1.2 eq.) and K₂CO₃ (112.05 mg, 810.71 μmol, 2 eq.) in dioxane (3 mL) and H₂O (0.5 mL) was added Pd(dppf)Cl₂ (29.66 mg, 40.54 μmol, 0.1 eq.) under N₂. The mixture was stirred at 80° C. for 16 hrs. The reaction mixture was concentrated under vacuum. The residue was poured into water (20 mL). The aqueous phase was extracted with ethyl acetate (100 mL×3). The combined organic phase was dried with anhydrous Na₂SO₄, filtered and concentrated under vacuum. The residue was purified by flash silica gel chromatography (ISCO®; 20 g SepaFlash® Silica Flash Column, eluent of 0˜50% ethyl acetate/petroleum ether gradient at 40 mL/min). Tert-butyl (2R)-2-[[2-(3,5-dimethylisoxazol-4-yl)-4-[(2-methylpyridinerazole-3-carbonyl)amino]phenoxy]methyl]piperidine-1-carboxylate ((40), 157 mg, 308.09 μmol, 76.00% yield) was obtained as a yellow gum.

LCMS (ESI): m/z [M+H] calcd for C₂₇H₃₆N₅O₅: 510.3; found: 454.2/510.3.

### Preparation of N-[3-(3,5-dimethylisoxazol-4-yl)-4-[[(2R)-2-piperidyl]methoxy]phenyl]-2-methyl-pyridinerazole-3-carboxamide (177)

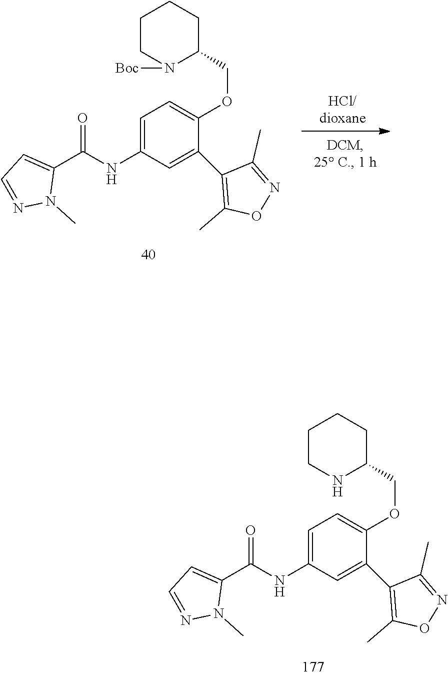 [SMILES: Cc1noc(C)c1-c1cc(NC(=O)c2ccnn2C)ccc1OC[C@H]1CCCCN1C(=O)OC(C)(C)C ; Cc1noc(C)c1-c1cc(NC(=O)c2ccnn2C)ccc1OC[C@H]1CCCCN1]

To a mixture of tert-butyl (2R)-2-[[2-(3,5-dimethylisoxazol-4-yl)-4-[(2-methylpyridinerazole-3-carbonyl)amino]phenoxy]methyl]piperidine-1-carboxylate ((40), 157 mg, 308.09 μmol, 1 eq.) in DCM (2 mL) was added HCl/dioxane (4 M, 2 mL, 25.97 eq.). The mixture was stirred at 25° C. for 1 hr. The reaction mixture was concentrated under vacuum. The residue was purified by prep-HPLC (column: Phenomenex Luna C18 150*25 mm*10 μm; mobile phase: [water (0.1% TFA)-ACN]; B %: 10%-40%, 10 min). N-[3-(3,5-dimethylisoxazol-4-yl)-4-[[(2R)-2-piperidyl]methoxy]phenyl]-2-methyl-pyridinerazole-3-carboxamide (92.26 mg, 223.06 μmol, 72.40% yield, 99% purity was obtained as an off white solid.

LCMS (ESI): m/z [M+H] calcd for C₂₂H₂₇N₅O₃: 410.4; found: 410.3.

¹H NMR (400 MHZ, methanol-d₄) δ ppm 1.49-1.72 (m, 3H) 1.86-1.98 (m, 3H) 2.18 (s, 3H) 2.34 (s, 3H) 2.98-3.08 (m, 1H) 3.39 (br d, J=12.76 Hz, 1H) 3.43-3.51 (m, 1H) 4.12 (d, J=5.63 Hz, 2H) 4.14 (s, 3H) 6.97 (d, J=2.13 Hz, 1H) 7.22 (d, J=9.01 Hz, 1H) 7.50 (d, J=2.13 Hz, 1H) 7.57 (d, J=2.63 Hz, 1H) 7.72 (dd, J=8.94, 2.69 Hz, 1H).

### N-[3-(3,5-dimethylisoxazol-4-yl)-4-[[(3S)-3-piperidyl]oxy]phenyl]cyclopropanecarboxamide (178)

### Preparation of tert-butyl (3S)-3-[2-bromo-4-(cyclopropanecarbonylamino)phenoxy]piperidine-1-carboxylate (41)

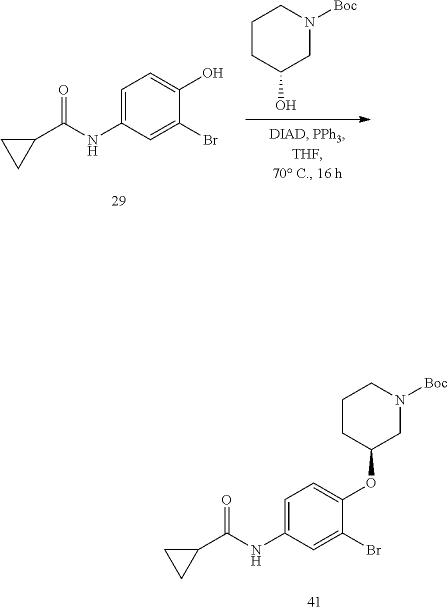 [SMILES: O=C(Nc1ccc(O)c(Br)c1)C1CC1 ; CC(C)(C)OC(=O)N1CCC[C@@H](O)C1 ; CC(C)(C)OC(=O)N1CCC[C@H](Oc2ccc(NC(=O)C3CC3)cc2Br)C1]

To a solution of N-(3-bromo-4-hydroxy-phenyl)cyclopropanecarboxamide ((29), 1 g, 3.90 mmol, 1 eq.) and tert-butyl (3R)-3-hydroxypiperidine-1-carboxylate (1.57 g, 7.81 mmol, 2 eq.) in THF (20 mL) was added PPh₃ (3.07 g, 11.71 mmol, 3 eq.) and DIAD (2.37 g, 11.71 mmol, 2.28 mL, 3 eq.) at 0° C. The mixture was stirred at 70° C. for 16 hrs. The mixture was cooled to 25° C., was poured into 0.5 M NaOH (30 mL) and extracted with ethyl acetate (50 mL). The aqueous phase was extracted with ethyl acetate (30 mL×2). The combined organic phase was washed with brine (30 mL×2), dried with anhydrous Na₂SO₄, filtered, and concentrated under vacuum. The residue was purified by silica gel chromatography (column height: 250 mm, diameter: 100 mm, 100-200 mesh silica gel, petroleum ether:ethyl acetate=20:1 to 1:1). Tert-butyl (3S)-3-[2-bromo-4-(cyclopropanecarbonylamino)phenoxy]piperidine-1-carboxylate ((41), 660 mg, 1.50 mmol, 38.47% yield) was obtained as a white solid.

¹H NMR (400 MHZ, chloroform-d) δ=0.75-0.88 (m, 2H), 1.05 (br d, J=3.00 Hz, 2H), 1.19-1.30 (m, 2H), 1.31-1.46 (m, 9H), 1.86 (br d, J=5.50 Hz, 2H), 1.98 (br s, 1H), 3.04-4.01 (m, 4H), 4.19 (br s, 1H), 6.90 (br s, 1H), 7.29-7.44 (m, 1H), 7.74 (br s, 1H).

### Preparation of tert-butyl (3S)-3-[4-(cyclopropanecarbonylamino)-2-(3,5-dimethylisoxazol-4-yl)phenoxy]piperidine-1-carboxylate (42)

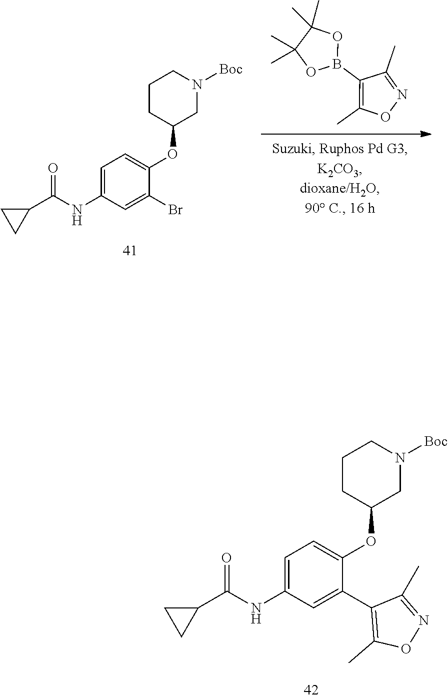 [SMILES: CC(C)(C)OC(=O)N1CCC[C@H](Oc2ccc(NC(=O)C3CC3)cc2Br)C1 ; Cc1noc(C)c1B1OC(C)(C)C(C)(C)O1 ; Cc1noc(C)c1-c1cc(NC(=O)C2CC2)ccc1O[C@H]1CCCN(C(=O)OC(C)(C)C)C1]

To a mixture of tert-butyl (3S)-3-[2-bromo-4-(cyclopropanecarbonylamino)phenoxy]piperidine-1-carboxylate ((41), 300 mg, 682.84 μmol, 1 eq.) and 3,5-dimethyl-4-(4,4,5,5-tetramethyl-1,3,2-dioxaborolan-2-yl)isoxazole (456.97 mg, 2.05 mmol, 3 eq.) in dioxane (6 mL) and H₂O (0.6 mL) were added [2-(2-aminophenyl)phenyl]-methylsulfonyloxy-palladium; dicyclohexyl-[2-(2,6-diisopropoxyphenyl)phenyl]phosphane (57.11 mg, 68.28 μmol, 0.1 eq.) and K₂CO₃ (188.75 mg, 1.37 mmol, 2 eq.) at 25° C. under N₂. Then the mixture was heated to 90° C. and stirred for 16 hrs. The mixture was cooled to 25° C. The mixture was poured into water (30 mL) and the aqueous phase was extracted with ethyl acetate (30 mL×3). The combined organic phase was washed with brine (25 mL×3), dried with anhydrous Na₂SO₄, filtered and concentrated under vacuum. The residue was purified by silica gel chromatography (petroleum ether/ethyl acetate=5:1 to 1:1). Tert-butyl (3S)-3-[4-(cyclopropanecarbonylamino)-2-(3,5-dimethylisoxazol-4-yl)phenoxy]piperidine-1-carboxylate ((42), 170 mg, 186.59 μmol, 27.33% yield, 50% purity) was obtained as a colorless oil.

### Preparation of N-[3-(3,5-dimethylisoxazol-4-yl)-4-[[(3S)-3-piperidyl]oxy]phenyl]cyclopropanecarboxamide (178)

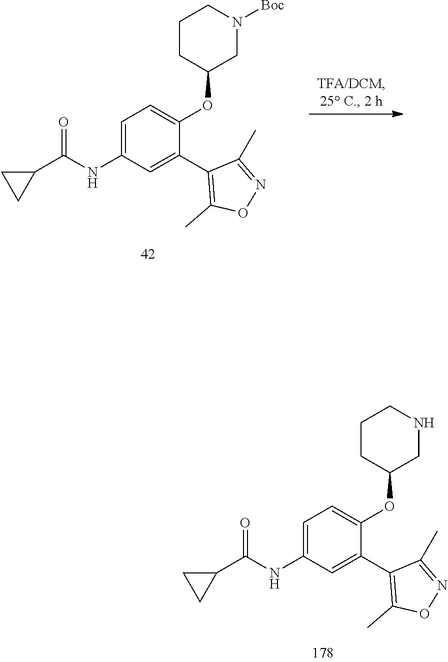 [SMILES: Cc1noc(C)c1-c1cc(NC(=O)C2CC2)ccc1O[C@H]1CCCN(C(=O)OC(C)(C)C)C1 ; Cc1noc(C)c1-c1cc(NC(=O)C2CC2)ccc1O[C@H]1CCCNC1]

A mixture of tert-butyl (3S)-3-[4-(cyclopropanecarbonylamino)-2-(3,5-dimethylisoxazol-4-yl)phenoxy]piperidine-1-carboxylate ((42), 150 mg, 329.28 μmol, 1 eq.) in DCM (5 mL) and TFA (0.5 mL) was stirred at 25° C. for 2 hrs. The mixture was concentrated under vacuum. The residue was purified by prep-HPLC (TFA conditions). N-[3-(3,5-dimethylisoxazol-4-yl)-4-[[(3S)-3-piperidyl]oxy]phenyl]cyclopropanecarboxamide (65 mg, 181.05 μmol, 54.98% yield, 99% purity) was obtained as a white solid.

LCMS (ESI): m/z [M+H] calcd for C₂₀H₂₆N₃O₃: 356.19; found: 356.2.

¹H NMR (400 MHZ, methanol-d₄) δ=0.81-0.89 (m, 2H), 0.9-0.97 (m, 2H), 1.64-1.80 (m, 3H), 1.87-2.01 (m, 2H), 2.19 (s, 3H), 2.34 (s, 3H), 3.05-3.20 (m, 3H), 3.32-3.37 (m, 1H), 4.31 (br s, J=2.75 Hz, 1H), 7.18 (d, J=8.88 Hz, 1H), 7.47 (d, J=2.25 Hz, 1H), 7.57 (dd, J=8.88, 2.50 Hz, 1H).

### (R)—N-(3-(3,5-dimethylisoxazol-4-yl)-4-(piperidin-3-yloxy)phenyl)cyclopropanecarboxamide (179)

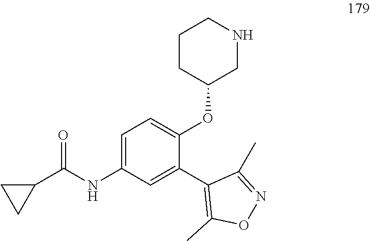 [SMILES: Cc1noc(C)c1-c1cc(NC(=O)C2CC2)ccc1O[C@@H]1CCCNC1]

Compound 179 was prepared according to the synthesis described for compound 178, substituting tert-butyl (3S)-3-hydroxypiperidine-1-carboxylate for tert-butyl (3R)-3-hydroxypiperidine-1-carboxylate.

¹H NMR (400 MHZ, methanol-d₄) δ=0.82-0.89 (m, 2H), 0.90-0.98 (m, 2H), 1.63-1.79 (m, 3H), 1.84-2.02 (m, 2H), 2.18 (s, 3H), 2.34 (s, 3H), 3.05-3.19 (m, 3H), 3.33 (br d, J=2.93 Hz, 1H), 4.25-4.34 (m, 1H), 7.17 (d, J=8.80 Hz, 1H), 7.45 (d, J=2.69 Hz, 1H), 7.58 (dd, J=8.80, 2.69 Hz, 1H).

LCMS (ESI): m/z [M+H] calcd for C₂₀H₂₆N₃O₃: 356.19; found: 356.2.

### (S)—N-(3-(3,5-dimethylisoxazol-4-yl)-4-(pyrrolidin-3-yloxy)phenyl)cyclopropanecarboxamide (180)

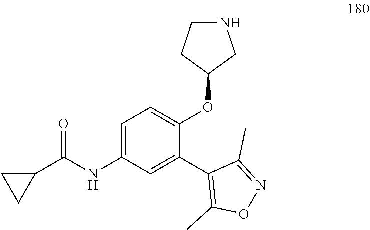 [SMILES: Cc1noc(C)c1-c1cc(NC(=O)C2CC2)ccc1O[C@H]1CCNC1]

Compound 180 was prepared according to the synthesis described for compound 178, substituting tert-butyl (R)-3-hydroxypyrrolidine-1-carboxylate for tert-butyl (3R)-3-hydroxypiperidine-1-carboxylate.

LCMS (ESI): m/z [M+H] calcd for C₁₉H₂₄N₃O₃: 342.17; found: 342.2.

¹H NMR (400 MHZ, methanol-d₄) δ=7.61-7.56 (m, 1H), 7.44 (d, J=2.6 Hz, 1H), 7.11 (d, J=9.0 Hz, 1H), 5.09-5.03 (m, 1H), 3.60-3.53 (m, 1H), 3.44-3.37 (m, 2H), 3.18 (dt, J=7.4, 10.8 Hz, 1H), 2.32 (s, 3H), 2.28-2.18 (m, 2H), 2.16 (s, 3H), 1.80-1.70 (m, 1H), 0.96-0.92 (m, 2H), 0.88-0.82 (m, 2H).

### (R)—N-(3-(3,5-dimethylisoxazol-4-yl)-4-(pyrrolidin-3-yloxy)phenyl)cyclopropanecarboxamide (181)

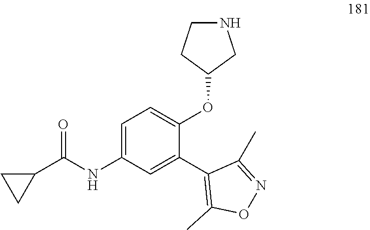

Compound 181 was prepared according to the synthesis described for compound 178, substituting tert-butyl (S)-3-hydroxypyrrolidine-1-carboxylate for tert-butyl (3R)-3-hydroxypiperidine-1-carboxylate.

LCMS (ESI): m/z [M+H] calcd for C₁₉H₂₄N₃O₃: 342.3; found: 342.3.

¹H NMR (400 MHZ, methanol-d₄) δ=7.58 (dd, J=2.7, 8.9 Hz, 1H), 7.45 (d, J=2.7 Hz, 1H), 7.11 (d, J=8.9 Hz, 1H), 5.09-5.01 (m, 1H), 3.63-3.52 (m, 1H), 3.44-3.34 (m, 2H), 3.23-3.14 (m, 1H), 2.32 (s, 3H), 2.29-2.17 (m, 2H), 2.16 (s, 3H), 1.79-1.71 (m, 1H), 0.96-0.80 (m, 4H).

### N-[3-(3,5-dimethylisoxazol-4-yl)-4-[[(2R)-2-piperidyl]methoxy]phenyl]isoxazole-4-carboxamide (182)

### Preparation of tert-butyl (2R)-2-[[2-(3,5-dimethylisoxazol-4-yl)-4-(isoxazole-4-carbonylamino)phenoxy]methyl]piperidine-1-carboxylate (43)

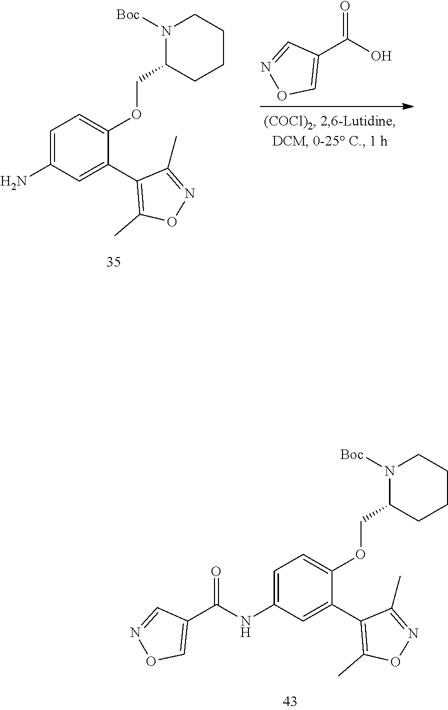

To a mixture of isoxazole-4-carboxylic acid (30.98 mg, 273.97 μmol, 1.1 eq.) and oxalyl dichloride (94.84 mg, 747.20 μmol, 65.41 μL, 3 eq.) in DCM (1 mL) was added DMF (182.05 μg, 2.49 μmol, 1.92e-1 μL, 0.01 eq.) at 0° C. The mixture was stirred at 25° C. for 16 hrs. The mixture was concentrated under reduced pressure to give a residue. The residue was dissolved in MeCN (2 mL) and added to a mixture of tert-butyl (2R)-2-[[4-amino-2-(3,5-dimethylisoxazol-4-yl)phenoxy]methyl]piperidine-1-carboxylate ((35), 100 mg, 249.07 μmol, 1 eq.) and 2,6-Lutidine (53.38 mg, 498.13 μmol, 58.02 μL, 2 eq.) in DCM (1 mL). The mixture was stirred at 25° C. for 10 min (monitored by LC-MS). The reaction mixture was concentrated under reduced pressure to give a residue. Tert-butyl (2R)-2-[[2-(3,5-dimethylisoxazol-4-yl)-4-(isoxazole-4-carbonylamino)phenoxy]methyl]piperidine-1-carboxylate ((43), 100 mg, crude) was obtained as a white solid.

LCMS (ESI): m/z [M −56+H] & [M −100+H] calcd for C₂₂H₂₅N₄O₆ and C₂₁H₂₅N₄O₄: 441.17 and 397.18; found: 441.1 and 397.1.

### Preparation of N-[3-(3,5-dimethylisoxazol-4-yl)-4-[[(2R)-2-piperidyl]methoxy]phenyl]isoxazole-4-carboxamide (182)

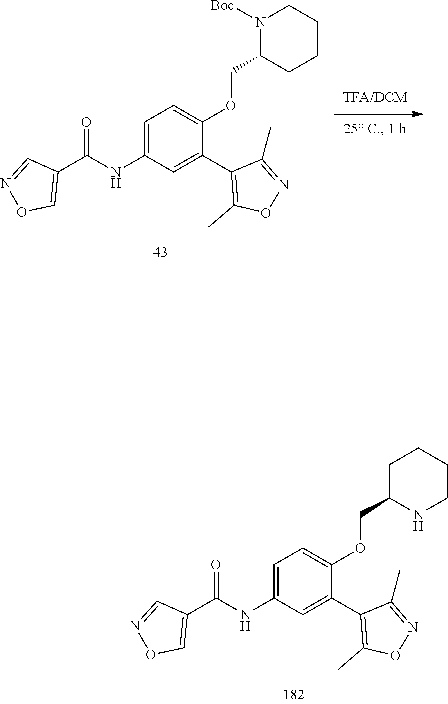 [SMILES: Cc1noc(C)c1-c1cc(NC(=O)c2cnoc2)ccc1OC[C@H]1CCCCN1C(=O)OC(C)(C)C ; Cc1noc(C)c1-c1cc(NC(=O)c2cnoc2)ccc1OC[C@H]1CCCCN1]

A mixture of tert-butyl (2R)-2-[[2-(3,5-dimethylisoxazol-4-yl)-4-(isoxazole-4-carbonylamino)phenoxy]methyl]piperidine-1-carboxylate ((43), 100 mg, 201.39 μmol, 1 eq.) in TFA (0.5 mL) and DCM (0.5 mL) was stirred at 25° C. for 10 min. The reaction mixture was concentrated under reduced pressure to give a residue. The residue was purified by prep-HPLC (column: Phenomenex Gemini-NX C18 75*30 mm*3 μm; mobile phase: [water (0.1% TFA)-ACN]; B %: 20%-30%, 7 min). N-[3-(3,5-dimethylisoxazol-4-yl)-4-[[(2R)-2-piperidyl]methoxy]phenyl]isoxazole-4-carboxamide (53.85 mg, 105.49 μmol, 52.38% yield, 100% purity, TFA) was obtained as a yellow solid.

LCMS (ESI): m/z [M+H] calcd for C₂₁H₂₅N₄O₄: 397.18; found: 397.2.

¹H NMR (400 MHZ, methanol-d₄) δ=9.30 (s, 1H), 8.85 (s, 1H), 7.73 (dd, J=2.7, 8.9 Hz, 1H), 7.54 (d, J=2.7 Hz, 1H), 7.22 (d, J=8.9 Hz, 1H), 4.15-4.03 (m, 2H), 3.52-3.43 (m, 1H), 3.42-3.35 (m, 1H), 3.10-2.96 (m, 1H), 2.35 (s, 3H), 2.19 (s, 3H), 1.97-1.86 (m, 3H), 1.74-1.44 (m, 3H).

### (R)—N-(3-(3,5-dimethylisoxazol-4-yl)-4-(piperidin-2-ylmethoxy)phenyl)-3-methylisoxazole-4-carboxamide (183)

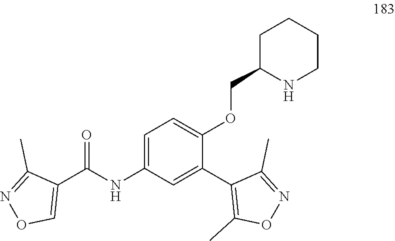 [SMILES: Cc1nocc1C(=O)Nc1ccc(OC[C@H]2CCCCN2)c(-c2c(C)noc2C)c1]

Compound 183 was prepared according to the synthesis described for compound 182, substituting 3-methylisoxazole-4-carboxylic acid for isoxazole-4-carboxylic acid.

LCMS (ESI): m/z [M+H] calcd for C₂₂H₂₇N₄O₄:411.47; found: 411.1.

¹H NMR (400 MHZ, methanol-d₄) δ=9.17 (s, 1H), 7.70 (dd, J=2.6, 8.9 Hz, 1H), 7.51 (d, J=2.6 Hz, 1H), 7.21 (d, J=8.9 Hz, 1H), 4.15-4.02 (m, 2H), 3.53-3.42 (m, 1H), 3.38 (br d, J=12.8 Hz, 1H), 3.12-2.97 (m, 1H), 2.49 (s, 3H), 2.35 (s, 3H), 2.19 (s, 3H), 1.98-1.86 (m, 3H), 1.75-1.46 (m, 3H).

### (R)—N-(3-(3,5-dimethylisoxazol-4-yl)-4-(piperidin-2-ylmethoxy)phenyl)-5-methylisoxazole-4-carboxamide (184)

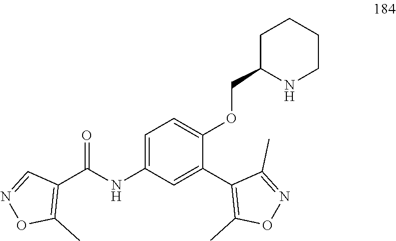 [SMILES: Cc1noc(C)c1-c1cc(NC(=O)c2cnoc2C)ccc1OC[C@H]1CCCCN1]

Compound 184 was prepared according to the synthesis described for compound 182, substituting 5-methylisoxazole-4-carboxylic acid for isoxazole-4-carboxylic acid.

LCMS (ESI): m/z [M+H] calcd for C₂₂H₂₇N₄O₄: 411.2; found: 411.7.

¹H NMR (400 MHz, methanol-d₄) δ=8.82 (s, 1H), 7.70 (dd, J=2.7, 8.9 Hz, 1H), 7.53 (d, J=2.6 Hz, 1H), 7.21 (d, J=8.9 Hz, 1H), 4.18-3.98 (m, 2H), 3.47 (br dd, J=4.2, 6.7 Hz, 1H), 3.39 (br d, J=12.8 Hz, 1H), 3.10-2.97 (m, 1H), 2.71 (s, 3H), 2.37-2.34 (m, 3H), 2.19 (s, 3H), 1.92 (dt, J=3.3, 6.5 Hz, 3H), 1.70-1.50 (m, 3H).

### (R)—N-(3-(3,5-dimethylisoxazol-4-yl)-4-(piperidin-2-ylmethoxy)phenyl)-5-methyloxazole-4-carboxamide (185)

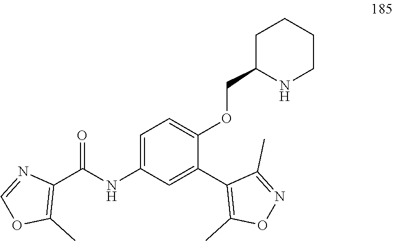 [SMILES: Cc1noc(C)c1-c1cc(NC(=O)c2ncoc2C)ccc1OC[C@H]1CCCCN1]

Compound 185 was prepared according to the synthesis described for compound 182, substituting 5-methyloxazole-4-carboxylic acid for isoxazole-4-carboxylic acid.

LCMS (ESI): m/z [M+H] calcd for C₂₂H₂₇N₄O₄: 411.2; found: 411.8.

¹H NMR (400 MHZ, methanol-d₄) δ=8.07 (s, 1H), 7.74 (dd, J=2.7, 8.9 Hz, 1H), 7.61 (d, J=2.6 Hz, 1H), 7.21 (d, J=8.9 Hz, 1H), 4.15-4.04 (m, 2H), 3.47 (td, J=2.9, 5.3 Hz, 1H), 3.39 (br d, J=12.7 Hz, 1H), 3.09-2.97 (m, 1H), 2.66 (s, 3H), 2.35 (s, 3H), 2.19 (s, 3H), 1.97-1.87 (m, 3H), 1.72-1.51 (m, 3H).

### (R)—N-(3-(3,5-dimethylisoxazol-4-yl)-4-(piperidin-2-ylmethoxy)phenyl)isothiazole-4-carboxamide (186)

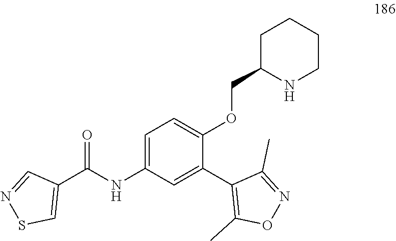 [SMILES: Cc1noc(C)c1-c1cc(NC(=O)c2cnsc2)ccc1OC[C@H]1CCCCN1]

Compound 186 was prepared according to the synthesis described for compound 182, substituting isothiazole-4-carboxylic acid for isoxazole-4-carboxylic acid.

LCMS (ESI): m/z [M+H] calcd for C₂₁H₂₅N₄O₃S: 413.16; found: 413.7.

¹H NMR (400 MHZ, methanol-d₄) δ=9.57 (s, 1H), 8.97 (s, 1H), 7.74 (dd, J=2.2, 8.9 Hz, 1H), 7.59 (d, J=2.2 Hz, 1H), 7.23 (d, J=8.9 Hz, 1H), 4.24-3.97 (m, 2H), 3.54-3.34 (m, 2H), 3.12-2.94 (m, 1H), 2.35 (d, J=1.3 Hz, 3H), 2.19 (d, J=1.0 Hz, 3H), 2.01-1.81 (m, 3H), 1.77-1.47 (m, 3H).

### (R)—N-(3-(3,5-dimethylisoxazol-4-yl)-4-(piperidin-2-ylmethoxy)phenyl)-3-methylisothiazole-4-carboxamide (187)

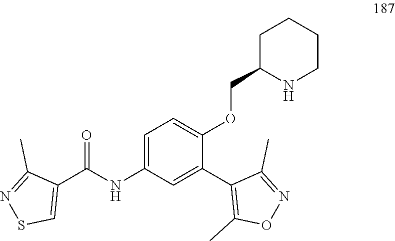 [SMILES: Cc1nscc1C(=O)Nc1ccc(OC[C@H]2CCCCN2)c(-c2c(C)noc2C)c1]

Compound 187 was prepared according to the synthesis described for compound 182, substituting 3-methylisothiazole-4-carboxylic acid for isoxazole-4-carboxylic acid.

LCMS (ESI): m/z [M+H] calcd for C₂₁H₂₅N₄O₃S: 427.17; found: 427.7.

¹H NMR (400 MHZ, methanol-d₄) δ=9.37 (s, 1H), 7.72 (dd, J=2.6, 8.9 Hz, 1H), 7.56 (d, J=2.6 Hz, 1H), 7.22 (d, J=8.9 Hz, 1H), 4.11 (d, J=5.5 Hz, 2H), 3.54-3.34 (m, 2H), 3.12-2.90 (m, 1H), 2.64 (s, 3H), 2.35 (s, 3H), 2.19 (s, 3H), 1.98-1.81 (m, 3H), 1.74-1.48 (m, 3H).

### (R)—N-(3-(3,5-dimethylisoxazol-4-yl)-4-(piperidin-2-ylmethoxy)phenyl)-5-methylthiazole-4-carboxamide (188)

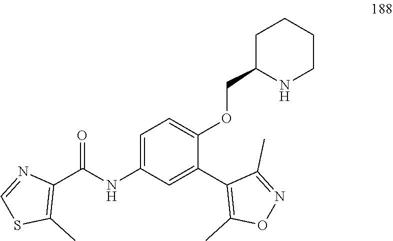 [SMILES: Cc1noc(C)c1-c1cc(NC(=O)c2ncsc2C)ccc1OC[C@H]1CCCCN1]

Compound 188 was prepared according to the synthesis described for compound 182, substituting 5-methylthiazole-4-carboxylic acid for isoxazole-4-carboxylic acid.

LCMS (ESI): m/z [M+H] calcd for C₂₁H₂₅N₄O₃S: 427.17; found: 427.8.

¹H NMR (400 MHZ, methanol-d₄) δ=8.77 (s, 1H), 7.75 (dd, J=2.7, 8.9 Hz, 1H), 7.63 (d, J=2.6 Hz, 1H), 7.21 (d, J=8.9 Hz, 1H), 4.10 (d, J=5.5 Hz, 2H), 3.54-3.34 (m, 2H), 3.11-2.96 (m, 1H), 2.83 (s, 3H), 2.35 (s, 3H), 2.19 (s, 3H), 1.91 (dt, J=3.9, 6.4 Hz, 3H), 1.76-1.48 (m, 3H).

### (R)—N-(3-(3,5-dimethylisoxazol-4-yl)-4-(piperidin-2-ylmethoxy)phenyl)-1-methyl-1H-pyrrole-2-carboxamide (189)

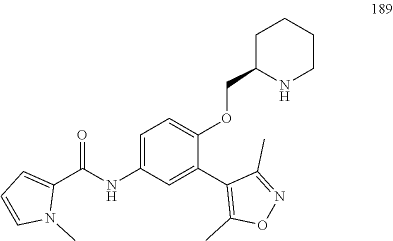 [SMILES: Cc1noc(C)c1-c1cc(NC(=O)c2cccn2C)ccc1OC[C@H]1CCCCN1]

Compound 189 was prepared according to the synthesis described for compound 182, substituting 1-methyl-1H-pyrrole-2-carboxylic acid for isoxazole-4-carboxylic acid.

LCMS (ESI): m/z [M+H] calcd for C₂₁H₂₅N₄O₃S: 409.22; found: 409.8.

¹H NMR (400 MHZ, methanol-d₄) δ=7.67 (dd, J=2.7, 8.9 Hz, 1H), 7.50 (d, J=2.6 Hz, 1H), 7.18 (d, J=8.9 Hz, 1H), 7.06-6.81 (m, 2H), 6.11 (dd, J=2.6, 4.0 Hz, 1H), 4.16-4.01 (m, 2H), 3.92 (s, 3H), 3.52-3.34 (m, 2H), 3.11-2.93 (m, 1H), 2.34 (s, 3H), 2.18 (s, 3H), 1.96-1.86 (m, 3H), 1.78-1.46 (m, 3H).

### (1S,2R)—N-(3-(3,5-dimethylisoxazol-4-yl)-4-(((R)-piperidin-2-yl)methoxy)phenyl)-2-fluorocyclopropane-1-carboxamide (190)

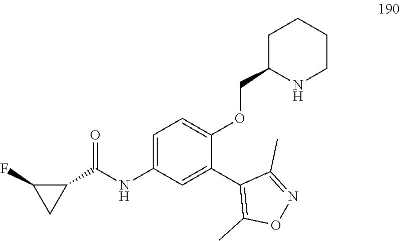 [SMILES: Cc1noc(C)c1-c1cc(NC(=O)[C@@H]2C[C@H]2F)ccc1OC[C@H]1CCCCN1]

Compound 190 was prepared according to the synthesis described for compound 182, substituting (1S,2R)-2-fluorocyclopropane-1-carboxylic acid for isoxazole-4-carboxylic acid.

LCMS (ESI): m/z [M+H] calcd for C₂₁H₂₇FN₃O₃: 388.20; found: 388.8.

¹H NMR (400 MHz, methanol-d₄) δ=7.56 (br dd, J=2.3, 8.6 Hz, 1H), 7.49-7.38 (m, 1H), 7.16 (d, J=8.9 Hz, 1H), 4.73 (ddd, J=1.6, 3.2, 4.5 Hz, 1H), 4.13-3.98 (m, 2H), 3.49-3.34 (m, 2H), 3.02 (br t, J=12.4 Hz, 1H), 2.32 (s, 3H), 2.27-2.13 (m, 1H), 2.16 (s, 3H), 1.91 (br d, J=11.5 Hz, 3H), 1.70-1.40 (m, 4H), 1.32 (qd, J=6.4, 12.7 Hz, 1H).

### (1R,2R)—N-(3-(3,5-dimethylisoxazol-4-yl)-4-(((R)-piperidin-2-yl)methoxy)phenyl)-2-fluorocyclopropane-1-carboxamide (191)

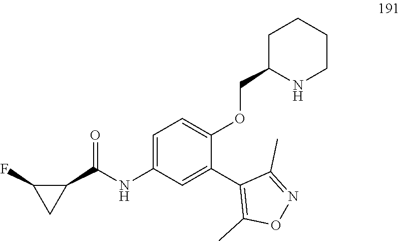 [SMILES: Cc1noc(C)c1-c1cc(NC(=O)[C@H]2C[C@H]2F)ccc1OC[C@H]1CCCCN1]

Compound 191 was prepared according to the synthesis described for compound 182, substituting (1R,2R)-2-fluorocyclopropane-1-carboxylic acid for isoxazole-4-carboxylic acid.

LCMS (ESI): m/z [M+H] calcd for C₂₁H₂₇FN₃O₃: 388.20; found: 388.7.

¹H NMR (400 MHZ, methanol-d₄) δ=7.67-7.54 (m, 1H), 7.45 (d, J=2.6 Hz, 1H), 7.17 (d, J=8.9 Hz, 1H), 4.91-4.74 (m, 1H), 4.12-4.00 (m, 2H), 3.51-3.34 (m, 2H), 3.10-2.97 (m, 1H), 2.33 (s, 3H), 2.17 (s, 3H), 2.02-1.83 (m, 4H), 1.81-1.49 (m, 4H), 1.16 (tdd, J=6.4, 9.1, 12.4 Hz, 1H).

### (1S,2S)—N-(3-(3,5-dimethylisoxazol-4-yl)-4-(((R)-piperidin-2-yl)methoxy)phenyl)-2-fluorocyclopropane-1-carboxamide (192)

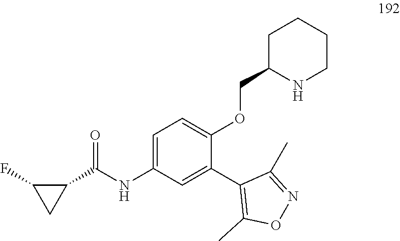 [SMILES: Cc1noc(C)c1-c1cc(NC(=O)[C@@H]2C[C@@H]2F)ccc1OC[C@H]1CCCCN1]

Compound 192 was prepared according to the synthesis described for compound 182, substituting (1S,2S)-2-fluorocyclopropane-1-carboxylic acid for isoxazole-4-carboxylic acid.

LCMS (ESI): m/z [M+H] calcd for C₂₁H₂₇FN₃O₃: 388.20; found: 388.7.

¹H NMR (400 MHZ, methanol-d₄) δ=7.61 (dd, J=2.6, 8.9 Hz, 1H), 7.45 (d, J=2.5 Hz, 1H), 7.17 (d, J=9.0 Hz, 1H), 4.89-4.71 (m, 1H), 4.14-3.97 (m, 2H), 3.50-3.34 (m, 2H), 3.12-2.94 (m, 1H), 2.33 (s, 3H), 2.17 (s, 3H), 2.02-1.84 (m, 4H), 1.82-1.49 (m, 4H), 1.16 (tdd, J=6.4, 9.2, 12.4 Hz, 1H).

### (R)—N-(3-(3,5-dimethylisoxazol-4-yl)-4-(piperidin-2-ylmethoxy)phenyl)-4-methylthiazole-5-carboxamide (193)

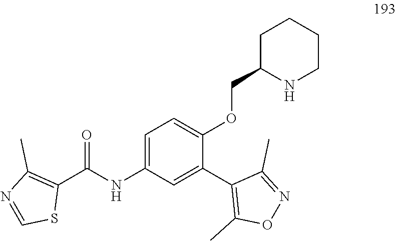 [SMILES: Cc1ncsc1C(=O)Nc1ccc(OC[C@H]2CCCCN2)c(-c2c(C)noc2C)c1]

Compound 193 was prepared according to the synthesis described for compound 182, substituting 4-methylthiazole-5-carboxylic acid for isoxazole-4-carboxylic acid.

LCMS (ESI): m/z [M+H] calcd for C₂₂H₂₇N₄O₃S: 427.17; found: 427.8.

¹H NMR (400 MHZ, methanol-d₄) δ=9.02 (s, 1H), 7.69 (dt, J=2.5, 5.7 Hz, 1H), 7.57-7.44 (m, 1H), 7.22 (d, J=8.9 Hz, 1H), 4.19-3.98 (m, 2H), 3.55-3.34 (m, 2H), 3.04 (br t, J=12.3 Hz, 1H), 2.69 (s, 3H), 2.35 (s, 3H), 2.19 (s, 3H), 1.99-1.82 (m, 3H), 1.76-1.50 (m, 3H).

### (R)—N-(3-(3,5-dimethylisoxazol-4-yl)-4-(piperidin-2-ylmethoxy)phenyl)-2,4-dimethylthiazole-5-carboxamide (194)

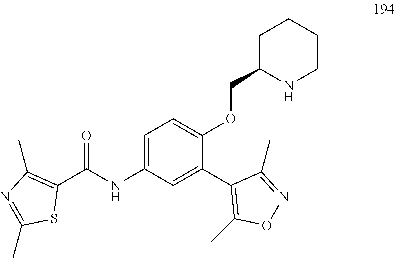 [SMILES: Cc1nc(C)c(C(=O)Nc2ccc(OC[C@H]3CCCCN3)c(-c3c(C)noc3C)c2)s1]

Compound 194 was prepared according to the synthesis described for compound 182, substituting 2,4-dimethylthiazole-5-carboxylic acid for isoxazole-4-carboxylic acid.

LCMS (ESI): m/z [M+H] calcd for C₂₃H₂₉N₄O₃S: 441.19; found: 441.7.

¹H NMR (400 MHZ, methanol-d₄) δ=7.74-7.57 (m, 1H), 7.50 (br d, J=2.3 Hz, 1H), 7.21 (d, J=8.9 Hz, 1H), 4.20-4.02 (m, 2H), 3.56-3.34 (m, 2H), 3.13-2.94 (m, 1H), 2.75-2.55 (m, 6H), 2.35 (s, 3H), 2.18 (s, 3H), 2.00-1.80 (m, 3H), 1.77-1.51 (m, 3H).

### (R)—N-(3-(3,5-dimethylisoxazol-4-yl)-4-(piperidin-2-ylmethoxy)phenyl)-4-methylisoxazole-5-carboxamide (195)

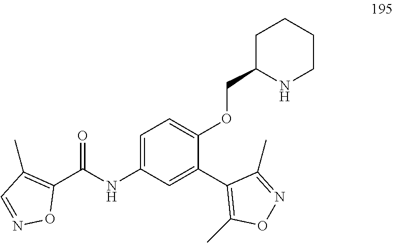 [SMILES: Cc1cnoc1C(=O)Nc1ccc(OC[C@H]2CCCCN2)c(-c2c(C)noc2C)c1]

Compound 195 was prepared according to the synthesis described for compound 182, substituting 4-methylisoxazole-5-carboxylic acid for isoxazole-4-carboxylic acid.

LCMS (ESI): m/z [M+H] calcd for C₂₂H₂₇N₄O₄:411.20; found: 411.8.

¹H NMR (400 MHZ, methanol-d₄) δ=8.43 (s, 1H), 7.78 (dd, J=2.6, 8.9 Hz, 1H), 7.59 (d, J=2.6 Hz, 1H), 7.22 (d, J=9.0 Hz, 1H), 4.16-4.04 (m, 2H), 3.52-3.44 (m, 1H), 3.38 (br d, J=12.8 Hz, 1H), 3.11-2.94 (m, 1H), 2.35 (d, J=1.1 Hz, 6H), 2.19 (s, 3H), 2.02-1.88 (m, 3H), 1.71-1.50 (m, 3H).

### (R)—N-(3-(3,5-dimethylisoxazol-4-yl)-4-(piperidin-2-ylmethoxy)phenyl)-4-methyloxazole-5-carboxamide (196)

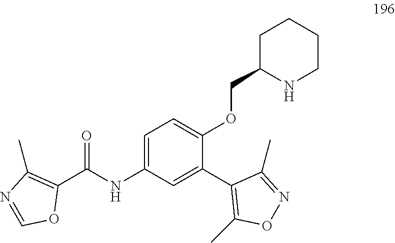 [SMILES: Cc1ncoc1C(=O)Nc1ccc(OC[C@H]2CCCCN2)c(-c2c(C)noc2C)c1]

Compound 196 was prepared according to the synthesis described for compound 182, substituting 4-methyloxazole-5-carboxylic acid for isoxazole-4-carboxylic acid.

LCMS (ESI): m/z [M+H] calcd for C₂₂H₂₇N₄O₄:411.20; found: 411.8.

¹H NMR (400 MHZ, methanol-d₄) δ=8.25 (s, 1H), 7.84-7.66 (m, 1H), 7.56 (d, J=2.6 Hz, 1H), 7.22 (d, J=8.9 Hz, 1H), 4.18-4.02 (m, 2H), 3.56-3.34 (m, 2H), 3.10-2.96 (m, 1H), 2.51 (s, 3H), 2.35 (s, 3H), 2.19 (s, 3H), 2.01-1.82 (m, 3H), 1.77-1.50 (m, 3H).

### N-[3-(3,5-dimethylisoxazol-4-yl)-4-[2-(2-methoxyethylamino)ethoxy]phenyl]cyclopropanecarboxamide (197)

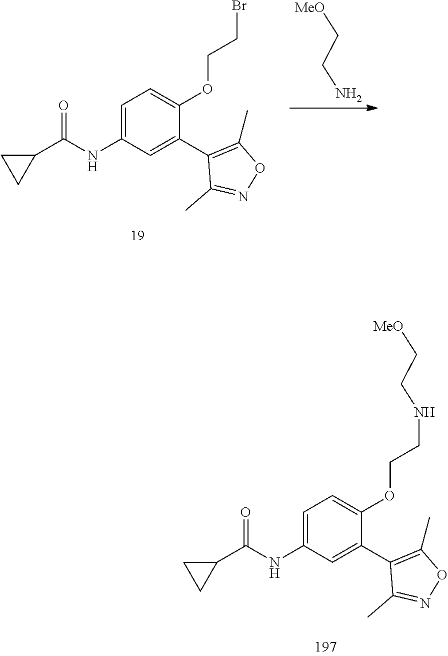 [SMILES: Cc1noc(C)c1-c1cc(NC(=O)C2CC2)ccc1OCCBr ; COCCN ; COCCNCCOc1ccc(NC(=O)C2CC2)cc1-c1c(C)noc1C]

To a solution of N-[4-(2-bromoethoxy)-3-(3,5-dimethylisoxazol-4-yl)phenyl]cyclopropanecarboxamide ((19), 80 mg, 210.94 μmol, 1 eq.) in MeCN (1 mL) was added K₂CO₃ (87.46 mg, 632.83 μmol, 3 eq.) and 2-methoxyethanamine (23.77 mg, 316.42 μmol, 27.51 μL, 1.5 eq.). The mixture was stirred at 60° C. for 16 hrs. The mixture was filtered and concentrated under vacuum to give a residue. The residue was purified by prep-HPLC (TFA conditions, column: Phenomenex Gemini-NX C18 75*30 mm*3 μm; mobile phase: [water (0.1% TFA)-ACN]; B %: 20%-30%, 7 min). N-[3-(3,5-dimethylisoxazol-4-yl)-4-[2-(2-methoxyethylamino)ethoxy]phenyl]cyclopropanecarboxamide (51.41 mg, 105.46 μmol, 50.00% yield, 100% purity, TFA) was obtained as a white solid.

LCMS (ESI): m/z [M+H] calcd for C₂₀H₂₈N₃O₄: 374.0, found: 374.2.

¹H NMR (400 MHz, methanol-d₄) δ 7.58 (dt, J=8.8, 2.6 Hz, 1H), 7.51-7.33 (m, 1H), 7.12 (d, J=9.0 Hz, 1H), 4.27 (t, J=5.1 Hz, 2H), 3.66-3.49 (m, 2H), 3.42 (t, J=5.2 Hz, 2H), 3.38 (s, 3H), 3.18-3.11 (m, 2H), 2.32 (s, 3H), 2.16 (s, 3H), 1.80-1.69 (m, 1H), 1.01-0.91 (m, 2H), 0.90-0.80 (m, 2H).

### N-(4-(2-acetimidamidoethoxy)-3-(3,5-dimethylisoxazol-4-yl)phenyl)cyclopropanecarboxamide (198)

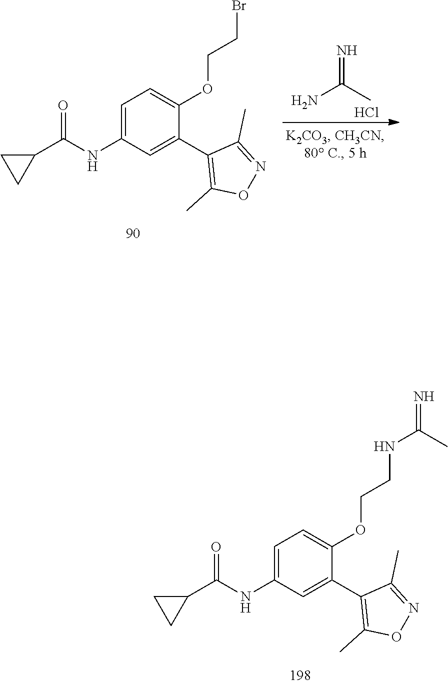

Compound 198 was prepared according to the synthesis described for compound 197, substituting acetimidamide for 2-methoxyethanamine.

LCMS (ESI): m/z [M+H] calcd for C₂₀H₂₈N₃O₄:357.0, found: 357.2.

¹H NMR (400 MHZ, methanol-d₄) δ 7.56 (dd, J=8.9, 2.6 Hz, 1H), 7.41 (d, J=2.6 Hz, 1H), 7.10 (d, J=8.9 Hz, 1H), 4.16 (t, J=5.1 Hz, 2H), 3.58 (t, J=5.1 Hz, 2H), 2.30 (s, 3H), 2.15 (s, 3H), 2.13 (s, 3H), 1.74 (tt, J=7.9, 4.6 Hz, 1H), 0.99-0.89 (m, 2H), 0.89-0.79 (m, 2H).

### N-[3-(3,5-dimethylisoxazol-4-yl)-4-[[(2R)-2-piperidyl]methoxy]phenyl]-2-fluoro-2-methyl-propanamide (199)

### Preparation of (2R)-tert-butyl 2-((2-(3,5-dimethylisoxazol-4-yl)-4-(2-fluoro-2-methylpropanamido)phenoxy)methyl)piperidine-1-carboxylate (44)

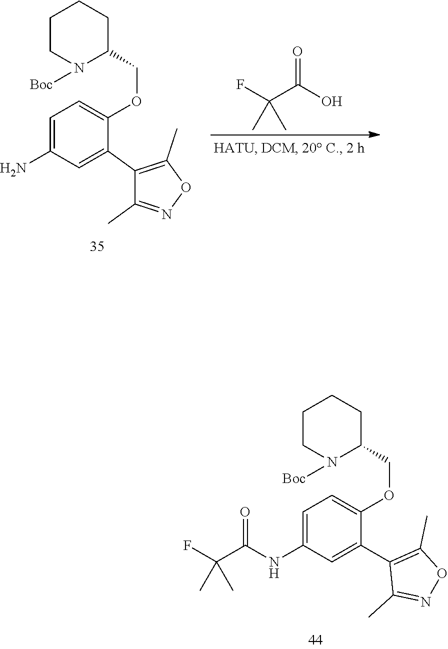

To a solution of 2-fluoro-2-methylpropanoic acid (23.78 mg, 224.16 μmol, 1.5 eq.) in DCM (1 mL) was added HATU (85.23 mg, 224.16 μmol, 1.5 eq.) and TEA (45.37 mg, 448.32 μmol, 62.40 μL, 3 eq.) and was stirred at 25° C. for 5 mins, then (2R)-tert-butyl 2-((4-amino-2-(3,5-dimethylisoxazol-4-yl)phenoxy)methyl)piperidine-1-carboxylate ((35), 60 mg, 149.44 μmol, 1 eq.) was added. The mixture was stirred at 25° C. for 2 hrs (monitored by LCMS). The mixture was concentrated under vacuum. Crude (2R)-tert-butyl 2-((2-(3,5-dimethylisoxazol-4-yl)-4-(2-fluoro-2-methylpropanamido)phenoxy)methyl)piperidine-1-carboxylate (70 mg, crude) was obtained as a yellow oil and used in the next step without further purification.

LCMS (ESI): m/z [M+H] calcd for C₂₆H₃₇FN₃O₅: 490.3, found: 490.1.

### Preparation of N-[3-(3,5-dimethylisoxazol-4-yl)-4-[[(2R)-2-piperidyl]methoxy]phenyl]-2-fluoro-2-methyl-propanamide (199)

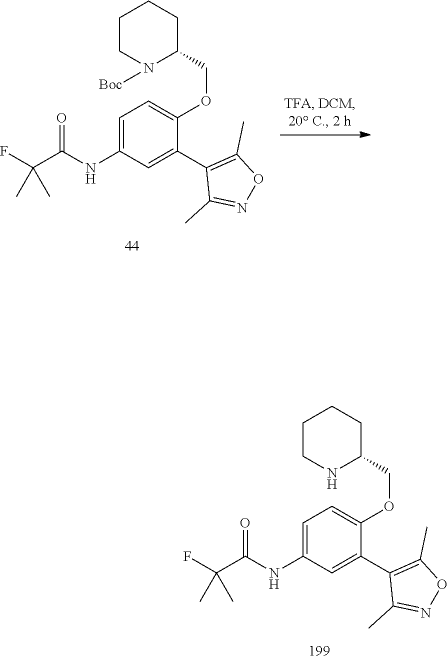 [SMILES: Cc1noc(C)c1-c1cc(NC(=O)C(C)(C)F)ccc1OC[C@H]1CCCCN1C(=O)OC(C)(C)C ; Cc1noc(C)c1-c1cc(NC(=O)C(C)(C)F)ccc1OC[C@H]1CCCCN1]

To a solution of tert-butyl (2R)-2-[[2-(3,5-dimethylisoxazol-4-yl)-4-[(2-fluoro-2-methyl-propanoyl)amino]phenoxy]methyl]piperidine-1-carboxylate ((44), 70 mg, 142.98 μmol, 1 eq.) in DCM (1 mL) was added TFA (1 mL). The mixture was stirred at 20° C. for 2 hrs. The mixture was concentrated under vacuum. The residue was purified by prep-HPLC (TFA conditions, column: Phenomenex Gemini-NX C18 75*30 mm*3 μm; mobile phase: [water (0.1% TFA)-ACN]; B %: 25%-35%, 7 min). N-[3-(3,5-dimethylisoxazol-4-yl)-4-[[(2R)-2-piperidyl]methoxy]phenyl]-2-fluoro-2-methyl-propanamide (36.57 mg, 72.63 μmol, 50.80% yield, 100% purity, TFA) was obtained as a white solid.

LCMS (ESI): m/z [M+H] calcd for C₂₁H₂₈FN₃O₃: 390.0, found: 390.2.

¹H NMR (400 MHZ, methanol-d₄) δ=7.72-7.65 (m, 1H), 7.51-7.45 (m, 1H), 7.18 (d, J=8.9 Hz, 1H), 4.13-4.00 (m, 2H), 3.50-3.35 (m, 2H), 3.03 (br t, J=12.6 Hz, 1H), 2.34 (s, 3H), 2.17 (s, 3H), 1.99-1.85 (m, 3H), 1.67-1.49 (m, 9H).

### (R)—N-(3-(3,5-dimethylisoxazol-4-yl)-4-(piperidin-2-ylmethoxy)phenyl)-1-(fluoromethyl)cyclopropane-1-carboxamide (200)

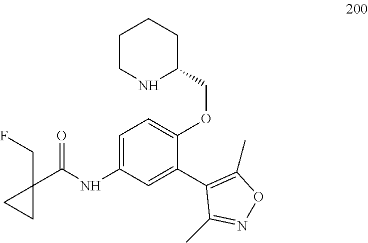 [SMILES: Cc1noc(C)c1-c1cc(NC(=O)C2(CF)CC2)ccc1OC[C@H]1CCCCN1]

Compound 200 was prepared according to the synthesis described for compound 199, substituting 1-(fluoromethyl)cyclopropane-1-carboxylic acid for 2-fluoro-2-methylpropanoic acid.

LCMS (ESI): m/z [M+H] calcd for C₂₂H₂₉FN₃O₃: 402.0, found: 402.0.

¹H NMR (400 MHZ, methanol-d₄) δ=7.57 (br dd, J=2.6, 8.9 Hz, 1H), 7.40 (d, J=2.6 Hz, 1H), 7.17 (d, J=9.0 Hz, 1H), 4.70 (s, 1H), 4.58 (s, 1H), 4.12-4.01 (m, 2H), 3.44 (br d, J=5.1 Hz, 1H), 3.37 (br d, J=12.8 Hz, 1H), 3.02 (br t, J=11.9 Hz, 1H), 2.33 (s, 3H), 2.17 (s, 3H), 1.91 (br d, J=9.9 Hz, 3H), 1.73-1.46 (m, 3H), 1.39-1.27 (m, 2H), 1.03-0.95 (m, 2H).

### N-(3-(3,5-dimethylisoxazol-4-yl)-4-(((R)-piperidin-2-yl)methoxy)phenyl)-2-((S)-tetrahydrofuran-3-yl)acetamide (201)

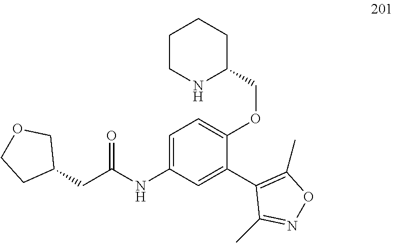 [SMILES: Cc1noc(C)c1-c1cc(NC(=O)C[C@@H]2CCOC2)ccc1OC[C@H]1CCCCN1]

Compound 201 was prepared according to the synthesis described for compound 199, substituting (S)-2-(tetrahydrofuran-3-yl)acetic acid for 2-fluoro-2-methylpropanoic acid.

LCMS (ESI): m/z [M+H] calcd for C₂₃H₃₂N₃O₄: 414.0, found: 414.3.

¹H NMR (400 MHZ, methanol-d₄) δ=7.66-7.61 (m, 1H), 7.42-7.41 (d, J=2.8 Hz, 1H), 7.17-7.15 (d, J=8.8 Hz, 1H), 4.15-4.05 (m, 2H), 3.94-3.93 (m, 2H), 3.78 (q, J=8.0 Hz, 1H), 3.48-3.45 (m, 2H), 3.40-3.34 (m, 1H), 3.09-2.97 (m, 1H), 2.69-2.68 (m, 1H), 2.48-2.46 (m, 2H), 2.33 (s, 3H), 2.16 (s, 3H), 2.16-2.13 (m, 1H), 1.92-1-90 (m, 3H), 1.68-1.63 (m, 3H), 1.61-1.52 (m, 1H).

### N-(3-((E)-2-(aminooxy)pent-2-en-3-yl)-4-(((R)-piperidin-2-yl)methoxy)phenyl)-2-((R)-tetrahydrofuran-3-yl)acetamide (202)

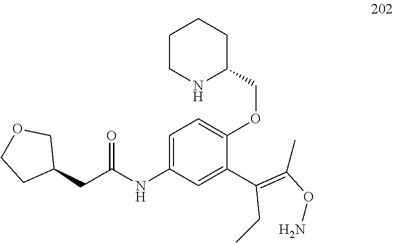 [SMILES: CC/C(=C(/C)ON)c1cc(NC(=O)C[C@H]2CCOC2)ccc1OC[C@H]1CCCCN1]

Compound 202 was prepared according to the synthesis described for compound 199, substituting (R)-2-(tetrahydrofuran-3-yl)acetic acid for 2-fluoro-2-methylpropanoic acid.

LCMS (ESI): m/z [M+H] calcd for C₂₃H₃₂N₃O₄: 414.0, found: 414.3.

¹H NMR (400 MHZ, methanol-d₄) δ=7.66-7.57 (m, 1H), 7.46-7.37 (m, 1H), 7.16 (d, J=9.0 Hz, 1H), 4.11-3.98 (m, 2H), 3.97-3.82 (m, 2H), 3.77 (q, J=7.5 Hz, 1H), 3.53-3.40 (m, 2H), 3.40-3.34 (m, 1H), 3.09-2.97 (m, 1H), 2.69 (td, J=7.4, 14.3 Hz, 1H), 2.47 (dd, J=1.9, 7.5 Hz, 2H), 2.33 (s, 3H), 2.17 (s, 3H), 2.16-2.09 (m, 1H), 1.92 (br d, J=10.5 Hz, 3H), 1.71-1.57 (m, 3H), 1.57-1.46 (m, 1H).

### N-(3-(3,5-dimethylisoxazol-4-yl)-4-(((R)-piperidin-2-yl)methoxy)phenyl)-6-oxaspiro[2.5]octane-1-carboxamide (203)

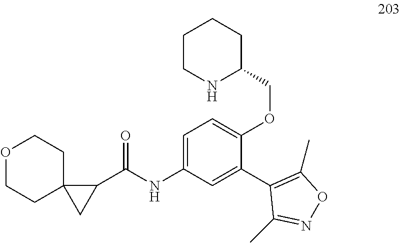 [SMILES: Cc1noc(C)c1-c1cc(NC(=O)C2CC23CCOCC3)ccc1OC[C@H]1CCCCN1]

Compound 203 was prepared according to the synthesis described for compound 199, substituting 6-oxaspiro[2.5]octane-1-carboxylic acid for 2-fluoro-2-methylpropanoic acid.

LCMS (ESI): m/z [M+H] calcd for C₂₅H₃₄N₃O₄: 440.0, found: 440.3.

¹H NMR (400 MHZ, methanol-d₄) δ=7.60 (br dd, J=2.8, 5.9 Hz, 1H), 7.45-7.37 (m, 1H), 7.16 (d, J=9.0 Hz, 1H), 4.12-3.95 (m, 2H), 3.86-3.73 (m, 2H), 3.73-3.55 (m, 2H), 3.51-3.34 (m, 2H), 3.08-2.97 (m, 1H), 2.33 (s, 3H), 2.17 (s, 3H), 1.91 (br d, J=11.1 Hz, 3H), 1.76 (t, J=5.3 Hz, 2H), 1.71 (dd, J=5.6, 7.7 Hz, 1H), 1.69-1.57 (m, 2H), 1.57-1.46 (m, 3H), 1.22 (t, J=4.9 Hz, 1H), 0.95 (dd, J=4.4, 7.9 Hz, 1H).

### (R)—N-(3-(3,5-dimethylisoxazol-4-yl)-4-(piperidin-2-ylmethoxy)phenyl)-1-(methoxymethyl)cyclopropane-1-carboxamide (204)

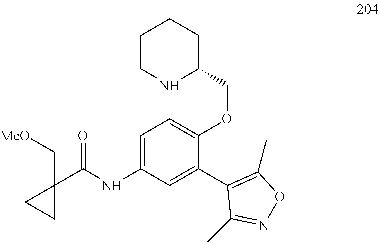 [SMILES: COCC1(C(=O)Nc2ccc(OC[C@H]3CCCCN3)c(-c3c(C)noc3C)c2)CC1]

Compound 204 was prepared according to the synthesis described for compound 199, substituting 1-(methoxymethyl)cyclopropane-1-carboxylic acid for 2-fluoro-2-methylpropanoic acid.

LCMS (ESI): m/z [M+H] calcd for C₂₂H₂₉N₃O₃: 414.0, found: 414.3.

¹H NMR (400 MHZ, methanol-d₄) δ=7.57-7.49 (m, 1H), 7.43-7.38 (m, 1H), 7.17 (d, J=8.9 Hz, 1H), 4.11-3.99 (m, 2H), 3.61 (s, 2H), 3.46 (s, 3H), 3.37 (br d, J=13.0 Hz, 2H), 3.09-2.97 (m, 1H), 2.33 (s, 3H), 2.17 (s, 3H), 1.97-1.85 (m, 3H), 1.70-1.46 (m, 3H), 1.28-1.18 (m, 2H), 0.89-0.80 (m, 2H).

### (R)—N-(3-(3,5-dimethylisoxazol-4-yl)-4-(piperidin-2-ylmethoxy)phenyl)-2-(1-methoxycyclobutyl)acetamide (205)

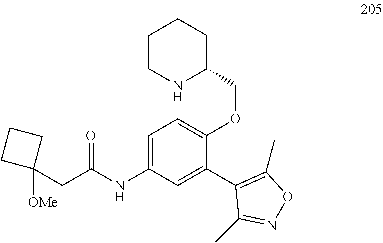 [SMILES: COC1(CC(=O)Nc2ccc(OC[C@H]3CCCCN3)c(-c3c(C)noc3C)c2)CCC1]

Compound 205 was prepared according to the synthesis described for compound 199, substituting 2-(1-methoxycyclobutyl)acetic acid for 2-fluoro-2-methylpropanoic acid.

LCMS (ESI): m/z [M+H] calcd for C₂₄H₃₄N₃O₄: 428.0, found: 428.3.

¹H NMR (400 MHZ, methanol-d₄) δ=7.63-7.54 (m, 1H), 7.43 (br s, 1H), 7.16 (d, J=9.0 Hz, 1H), 4.12-3.99 (m, 2H), 3.49-3.41 (m, 1H), 3.37 (br d, J=12.8 Hz, 1H), 3.28 (s, 3H), 3.09-2.97 (m, 1H), 2.73 (s, 2H), 2.33 (s, 3H), 2.31-2.21 (m, 2H), 2.19-2.10 (m, 5H), 1.91 (br d, J=11.3 Hz, 3H), 1.84-1.48 (m, 5H).

### N-(3-(3,5-dimethylisoxazol-4-yl)-4-(((R)-piperidin-2-yl)methoxy)phenyl)-2-fluoro-2-phenylacetamide (206)

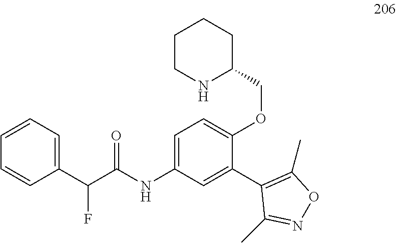 [SMILES: Cc1noc(C)c1-c1cc(NC(=O)C(F)c2ccccc2)ccc1OC[C@H]1CCCCN1]

Compound 206 was prepared according to the synthesis described for compound 199, substituting 2-fluoro-2-phenylacetic acid for 2-fluoro-2-methylpropanoic acid.

LCMS (ESI): m/z [M+H] calcd for C₂₅H₂₉N₃O₃: 438.0, found: 438.2.

¹H NMR (400 MHz, methanol-d₄) δ=7.62 (dd, J=2.8, 9.0 Hz, 1H), 7.58-7.51 (m, 2H), 7.51-7.38 (m, 4H), 7.11 (d, J=8.9 Hz, 1H), 5.99-5.84 (m, 1H), 3.97-3.79 (m, 2H), 3.00 (br d, J=11.6 Hz, 1H), 2.93-2.75 (m, 1H), 2.66-2.52 (m, 1H), 2.30 (s, 3H), 2.15 (s, 3H), 1.87-1.76 (m, 1H), 1.71-1.58 (m, 2H), 1.49-1.29 (m, 2H), 1.29-1.08 (m, 1H).

### (R)—N-(3-(3,5-dimethylisoxazol-4-yl)-4-(piperidin-2-ylmethoxy)phenyl)-2,4-dimethyloxazole-5-carboxamide (207)

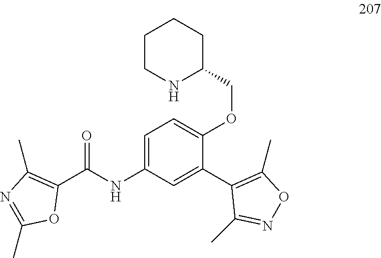 [SMILES: Cc1nc(C)c(C(=O)Nc2ccc(OC[C@H]3CCCCN3)c(-c3c(C)noc3C)c2)o1]

Compound 207 was prepared according to the synthesis described for compound 199, substituting 2,4-dimethyloxazole-5-carboxylic acid for 2-fluoro-2-methylpropanoic acid.

LCMS (ESI): m/z [M+H] calcd for C₂₃H₂₉N₄O₄: 425.0, found: 425.3.

¹H NMR (400 MHZ, methanol-d₄) δ=7.74 (td, J=2.1, 8.8 Hz, 1H), 7.55 (d, J=1.5 Hz, 1H), 7.21 (d, J=9.0 Hz, 1H), 4.13-4.05 (m, 2H), 3.52-3.42 (m, 1H), 3.38 (br d, J=12.5 Hz, 1H), 3.09-2.99 (m, 1H), 2.53 (s, 3H), 2.45 (s, 3H), 2.35 (s, 3H), 2.19 (s, 3H), 1.98-1.86 (m, 3H), 1.70-1.47 (m, 3H).

### (R)—N-(3-(3,5-dimethylisoxazol-4-yl)-4-(piperidin-2-ylmethoxy)phenyl)-1,4-dimethyl-1H-pyrazole-5-carboxamide (208)

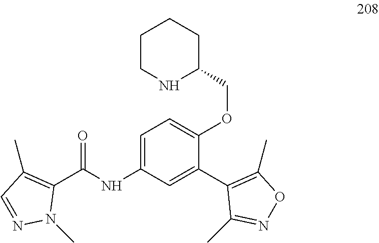 [SMILES: Cc1cnn(C)c1C(=O)Nc1ccc(OC[C@H]2CCCCN2)c(-c2c(C)noc2C)c1]

Compound 208 was prepared according to the synthesis described for compound 199, substituting 1,4-dimethyl-1H-pyrazole-5-carboxylic acid for 2-fluoro-2-methylpropanoic acid.

LCMS (ESI): m/z [M+H] calcd for C₂₃H₃₀N₅O₃: 424.0, found: 424.3.

¹H NMR (400 MHZ, methanol-d₄) δ=7.73 (br d, J=6.9 Hz, 1H), 7.55 (br s, 1H), 7.34 (s, 1H), 7.23 (d, J=9.0 Hz, 1H), 4.14-4.04 (m, 2H), 3.96 (s, 3H), 3.51-3.43 (m, 1H), 3.38 (br d, J=12.5 Hz, 1H), 3.09-2.99 (m, 1H), 2.35 (s, 3H), 2.23 (s, 3H), 2.19 (s, 3H), 2.00-1.85 (m, 3H), 1.73-1.47 (m, 3H).

### (R)—N-(3-(3,5-dimethylisoxazol-4-yl)-4-(piperidin-2-ylmethoxy)phenyl)-3-ethyl-1-methyl-1H-pyrazole-5-carboxamide (209)

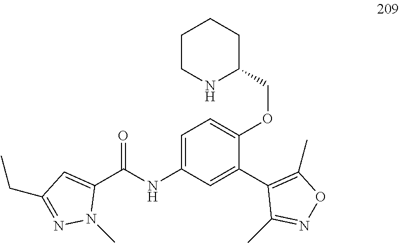 [SMILES: CCc1cc(C(=O)Nc2ccc(OC[C@H]3CCCCN3)c(-c3c(C)noc3C)c2)n(C)n1]

Compound 209 was prepared according to the synthesis described for compound 199, substituting 3-ethyl-1-methyl-1H-pyrazole-5-carboxylic acid for 2-fluoro-2-methylpropanoic acid.

LCMS (ESI): m/z [M+H] calcd for C₂₄H₃₂N₅O₃: 438.0, found: 438.1.

¹H NMR (400 MHZ, methanol-d₄) δ=7.75 (td, J=1.2, 8.9 Hz, 1H), 7.54 (d, J=2.4 Hz, 1H), 7.21 (d, J=9.0 Hz, 1H), 6.78 (s, 1H), 4.14-4.03 (m, 5H), 3.57-3.41 (m, 1H), 3.38 (br d, J=13.1 Hz, 1H), 3.11-2.97 (m, 1H), 2.65 (q, J=7.7 Hz, 2H), 2.35 (s, 3H), 2.19 (s, 3H), 1.99-1.87 (m, 3H), 1.72-1.47 (m, 3H), 1.27 (t, J=7.6 Hz, 3H).

### (R)—N-(3-(3,5-dimethylisoxazol-4-yl)-4-(piperidin-2-ylmethoxy)phenyl)-2,5-dimethylthiazole-4-carboxamide (210)

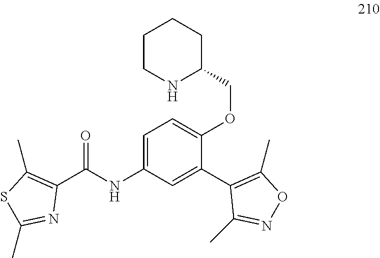 [SMILES: Cc1nc(C(=O)Nc2ccc(OC[C@H]3CCCCN3)c(-c3c(C)noc3C)c2)c(C)s1]

Compound 210 was prepared according to the synthesis described for compound 199, substituting 2,5-dimethylthiazole-4-carboxylic acid for 2-fluoro-2-methylpropanoic acid.

LCMS (ESI): m/z [M+H] calcd for C₂₃H₂₉N₄O₃S: 441.0, found: 441.0.

¹H NMR (400 MHZ, methanol-d₄) δ=7.81-7.73 (m, 1H), 7.60 (d, J=2.6 Hz, 1H), 7.21 (d, J=8.9 Hz, 1H), 4.12-4.01 (m, 2H), 3.51-3.42 (m, 1H), 3.38 (br d, J=12.3 Hz, 1H), 3.04 (br t, J=12.1 Hz, 1H), 2.77 (s, 3H), 2.67 (s, 3H), 2.36 (s, 3H), 2.20 (s, 3H), 1.98-1.87 (m, 3H), 1.73-1.47 (m, 3H).

### (R)—N-(3-(3,5-dimethylisoxazol-4-yl)-4-(piperidin-2-ylmethoxy)phenyl)-1,5-dimethyl-1H-pyrazole-4-carboxamide (211)

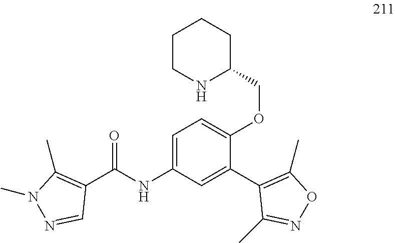 [SMILES: Cc1noc(C)c1-c1cc(NC(=O)c2cnn(C)c2C)ccc1OC[C@H]1CCCCN1]

Compound 211 was prepared according to the synthesis described for compound 199, substituting 1,5-dimethyl-1H-pyrazole-4-carboxylic acid for 2-fluoro-2-methylpropanoic acid.

¹H NMR (400 MHZ, methanol-d₄) δ=7.97 (s, 1H), 7.77-7.64 (m, 1H), 7.56-7.45 (m, 1H), 7.19 (d, J=9.0 Hz, 1H), 4.13-3.98 (m, 2H), 3.83 (s, 3H), 3.53-3.42 (m, 1H), 3.38 (br d, J=13.0 Hz, 1H), 3.04 (br t, J=12.0 Hz, 1H), 2.58 (s, 3H), 2.35 (s, 3H), 2.19 (s, 3H), 1.92 (br d, J=10.1 Hz, 3H), 1.74-1.45 (m, 3H).

LCMS (ESI): m/z [M+H] calcd for C₂₃H₃₀N₅O₃: 424.0, found: 424.3.

### (R)—N-(3-(3,5-dimethylisoxazol-4-yl)-4-(piperidin-2-ylmethoxy)phenyl)-1-methyl-1H-1,2,4-triazole-5-carboxamide (212)

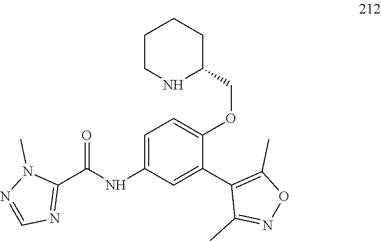 [SMILES: Cc1noc(C)c1-c1cc(NC(=O)c2ncnn2C)ccc1OC[C@H]1CCCCN1]

Compound 212 was prepared according to the synthesis described for compound 199, substituting 1-methyl-1H-1,2,4-triazole-5-carboxylic acid for 2-fluoro-2-methylpropanoic acid.

¹H NMR (400 MHZ, methanol-d₄) δ=7.99 (s, 1H), 7.81 (br dd, J=1.9, 7.0 Hz, 1H), 7.64 (d, J=2.7 Hz, 1H), 7.23 (d, J=9.0 Hz, 1H), 4.26 (s, 3H), 4.16-4.03 (m, 2H), 3.54-3.43 (m, 1H), 3.38 (br d, J=13.0 Hz, 1H), 3.09-2.98 (m, 1H), 2.36 (s, 3H), 2.19 (s, 3H), 2.00-1.86 (m, 3H), 1.73-1.45 (m, 3H).

LCMS (ESI): m/z [M+H] calcd for C₂₁H₂₇N₆O₃: 411.0, found: 411.0.

### (R)—N-(3-(3,5-dimethylisoxazol-4-yl)-4-(piperidin-2-ylmethoxy)phenyl)-2-(thiazol-2-yl)acetamide (213)

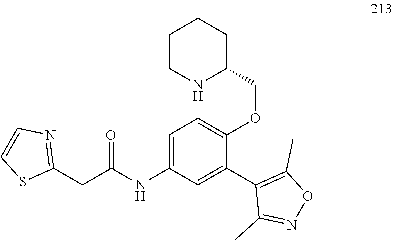 [SMILES: Cc1noc(C)c1-c1cc(NC(=O)Cc2nccs2)ccc1OC[C@H]1CCCCN1]

Compound 213 was prepared according to the synthesis described for compound 199, substituting 2-(thiazol-2-yl)acetic acid for 2-fluoro-2-methylpropanoic acid.

LCMS (ESI): m/z [M+H] calcd for C₂₂H₂₆N₄O₃S: 427.0, found: 427.0.

¹H NMR (400 MHZ, methanol-d₄) δ=7.82-7.74 (m, 1H), 7.67-7.60 (m, 1H), 7.60-7.55 (m, 1H), 7.50-7.43 (m, 1H), 7.18 (d, J=8.8 Hz, 1H), 4.89 (br s, 2H), 4.12-3.99 (m, 2H), 3.52-3.41 (m, 1H), 3.41-3.34 (m, 1H), 3.09-2.98 (m, 1H), 2.33 (s, 3H), 2.17 (s, 3H), 1.91-1.88 (m, 1H), 1.92 (br d, J=9.7 Hz, 3H), 1.72-1.45 (m, 3H).

### (R)—N-(3-(3,5-dimethylisoxazol-4-yl)-4-(piperidin-2-ylmethoxy)phenyl)-4-methylisothiazole-5-carboxamide (214)

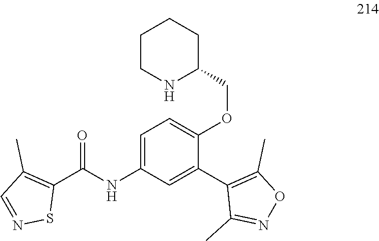 [SMILES: Cc1cnsc1C(=O)Nc1ccc(OC[C@H]2CCCCN2)c(-c2c(C)noc2C)c1]

Compound 214 was prepared according to the synthesis described for compound 199, substituting 4-methylisothiazole-5-carboxylic acid for 2-fluoro-2-methylpropanoic acid.

LCMS (ESI): m/z [M+H] calcd for C₂₂H₂₆N₄O₃S: 427.0, found: 427.0.

¹H NMR (400 MHz, methanol-d₄) δ=8.38 (s, 1H), 7.78-7.69 (m, 1H), 7.53 (br s, 1H), 7.23 (d, J=9.0 Hz, 1H), 4.18-4.02 (m, 2H), 3.54-3.42 (m, 1H), 3.38 (br d, J=13.0 Hz, 1H), 3.04 (br t, J=11.8 Hz, 1H), 2.50 (s, 3H), 2.35 (s, 3H), 2.19 (s, 3H), 1.99-1.86 (m, 3H), 1.73-1.47 (m, 3H).

### (R)—N-(3-(3,5-dimethylisoxazol-4-yl)-4-(piperidin-2-ylmethoxy)phenyl)-4-methyl-1,2,3-thiadiazole-5-carboxamide (215)

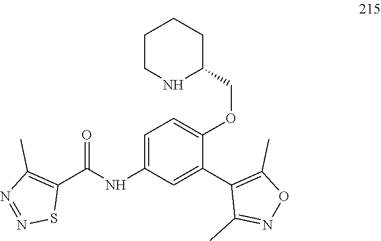 [SMILES: Cc1nnsc1C(=O)Nc1ccc(OC[C@H]2CCCCN2)c(-c2c(C)noc2C)c1]

Compound 215 was prepared according to the synthesis described for compound 199, substituting 4-methyl-1,2,3-thiadiazole-5-carboxylic acid for 2-fluoro-2-methylpropanoic acid.

LCMS (ESI): m/z [M+H] calcd for C₂₁H₂₆N₅O₃S₂: 428.0, found: 428.2.

¹H NMR (400 MHZ, methanol-d₄) δ=7.80-7.69 (m, 1H), 7.54 (br s, 1H), 7.24 (d, J=8.9 Hz, 1H), 4.16-4.05 (m, 2H), 3.53-3.35 (m, 2H), 3.09-2.98 (m, 1H), 2.87 (s, 3H), 2.35 (s, 3H), 2.19 (s, 3H), 2.00-1.86 (m, 3H), 1.70-1.47 (m, 3H).

### (R)—N-(3-(3,5-dimethylisoxazol-4-yl)-4-(piperidin-2-ylmethoxy)phenyl)-2,5-dimethyloxazole-4-carboxamide (216)

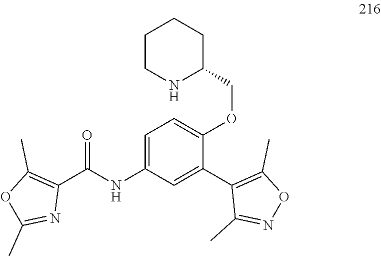 [SMILES: Cc1nc(C(=O)Nc2ccc(OC[C@H]3CCCCN3)c(-c3c(C)noc3C)c2)c(C)o1]

Compound 216 was prepared according to the synthesis described for compound 199, substituting 2,5-dimethyloxazole-4-carboxylic acid for 2-fluoro-2-methylpropanoic acid.

LCMS (ESI): m/z [M+H] calcd for C₂₃H₂₉N₄O₄: 425.0, found: 425.3.

¹H NMR (400 MHZ, methanol-d₄) δ=7.74 (dd, J=2.7, 8.9 Hz, 1H), 7.58 (d, J=2.6 Hz, 1H), 7.20 (d, J=8.9 Hz, 1H), 4.13-4.01 (m, 2H), 3.52-3.42 (m, 1H), 3.42-3.34 (m, 1H), 3.04 (br t, J=12.5 Hz, 1H), 2.61 (s, 3H), 2.45 (s, 3H), 2.35 (s, 3H), 2.19 (s, 3H), 1.98-1.88 (m, 3H), 1.72-1.47 (m, 3H).

### (R)—N-(3-(3,5-dimethylisoxazol-4-yl)-4-(piperidin-2-ylmethoxy)phenyl)-1-fluorocyclopropane-1-carboxamide (217)

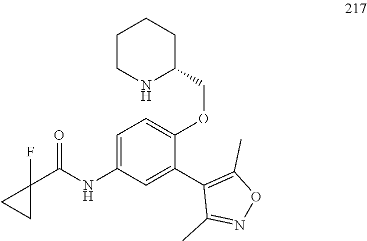 [SMILES: Cc1noc(C)c1-c1cc(NC(=O)C2(F)CC2)ccc1OC[C@H]1CCCCN1]

Compound 217 was prepared according to the synthesis described for compound 199, substituting 1-fluorocyclopropane-1-carboxylic acid for 2-fluoro-2-methylpropanoic acid.

LCMS (ESI): m/z [M+H] calcd for C₂₁H₂₆FN₃O₃: 388.20; found: 388.2.

¹H NMR (400 MHZ, methanol-d₄) δ=10.07 (br s, 1H), 7.71-7.64 (m, 1H), 7.49 (t, J=2.8 Hz, 1H), 7.19 (d, J=9.0 Hz, 1H), 4.13-4.00 (m, 2H), 3.46 (br dd, J=4.5, 6.9 Hz, 1H), 3.38 (br d, J=12.9 Hz, 1H), 3.09-2.95 (m, 1H), 2.34 (s, 3H), 2.17 (s, 3H), 1.98-1.83 (m, 3H), 1.75-1.47 (m, 3H), 1.44-1.31 (m, 4H).

### (R)—N-(3-(3,5-dimethylisoxazol-4-yl)-4-(piperidin-2-ylmethoxy)phenyl)benzamide (218)

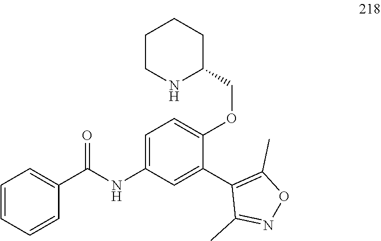 [SMILES: Cc1noc(C)c1-c1cc(NC(=O)c2ccccc2)ccc1OC[C@H]1CCCCN1]

Compound 218 was prepared according to the synthesis described for compound 199, substituting benzoic acid for 2-fluoro-2-methylpropanoic acid.

LCMS (ESI): m/z [M+H] calcd for C₂₄H₂₈N₃O₃: 406.0, found: 406.2.

¹H NMR (400 MHZ, methanol-d₄) δ=8.04-7.84 (m, 2H), 7.83-7.72 (m, 1H), 7.69-7.38 (m, 4H), 7.22 (d, J=9.0 Hz, 1H), 4.19-3.96 (m, 2H), 3.57-3.35 (m, 2H), 3.04 (br t, J=12.2 Hz, 1H), 2.36 (s, 3H), 2.20 (s, 3H), 1.99-1.83 (m, 3H), 1.78-1.47 (m, 3H).

### (R)—N-(3-(3,5-dimethylisoxazol-4-yl)-4-(piperidin-2-ylmethoxy)phenyl)-4-fluorobenzamide (219)

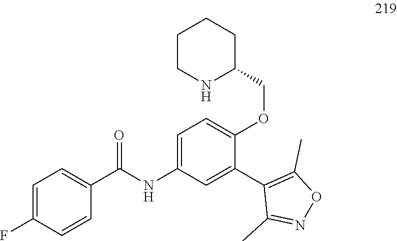 [SMILES: Cc1noc(C)c1-c1cc(NC(=O)c2ccc(F)cc2)ccc1OC[C@H]1CCCCN1]

Compound 219 was prepared according to the synthesis described for compound 199, substituting 4-fluorobenzoic acid for 2-fluoro-2-methylpropanoic acid.

LCMS (ESI): m/z [M+H] calcd for C₂₅H₃₁FN₃O₃: 424.0, found: 424.2.

¹H NMR (400 MHZ, methanol-d₄) δ=8.11-7.92 (m, 2H), 7.81-7.69 (m, 1H), 7.56 (d, J=2.5 Hz, 1H), 7.31-7.13 (m, 3H), 4.18-3.99 (m, 2H), 3.54-3.34 (m, 2H), 3.04 (br t, J=12.4 Hz, 1H), 2.36 (s, 3H), 2.20 (s, 3H), 1.99-1.81 (m, 3H), 1.76-1.39 (m, 3H).

### (R)—N-(3-(3,5-dimethylisoxazol-4-yl)-4-(piperidin-2-ylmethoxy)phenyl)-3-methoxybenzamide (220)

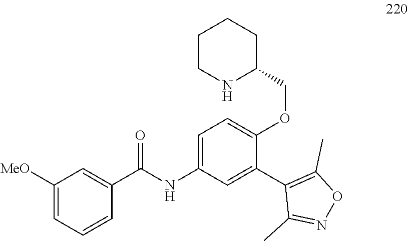 [SMILES: COc1cccc(C(=O)Nc2ccc(OC[C@H]3CCCCN3)c(-c3c(C)noc3C)c2)c1]

Compound 220 was prepared according to the synthesis described for compound 199, substituting 3-methoxybenzoic acid for 2-fluoro-2-methylpropanoic acid.

LCMS (ESI): m/z [M+H] calcd for C₂₅H₂₉N₃O₄: 436.0, found: 436.2.

¹H NMR (400 MHZ, methanol-d₄) δ=7.77 (dd, J=2.7, 8.9 Hz, 1H), 7.57 (d, J=2.6 Hz, 1H), 7.53-7.45 (m, 2H), 7.42 (t, J=7.9 Hz, 1H), 7.22 (d, J=8.9 Hz, 1H), 7.15 (ddd, J=0.9, 2.6, 8.2 Hz, 1H), 4.15-4.03 (m, 2H), 3.87 (s, 3H), 3.54-3.35 (m, 2H), 3.12-2.97 (m, 1H), 2.36 (s, 3H), 2.20 (s, 3H), 2.01-1.86 (m, 3H), 1.74-1.44 (m, 3H).

### (R)—N-(3-(3,5-dimethylisoxazol-4-yl)-4-(piperidin-2-ylmethoxy)phenyl)-2-phenylacetamide (221)

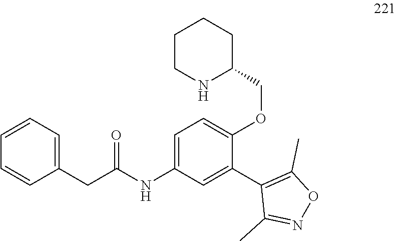 [SMILES: Cc1noc(C)c1-c1cc(NC(=O)Cc2ccccc2)ccc1OC[C@H]1CCCCN1]

Compound 221 was prepared according to the synthesis described for compound 199, substituting 2-phenylacetic acid for 2-fluoro-2-methylpropanoic acid.

LCMS (ESI): m/z [M+H] calcd for C₂₆H₃₄N₃O₃: 420.0, found: 420.3.

¹H NMR (400 MHZ, methanol-d₄) δ=7.67-7.54 (m, 1H), 7.44 (t, J=3.1 Hz, 1H), 7.40-7.20 (m, 5H), 7.16 (d, J=8.9 Hz, 1H), 4.13-3.95 (m, 2H), 3.67 (s, 2H), 3.49-3.34 (m, 2H), 3.02 (br t, J=12.0 Hz, 1H), 2.32 (s, 3H), 2.16 (s, 3H), 1.91 (br d, J=11.3 Hz, 3H), 1.69-1.44 (m, 3H).

### (R)—N-(3-(3,5-dimethylisoxazol-4-yl)-4-(piperidin-2-ylmethoxy)phenyl)-2-(3-methoxyphenyl)acetamide (222)

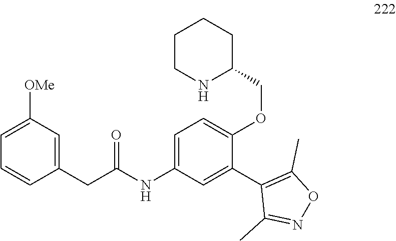 [SMILES: COc1cccc(CC(=O)Nc2ccc(OC[C@H]3CCCCN3)c(-c3c(C)noc3C)c2)c1]

Compound 222 was prepared according to the synthesis described for compound 199, substituting 2-(3-methoxyphenyl)acetic acid for 2-fluoro-2-methylpropanoic acid.

LCMS (ESI): m/z [M+H] calcd for C₂₆H₃₂N₃O₄: 450.0, found: 450.2.

¹H NMR (400 MHZ, methanol-d₄) δ=7.65-7.57 (m, 1H), 7.47-7.39 (m, 1H), 7.27-7.19 (m, 1H), 7.16 (d, J=8.9 Hz, 1H), 6.98-6.88 (m, 2H), 6.88-6.73 (m, 1H), 4.10-3.98 (m, 2H), 3.79 (s, 3H), 3.64 (s, 2H), 3.48-3.34 (m, 2H), 3.09-2.96 (m, 1H), 2.32 (s, 3H), 2.16 (s, 3H), 1.91 (br d, J=11.0 Hz, 3H), 1.69-1.47 (m, 3H).

### (R)—N-(3-(3,5-dimethylisoxazol-4-yl)-4-(piperidin-2-ylmethoxy)phenyl)-2-(3-fluorophenyl)acetamide (223)

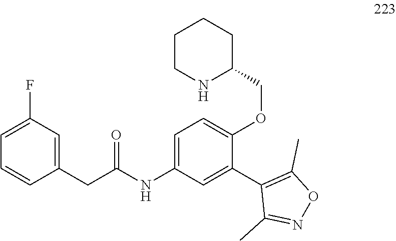 [SMILES: Cc1noc(C)c1-c1cc(NC(=O)Cc2cccc(F)c2)ccc1OC[C@H]1CCCCN1]

Compound 223 was prepared according to the synthesis described for compound 199, substituting 2-(3-fluorophenyl)acetic acid for 2-fluoro-2-methylpropanoic acid.

LCMS (ESI): m/z [M+H] calcd for C₂₅H₂₉FN₃O₃: 438.0, found: 438.2.

¹H NMR (400 MHZ, methanol-d₄) δ=7.61 (dd, J=2.6, 8.9 Hz, 1H), 7.50-7.40 (m, 1H), 7.34 (dt, J=6.1, 8.0 Hz, 1H), 7.16 (d, J=8.9 Hz, 2H), 7.13-7.06 (m, 1H), 7.00 (dt, J=2.1, 8.6 Hz, 1H), 4.11-3.98 (m, 2H), 3.70 (s, 2H), 3.48-3.34 (m, 2H), 3.09-2.95 (m, 1H), 2.32 (s, 3H), 2.16 (s, 3H), 1.91 (br d, J=11.0 Hz, 3H), 1.68-1.46 (m, 3H).

### (R)—N-(3-(3,5-dimethylisoxazol-4-yl)-4-(piperidin-2-ylmethoxy)phenyl)-2-(4-methoxyphenyl)acetamide (224)

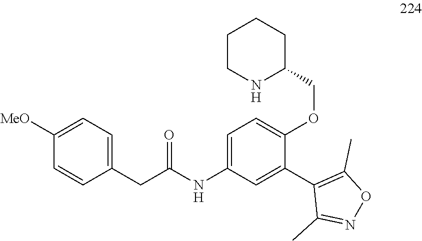 [SMILES: COc1ccc(CC(=O)Nc2ccc(OC[C@H]3CCCCN3)c(-c3c(C)noc3C)c2)cc1]

Compound 224 was prepared according to the synthesis described for compound 199, substituting 2-(4-methoxyphenyl)acetic acid for 2-fluoro-2-methylpropanoic acid.

LCMS (ESI): m/z [M+H] calcd for C₂₆H₃₂N₃O₄: 450.0, found: 450.3.

¹H NMR (400 MHZ, methanol-d₄) δ=7.62 (dd, J=2.4, 8.5 Hz, 1H), 7.42 (d, J=2.8 Hz, 1H), 7.26 (d, J=8.6 Hz, 2H), 7.15 (d, J=9.0 Hz, 1H), 6.92-6.85 (m, 2H), 4.09-3.98 (m, 2H), 3.77 (s, 3H), 3.60 (s, 2H), 3.51-3.36 (m, 2H), 3.07-2.97 (m, 1H), 2.32 (s, 3H), 2.16 (s, 3H), 1.91 (br d, J=10.4 Hz, 3H), 1.68-1.46 (m, 3H).

### (R)—N-(3-(3,5-dimethylisoxazol-4-yl)-4-(piperidin-2-ylmethoxy)phenyl)-2-(4-methylthiazol-5-yl)acetamide (225)

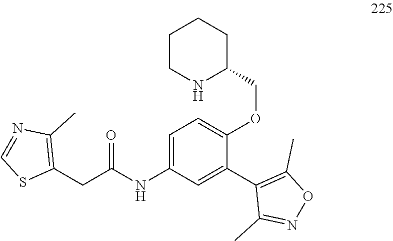 [SMILES: Cc1ncsc1CC(=O)Nc1ccc(OC[C@H]2CCCCN2)c(-c2c(C)noc2C)c1]

Compound 225 was prepared according to the synthesis described for compound 199, substituting 2-(4-methylthiazol-5-yl)acetic acid for 2-fluoro-2-methylpropanoic acid.

LCMS (ESI): m/z [M+H] calcd for C₂₃H₂₉N₄O₃S: 441.0, found: 441.2.

¹H NMR (400 MHZ, methanol-d₄) δ=9.22-8.74 (m, 1H), 7.61 (dd, J=2.6, 8.9 Hz, 1H), 7.46 (br d, J=2.3 Hz, 1H), 7.18 (d, J=8.9 Hz, 1H), 4.07 (br d, J=3.9 Hz, 2H), 3.97 (br s, 2H), 3.52-3.35 (m, 2H), 3.13-2.95 (m, 1H), 2.47 (s, 3H), 2.32 (s, 3H), 2.16 (s, 3H), 1.91 (br d, J=10.4 Hz, 3H), 1.73-1.40 (m, 3H).

### (R)—N-(3-(3,5-dimethylisoxazol-4-yl)-4-(piperidin-2-ylmethoxy)phenyl)-2-(5-fluoropyridin-3-yl)acetamide (226)

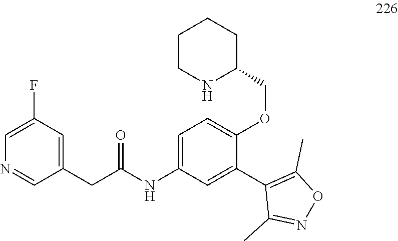 [SMILES: Cc1noc(C)c1-c1cc(NC(=O)Cc2cncc(F)c2)ccc1OC[C@H]1CCCCN1]

Compound 226 was prepared according to the synthesis described for compound 199, substituting 2-(5-fluoropyridin-3-yl)acetic acid for 2-fluoro-2-methylpropanoic acid.

LCMS (ESI): m/z [M+H] calcd for C₂₄H₂₈FN₄O₃: 439.0, found: 439.2.

¹H NMR (400 MHz, methanol-d₄) δ=8.40 (d, J=2.8 Hz, 2H), 7.77-7.55 (m, 2H), 7.44 (br d, J=2.4 Hz, 1H), 7.17 (d, J=8.9 Hz, 1H), 4.10-3.99 (m, 2H), 3.81 (s, 2H), 3.49-3.34 (m, 2H), 3.11-2.98 (m, 1H), 2.32 (s, 3H), 2.16 (s, 3H), 1.91 (br d, J=10.9 Hz, 3H), 1.69-1.47 (m, 3H).

### (R)—N-(3-(3,5-dimethylisoxazol-4-yl)-4-(piperidin-2-ylmethoxy)phenyl)-2-(5-fluoropyridin-2-yl)acetamide (227)

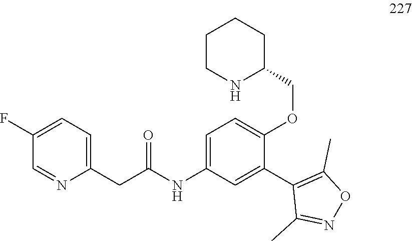 [SMILES: Cc1noc(C)c1-c1cc(NC(=O)Cc2ccc(F)cn2)ccc1OC[C@H]1CCCCN1]

Compound 227 was prepared according to the synthesis described for compound 199, substituting 2-(5-fluoropyridin-2-yl)acetic acid for 2-fluoro-2-methylpropanoic acid.

LCMS (ESI): m/z [M+H] calcd for C₂₄H₂₈FN₄O₃: 439.0, found: 439.2.

¹H NMR (400 MHZ, methanol-d₄) δ=8.45 (br s, 1H), 7.78-7.58 (m, 2H), 7.58-7.44 (m, 2H), 7.19 (d, J=8.9 Hz, 1H), 4.28-4.06 (m, 2H), 4.06-3.75 (m, 2H), 3.50-3.37 (m, 2H), 3.09-2.99 (m, 1H), 2.34 (s, 3H), 2.18 (s, 3H), 1.93 (br d, J=11.6 Hz, 3H), 1.73-1.51 (m, 3H).

### (R)—N-(3-(3,5-dimethylisoxazol-4-yl)-4-(piperidin-2-ylmethoxy)phenyl)-2-(1-methoxycyclopropyl)acetamide (228)

### Preparation of methyl 1-methoxycyclopropanecarboxylate (45)

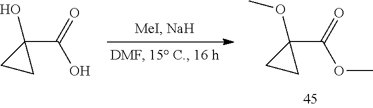

To a solution of 1-hydroxycyclopropanecarboxylic acid (2 g, 19.59 mmol, 1 eq.) in DMF (20 mL) was added NaH (1.96 g, 48.98 mmol, 60% purity, 2.5 eq.). The reaction mixture was stirred at 15° C. for 0.5 hrs. Mel (6.95 g, 48.98 mmol, 3.05 mL, 2.5 eq.) was added to the reaction mixture. The reaction mixture was stirred at 15° C. for 16 hrs (monitored by TLC). The residue was poured into sat. NH₄Cl (100 mL). The aqueous phase was extracted with ethyl acetate (100 mL×3). The combined organic phase was washed with brine (60 mL), dried with anhydrous Na₂SO₄, filtered, and concentrated under vacuum. Methyl 1-methoxycyclopropanecarboxylate ((45), 2 g, crude) was obtained as a yellow oil.

¹H NMR (400 MHZ, DMSO-d₆) δ=3.68-3.62 (m, 3H), 3.29 (s, 3H), 1.17-1.12 (m, 4H).

### Preparation of 1-methoxycyclopropanecarboxylic acid (46)

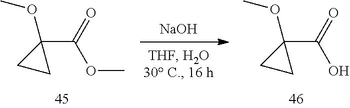

To a solution of methyl 1-methoxycyclopropanecarboxylate ((45), 2 g, 15.37 mmol, 1 eq.) in THF (10 mL) was added NaOH (2 M, 10 mL, 1.30 eq.). The reaction mixture was stirred at 30° C. for 16 hrs (monitored by TLC) and adjusted to pH 5 with 1 M HCl aq.). The aqueous phase was extracted with ethyl acetate (50 mL×4). The combined organic phase was washed with brine (40 mL), dried with anhydrous Na₂SO₄, filtered, and concentrated under vacuum. The residue was purified by prep-HPLC (column: Shim-pack C18 150*25*10 μm; mobile phase: [water (0.225% FA)-ACN]; B %: 1%-20%, 10 min) followed by lyophilization. 1-methoxycyclopropanecarboxylic acid ((46), 340 mg, 2.93 mmol, 19.05% yield) was obtained as a colorless oil.

¹H NMR (400 MHZ, chloroform-d) 8=10.77 (br s, 1H), 3.45 (s, 3H), 1.42-1.34 (m, 2H), 1.27-1.20 (m, 2H).

### Preparation of 1-methoxycyclopropanecarbonyl chloride (47)

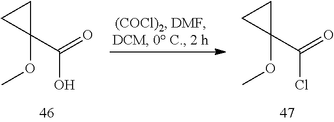

To a solution of oxalyl dichloride (240.14 mg, 1.89 mmol, 165.62 μL, 1.1 eq.) in DCM (3 mL) was added 1-methoxycyclopropanecarboxylic acid ((46), 200 mg, 1.72 mmol, 1 eq.) and DMF (0.05 mL) at 0° C. The mixture was stirred at 0° C. for 1 hr (monitored by TLC). The reaction mixture was concentrated under vacuum. 1-methoxycyclopropanecarbonyl chloride ((47), 200 mg, crude) was obtained as a yellow solid.

### Preparation of 2-(1-methoxycyclopropyl)acetic acid (48)

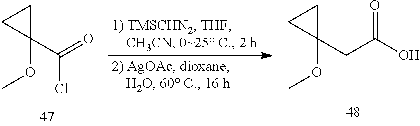

To a mixture of diazomethyl(trimethyl)silane (2 M, 1.49 mL, 2 eq.) in THF (2 mL) and ACN (2 mL) was added 1-methoxycyclopropanecarbonyl chloride ((47), 200 mg, 1.49 mmol, 1 eq.) at 0° C. under N₂. The mixture was stirred at 25° C. for 2 hrs. Then the reaction mixture was concentrated under reduced pressure, and the residue was diluted with dioxane (2 mL) and H₂O (2 mL), then benzoyloxysilver (102.35 mg, 447.00 μmol, 0.3 eq.) was added and the mixture was stirred at 80° C. for 16 hrs (monitored by LCMS). The reaction mixture was filtered, and the filtrate was concentrated under vacuum. The crude product was purified by reversed-phase HPLC (0.1% FA conditions). 2-(1-methoxycyclopropyl)acetic acid ((48), 20 mg, 153.68 μmol, 10.31% yield) and 1-methoxycyclopropanecarboxylic acid (20 mg, 172.24 μmol, 11.56% yield) were obtained as colorless oils.

¹H NMR (400 MHZ, chloroform-d) 8=3.46-3.38 (m, 4H), 3.31 (s, 3H), 2.68-2.53 (m, 2H), 1.41-1.28 (m, 3H), 1.23-1.12 (m, 3H), 0.95-0.82 (m, 2H), 0.69-0.55 (m, 2H).

### Preparation of (R)—N-(3-(3,5-dimethylisoxazol-4-yl)-4-(piperidin-2-ylmethoxy)phenyl)-2-(1-methoxycyclopropyl)acetamide (228)

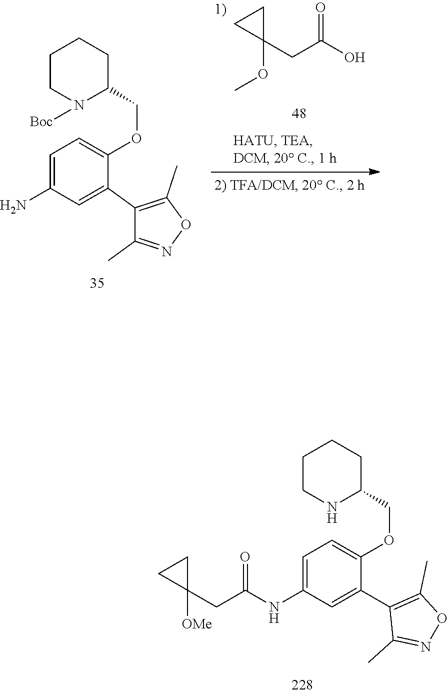

Compound 228 was prepared according to the synthesis described for compound 199, substituting 2-(1-methoxycyclopropyl)acetic acid (48) for 2-fluoro-2-methylpropanoic acid.

LCMS (ESI): m/z [M+H] calcd for C₂₃H₃₂N₃O₄: 414.23; found: 414.2.

¹H NMR (400 MHZ, methanol-d₄) δ=7.57 (dd, J=2.7, 8.9 Hz, 1H), 7.45 (d, J=2.6 Hz, 1H), 7.13 (d, J=8.9 Hz, 1H), 4.02-3.86 (m, 2H), 3.35 (s, 3H), 3.20-3.02 (m, 2H), 2.81-2.71 (m, 1H), 2.67 (s, 2H), 2.32 (s, 3H), 2.16 (s, 3H), 1.85 (br d, J=6.5 Hz, 1H), 1.80-1.69 (m, 2H), 1.58-1.39 (m, 2H), 1.37-1.24 (m, 1H), 0.90-0.83 (m, 2H), 0.74-0.66 (m, 2H).

### N-(3-(3,5-dimethylisoxazol-4-yl)-4-(((R)-piperidin-2-yl)methoxy)phenyl)-2-ethoxycyclopropane-1-carboxamide (229)

### Preparation of ethyl 2-ethoxycyclopropanecarboxylate (49)

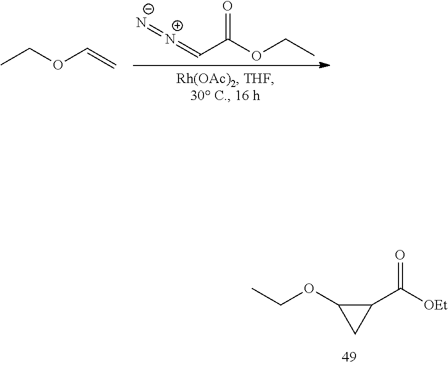

To a mixture of vinyloxyethane (1 g, 13.87 mmol, 1.33 mL, 1 eq.) and diacetoxyrhodium (306.49 mg, 693.43 μmol, 0.05 eq.) in THF (10 mL) was added ethyl 2-diazoacetate (1.58 g, 13.87 mmol, 1.45 mL, 1 eq.) in THF (10 mL) in portions under N₂. The mixture was stirred at 30° C. for 16 hrs under N₂ (monitored by LCMS). The mixture was filtered and concentrated under vacuum. The residue was purified by silica gel chromatography (100-200 mesh silica gel, petroleum ether/ethyl acetate=100/1, 50/1). Ethyl 2-ethoxycyclopropanecarboxylate ((49), 0.52 g, 3.29 mmol, 23.70% yield) was obtained as a colorless oil.

¹H NMR (400 MHZ, chloroform-d) 8=4.05 (q, J=7.1 Hz, 2H), 3.59-3.48 (m, 3H), 1.68 (ddd, J=2.0, 6.1, 9.5 Hz, 1H), 1.22-1.16 (m, 5H), 1.13 (t, J=7.0 Hz, 3H).

### Preparation of 2-ethoxycyclopropanecarboxylic acid (50)

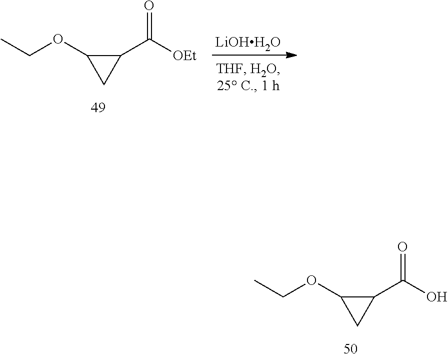

To a mixture of ethyl 2-ethoxycyclopropanecarboxylate ((49), 150 mg, 948.20 μmol, 1 eq.) in EtOH (3 mL) and H₂O (1 mL) was added LiOH·H₂O (79.58 mg, 1.90 mmol, 2 eq.). The mixture was stirred at 25° C. for 1 hr (monitored by LCMS). The mixture was concentrated to remove excess EtOH. The residue was acidified with 1M HCl aq., the pH was adjusted to 6-7, and then the mixture was concentrated. 2-ethoxycyclopropanecarboxylic acid ((50), 90 mg, crude) was obtained as a colorless oil.

### Preparation of N-(3-(3,5-dimethylisoxazol-4-yl)-4-(((R)-piperidin-2-yl)methoxy)phenyl)-2-ethoxycyclopropane-1-carboxamide (229)

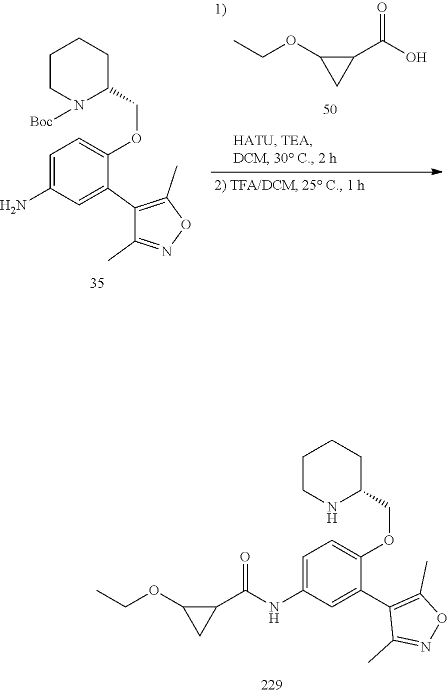

Compound 229 was prepared according to the synthesis described for compound 199, substituting 2-ethoxycyclopropanecarboxylic acid (50) for 2-fluoro-2-methylpropanoic acid.

LCMS (ESI): m/z [M+H] calcd for C₂₃H₃₂N₃O₄: 414.23; found: 414.3.

¹H NMR (400 MHZ, methanol-d₄) δ=7.51 (dd, J=2.7, 8.9 Hz, 1H), 7.41 (d, J=2.6 Hz, 1H), 7.08 (d, J=9.0 Hz, 1H), 3.93-3.87 (m, 1H), 3.85-3.79 (m, 1H), 3.66-3.56 (m, 3H), 3.00 (br d, J=10.6 Hz, 1H), 2.88-2.79 (m, 1H), 2.59 (dt, J=2.6, 11.9 Hz, 1H), 2.30 (s, 3H), 2.15 (s, 3H), 1.88 (ddd, J=2.0, 5.9, 9.5 Hz, 1H), 1.84-1.75 (m, 1H), 1.70-1.58 (m, 2H), 1.48-1.33 (m, 2H), 1.31-1.09 (m, 6H).

### (R)—N-(3-(3,5-dimethylisoxazol-4-yl)-4-(piperidin-2-ylmethoxy)phenyl)-4-fluoro-1-methyl-1H-pyrazole-5-carboxamide (230)

### Preparation of methyl 4-fluoro-2-methyl-pyridinerazole-3-carboxylate (51)

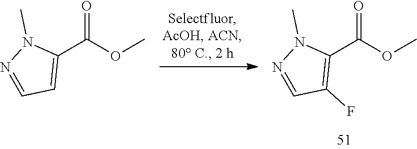

To a mixture of methyl 2-methylpyridinerazole-3-carboxylate (500 mg, 3.57 mmol, 1 eq.) in ACN (10 mL) and HOAc (1 mL) was added Select F (1.52 g, 4.28 mmol, and 1.2 eq.). The mixture was stirred at 80° C. for 2 hrs (monitored by LCMS). The mixture was concentrated under reduced pressure. The residue was purified by silica gel chromatography (100-200 mesh silica gel, petroleum ether/ethyl acetate=10/1, 5/1) TLC (petroleum ether:ethyl acetate=5:1). Methyl 4-fluoro-2-methyl-pyridinerazole-3-carboxylate ((51), 180 mg, 1.14 mmol, 31.90% yield) was obtained as a colorless oil.

¹H NMR (400 MHZ, CDCl3) δ=7.34 (d, J=4.0 Hz, 1H), 4.11 (s, 3H), 3.93 (s, 3H).

### Preparation of 4-fluoro-2-methyl-pyridinerazole-3-carboxylic acid (52)

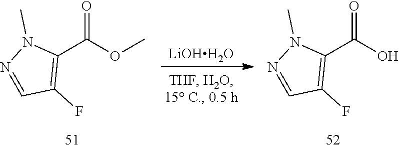

To a mixture of methyl 4-fluoro-2-methyl-pyridinerazole-3-carboxylate ((51), 180 mg, 1.14 mmol, 1 eq.) in MeOH (3 mL) and H₂O (1 mL) was added LiOH·H₂O (95.53 mg, 2.28 mmol, 2 eq.). The mixture was stirred at 15° C. for 30 min (monitored by LCMS). The mixture was concentrated to remove excess EtOH. The residue was acidified with 1 M HCl aq., and the pH was adjusted to 6-7. The mixture was concentrated. 4-fluoro-2-methyl-pyridinerazole-3-carboxylic acid ((52), 140 mg, crude) was obtained as a white solid.

### Preparation of (R)—N-(3-(3,5-dimethylisoxazol-4-yl)-4-(piperidin-2-ylmethoxy)phenyl)-4-fluoro-1-methyl-1H-pyrazole-5-carboxamide (230)

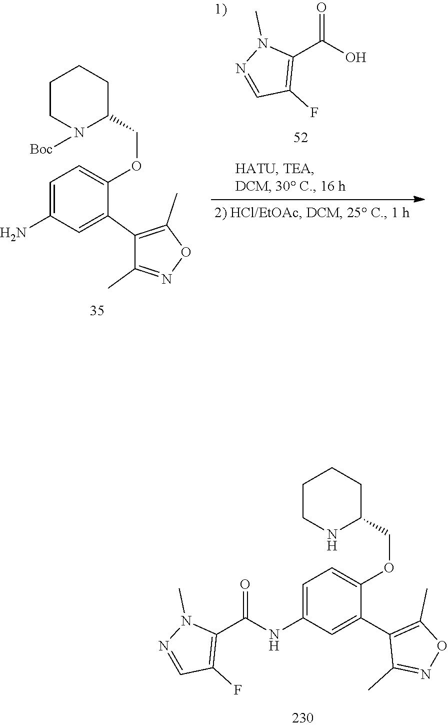

Compound 230 was prepared according to the synthesis described for compound 199, substituting 4-fluoro-2-methyl-pyridinerazole-3-carboxylic acid (52) for 2-fluoro-2-methylpropanoic acid.

¹H NMR (400 MHZ, methanol-d₄) δ=7.72 (dd, J=2.4, 9.0 Hz, 1H), 7.55 (d, J=2.6 Hz, 1H), 7.47 (d, J=4.3 Hz, 1H), 7.22 (d, J=9.0 Hz, 1H), 4.16-4.06 (m, 2H), 4.05 (d, J=0.7 Hz, 3H), 3.52-3.43 (m, 1H), 3.38 (br d, J=12.6 Hz, 1H), 3.09-2.98 (m, 1H), 2.35 (s, 3H), 2.19 (s, 3H), 1.98-1.88 (m, 3H), 1.73-1.48 (m, 3H).

LCMS (ESI): m/z [M+H] calcd for C₂₂H₂₇FN₅O₃: 428.20; found: 428.3.

### (R)—N-(3-(3,5-dimethylisoxazol-4-yl)-4-(piperidin-2-ylmethoxy)phenyl)-2-isopropyl-4-methylthiazole-5-carboxamide (231)

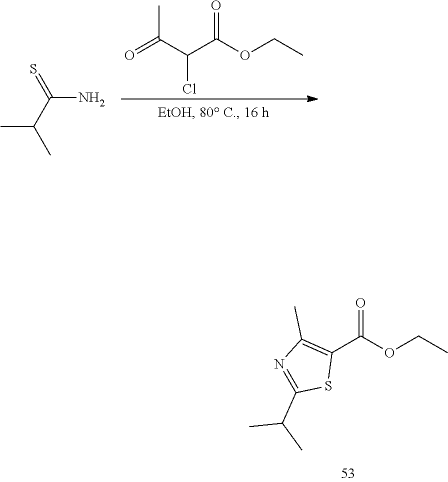

A mixture of 2-methylpropanethioamide (0.5 g, 4.85 mmol, 1 eq.) and ethyl 2-chloro-3-oxo-butanoate (957.03 mg, 5.81 mmol, 804.23 μL, 1.2 eq.) in EtOH (10 mL) was stirred at 80° C. for 16 hrs (monitored by LCMS and TLC). The mixture was concentrated under reduced pressure. The residue was purified by silica gel chromatography (100-200 mesh silica gel, petroleum ether/ethyl acetate=100/1, 10/1). Ethyl 2-isopropyridinel-4-methyl-thiazole-5-carboxylate ((53), 820 mg, 3.84 mmol, 79.34% yield) was obtained as a yellow oil.

¹H NMR (400 MHZ, chloroform-d) 8=4.32 (q, J=7.1 Hz, 2H), 3.26 (spt, J=6.9 Hz, 1H), 2.71 (s, 3H), 1.43-1.34 (m, 9H).

### Preparation of 2-isopropyridinel-4-methyl-thiazole-5-carboxylic acid (54)

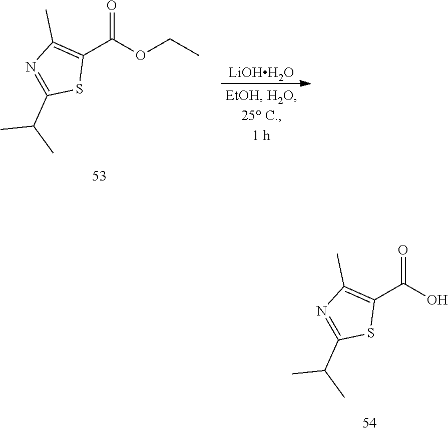

To a mixture of ethyl 2-isopropyridinel-4-methyl-thiazole-5-carboxylate ((53), 0.2 g, 937.66 μmol, 1 eq.) in EtOH (3 mL) and H₂O (1 mL) was added LiOH·H₂O (78.70 mg, 1.88 mmol, 2 eq.). The mixture was stirred at 25° C. for 1 hr (monitored by LCMS). The mixture was concentrated to remove excess EtOH. The residue was acidified with 1M HCl aq., the pH was adjusted to 6-7, then the mixture was filtered and the cake was dried. 2-isopropyridinel-4-methyl-thiazole-5-carboxylic acid ((54), 120 mg, crude) was obtained as a white solid.

¹H NMR (400 MHZ, DMSO-d₆) δ=13.77-12.43 (m, 1H), 3.27-3.17 (m, 1H), 2.57 (s, 3H), 1.30 (d, J=6.9 Hz, 6H).

### Preparation of (R)—N-(3-(3,5-dimethylisoxazol-4-yl)-4-(piperidin-2-ylmethoxy)phenyl)-2-isopropyl-4-methylthiazole-5-carboxamide (231)

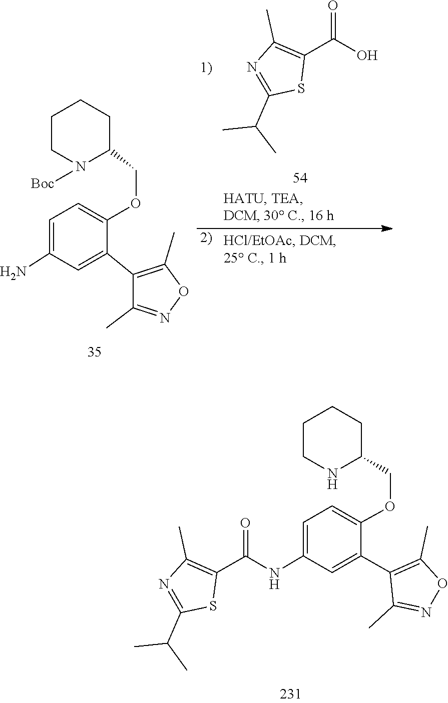

Compound 231 was prepared according to the synthesis described for compound 199, substituting 2-isopropyridinel-4-methyl-thiazole-5-carboxylic acid (54) for 2-fluoro-2-methylpropanoic acid.

¹H NMR (400 MHZ, methanol-d₄) δ=7.68 (dd, J=2.7, 8.9 Hz, 1H), 7.48 (d, J=2.6 Hz, 1H), 7.21 (d, J=8.9 Hz, 1H), 4.15-4.03 (m, 2H), 3.47 (br dd, J=4.4, 7.1 Hz, 1H), 3.42-3.33 (m, 1H), 3.30-3.24 (m, 1H), 3.09-2.98 (m, 1H), 2.62 (s, 3H), 2.35 (s, 3H), 2.19 (s, 3H), 1.98-1.87 (m, 3H), 1.73-1.47 (m, 3H), 1.41 (d, J=6.9 Hz, 6H).

LCMS (ESI): m/z [M+H] calcd for C₂₅H₃₃N₄O₃S: 469.22; found: 469.3.

### (R)—N-(3-(3,5-dimethylisoxazol-4-yl)-4-(piperidin-2-ylmethoxy)phenyl)-4-methyl-1,2,5-oxadiazole-3-carboxamide (232)

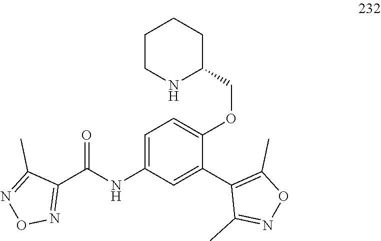 [SMILES: Cc1nonc1C(=O)Nc1ccc(OC[C@H]2CCCCN2)c(-c2c(C)noc2C)c1]

Compound 232 was prepared according to the synthesis described for compound 199, substituting 4-methyl-1,2,5-oxadiazole-3-carboxylic acid for 2-fluoro-2-methylpropanoic acid.

¹H NMR (400 MHZ, methanol-d₄) δ=7.81 (br d, J=8.9 Hz, 1H), 7.60 (d, J=2.7 Hz, 1H), 7.23 (d, J=8.9 Hz, 1H), 4.15-4.04 (m, 2H), 3.51-3.37 (m, 2H), 3.04 (br s, 1H), 2.60 (s, 3H), 2.36 (s, 3H), 2.19 (s, 3H), 1.94 (br s, 3H), 1.73-1.46 (m, 3H).

LCMS (ESI): m/z [M+H] calcd for C₂₁H₂₆N₅O₄: 412.2; found: 412.3.

### (R)—N-(3-(3,5-dimethylisoxazol-4-yl)-4-(piperidin-2-ylmethoxy)phenyl)-2-(isothiazol-5-yl)acetamide (233)

### Preparation of isothiazole-5-carbonyl chloride (55)

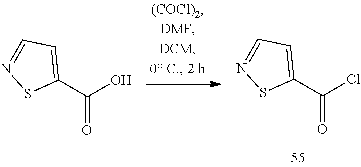 [SMILES: O=C(O)c1ccns1 ; O=C(Cl)c1ccns1]

To a solution of isothiazole-5-carboxylic acid (200 mg, 1.55 mmol, 1 eq.) in DCM (4 mL) was added oxalyl dichloride (216.23 mg, 1.70 mmol, 149.13 μL, 1.1 eq.) and DMF (0.05 mL) at 0° C. The mixture was stirred at 0° C. for 1 hr. The reaction mixture was concentrated under vacuum. Isothiazole-5-carbonyl chloride ((55), 220 mg, crude) was obtained as a yellow oil.

### Preparation of 2-isothiazol-5-ylacetic acid (56)

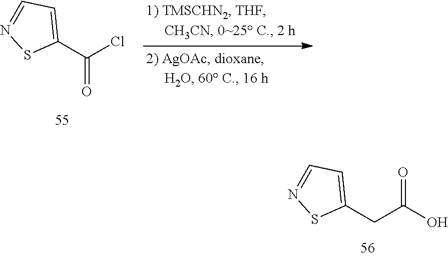 [SMILES: O=C(Cl)c1ccns1 ; O=C(O)Cc1ccns1]

To a mixture of isothiazole-5-carbonyl chloride ((55), 220 mg, 1.49 mmol, 1 eq.) in THF (2 mL) and ACN (2 mL) was added diazomethyl(trimethyl)silane (2 M, 1.49 mL, 2 eq.) at 0° C. under N₂. The mixture was stirred at 25° C. for 2 hrs. Then the reaction mixture was concentrated under reduced pressure and the residue was diluted with dioxane (2 mL), H₂O (2 mL), and then AgOAc (74.64 mg, 447.21 μmol, 22.90 μL, 0.3 eq.) was added and the mixture was stirred at 60° C. for 16 hrs. The residue was poured into sat. NaHCO₃ aq. (100 mL). The aqueous phase was extracted with ethyl acetate (30 mL). The pH of the aqueous phase was adjusted to 5 with 1 M HCl. The aqueous phase was extracted with ethyl acetate (50 mL×3). The combined organic phase was washed with brine (20 mL), dried with anhydrous Na₂SO₄, filtered and concentrated under vacuum. 2-isothiazol-5-ylacetic acid ((56), 70 mg, 488.95 μmol, 32.80% yield) was obtained as a brown solid.

### Preparation of (R)—N-(3-(3,5-dimethylisoxazol-4-yl)-4-(piperidin-2-ylmethoxy)phenyl)-2-(isothiazol-5-yl)acetamide (233)

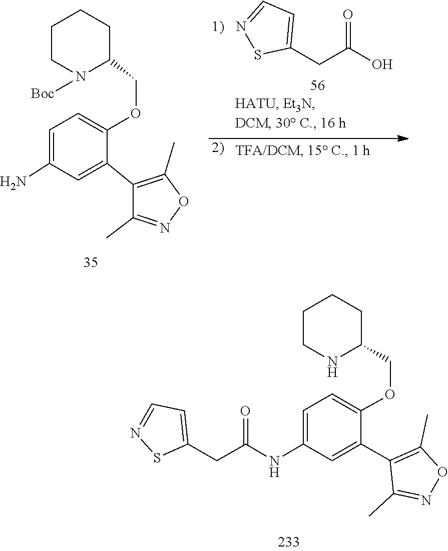

Compound 233 was prepared according to the synthesis described for compound 199, substituting 2-isothiazol-5-ylacetic acid (56) for 2-fluoro-2-methylpropanoic acid.

¹H NMR (400 MHZ, methanol-d₄) δ=8.40 (d, J=1.6 Hz, 1H), 7.64 (dd, J=2.8, 8.9 Hz, 1H), 7.45 (d, J=2.6 Hz, 1H), 7.29-7.24 (m, 1H), 7.19 (d, J=9.0 Hz, 1H), 4.17 (s, 2H), 4.12-4.00 (m, 2H), 3.50-3.42 (m, 1H), 3.37 (br d, J=12.8 Hz, 1H), 3.09-2.97 (m, 1H), 2.33 (s, 3H), 2.17 (s, 3H), 1.91 (br d, J=10.6 Hz, 3H), 1.74-1.43 (m, 3H).

LCMS (ESI): m/z [M+H] calcd for C₂₂H₂₇N₄O₃S: 427.17; found: 427.2.

### (R)—N-(3-(3,5-dimethylisoxazol-4-yl)-4-(piperidin-2-ylmethoxy)phenyl)-2-(isothiazol-4-yl)acetamide (234)

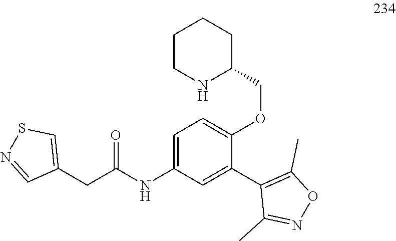 [SMILES: Cc1noc(C)c1-c1cc(NC(=O)Cc2cnsc2)ccc1OC[C@H]1CCCCN1]

Compound 234 was prepared according to the synthesis described for compound 199, substituting 2-(isothiazol-4-yl)acetic acid for 2-fluoro-2-methylpropanoic acid.

¹H NMR (400 MHZ, methanol-d₄) δ=8.76 (s, 1H), 8.51 (s, 1H), 7.54 (dd, J=2.7, 8.9 Hz, 1H), 7.43 (d, J=2.7 Hz, 1H), 7.09 (d, J=8.9 Hz, 1H), 3.95-3.87 (m, 1H), 3.87-3.75 (m, 3H), 3.06-2.93 (m, 1H), 2.89-2.77 (m, 1H), 2.58 (dt, J=2.6, 11.8 Hz, 1H), 2.30 (s, 3H), 2.14 (s, 3H), 1.85-1.74 (m, 1H), 1.69-1.55 (m, 2H), 1.47-1.32 (m, 2H), 1.21-1.09 (m, 1H).

LCMS (ESI): m/z [M+H] calcd for C₂₂H₂₇N₄O₃S: 427.17; found: 427.3.

### (R)—N-(3-(3,5-dimethylisoxazol-4-yl)-4-(piperidin-3-yloxy)phenyl)-1-fluorocyclopropane-1-carboxamide (235)

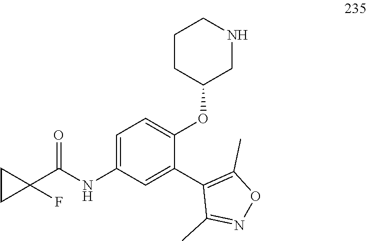 [SMILES: Cc1noc(C)c1-c1cc(NC(=O)C2(F)CC2)ccc1O[C@@H]1CCCNC1]

Compound 235 was prepared according to the synthesis described for compound 217, substituting tert-butyl (R)-3-hydroxypiperidine-1-carboxylate for tert-butyl (2R)-2-(hydroxymethyl)piperidine-1-carboxylate.

¹H NMR (400 MHZ, methanol-d₄) δ=7.67 (dd, J=2.8, 8.9 Hz, 1H), 7.51 (d, J=2.6 Hz, 1H), 7.21 (d, J=8.9 Hz, 1H), 4.35 (td, J=3.3, 6.3 Hz, 1H), 3.38-3.33 (m, 1H), 3.29-3.09 (m, 3H), 2.38-2.28 (m, 3H), 2.19 (s, 3H), 2.03-1.89 (m, 2H), 1.82-1.65 (m, 2H), 1.45-1.35 (m, 4H).

LCMS (ESI): m/z [M+H] calcd for C₂₀H₂₅FN₃O₃: 374.2; found: 374.2.

### N-[3-(3,5-dimethylisoxazol-4-yl)-4-[[(2R)-2-piperidyl]methoxy]phenyl]-N-methyl-cyclopropanecarboxamide (236)

### Preparation of tert-butyl (2R)-2-[[2-(3,5-dimethylisoxazol-4-yl)-4-(methylamino)phenoxy]methyl]piperidine-1-carboxylate (57)

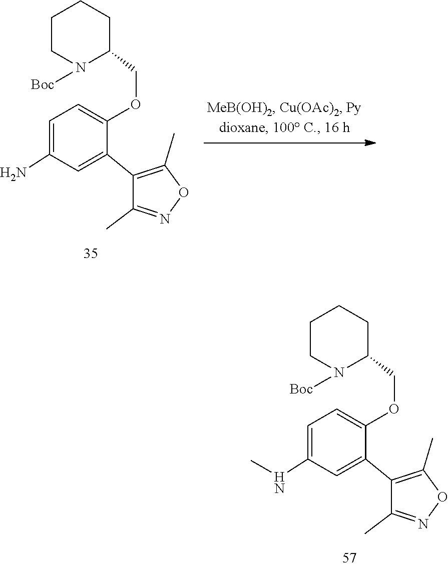 [SMILES: Cc1noc(C)c1-c1cc(N)ccc1OC[C@H]1CCCCN1C(=O)OC(C)(C)C ; CNc1ccc(OC[C@H]2CCCCN2C(=O)OC(C)(C)C)c(-c2c(C)noc2C)c1]

A mixture of tert-butyl (2R)-2-[[4-amino-2-(3,5-dimethylisoxazol-4-yl)phenoxy]methyl]piperidine-1-carboxylate ((35), 150 mg, 373.60 μmol, 1 eq.), methylboronic acid (55.91 mg, 934.00 μmol, 2.5 eq.), Cu(OAc)₂ (169.65 mg, 934.00 μmol, 2.5 eq.), and pyridine (103.43 mg, 1.31 mmol, 105.54 μL, 3.5 eq.) in dioxane (3 mL) was degassed and purged with O₂ three times, and then the mixture was stirred at 100° C. for 16 hrs under an O₂ atmosphere (15 psi). The reaction mixture was poured into water (10 mL). The aqueous phase was extracted with ethyl acetate (10 mL×3). The combined organic phase was washed with brine and filtered and the filtrate was concentrated under vacuum to give a residue. The residue was purified by flash silica gel chromatography (ISCO®; 24 g SepaFlash® Silica Flash Column, eluent of 0˜40% ethyl acetate/petroleum ether gradient). Tert-butyl (2R)-2-[[2-(3,5-dimethylisoxazol-4-yl)-4-(methylamino)phenoxy]methyl]piperidine-1-carboxylate ((57), 40 mg, 77.97 μmol, 20.87% yield, 81% purity) was obtained as a yellow oil.

LCMS (ESI): m/z [M+H] calcd for C₄₅H₆₅N₆O₈: 416.25; found: 416.3.

### Preparation of tert-butyl (2R)-2-[[4-[cyclopropanecarbonyl(methyl)amino]-2-(3,5-dimethylisoxazol-4-yl)phenoxy]methyl]piperidine-1-carboxylate (58)

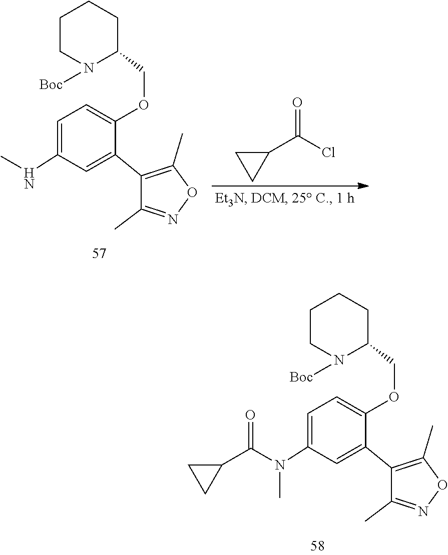 [SMILES: CNc1ccc(OC[C@H]2CCCCN2C(=O)OC(C)(C)C)c(-c2c(C)noc2C)c1 ; O=C(Cl)C1CC1 ; Cc1noc(C)c1-c1cc(N(C)C(=O)C2CC2)ccc1OC[C@H]1CCCCN1C(=O)OC(C)(C)C]

To a solution of tert-butyl (2R)-2-[[2-(3,5-dimethylisoxazol-4-yl)-4-(methylamino)phenoxy]methyl]piperidine-1-carboxylate ((57), 40 mg, 77.97 μmol, 1 eq.) and Et₃N (15.78 mg, 155.95 μmol, 21.71 μL, 2 eq.) in DCM (2 ml) was added cyclopropanecarbonyl chloride (8.97 mg, 85.77 μmol, 7.80 μL, 1.1 eq.) in DCM (0.5 ml) dropwise. The reaction mixture was stirred at 25° C. for 0.5 hrs. To the reaction mixture was added cyclopropanecarbonyl chloride (8.97 mg, 1.1 eq.) in DCM (0.5 mL). The reaction mixture was stirred at 25° C. for 0.5 hrs. The reaction mixture was concentrated under vacuum to give a residue without further purification. Tert-butyl (2R)-2-[[4-[cyclopropanecarbonyl(methyl)amino]-2-(3,5-dimethylisoxazol-4-yl)phenoxy]methyl]piperidine-1-carboxylate ((58), 35 mg, crude) was obtained as a yellow oil.

LCMS (ESI): m/z [M+H] calcd for C₂₇H₃₈N₃O₅: 484.27; found: 484.4.

### Preparation of N-[3-(3,5-dimethylisoxazol-4-yl)-4-[[(2R)-2-piperidyl]methoxy]phenyl]-N-methyl-cyclopropanecarboxamide (236)

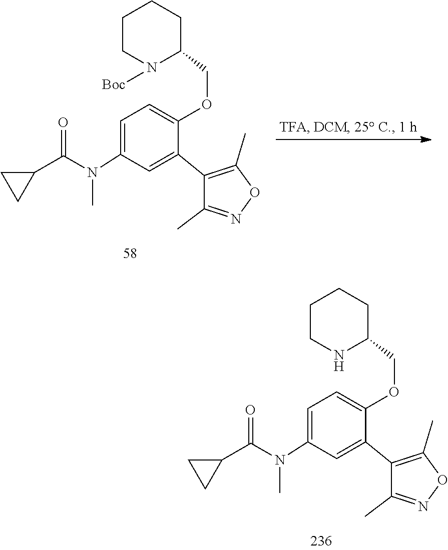 [SMILES: Cc1noc(C)c1-c1cc(N(C)C(=O)C2CC2)ccc1OC[C@H]1CCCCN1C(=O)OC(C)(C)C ; Cc1noc(C)c1-c1cc(N(C)C(=O)C2CC2)ccc1OC[C@H]1CCCCN1]

To a solution of tert-butyl (2R)-2-[[4-[cyclopropanecarbonyl(methyl)amino]-2-(3,5-dimethylisoxazol-4-yl)phenoxy]methyl]piperidine-1-carboxylate ((58), 35 mg, 72.37 μmol, 1 eq.) in DCM (3 mL) was added TFA (2.02 g, 17.73 mmol, 1.31 ml, 244.93 eq.). The reaction mixture was stirred at 25° C. for 1 hr. The reaction mixture was concentrated under vacuum to give a residue. The residue was purified by prep-HPLC (column: Phenomenex Gemini-NX C_{18 75*30} mm*3 um; mobile phase: [water (0.1% TFA)-ACN]; B %: 12%-22%, 7 min) followed by lyophilization. N-[3-(3,5-dimethylisoxazol-4-yl)-4-[[(2R)-2-piperidyl]methoxy]phenyl]-N-methyl-cyclopropanecarboxamide (21.78 mg, 43.78 μmol, 60.49% yield, 100% purity, TFA) was obtained as a yellow gum.

LCMS (ESI): m/z [M+H] calcd for C₂₂H₂₉N₃O₃: 384.22; found: 384.2.

¹H NMR (400 MHZ, methanol-d₄) δ=7.43 (br d, J=7.7 Hz, 1H), 7.36-7.21 (m, 2H), 4.23-4.11 (m, 2H), 3.50 (br dd, J=2.4, 5.1 Hz, 1H), 3.38 (br d, J=12.5 Hz, 1H), 3.27 (br s, 3H), 3.09-2.99 (m, 1H), 2.34 (s, 3H), 2.17 (s, 3H), 2.00-1.88 (m, 3H), 1.74-1.40 (m, 4H), 0.93 (quin, J=3.7 Hz, 2H), 0.69 (br s, 2H).

### N-[3-(3,5-dimethylisoxazol-4-yl)-4-[2-(2-methoxyethylamino)ethoxy]phenyl]-1-fluoro-cyclopropanecarboxamide (237)

### Preparation of N-[3-(3,5-dimethylisoxazol-4-yl)-4-(2-hydroxyethoxy)phenyl]-1-fluoro-cyclopropanecarboxamide (59)

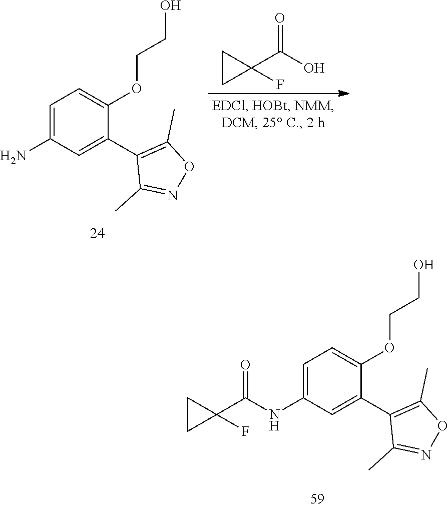 [SMILES: Cc1noc(C)c1-c1cc(N)ccc1OCCO ; O=C(O)C1(F)CC1 ; Cc1noc(C)c1-c1cc(NC(=O)C2(F)CC2)ccc1OCCO]

To a solution of 2-[4-amino-2-(3,5-dimethylisoxazol-4-yl)phenoxy]ethanol ((24), 290 mg, 1.17 mmol, 1 eq.) in DCM (5 mL) was added EDCl (335.88 mg, 1.75 mmol, 1.5 eq.) and HOBt (236.75 mg, 1.75 mmol, 1.5 eq.) then added NMM (11.81 mg, 116.80 μmol, 12.84 L, 0.1 eq.) and 1-fluorocyclopropanecarboxylic acid (182.36 mg, 1.75 mmol, 1.5 eq.). The mixture was stirred at 30° C. for 16 hrs (monitored by LCMS). The mixture was concentrated under vacuum. The crude product was purified by reversed-phase HPLC (0.1% FA conditions, column: Phenomenex Gemini-NX C18 75*30 mm*3 μm; mobile phase: [water (0.1% TFA)-ACN]; B %: 28%-38%, 7 min). N-[3-(3,5-dimethylisoxazol-4-yl)-4-(2-hydroxyethoxy)phenyl]-1-fluoro-cyclopropanecarboxamide ((59), 150 mg, 448.64 μmol, 38.41% yield) was obtained as a white solid.

LCMS (ESI): m/z [M+H] calcd for C₁₇H₂₀FN₂O₄: 335.0, found: 335.1.

### Preparation of 2-[2-(3,5-dimethylisoxazol-4-yl)-4-[(1-fluorocyclopropanecarbonyl)amino]phenoxy]ethyl 4-methylbenzenesulfonate (60)

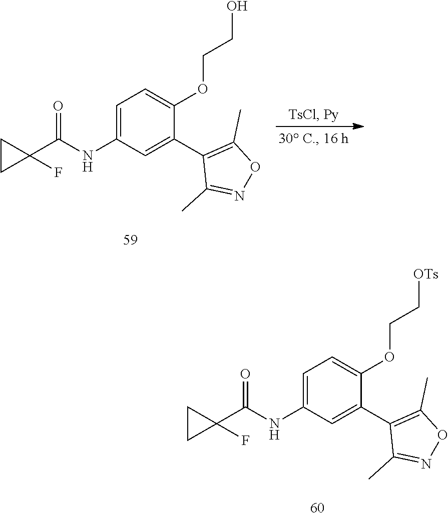 [SMILES: Cc1noc(C)c1-c1cc(NC(=O)C2(F)CC2)ccc1OCCO ; Cc1ccc(S(=O)(=O)OCCOc2ccc(NC(=O)C3(F)CC3)cc2-c2c(C)noc2C)cc1]

To a solution of N-[3-(3,5-dimethylisoxazol-4-yl)-4-(2-hydroxyethoxy)phenyl]-1-fluoro-cyclopropanecarboxamide ((59), 110 mg, 329.01 μmol, 1 eq.) in DCM (1 mL) was added TsCl (94.09 mg, 493.51 μmol, 1.5 eq.) and pyridine (1 mL). The mixture was stirred at 30° C. for 16 hrs (monitored by LCMS). The mixture was diluted with HCl (1 mol, 20 mL) and then extracted with DCM (30 mL×2). The combined organic phase was washed with brine (15 mL), dried over anhydrous Na₂SO₄, filtered and concentrated under vacuum. The residue was purified by flash silica gel chromatography (ISCO®; 12 g SepaFlash® Silica Flash Column, eluent of 0˜90% ethyl acetate/petroleum ether gradient at 25 mL/min). 2-[2-(3,5-dimethylisoxazol-4-yl)-4-[(1-fluorocyclopropanecarbonyl)amino]phenoxy]ethyl 4-methylbenzenesulfonate ((60), 140 mg, 286.58 μmol, 87.10% yield) was obtained as a colorless oil.

LCMS (ESI): m/z [M+H] calcd for C₂₄H₂₆N₂O₆: 489.0, found: 489.0.

### Preparation of N-[3-(3,5-dimethylisoxazol-4-yl)-4-[2-(2-methoxyethylamino)ethoxy]phenyl]-1-fluoro-cyclopropanecarboxamide (237)

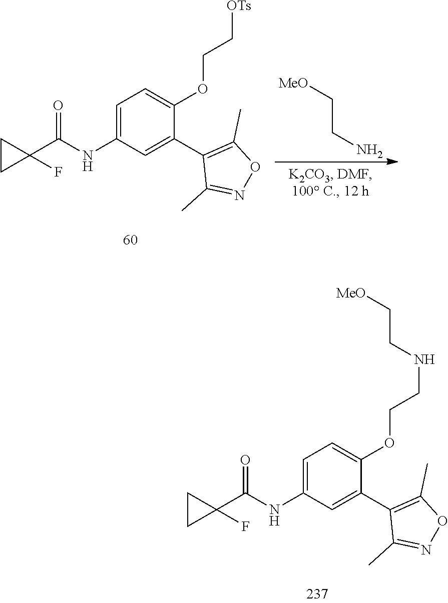 [SMILES: Cc1ccc(S(=O)(=O)OCCOc2ccc(NC(=O)C3(F)CC3)cc2-c2c(C)noc2C)cc1 ; COCCN ; COCCNCCOc1ccc(NC(=O)C2(F)CC2)cc1-c1c(C)noc1C]

To a solution of 2-[2-(3,5-dimethylisoxazol-4-yl)-4-[(1-fluorocyclopropanecarbonyl)amino]phenoxy]ethyl 4-methylbenzenesulfonate ((60), 140 mg, 286.58 μmol, 1 eq.) in DMF (1.5 mL) was added K₂CO₃ (79.21 mg, 573.15 μmol, 2 eq.) and 2-methoxyethanamine (25.83 mg, 343.89 μmol, 29.90 μL, 1.2 eq.). The mixture was stirred at 100° C. for 16 hrs. The mixture was filtered and concentrated under vacuum. The residue was purified by prep-HPLC (TFA conditions, column: Phenomenex Gemini-NX C18 75*30 mm*3 μm; mobile phase: [water (0.1% TFA)-ACN]; B %: 22%-32%, 7 min). N-[3-(3,5-dimethylisoxazol-4-yl)-4-[2-(2-methoxyethylamino)ethoxy]phenyl]-1-fluoro-cyclopropanecarboxamide (34.78 mg, 68.81 μmol, 24.01% yield, 100% purity, TFA) was obtained as a yellow solid.

¹H NMR (400 MHZ, methanol-d₄) δ=7.70-7.64 (m, 1H), 7.49 (d, J=2.8 Hz, 1H), 7.16 (d, J=9.0 Hz, 1H), 4.29 (t, J=5.2 Hz, 2H), 3.58-3.51 (m, 2H), 3.43 (t, J=5.1 Hz, 2H), 3.39 (s, 3H), 3.18-3.11 (m, 2H), 2.33 (s, 3H), 2.17 (s, 3H), 1.44-1.35 (m, 4H).

LCMS (ESI): m/z [M+H] calcd for C₂₀H₂₇N₃O₄: 392.0, found: 392.0.

### (R)—N-(3-(3,5-dimethylisoxazol-4-yl)-4-(pyrrolidin-3-yloxy)phenyl)-1-fluorocyclopropane-1-carboxamide (238)

### Preparation of tert-butyl (3R)-3-[2-(3,5-dimethylisoxazol-4-yl)-4-nitro-phenoxy]pyridinerrolidine-1-carboxylate (61)

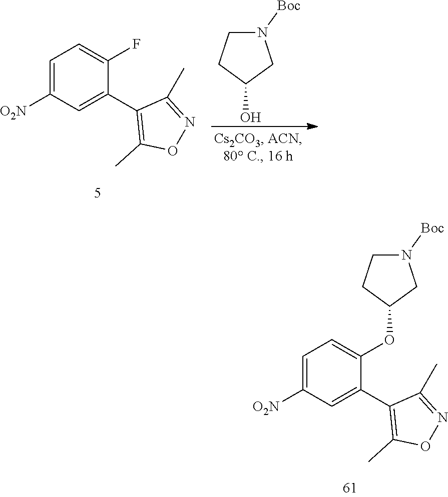

A mixture of 4-(2-fluoro-5-nitro-phenyl)-3,5-dimethyl-isoxazole ((5), 200 mg, 846.74 μmol, 1 eq.), tert-butyl (3R)-3-hydroxypyridinerrolidine-1-carboxylate (174.39 mg, 931.41 μmol, 1.1 eq.) and Cs₂CO₃ (551.77 mg, 1.69 mmol, 2 eq.) in ACN (5 mL) was stirred at 80° C. for 16 hrs (monitored by TLC). The reaction mixture was filtered and concentrated under reduced pressure to give a residue. The residue was purified by column chromatography (SiO₂, petroleum ether/ethyl acetate=3/1 to 1/1). Tert-butyl (3R)-3-[2-(3,5-dimethylisoxazol-4-yl)-4-nitro-phenoxy]pyridinerrolidine-1-carboxylate ((61), 300 mg, 743.63 μmol, 87.82% yield) was obtained as a yellow oil.

### Preparation of tert-butyl (3R)-3-[4-amino-2-(3,5-dimethylisoxazol-4-yl)phenoxy]pyridinerrolidine-1-carboxylate (62)

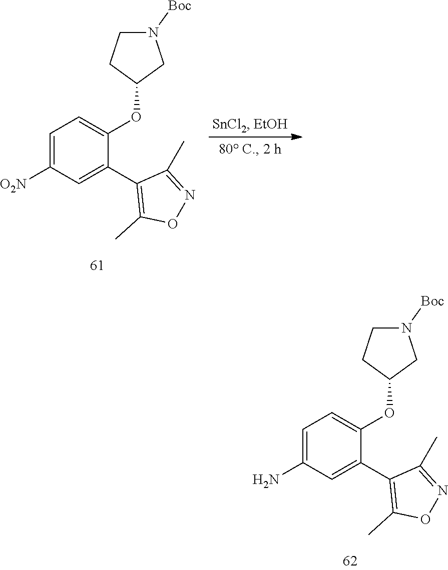 [SMILES: Cc1noc(C)c1-c1cc([N+](=O)[O-])ccc1O[C@@H]1CCN(C(=O)OC(C)(C)C)C1 ; Cc1noc(C)c1-c1cc(N)ccc1O[C@@H]1CCN(C(=O)OC(C)(C)C)C1]

A mixture of tert-butyl (3R)-3-[2-(3,5-dimethylisoxazol-4-yl)-4-nitro-phenoxy]pyridinerrolidine-1-carboxylate ((61), 300 mg, 743.63 μmol, 1 eq.) and SnCl₂·2H₂O (335.60 mg, 1.49 mmol, 2 eq.) in EtOH (3 mL) was stirred at 80° C. for 16 hrs (monitored by LC-MS). The reaction mixture was concentrated under reduced pressure to give a residue. The crude product tert-butyl (3R)-3-[4-amino-2-(3,5-dimethylisoxazol-4-yl)phenoxy]pyridinerrolidine-1-carboxylate ((62), 250 mg, crude) was used in the next step without further purification as a yellow oil.

LCMS (ESI): m/z [M+H] calcd for C₂₀H₂₈N₃O₄: 374.20; found: 374.2.

### Preparation of tert-butyl (3R)-3-[2-(3,5-dimethylisoxazol-4-yl)-4-[(1-fluorocyclopropanecarbonyl)amino]phenoxy]pyridinerrolidine-1-carboxylate (63)

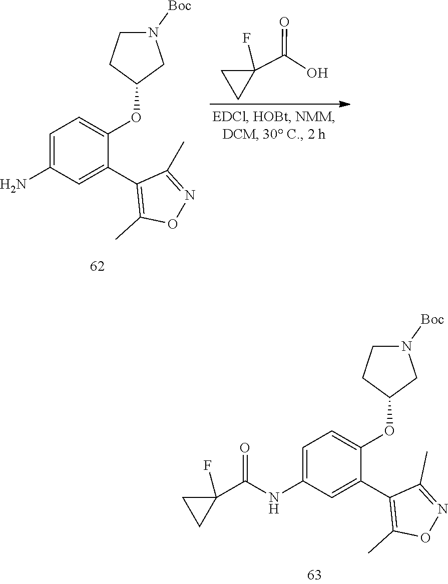 [SMILES: Cc1noc(C)c1-c1cc(N)ccc1O[C@@H]1CCN(C(=O)OC(C)(C)C)C1 ; O=C(O)C1(F)CC1 ; Cc1noc(C)c1-c1cc(NC(=O)C2(F)CC2)ccc1O[C@@H]1CCN(C(=O)OC(C)(C)C)C1]

A mixture of 1-fluorocyclopropanecarboxylic acid (83.61 mg, 803.33 μmol, 1.2 eq.), tert-butyl (3R)-3-[4-amino-2-(3,5-dimethylisoxazol-4-yl)phenoxy]pyridinerrolidine-1-carboxylate ((62), 250.00 mg, 669.44 μmol, 1 eq.), EDCl (154.00 mg, 803.33 μmol, 1.2 eq.), HOBt (45.23 mg, 334.72 μmol, 0.5 eq.), and NMM (135.43 mg, 1.34 mmol, 147.20 μL, 2 eq.) in DCM (3 mL) was stirred at 30° C. for 2 hrs (monitored by TLC). The reaction mixture was concentrated under reduced pressure to give a residue. The residue was purified by prep-TLC (SiO₂, petroleum ether:ethyl acetate=1:1). Tert-butyl (3R)-3-[2-(3,5-dimethylisoxazol-4-yl)-4-[(1-fluorocyclopropanecarbonyl)amino]phenoxy]pyridinerrolidine-1-carboxylate ((63), 300 mg, 652.87 μmol, 97.52% yield) was obtained as a white solid.

LCMS (ESI): m/z [M+H] calcd for C₂₄H₃₁FN₃O₅: 460.22; found: 460.3.

### Preparation of (R)—N-(3-(3,5-dimethylisoxazol-4-yl)-4-(pyrrolidin-3-yloxy)phenyl)-1-fluorocyclopropane-1-carboxamide (238)

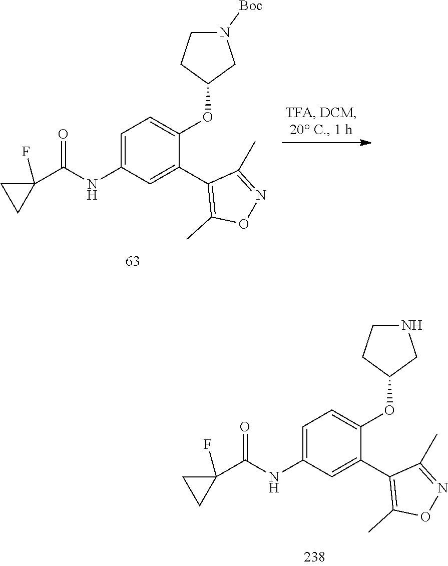 [SMILES: Cc1noc(C)c1-c1cc(NC(=O)C2(F)CC2)ccc1O[C@@H]1CCN(C(=O)OC(C)(C)C)C1 ; Cc1noc(C)c1-c1cc(NC(=O)C2(F)CC2)ccc1O[C@@H]1CCNC1]

A mixture of tert-butyl (3R)-3-[2-(3,5-dimethylisoxazol-4-yl)-4-[(1-fluorocyclopropanecarbonyl)amino]phenoxy]pyridinerrolidine-1-carboxylate ((63), 75.00 mg, 163.22 μmol, 1 eq.) and TFA (558.32 mg, 4.90 mmol, 362.54 μL, 30 eq.) in DCM (1 mL) was stirred at 20° C. for 1 hr (monitored by LC-MS). The reaction mixture was concentrated under reduced pressure to give a residue. The residue was purified by prep-HPLC (column: Phenomenex Gemini-NX C18 75*30 mm*3 μm; mobile phase: [water (0.1% TFA)-ACN]; B %: 20%-30%, 7 min). N-[3-(3,5-dimethylisoxazol-4-yl)-4-[(3R)-pyridinerrolidin-3-yl]oxy-phenyl]-1-fluoro-cyclopropanecarboxamide (32.67 mg, 68.32 μmol, 41.86% yield, 99% purity, TFA) was obtained as an off-white solid.

LCMS (ESI): m/z [M+H] calcd for C₁₉H₂₃FN₃O₃: 360.16; found: 360.1.

¹H NMR (400 MHZ, methanol-d₄) δ=7.67 (dd, J=2.2, 8.8 Hz, 1H), 7.50 (d, J=2.4 Hz, 1H), 7.15 (d, J=9.0 Hz, 1H), 5.10 (br t, J=4.8 Hz, 1H), 3.62-3.53 (m, 1H), 3.46-3.36 (m, 2H), 3.19 (dt, J=7.4, 10.8 Hz, 1H), 2.32 (s, 3H), 2.22-2.17 (m, 5H), 1.45 (br s, 1H), 1.45-1.39 (m, 1H), 1.44-1.38 (m, 1H), 1.38-1.35 (m, 1H).

### (S)—N-(3-(3,5-dimethylisoxazol-4-yl)-4-(pyrrolidin-3-yloxy)phenyl)-1-fluorocyclopropane-1-carboxamide (239)

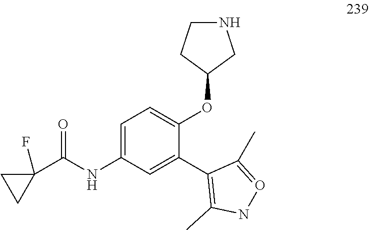

Compound 239 was prepared according to the synthesis described for compound 238, substituting tert-butyl (S)-3-hydroxypyrrolidine-1-carboxylate for tert-butyl (R)-3-hydroxypyridinerrolidine-1-carboxylate.

LCMS (ESI): m/z [M+H] calcd for C₁₉H₂₃FN₃O₃: 360.16; found: 360.2.

¹H NMR (400 MHZ, methanol-d₄) δ=7.67 (d, J=8.9 Hz, 1H), 7.54-7.46 (m, 1H), 7.15 (d, J=8.9 Hz, 1H), 5.10 (br t, J=4.8 Hz, 1H), 3.58 (dd, J=4.9, 13.2 Hz, 1H), 3.45-3.38 (m, 2H), 3.29-3.15 (m, 1H), 2.32 (s, 3H), 2.26-2.23 (m, 2H), 2.17 (s, 3H), 1.45-1.35 (m, 4H).

### N-(4-(azetidin-3-yloxy)-3-(3,5-dimethylisoxazol-4-yl)phenyl)-4-fluoro-1-methyl-1H-pyrazole-5-carboxamide (240)

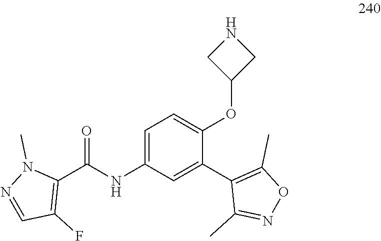 [SMILES: Cc1noc(C)c1-c1cc(NC(=O)c2c(F)cnn2C)ccc1OC1CNC1]

Compound 240 was prepared according to the synthesis described for compound 130, substituting tert-butyl 3-hydroxyazetidine-1-carboxylate for 2-(azetidin-1-yl)ethanol.

LCMS (ESI): m/z [M+H] calcd for C₁₉H₂₁FN₅O₃: 386.16; found: 386.2.

¹H NMR (400 MHZ, methanol-d₄) δ=7.69 (dd, J=2.6, 8.9 Hz, 1H), 7.59 (d, J=2.5 Hz, 1H), 7.46 (d, J=4.4 Hz, 1H), 6.86 (d, J=8.9 Hz, 1H), 5.23-5.13 (m, 1H), 4.55 (dd, J=6.6, 12.6 Hz, 2H), 4.10 (dd, J=4.9, 12.6 Hz, 2H), 4.04 (s, 3H), 2.35 (s, 3H), 2.20 (s, 3H).

### N-(4-(azetidin-3-yloxy)-3-(3,5-dimethylisoxazol-4-yl)phenyl)benzamide (241)

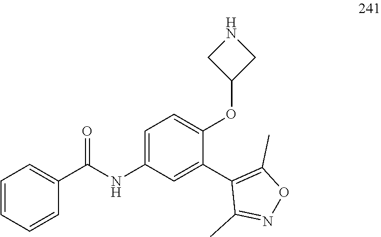 [SMILES: Cc1noc(C)c1-c1cc(NC(=O)c2ccccc2)ccc1OC1CNC1]

Compound 241 was prepared according to the synthesis described for compound 218, substituting tert-butyl 2-(hydroxymethyl)azetidine-1-carboxylate for tert-butyl (R)-2-(hydroxymethyl)piperidine-1-carboxylate.

LCMS (ESI): m/z [M+H] calcd for C₂₁H₂₂N₃O₃: 364.16; found: 364.2.

¹H NMR (400 MHZ, methanol-d₄) δ=8.01-7.89 (m, 2H), 7.75 (dd, J=2.6, 8.9 Hz, 1H), 7.69-7.47 (m, 4H), 6.86 (d, J=9.0 Hz, 1H), 5.23-5.13 (m, 1H), 4.61-4.50 (m, 2H), 4.09 (dd, J=4.9, 12.7 Hz, 2H), 2.36 (s, 3H), 2.21 (s, 3H).

### N-(4-(azetidin-3-yloxy)-3-(3,5-dimethylisoxazol-4-yl)phenyl)-4-fluorobenzamide (242)

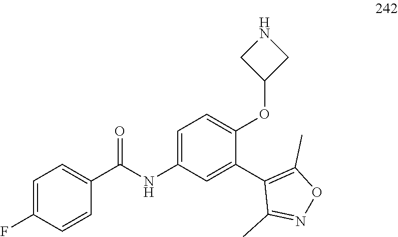 [SMILES: Cc1noc(C)c1-c1cc(NC(=O)c2ccc(F)cc2)ccc1OC1CNC1]

Compound 242 was prepared according to the synthesis described for compound 219, substituting tert-butyl 2-(hydroxymethyl)azetidine-1-carboxylate for tert-butyl (R)-2-(hydroxymethyl)piperidine-1-carboxylate.

LCMS (ESI): m/z [M+H] calcd for C₂₁H₂₁FN₃O₃: 382.15; found: 382.2.

¹H NMR (400 MHZ, methanol-d₄) δ=8.05-7.94 (m, 2H), 7.72 (dd, J=2.6, 8.9 Hz, 1H), 7.60 (d, J=2.6 Hz, 1H), 7.29-7.17 (m, 2H), 6.86 (d, J=8.9 Hz, 1H), 5.23-5.13 (m, 1H), 4.55 (dd, J=6.8, 12.6 Hz, 2H), 4.10 (dd, J=5.0, 12.6 Hz, 2H), 2.36 (s, 3H), 2.21 (s, 3H).

### N-(4-(azetidin-3-yloxy)-3-(3,5-dimethylisoxazol-4-yl)phenyl)-3-fluorobenzamide (243)

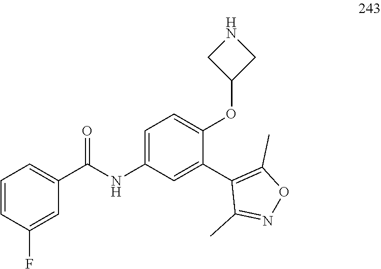 [SMILES: Cc1noc(C)c1-c1cc(NC(=O)c2cccc(F)c2)ccc1OC1CNC1]

Compound 243 was prepared according to the synthesis described for compound 242, substituting 3-fluorobenzoic acid for 4-fluorobenzoic acid.

LCMS (ESI): m/z [M+H] calcd for C₂₁H₂₁FN₃O₃: 382.15; found: 382.2.

¹H NMR (400 MHZ, methanol-d₄) δ=7.83-7.72 (m, 2H), 7.68 (td, J=2.0, 9.6 Hz, 1H), 7.61 (d, J=2.6 Hz, 1H), 7.54 (dt, J=5.6, 8.0 Hz, 1H), 7.39-7.29 (m, 1H), 6.86 (d, J=8.9 Hz, 1H), 5.23-5.13 (m, 1H), 4.55 (dd, J=6.6, 12.6 Hz, 2H), 4.09 (dd, J=5.0, 12.6 Hz, 2H), 2.36 (s, 3H), 2.21 (s, 3H).

### N-(4-(azetidin-3-yloxy)-3-(3,5-dimethylisoxazol-4-yl)phenyl)-3-methoxybenzamide (244)

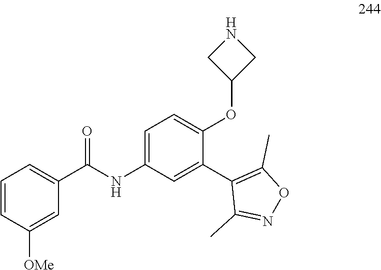 [SMILES: COc1cccc(C(=O)Nc2ccc(OC3CNC3)c(-c3c(C)noc3C)c2)c1]

Compound 244 was prepared according to the synthesis described for compound 220, substituting tert-butyl 2-(hydroxymethyl)azetidine-1-carboxylate for tert-butyl (R)-2-(hydroxymethyl)piperidine-1-carboxylate.

LCMS (ESI): m/z [M+H] calcd for C₂₂H₂₄N₃O₄: 394.17; found: 394.2.

¹H NMR (400 MHZ, methanol-d₄) δ=7.74 (dd, J=2.8, 8.9 Hz, 1H), 7.61 (d, J=2.6 Hz, 1H), 7.56-7.46 (m, 2H), 7.42 (t, J=7.9 Hz, 1H), 7.20-7.08 (m, 1H), 6.86 (d, J=9.0 Hz, 1H), 5.25-5.12 (m, 1H), 4.55 (dd, J=6.7, 12.6 Hz, 2H), 4.10 (dd, J=5.0, 12.5 Hz, 2H), 3.86 (s, 3H), 2.36 (s, 3H), 2.21 (s, 3H).

### N-(4-(azetidin-3-yloxy)-3-(3,5-dimethylisoxazol-4-yl)phenyl)-1-fluorocyclopropane-1-carboxamide (245)

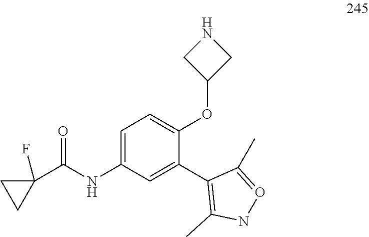

Compound 245 was prepared according to the synthesis described for compound 217, substituting tert-butyl 2-(hydroxymethyl)azetidine-1-carboxylate for tert-butyl (R)-2-(hydroxymethyl)piperidine-1-carboxylate.

LCMS (ESI): m/z [M+H] calcd for C₁₈H₂₁FN₃O₃: 346.15; found: 346.2.

¹H NMR (400 MHZ, methanol-d₄) δ=7.66 (dd, J=2.8, 8.9 Hz, 1H), 7.53 (d, J=2.6 Hz, 1H), 6.83 (d, J=9.0 Hz, 1H), 5.20-5.11 (m, 1H), 4.53 (dd, J=6.6, 12.6 Hz, 2H), 4.08 (dd, J=5.0, 12.6 Hz, 2H), 2.34 (s, 3H), 2.19 (s, 3H), 1.44-1.31 (m, 4H).

### Example 2—In Vitro Binding Assays

### Radioligand Binding Assay Protocol

Radioligand binding assays were performed using [3H]5-HT as the radioligand using cell membranes prepared from HEK293 cells expressing recombinant 5-HT_{2A}, 5-HT_{2B} and 5-HT_{2C(INI)} receptors. Competition binding experiments consisted of addition of 5 μL of serially diluted test compound, 50 μL of radioligand stock diluted in Assay Buffer (20 mM HEPES, pH 7.4, 10 mM MgCl₂), and 145 μL of diluted membrane expressing the receptor of interest to 96-well microtiter plates, which were then incubated for one hour at room temperature. Assay incubations were terminated by rapid filtration through Perkin Elmer GF/C filtration plates under vacuum pressure using a 96-well Packard filtration apparatus, followed by washing the filter plates several times with ice cold Assay Buffer. Plates were then dried at 45° C. for a minimum of four hours. Finally, 25 μL of BetaScint scintillation cocktail was added to each well and the plates were counted in a Packard TopCount scintillation counter. In each competition study, test compounds were assayed at 10 concentrations with three replicates at each test concentration. Dose-response curves were constructed to determine IC₅₀ values and these were converted to corresponding Ki values using the Cheng-Prusoff equation.

TABLE 2
Assay Conditions for 5-HT2 Receptor Binding Assays

| | | | Measured |
|---|---|---|---|
| | Final [³H]5-HT Assay | Membrane Protein | Radioligand |
| Receptor | Concentration (nM) | Amount (μg) | K_{d} (nM) |
| 5-HT_{2A} | 2.7 | 12 | 4.79 |
| 5-HT_{2B} | 5.4 | 40 | 12.1 |
| 5-HT_{2C(INI)} | 2.7 | 12 | 0.53 |

TABLE 3
5-HT_{2A} serotonin binding

| Compound | Kᵢ value (nM) | Compound | Kᵢ value (nM) |
|---|---|---|---|
| 100 | 2.89 | 121 | 4.67 |
| 101 | 78.3 | 122 | 2.7 |
| 102 | 119 | 123 | 11.8 |
| 103 | 50.8 | 124 | 10.6 |
| 104 | 19.6 | 125 | 4.5 |
| 105 | 3.94 | 126 | 5.28 |
| 106 | 1.13 | 127 | 8.51 |
| 107 | 15.1 | 128 | 14.6 |
| 108 | 6.21 | 129 | 182 |
| 109 | 3.78 | 130 | 12.8 |
| 110 | 110 | 131 | 77.7 |
| 111 | 23.8 | 132 | 46.1 |
| 112 | 14.1 | 133 | 51.3 |
| 113 | 2.66 | 134 | 5.86 |
| 114 | 2.6 | 135 | 20.1 |
| 115 | 8.52 | 136 | 84.1 |
| 116 | 6.15 | 137 | 551 |
| 117 | 10.3 | 138 | 24.6 |
| 118 | 18.9 | 139 | 124 |
| 119 | 9.58 | 140 | 228 |
| 120 | 9.41 | 141 | 9.47 |
| 142 | 67.4 | 172 | 6.5 |
| 143 | 84 | 173 | 96.9 |
| 144 | 14.9 | 174 | 48 |
| 145 | 5.51 | 175 | 41.6 |
| 146 | 5.08 | 176 | 395 |
| 147 | 25.2 | 177 | 4.22 |
| 148 | 71.6 | 178 | 718 |
| 149 | 13.9 | 179 | 85.9 |
| 150 | 13.5 | 180 | 186 |
| 151 | 47.1 | 181 | 10 |
| 152 | 41.7 | 182 | 61.5 |
| 153 | 109 | 183 | 5.09 |
| 154 | 8.12 | 184 | 10.5 |
| 155 | 22 | 185 | 8.77 |
| 156 | 145 | 186 | 7.71 |
| 157 | 2070 | 187 | 4.01 |
| 158 | 233 | 188 | 4.5 |
| 159 | 1.61 | 189 | 3.92 |
| 160 | 4.07 | 190 | 1.19 |
| 161 | 6.4 | 191 | 7.61 |
| 162 | 4.7 | 192 | 4.91 |
| 163 | 6.73 | 193 | 2.69 |
| 164 | 13.8 | 194 | 32.2 |
| 165 | 1.08 | 195 | 15.2 |
| 166 | 6.54 | 196 | 10.2 |
| 167 | 7.71 | 197 | 1.08 |
| 168 | 12.2 | 198 | 5.39 |
| 169 | 2.16 | 199 | 35.6 |
| 170 | 49.6 | 200 | 9.08 |
| 171 | 184 | 201 | 11.9 |
| 202 | 9.81 | 224 | 1.53 |
| 203 | 27.8 | 225 | 3.18 |
| 204 | 25 | 226 | 10.4 |
| 205 | 105 | 227 | 91.6 |
| 206 | 3.4 | 228 | 66.4 |
| 207 | 235 | 229 | 4.72 |
| 208 | 9.66 | 230 | 6.17 |
| 209 | 36.3 | 231 | 16.8 |
| 210 | 86.3 | 232 | 3.86 |
| 211 | 40.9 | 233 | 1.64 |
| 212 | 38.6 | 234 | 0.657 |
| 213 | 4.73 | 235 | 45.2 |
| 214 | 3.05 | 236 | 63900 |
| 215 | 18.4 | 237 | 1.14 |
| 216 | 298 | 238 | 3.61 |
| 217 | 1.21 | 239 | 59.4 |
| 218 | 4.85 | 240 | 1.36 |
| 219 | 2.92 | 241 | 6.94 |
| 220 | 0.907 | 242 | 2.1 |
| 221 | 4.22 | 243 | 1.07 |
| 222 | 4.68 | 244 | 0.850 |
| 223 | 1.69 | 245 | 2.05 |

### HTRF IP-One Antagonist Assays

HEK293 cells stably expressing 5-HT₂ receptors were generated using standard procedures. Cells were dilution cloned and clonal cell lines stably expressing the receptors at low levels were selected for pharmacology studies. In radioligand binding studies using a variety of radiolabeled probes, receptor densities in the 2A, 2B and 2C cell lines were estimated to be 50-70, 150-200, and 10-20 fmol/mg, respectively.

Inositol phosphate accumulation assays were performed using the HTRF IP-One assay system developed by Cisbio and generally followed the manufacturer's instructions. Briefly, HEK293 cells stably expressing recombinant human receptors were harvested and resuspended in phenol-red free OptiMEM (ThermoFisher), plated into 384-well assay plates (Perkin Elmer, ProxiPlate-Plus® Cat #6008280) at 10,000 cells/well in a volume of 10 μL/well, and incubated overnight at 37° C., 5% CO₂. Test compounds were solubilized and serially diluted in DMSO, and then diluted into Assay Buffer (Tris HCl 20 mM, NaCl 150 mM, LiCl 40 mM, pargyline 25 μM, pH 8) containing 80 nM 5-HT. Test compounds were added to the cells (4 μL/well addition, maximal final assay concentration typically 10 μM) and plates, incubated for 2 hrs at 37° C./5% CO₂ and then removed from the incubator and allowed to cool to room temperature for 30 minutes prior to addition of IP-One detection reagents. IP-One detection reagents (6 μL/well) were added and plates incubated for 1 hr at room temperature before reading on an HTRF compatible reader such as a Perkin Elmer EnVision® or BMG PheraStar®. Dose-response experiments utilized 10-point curves, typically starting at 10 μM, with 5-fold serial dilutions. Experiments were performed with triplicate data points and independent replicate experiments were performed to ensure data consistency.

TABLE 4
5-HT₂ IC₅₀ values

| Compound | 5-HT_{2A} (nM) | 5-HT_{2B} (μM) | 5-HT_{2C} (μM) |
|---|---|---|---|
| 113 | 8.95 | 100 | 100 |
| 114 | 8.69 | 100 | 100 |
| 117 | 20.6 | 100 | 100 |
| 119 | 13 | 100 | 100 |
| 121 | 29.7 | | |
| 122 | 8.92 | | |
| 125 | 38.2 | 100 | |
| 141 | 49.7 | 100 | 100 |
| 144 | 85.5 | 100 | 100 |
| 146 | 15.7 | 100 | 100 |
| 169 | 13 | 100 | 100 |
| 172 | 109 | 100 | |
| 187 | 36.2 | 100 | 100 |
| 189 | 24.9 | 100 | 100 |
| 191 | 60.1 | 100 | 100 |
| 192 | 31.1 | 100 | |
| 196 | 45 | 100 | 100 |
| 197 | 4.95 | 100 | 100 |
| 198 | 22.8 | 100 | 3.73 |
| 217 | 5.19 | 100 | |
| 218 | 21.8 | 100 | 100 |
| 219 | 10.4 | 100 | 100 |
| 220 | 2.58 | 100 | 100 |
| 221 | 17.8 | 100 | 100 |
| 222 | 53.4 | 100 | 100 |
| 223 | 14.7 | 100 | 100 |
| 225 | 19.7 | 100 | |
| 226 | 10.9 | 100 | 100 |
| 229 | 20.7 | 100 | 100 |
| 230 | 6.17 | 100 | |
| 232 | 24.8 | 100 | 100 |
| 233 | 9.5 | 100 | 100 |
| 234 | 2.39 | 100 | 100 |
| 238 | 45.2 | 100 | 100 |
| 240 | 17.8 | 100 | 100 |
| 241 | 35 | 100 | 100 |
| 242 | 44.2 | 3.39 | 4.92 |
| 243 | 16.5 | 100 | 6.21 |
| 244 | 19 | 100 | 100 |
| 245 | 37.9 | 100 | 100 |

### OTHER EMBODIMENTS

It is to be understood that the foregoing description is intended to illustrate and not limit the scope of the disclosure, which is defined by the scope of the appended claims. Other aspects, advantages, and modifications are within the scope of the following claims.

1. A compound of Formula (I) 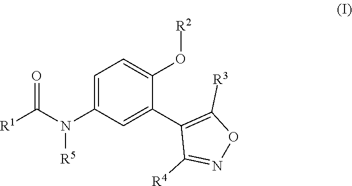
or a pharmaceutically acceptable salt thereof, wherein:
R¹ is selected from C₁-C₆ alkyl, C₃-C₆ cycloalkyl, phenyl, 5-10 membered heteroaryl, 5-9 membered heterocycloalkyl, (C₁-C₃ alkylene)-(C₃-C₆ cycloalkyl), (C₁-C₃ alkylene)-phenyl, (C₁-C₃ haloalkylene)-phenyl, (C₁-C₃ alkylene)-(5-10 membered heteroaryl), (C₁-C₃ alkylene)-(5-9 membered heterocycloalkyl), (C₁-C₃ alkylene)-O—(C₃-C₆ cycloalkyl), and (C₁-C₃ alkylene)-NH—(C₃-C₆ cycloalkyl), wherein the alkyl, alkylene, cycloalkyl, phenyl, heteroaryl, and heterocycloalkyl are each optionally substituted with one or more substituents independently selected from halogen, —CN, —OH, —NH₂, C₁-C₃ alkyl, (C₁-C₃ haloalkyl), (C₃-C₆ cycloalkyl), —O—(C₁-C₃ alkyl), (C₁-C₃ alkylene)-O—(C₁-C₃ alkyl), and phenyl;
R² is selected from 4-6 membered heterocycloalkyl, (C₁-C₃ alkylene)-(4-10 membered heterocycloalkyl), and (C₁-C₃ alkylene)-NR^{2A}R^{2B}, wherein the alkylene and heterocycloalkyl are each optionally substituted with one or more substituents independently selected from halogen, oxo, —OH, (C₁-C₃ alkyl), —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl);
R^{2A} and R^{2B} are each independently selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-S(═O)—(C₁-C₃ alkyl), (C₁-C₃ alkylene)-SO₂—(C₁-C₃ alkyl), and C(═NH)(C₁-C₃ alkyl);
or R^{2A} and R^{2B}, taken together with the nitrogen to which they are attached, form a 3-10 membered heterocycloalkyl ring optionally substituted with one or more substituents independently selected from halogen, oxo, —OH, C₁-C₃ alkyl, (C₁-C₃ haloalkyl), —O—(C₁-C₃ alkyl), (C₁-C₃ alkylene)-C(O)OH, —C(O)H, —C(O)(C₁-C₃ alkyl), —C(O)(C₁-C₃ alkylene)-OH, —C(O)C(O)OH, and —SO₂(C₁-C₃ alkyl), and optionally containing one additional heteroatom selected from the group of N, O, and S;
R³ and R⁴ are each independently selected from H, C₁-C₆ alkyl, and C₁-C₆ haloalkyl; and
R⁵ is selected from H and C₁-C₆ alkyl.

2. The compound of claim 1, or a pharmaceutically acceptable salt thereof, wherein R¹ is C₁-C₆ alkyl optionally substituted with one or more substituents independently selected from halogen and —O—(C₁-C₃ alkyl).

3. (canceled)

4. The compound of claim 1, or a pharmaceutically acceptable salt thereof, wherein R¹ is C₃-C₆ cycloalkyl optionally substituted with one or more substituents independently selected from halogen, —OH, C₁-C₃ haloalkyl, (C₃-C₆ cycloalkyl), —O—(C₁-C₃ alkyl), (C₁-C₃ alkylene)-O—(C₁-C₃ alkyl), and phenyl.

5-6. (canceled)

7. The compound of claim 1, or a pharmaceutically acceptable salt thereof, wherein R¹ is phenyl optionally substituted with one or more substituents independently selected from halogen, —CN, —NH₂, C₁-C₃ alkyl, (C₁-C₃ haloalkyl), and —O—(C₁-C₃ alkyl).

8-15. (canceled)

16. The compound of claim 1, or a pharmaceutically acceptable salt thereof, wherein R¹ is 5-10 membered heteroaryl optionally substituted with one or more substituents independently selected from halogen, C₁-C₃ alkyl, and —O—(C₁-C₃ alkyl).

17-23. (canceled)

24. The compound of claim 1, or a pharmaceutically acceptable salt thereof, wherein R¹ is 5-9 membered heterocycloalkyl optionally substituted with one or more substituents independently selected from halogen and C₁-C₃ alkyl.

25-30. (canceled)

31. The compound of claim 1, or a pharmaceutically acceptable salt thereof, wherein R² is 4-6 membered heterocycloalkyl optionally substituted with one or more substituents independently selected from halogen, oxo, —OH, (C₁-C₃ alkyl), —O—(C₁-C₃ alkyl), (C₁-C₃ alkylene)-C(O)OH, —C(O)H, —C(O)(C₁-C₃ alkyl), —C(O)(C₁-C₃ alkylene)-OH, —C(O)C(O)OH, and —SO₂(C₁-C₃ alkyl).

32-33. (canceled)

34. The compound of claim 1, or a pharmaceutically acceptable salt thereof, wherein R² is (C₁-C₃ alkylene)-(4-10 membered heterocycloalkyl), wherein the heterocycloalkyl is optionally substituted with one or more substituents independently selected from halogen, oxo, —OH, (C₁-C₃ alkyl), —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl).

35-36. (canceled)

37. The compound of claim 1, or a pharmaceutically acceptable salt thereof, wherein R² is (C₁-C₃ alkylene)-NR^{2A}R^{2B}.

38. The compound of claim 37, or a pharmaceutically acceptable salt thereof, wherein R^{2A} is selected from H and C₁-C₃ alkyl.

39. The compound of claim 37, or a pharmaceutically acceptable salt thereof, wherein R^{2B} is selected from C₁-C₃ alkyl, C₁-C₃ haloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-S(═O)—(C₁-C₃ alkyl), (C₁-C₃ alkylene)-SO₂—(C₁-C₃ alkyl), and C(═NH)(C₁-C₃ alkyl).

40-43. (canceled)

44. The compound of claim 1, or a pharmaceutically acceptable salt thereof, wherein R^{2A} and R^{2B}, taken together with the nitrogen to which they are attached, form a 3-10 membered heterocycloalkyl ring optionally substituted with one or more substituents independently selected from halogen, oxo, —OH, C₁-C₃ alkyl, (C₁-C₃ haloalkyl), —O—(C₁-C₃ alkyl), (C₁-C₃ alkylene)-C(O)OH, —C(O)H, —C(O)(C₁-C₃ alkyl), —C(O)(C₁-C₃ alkylene)-OH, —C(O)C(O)OH, and —SO₂(C₁-C₃ alkyl), and optionally containing one additional heteroatom selected from the group of N, O, and S.

45-61. (canceled)

62. The compound of claim 1, or a pharmaceutically acceptable salt thereof, wherein one of R³ and R⁴ is H and the other is C₁-C₆ alkyl.

63-66. (canceled)

67. The compound of claim 1, or a pharmaceutically acceptable salt thereof, wherein R⁵ is C₁-C₃ alkyl.

68. (canceled)

69. The compound of claim 1, having the structure of Formula (Ia): 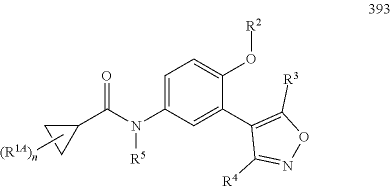
wherein:
each R^{1A} is independently selected from halogen, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl;
R² is selected from 4-6 membered heterocycloalkyl, (C₁-C₃ alkylene)-(4-10 membered heterocycloalkyl), and (C₁-C₃ alkylene)-NR^{2A}R^{2B}, wherein the alkylene and heterocycloalkyl are each optionally substituted with one or more substituents independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl);
R^{2A} and R^{2B} are each independently selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-SO₂—(C₁-C₃ alkyl), and C(═NH)(C₁-C₃ alkyl);
or R^{2A} and R^{2B}, taken together with the nitrogen to which they are attached, form a 3-10 membered heterocycloalkyl ring optionally substituted with one or more substituents independently selected from halogen, oxo, —OH, C₁-C₃ alkyl, C₁-C₃ haloalkyl, —O—(C₁-C₃ alkyl), (C₁-C₃ alkylene)-C(O)OH, —C(O)H, —C(O)(C₁-C₃ alkyl), —C(O)(C₁-C₃ alkylene)-OH, —C(O)C(O)OH, and —SO₂(C₁-C₃ alkyl), and optionally containing one additional heteroatom selected from the group of N, O, and S;
R³ and R⁴ are each independently selected from H, C₁-C₆ alkyl, and C₁-C₆ haloalkyl;
R⁵ is selected from H and C₁-C₆ alkyl; and
n is 0, 1, 2, 3, 4, or 5;
or a pharmaceutically acceptable salt thereof.

70. (canceled)

71. The compound of claim 1, having the structure of Formula (Ic): 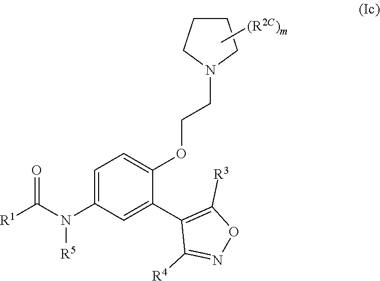
wherein:
R¹ is selected from C₁-C₆ alkyl, C₃-C₆ cycloalkyl, phenyl, 5-10 membered heteroaryl, 5-9 membered heterocycloalkyl, (C₁-C₃ alkylene)-(C₃-C₆ cycloalkyl), (C₁-C₃ alkylene)-phenyl, (C₁-C₃ haloalkylene)-phenyl, (C₁-C₃ alkylene)-(5-10 membered heteroaryl), (C₁-C₃ alkylene)-(5-9 membered heterocycloalkyl), (C₁-C₃ alkylene)-O—(C₃-C₆ cycloalkyl), and (C₁-C₃ alkylene)-NH—(C₃-C₆ cycloalkyl), wherein the alkyl, alkylene, cycloalkyl, phenyl, heteroaryl, and heterocycloalkyl are each optionally substituted with one or more substituents independently selected from halogen, —CN, —OH, —NH₂, C₁-C₃ alkyl, C₁-C₃ haloalkyl, C₃-C₆ cycloalkyl, —O—(C₁-C₃ alkyl), (C₁-C₃ alkylene)-O—(C₁-C₃ alkyl), and phenyl;
each R^{2C} is independently selected from halogen, oxo, —OH, C₁-C₃ alkyl, —O—(C₁-C₃ alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl);
R³ and R⁴ are each independently selected from H, C₁-C₆ alkyl, and C₁-C₆ haloalkyl;
R⁵ is selected from H and C₁-C₆ alkyl; and
m is 0, 1, 2, 3, 4, 5, or 6;
or a pharmaceutically acceptable salt thereof.

72-82. (canceled)

83. A method of treating a 5HT_{2A}-related disorder in an individual in need thereof, comprising prescribing and/or administering a therapeutically effective amount of a compound of Formula (I) 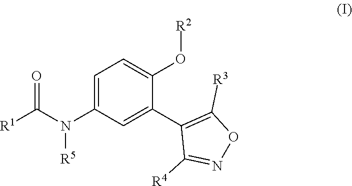
or a pharmaceutically acceptable salt thereof, wherein:
R¹ is selected from C₁-C₆ alkyl, C₃-C₆ cycloalkyl, phenyl, 5-10 membered heteroaryl, 5-9 membered heterocycloalkyl, (C₁-C₃alkylene)-(C₃-C₆cycloalkyl), (C₁-C₃alkylene)-phenyl, (C₁-C₃ haloalkylene)-phenyl, (C₁-C₃ alkylene)-(5-10 membered heteroaryl), (C₁-C₃ alkylene)-(5-9 membered heterocycloalkyl), (C₁-C₃ alkylene)-O—(C₃-C₆ cycloalkyl), and (C₁-C₃ alkylene)-NH—(C₃-C₆ cycloalkyl), wherein the alkyl, alkylene, cycloalkyl, phenyl, heteroaryl, and heterocycloalkyl are each optionally substituted with one or more substituents independently selected from halogen, —CN, —OH, —NH₂, C₁-C₃ alkyl, (C₁-C₃ haloalkyl), (C₃-C₆ cycloalkyl), —O—(C₁-C₃ alkyl), (C₁-C₃ alkylene)-O—(C₁-C₃ alkyl), and phenyl;
R² is selected from 4-6 membered heterocycloalkyl, (C₁-C₃alkylene)-(4-10 membered heterocycloalkyl), and (C₁-C₃ alkylene)-NR^{2A}R^{2B}, wherein the alkylene and heterocycloalkyl are each optionally substituted with one or more substituents independently selected from halogen, oxo, —OH, (C₁-C₃alkyl), —O—(C₁-C₃alkyl), (C₁-C₃ alkylene)-C(O)OH, —C(O)H, —C(O)OH, —C(O)(C₁-C₃ alkyl), —C(O)(C₁-C₃ alkylene)-OH, —C(O)C(O)OH, and —SO₂(C₁-C₃ alkyl);
R^{2A} and R^{2B} are each independently selected from H, C₁-C₃ alkyl, C₁-C₃ haloalkyl, C₃-C₆ cycloalkyl, (C₁-C₃ alkylene)-O—(C₁-C₃ alkyl), (C₁-C₃ alkylene)-OH, (C₁-C₃ alkylene)-(C₃-C₆ cycloalkyl), (C₁-C₃ alkylene)-S(═O)—(C₁-C₃ alkyl), (C₁-C₃ alkylene)-SO₂—(C₁-C₃ alkyl), and C(═NH)(C₁-C₃ alkyl);
or R^{2A} and R^{2B}, taken together with the nitrogen to which they are attached, form a 3-10 membered heterocycloalkyl ring optionally substituted with one or more substituents independently selected from halogen, oxo, —OH, C₁-C₃ alkyl, (C₁-C₃ haloalkyl), —O—(C₁-C₃ alkyl), (C₁-C₃ alkylene)-C(O)OH, —C(O)H, —C(O)(C₁-C₃ alkyl), —C(O)(C₁-C₃alkylene)-OH, —C(O)C(O)OH, and —SO₂(C₁-C₃ alkyl), and optionally containing one additional heteroatom selected from the group of N, O, and S;
R³ and R⁴ are each independently selected from H, C₁-C₆ alkyl, and C₁-C₆ haloalkyl; and R⁵ is selected from H and C₁-C₆ alkyl.

84. The method according to claim 83, wherein the 5HT_{2A}-related disorder is selected from a condition associated with platelet aggregation, coronary artery disease, myocardial infarction, transient ischemic attack, angina, stroke, atrial fibrillation, blood clot formation, or symptoms thereof.

85. The method according to claim 83, wherein the 5HT_{2A}-related disorder is an effect of PCI selected from microvascular obstruction (MVO), myocardial injury, reduced cardiac function, or a major adverse cardiac event (MACE).

86. The method according to claim 83, wherein the 5HT_{2A}-related disorder is Raynaud's.

87-91. (canceled)
